SEQUENCE LISTING (1264 sequences)

SEQ ID NO: 1 - DNA, 22 bp, synthetic construct
  source 1..22: mol_type = other DNA
ccctgcgtgtctccgactgcac

SEQ ID NO: 2 - DNA, 22 bp, synthetic construct
  source 1..22: mol_type = other DNA
atcaccgactgcccatagagag

SEQ ID NO: 3 - DNA, 27 bp, synthetic construct
  source 1..27: mol_type = other DNA
  modified_base 1: mod_base = OTHER; note = Uracil
  modified_base 5: mod_base = OTHER; note = Uracil
  modified_base 10: mod_base = OTHER; note = Uracil
  modified_base 22: mod_base = OTHER; note = Uracil
tccatctcatccctgcgtgtctccgac

SEQ ID NO: 4 - DNA, 47 bp, synthetic construct
  source 1..47: mol_type = other DNA
  misc_difference 1: note = a, c, t, g, unknown or other
nccctgtgtgccttggcagtctcagctctctatgggcagtcggtgat

SEQ ID NO: 5 - DNA, 30 bp, synthetic construct
  source 1..30: mol_type = other DNA
gaatttacatgctttaacaaccgtggcctc

SEQ ID NO: 6 - DNA, 30 bp, synthetic construct
  source 1..30: mol_type = other DNA
ctaacgatgaacaacttccaccactgggca

SEQ ID NO: 7 - DNA, 30 bp, synthetic construct
  source 1..30: mol_type = other DNA
atgtcgtctttgtccttgttgctaactaga

SEQ ID NO: 8 - DNA, 30 bp, synthetic construct
  source 1..30: mol_type = other DNA
acgctactgtcttggagggccacttcatat

SEQ ID NO: 9 - DNA, 30 bp, synthetic construct
  source 1..30: mol_type = other DNA
ctagatgtcgacgaacggcagcgctgatgt

SEQ ID NO: 10 - DNA, 30 bp, synthetic construct
  source 1..30: mol_type = other DNA
gtctgcattctggctataggttagagtcga

SEQ ID NO: 11 - DNA, 30 bp, synthetic construct
  source 1..30: mol_type = other DNA
cctcttgggagccgtctcgtaatccgatct

SEQ ID NO: 12 - DNA, 30 bp, synthetic construct
  source 1..30: mol_type = other DNA
aggtacgcgcaactgatcgaaaccaagatg

SEQ ID NO: 13 - DNA, 30 bp, synthetic construct
  source 1..30: mol_type = other DNA
atcgagaggaactacgtcgcccggtaaaga

SEQ ID NO: 14 - DNA, 30 bp, synthetic construct
  source 1..30: mol_type = other DNA
tatcgtaaatacaactgaccaacataagtc

SEQ ID NO: 15 - DNA, 30 bp, synthetic construct
  source 1..30: mol_type = other DNA
gggtccgttcctgatgcctaccgccaatgt

SEQ ID NO: 16 - DNA, 30 bp, synthetic construct
  source 1..30: mol_type = other DNA
gttggatgtgagttcactggtaccactaag

SEQ ID NO: 17 - DNA, 30 bp, synthetic construct
  source 1..30: mol_type = other DNA
tccgacgcgtctgtattgatttgtctctgg

SEQ ID NO: 18 - DNA, 30 bp, synthetic construct
  source 1..30: mol_type = other DNA
gaatgaggtcgagtaaccctgggcccgtgc

SEQ ID NO: 19 - DNA, 30 bp, synthetic construct
  source 1..30: mol_type = other DNA
cgtggtaacgtaggcgcgcacttctcagca

SEQ ID NO: 20 - DNA, 30 bp, synthetic construct
  source 1..30: mol_type = other DNA
gaaaacggcttgagctgagactctccaatt

SEQ ID NO: 21 - DNA, 30 bp, synthetic construct
  source 1..30: mol_type = other DNA
cgcgaatgttatttgggcgaatgactaaag

SEQ ID NO: 22 - DNA, 30 bp, synthetic construct
  source 1..30: mol_type = other DNA
ttaccgtatggcctatagaccgaggttctt

SEQ ID NO: 23 - DNA, 30 bp, synthetic construct
  source 1..30: mol_type = other DNA
acggcactcatccttacgtggtcgggtaga

SEQ ID NO: 24 - DNA, 30 bp, synthetic construct
  source 1..30: mol_type = other DNA
ggctcacaacgccacgctactctcaaccgg

SEQ ID NO: 25 - DNA, 30 bp, synthetic construct
  source 1..30: mol_type = other DNA
tgtaaacagcgcggtcatctgatagaggac

SEQ ID NO: 26 - DNA, 30 bp, synthetic construct
  source 1..30: mol_type = other DNA
ccttagtgtaaatcgaccacgtgagcatcg

SEQ ID NO: 27 - DNA, 30 bp, synthetic construct
  source 1..30: mol_type = other DNA
ggtcccagccacactcgcgagccttttagc

SEQ ID NO: 28 - DNA, 30 bp, synthetic construct
  source 1..30: mol_type = other DNA
tatgatacctaggctgcatcgttacccggc

SEQ ID NO: 29 - DNA, 30 bp, synthetic construct
  source 1..30: mol_type = other DNA
agcttctaaaatacgagccacccaatacga

SEQ ID NO: 30 - DNA, 30 bp, synthetic construct
  source 1..30: mol_type = other DNA
ggagtgtctaccgtaggaacgatccacagc

SEQ ID NO: 31 - DNA, 30 bp, synthetic construct
  source 1..30: mol_type = other DNA
ccgtgggatcgagatggctagtaggagaca

SEQ ID NO: 32 - DNA, 30 bp, synthetic construct
  source 1..30: mol_type = other DNA
ctaagccgattcgtgtctccacagaaatta

SEQ ID NO: 33 - DNA, 30 bp, synthetic construct
  source 1..30: mol_type = other DNA
tggacgctgggctaagaagttggccgatag

SEQ ID NO: 34 - DNA, 30 bp, synthetic construct
  source 1..30: mol_type = other DNA
ttcatgaagccgttatttcggacctactcg

SEQ ID NO: 35 - DNA, 30 bp, synthetic construct
  source 1..30: mol_type = other DNA
ctactgacgtattgagttgcctaaatctgg

SEQ ID NO: 36 - DNA, 30 bp, synthetic construct
  source 1..30: mol_type = other DNA
gcgaggtatgactcccgccgccttctcaag

SEQ ID NO: 37 - DNA, 30 bp, synthetic construct
  source 1..30: mol_type = other DNA
aaccgttcaaatttgtgccgataggtgtta

SEQ ID NO: 38 - DNA, 30 bp, synthetic construct
  source 1..30: mol_type = other DNA
atcagcgagccgagacacgagttcacttat

SEQ ID NO: 39 - DNA, 30 bp, synthetic construct
  source 1..30: mol_type = other DNA
aaaacttccctaatagcttaacgtatgagt

SEQ ID NO: 40 - DNA, 30 bp, synthetic construct
  source 1..30: mol_type = other DNA
attatgagcagagcaaacgtcactaagtgg

SEQ ID NO: 41 - DNA, 30 bp, synthetic construct
  source 1..30: mol_type = other DNA
cgcaccagaggtctcaatgctcatgtaaga

SEQ ID NO: 42 - DNA, 30 bp, synthetic construct
  source 1..30: mol_type = other DNA
tttaaagtggtctatgccgaatcgatctcc

SEQ ID NO: 43 - DNA, 30 bp, synthetic construct
  source 1..30: mol_type = other DNA
gtgtttaggattagggacatcacggcttct

SEQ ID NO: 44 - DNA, 30 bp, synthetic construct
  source 1..30: mol_type = other DNA
acccgtggaaaggtgagtatacagcagcta

SEQ ID NO: 45 - DNA, 30 bp, synthetic construct
  source 1..30: mol_type = other DNA
aacgaaaatatactgtcgccacaaggaaac

SEQ ID NO: 46 - DNA, 30 bp, synthetic construct
  source 1..30: mol_type = other DNA
ccgacggtccggactttttctcaccatcgt

SEQ ID NO: 47 - DNA, 30 bp, synthetic construct
  source 1..30: mol_type = other DNA
gcattagctcaaaggccagacttatccgtg

SEQ ID NO: 48 - DNA, 30 bp, synthetic construct
  source 1..30: mol_type = other DNA
aggcaactatgttcacggtacgtcggctcg

SEQ ID NO: 49 - DNA, 30 bp, synthetic construct
  source 1..30: mol_type = other DNA
cacctatctggatccagtgaatacctccga

SEQ ID NO: 50 - DNA, 30 bp, synthetic construct
  source 1..30: mol_type = other DNA
ttcggcgttcgagcagtaggacctaaccaa

SEQ ID NO: 51 - DNA, 30 bp, synthetic construct
  source 1..30: mol_type = other DNA
ccatcattggtttgcagggtatatagcgcc

SEQ ID NO: 52 - DNA, 30 bp, synthetic construct
  source 1..30: mol_type = other DNA
ggcgagtagccatagatcgtcaacgtcgtg

SEQ ID NO: 53 - DNA, 30 bp, synthetic construct
  source 1..30: mol_type = other DNA
gagcccgccagtcatcgaaacacgcatcat

SEQ ID NO: 54 - DNA, 30 bp, synthetic construct
  source 1..30: mol_type = other DNA
acggaggttggcccgcacgatattcagaca

SEQ ID NO: 55 - DNA, 30 bp, synthetic construct
  source 1..30: mol_type = other DNA
ataaccttaatgcacgagctgaaaccacag

SEQ ID NO: 56 - DNA, 30 bp, synthetic construct
  source 1..30: mol_type = other DNA
ttagccaggtggtccgctgccagaatggtc

SEQ ID NO: 57 - DNA, 30 bp, synthetic construct
  source 1..30: mol_type = other DNA
tttgtgtattttcactctgtctttagcgga

SEQ ID NO: 58 - DNA, 30 bp, synthetic construct
  source 1..30: mol_type = other DNA
cggatgggacgtgagcttaatggatctgat

SEQ ID NO: 59 - DNA, 30 bp, synthetic construct
  source 1..30: mol_type = other DNA
ctgaccggaaaagattcgagggcggagaac

SEQ ID NO: 60 - DNA, 30 bp, synthetic construct
  source 1..30: mol_type = other DNA
ctccatatctcaatcaactctcgtcgaatt

SEQ ID NO: 61 - DNA, 30 bp, synthetic construct
  source 1..30: mol_type = other DNA
ggcggcagtgccttccgggttcctttttag

SEQ ID NO: 62 - DNA, 30 bp, synthetic construct
  source 1..30: mol_type = other DNA
gtatttagcaaaagactctgagccgtcatt

SEQ ID NO: 63 - DNA, 30 bp, synthetic construct
  source 1..30: mol_type = other DNA
ttagctcaatacacacgtcaacgtacggca

SEQ ID NO: 64 - DNA, 30 bp, synthetic construct
  source 1..30: mol_type = other DNA
aagaaccatagcgcatgtatcccacgaccg

SEQ ID NO: 65 - DNA, 30 bp, synthetic construct
  source 1..30: mol_type = other DNA
cgagagacccacgtcagtcgtaactaatta

SEQ ID NO: 66 - DNA, 30 bp, synthetic construct
  source 1..30: mol_type = other DNA
ggatttacatgagtctgccaggtaccgatc

SEQ ID NO: 67 - DNA, 30 bp, synthetic construct
  source 1..30: mol_type = other DNA
tatgtgggagggttcgctacagtgatccat

SEQ ID NO: 68 - DNA, 30 bp, synthetic construct
  source 1..30: mol_type = other DNA
ggacccggaaggtgtaaactaccactgaca

SEQ ID NO: 69 - DNA, 30 bp, synthetic construct
  source 1..30: mol_type = other DNA
aggttgactacacggccgtccctaaaaggt

SEQ ID NO: 70 - DNA, 30 bp, synthetic construct
  source 1..30: mol_type = other DNA
acgagagcgcggtcgactagcaaattgccg

SEQ ID NO: 71 - DNA, 30 bp, synthetic construct
  source 1..30: mol_type = other DNA
gaagacgtcttgcgacctatgagccgtgcc

SEQ ID NO: 72 - DNA, 30 bp, synthetic construct
  source 1..30: mol_type = other DNA
agcggtttcaatgggaagagttggttgtcc

SEQ ID NO: 73 - DNA, 30 bp, synthetic construct
  source 1..30: mol_type = other DNA
attatagtaagcgttcatctggtttatgtc

SEQ ID NO: 74 - DNA, 30 bp, synthetic construct
  source 1..30: mol_type = other DNA
ttgtgtcgcttatacccatacatgcctacc

SEQ ID NO: 75 - DNA, 30 bp, synthetic construct
  source 1..30: mol_type = other DNA
gcattcgttacgggcctctctgaacaagaa

SEQ ID NO: 76 - DNA, 30 bp, synthetic construct
  source 1..30: mol_type = other DNA
agcaagttagatgtatgcaaccaatatgga

SEQ ID NO: 77 - DNA, 30 bp, synthetic construct
  source 1..30: mol_type = other DNA
ggcaagtcacacgtctataccggtcggtcc

SEQ ID NO: 78 - DNA, 30 bp, synthetic construct
  source 1..30: mol_type = other DNA
tgcaaacaagccgtagtagccaacctgcct

SEQ ID NO: 79 - DNA, 30 bp, synthetic construct
  source 1..30: mol_type = other DNA
tacatacgacatacggtaacttctacaggc

SEQ ID NO: 80 - DNA, 30 bp, synthetic construct
  source 1..30: mol_type = other DNA
cacccgataagatacgatgcctccgtcaat

SEQ ID NO: 81 - DNA, 30 bp, synthetic construct
  source 1..30: mol_type = other DNA
cagcgcgactaggcagtaatgactatccac

SEQ ID NO: 82 - DNA, 30 bp, synthetic construct
  source 1..30: mol_type = other DNA
acagtcgaccggattgtaactcatgtaaac

SEQ ID NO: 83 - DNA, 30 bp, synthetic construct
  source 1..30: mol_type = other DNA
tcgcttggcaatttcaccggaatatgtgta

SEQ ID NO: 84 - DNA, 30 bp, synthetic construct
  source 1..30: mol_type = other DNA
caatgttaaagatggaaatacgaggagcgt

SEQ ID NO: 85 - DNA, 30 bp, synthetic construct
  source 1..30: mol_type = other DNA
gtggacaaacagctgcagaatctctagtcg

SEQ ID NO: 86 - DNA, 30 bp, synthetic construct
  source 1..30: mol_type = other DNA
caaaaacgtgcctgagtcaatgggctttca

SEQ ID NO: 87 - DNA, 30 bp, synthetic construct
  source 1..30: mol_type = other DNA
aaagcagtggtacgagccggtgcaactaag

SEQ ID NO: 88 - DNA, 30 bp, synthetic construct
  source 1..30: mol_type = other DNA
cactgaaactcaacgagcgatctaaggcgg

SEQ ID NO: 89 - DNA, 30 bp, synthetic construct
  source 1..30: mol_type = other DNA
tgctcaacaaccatgccacatcgacgtacg

SEQ ID NO: 90 - DNA, 30 bp, synthetic construct
  source 1..30: mol_type = other DNA
gaggctatattcaagtttttacgggagaag

SEQ ID NO: 91 - DNA, 30 bp, synthetic construct
  source 1..30: mol_type = other DNA
gtttccgcgagctgaagcaatagaacgatg

SEQ ID NO: 92 - DNA, 30 bp, synthetic construct
  source 1..30: mol_type = other DNA
tatatactccgttcttgtttccggtgtggc

SEQ ID NO: 93 - DNA, 30 bp, synthetic construct
  source 1..30: mol_type = other DNA
aggtaggcatctcgattgatctataggctt

SEQ ID NO: 94 - DNA, 30 bp, synthetic construct
  source 1..30: mol_type = other DNA
gtcaggaaaagcaggcattatacgtaacag

SEQ ID NO: 95 - DNA, 30 bp, synthetic construct
  source 1..30: mol_type = other DNA
gggcaatgaacgcgatgggtctctttcgat

SEQ ID NO: 96 - DNA, 30 bp, synthetic construct
  source 1..30: mol_type = other DNA
gtgccgatagcccattccgtctacattgat

SEQ ID NO: 97 - DNA, 30 bp, synthetic construct
  source 1..30: mol_type = other DNA
gaatacataaaagggatcgttaacacccac

SEQ ID NO: 98 - DNA, 30 bp, synthetic construct
  source 1..30: mol_type = other DNA
tatgaccggtagacctgtctgaatccgctc

SEQ ID NO: 99 - DNA, 30 bp, synthetic construct
  source 1..30: mol_type = other DNA
gtttcttttactacgacaggcgataccgac

SEQ ID NO: 100 - DNA, 30 bp, synthetic construct
  source 1..30: mol_type = other DNA
tgcgcagatggcttttcggatagagaattg

SEQ ID NO: 101 - DNA, 30 bp, synthetic construct
  source 1..30: mol_type = other DNA
aaaatccctgggctttagctcgatccaaat

SEQ ID NO: 102 - DNA, 30 bp, synthetic construct
  source 1..30: mol_type = other DNA
ctgtcgccaaggactggtccgggaagtacg

SEQ ID NO: 103 - DNA, 30 bp, synthetic construct
  source 1..30: mol_type = other DNA
ccgatcctagaggtgacaataaataagagc

SEQ ID NO: 104 - DNA, 30 bp, synthetic construct
  source 1..30: mol_type = other DNA
tggttagttggtgtctgtctttctggattg

SEQ ID NO: 105 - DNA, 30 bp, synthetic construct
  source 1..30: mol_type = other DNA
agtattatggacatgtgctggttttctggc

SEQ ID NO: 106 - DNA, 30 bp, synthetic construct
  source 1..30: mol_type = other DNA
cctgatttacctagattatacatacttcac

SEQ ID NO: 107 - DNA, 30 bp, synthetic construct
  source 1..30: mol_type = other DNA
gccggatattagatgtctccaacgcacaac

SEQ ID NO: 108 - DNA, 30 bp, synthetic construct
  source 1..30: mol_type = other DNA
tataggattagctgcatcaccatgagtaac

SEQ ID NO: 109 - DNA, 30 bp, synthetic construct
  source 1..30: mol_type = other DNA
cataccatctgcctttcaagtggagtcacc

SEQ ID NO: 110 - DNA, 30 bp, synthetic construct
  source 1..30: mol_type = other DNA
gttaaagcctgtccagtgtttctccctacg

SEQ ID NO: 111 - DNA, 30 bp, synthetic construct
  source 1..30: mol_type = other DNA
cttaaagtcagggttagctcagggtacgtg

SEQ ID NO: 112 - DNA, 30 bp, synthetic construct
  source 1..30: mol_type = other DNA
gcatagttgtttgtcctgtcgagaagcgag

SEQ ID NO: 113 - DNA, 30 bp, synthetic construct
  source 1..30: mol_type = other DNA
gctatgacaacttaactcgctctctctgac

SEQ ID NO: 114 - DNA, 30 bp, synthetic construct
  source 1..30: mol_type = other DNA
gcgggaatcaccgagtactcagttaggtaa

SEQ ID NO: 115 - DNA, 30 bp, synthetic construct
  source 1..30: mol_type = other DNA
cataaacgaattacactctaggtagtatcg

SEQ ID NO: 116 - DNA, 30 bp, synthetic construct
  source 1..30: mol_type = other DNA
tatacaagcaacgttaacgattggcagcat

SEQ ID NO: 117 - DNA, 30 bp, synthetic construct
  source 1..30: mol_type = other DNA
ctgggacgctagatatcaagggctacaatc

SEQ ID NO: 118 - DNA, 30 bp, synthetic construct
  source 1..30: mol_type = other DNA
tgataagaattgatagcatgtgcagtgaga

SEQ ID NO: 119 - DNA, 30 bp, synthetic construct
  source 1..30: mol_type = other DNA
ggcaacgtgctttactactgacgcttctta

SEQ ID NO: 120 - DNA, 30 bp, synthetic construct
  source 1..30: mol_type = other DNA
cgttgcagctattcgtgtcgcttaatttgg

SEQ ID NO: 121 - DNA, 30 bp, synthetic construct
  source 1..30: mol_type = other DNA
cacttagtaatccagttcatccattggcga

SEQ ID NO: 122 - DNA, 30 bp, synthetic construct
  source 1..30: mol_type = other DNA
acccaaaacatcgaaaaacacctcagaacg

SEQ ID NO: 123 - DNA, 30 bp, synthetic construct
  source 1..30: mol_type = other DNA
actcgtcattatgcaaggatagagctagct

SEQ ID NO: 124 - DNA, 30 bp, synthetic construct
  source 1..30: mol_type = other DNA
aaatcatggtcaaccaatgtgttcgagact

SEQ ID NO: 125 - DNA, 30 bp, synthetic construct
  source 1..30: mol_type = other DNA
ggtattttggttgtatgccactgggtctga

SEQ ID NO: 126 - DNA, 30 bp, synthetic construct
  source 1..30: mol_type = other DNA
agtacgtacctattaatcaacttcgcctga

SEQ ID NO: 127 - DNA, 30 bp, synthetic construct
  source 1..30: mol_type = other DNA
agaggaccaccagcttgtaaattcactaat

SEQ ID NO: 128 - DNA, 30 bp, synthetic construct
  source 1..30: mol_type = other DNA
ccgacacataaggttggtgaagatagtcca

SEQ ID NO: 129 - DNA, 30 bp, synthetic construct
  source 1..30: mol_type = other DNA
gaagggatgggcggcactttggatatttag

SEQ ID NO: 130 - DNA, 30 bp, synthetic construct
  source 1..30: mol_type = other DNA
aaggcatacatagaaggtagcgtgatacgg

SEQ ID NO: 131 - DNA, 30 bp, synthetic construct
  source 1..30: mol_type = other DNA
gactcggcataaccaaaggtatccaacgaa

SEQ ID NO: 132 - DNA, 30 bp, synthetic construct
  source 1..30: mol_type = other DNA
tatatttgtgatcaggttctcatttatcgc

SEQ ID NO: 133 - DNA, 30 bp, synthetic construct
  source 1..30: mol_type = other DNA
tcgagtacgtcatattttagtagtctgcgt

SEQ ID NO: 134 - DNA, 30 bp, synthetic construct
  source 1..30: mol_type = other DNA
ccatgatagagagtagaatgaagagcacac

SEQ ID NO: 135 - DNA, 30 bp, synthetic construct
  source 1..30: mol_type = other DNA
gaattgactacaggttaagaatactataca

SEQ ID NO: 136 - DNA, 30 bp, synthetic construct
  source 1..30: mol_type = other DNA
cgaattatcttacgccgccgatgttttcta

SEQ ID NO: 137 - DNA, 30 bp, synthetic construct
  source 1..30: mol_type = other DNA
atctagttacagttccctacattcacaaga

SEQ ID NO: 138 - DNA, 30 bp, synthetic construct
  source 1..30: mol_type = other DNA
ggttataaatggtcttggttttggagtgct

SEQ ID NO: 139 - DNA, 30 bp, synthetic construct
  source 1..30: mol_type = other DNA
gagattatcgtacctccacttggatgaaca

SEQ ID NO: 140 - DNA, 30 bp, synthetic construct
  source 1..30: mol_type = other DNA
ctctcgccataagcttcccggatacactat

SEQ ID NO: 141 - DNA, 30 bp, synthetic construct
  source 1..30: mol_type = other DNA
ggagattatcagcttctatacccactgcgc

SEQ ID NO: 142 - DNA, 30 bp, synthetic construct
  source 1..30: mol_type = other DNA
ggctttatttggcgtactgtcatattcgat

SEQ ID NO: 143 - DNA, 30 bp, synthetic construct
  source 1..30: mol_type = other DNA
gtaccatcatcctaaagagctcttcagtgt

SEQ ID NO: 144 - DNA, 30 bp, synthetic construct
  source 1..30: mol_type = other DNA
gaaatgtatgagattggcaccttcttaaat

SEQ ID NO: 145 - DNA, 30 bp, synthetic construct
  source 1..30: mol_type = other DNA
tcaggacaacgcgaatcttatagcgcatag

SEQ ID NO: 146 - DNA, 30 bp, synthetic construct
  source 1..30: mol_type = other DNA
aactcgacgttccacatgatattgcctcaa

SEQ ID NO: 147 - DNA, 30 bp, synthetic construct
  source 1..30: mol_type = other DNA
cttgactgcattgcttttgtaacctactgc

SEQ ID NO: 148 - DNA, 30 bp, synthetic construct
  source 1..30: mol_type = other DNA
tttcacgaattcgaagggcactggcttatt

SEQ ID NO: 149 - DNA, 30 bp, synthetic construct
  source 1..30: mol_type = other DNA
gtgatcagataagtcgttaaagctccttct

SEQ ID NO: 150 - DNA, 30 bp, synthetic construct
  source 1..30: mol_type = other DNA
tcacttagtactcatgagaggtagtttcgg

SEQ ID NO: 151 - DNA, 30 bp, synthetic construct
  source 1..30: mol_type = other DNA
tcgtacctgctatatgttaaactttagaga

SEQ ID NO: 152 - DNA, 30 bp, synthetic construct
  source 1..30: mol_type = other DNA
ccctcaccttaaaaagacctacctgtattc

SEQ ID NO: 153 - DNA, 30 bp, synthetic construct
  source 1..30: mol_type = other DNA
caggttaatgatccatcaagacgaatccta

SEQ ID NO: 154 - DNA, 30 bp, synthetic construct
  source 1..30: mol_type = other DNA
tgaaatttaggtattatcttgctacttgga

SEQ ID NO: 155 - DNA, 30 bp, synthetic construct
  source 1..30: mol_type = other DNA
acgcaagggcatagttgagtaatatagaga

SEQ ID NO: 156 - DNA, 30 bp, synthetic construct
  source 1..30: mol_type = other DNA
gtcaccgaatgagtacagaaagctagagtg

SEQ ID NO: 157 - DNA, 30 bp, synthetic construct
  source 1..30: mol_type = other DNA
tgctactttacgaattaatatgtcttaccg

SEQ ID NO: 158 - DNA, 30 bp, synthetic construct
  source 1..30: mol_type = other DNA
aaggaaatgaggttcttgtgagttctagtc

SEQ ID NO: 159 - DNA, 30 bp, synthetic construct
  source 1..30: mol_type = other DNA
cctcaaggaaacgtacgatgtgacttactg

SEQ ID NO: 160 - DNA, 30 bp, synthetic construct
  source 1..30: mol_type = other DNA
cagtaccgctcttataaaacttagtcgaac

SEQ ID NO: 161 - DNA, 30 bp, synthetic construct
  source 1..30: mol_type = other DNA
atgatgtctaactggccatttgtccctttg

SEQ ID NO: 162 - DNA, 30 bp, synthetic construct
  source 1..30: mol_type = other DNA
ggaaattaggacgccatcttgactttatac

SEQ ID NO: 163 - DNA, 30 bp, synthetic construct
  source 1..30: mol_type = other DNA
tcgcaaattcattatcaaactcgccgtgag

SEQ ID NO: 164 - DNA, 30 bp, synthetic construct
  source 1..30: mol_type = other DNA
gaaatcatgcgtggcgttggttaaatacga

SEQ ID NO: 165 - DNA, 30 bp, synthetic construct
  source 1..30: mol_type = other DNA
cccgaaagagccgtaatccattgtaagctg

SEQ ID NO: 166 - DNA, 30 bp, synthetic construct
  source 1..30: mol_type = other DNA
atgattatcgctagtaccgtaagtattttc

SEQ ID NO: 167 - DNA, 30 bp, synthetic construct
  source 1..30: mol_type = other DNA
cattcgaataagatacacgaggaccatagg

SEQ ID NO: 168 - DNA, 30 bp, synthetic construct
  source 1..30: mol_type = other DNA
gtgtcagttggatattgtaacgtcgaaacc

SEQ ID NO: 169 - DNA, 30 bp, synthetic construct
  source 1..30: mol_type = other DNA
atgagagactataccgggtactgcaatatg

SEQ ID NO: 170 - DNA, 30 bp, synthetic construct
  source 1..30: mol_type = other DNA
gtctaatttgctcaactcctcttcacccaa

SEQ ID NO: 171 - DNA, 30 bp, synthetic construct
  source 1..30: mol_type = other DNA
agaaataccaatccacctcggaaatgtgta

SEQ ID NO: 172 - DNA, 30 bp, synthetic construct
  source 1..30: mol_type = other DNA
tagatgtcaacaaacaagccagtcttcgct

SEQ ID NO: 173 - DNA, 30 bp, synthetic construct
  source 1..30: mol_type = other DNA
cctagacttattgaccttgtttaacccggc

SEQ ID NO: 174 - DNA, 30 bp, synthetic construct
  source 1..30: mol_type = other DNA
taaataaggatatctgagtcaacggacgcc

SEQ ID NO: 175 - DNA, 30 bp, synthetic construct
  source 1..30: mol_type = other DNA
tctgagggattctacttatcgaagccaacc

SEQ ID NO: 176 - DNA, 30 bp, synthetic construct
  source 1..30: mol_type = other DNA
tccagttataagtaagaaaccgtccgtcgt

SEQ ID NO: 177 - DNA, 30 bp, synthetic construct
  source 1..30: mol_type = other DNA
attgacattaagccatgccagcccataact

SEQ ID NO: 178 - DNA, 30 bp, synthetic construct
  source 1..30: mol_type = other DNA
ggtgaaaagttcccactacccataacagtc

SEQ ID NO: 179 - DNA, 30 bp, synthetic construct
  source 1..30: mol_type = other DNA
tataggcatcacgcaaccgtttagcgaata

SEQ ID NO: 180 - DNA, 30 bp, synthetic construct
  source 1..30: mol_type = other DNA
caccctctctactccaactaatcctacgca

SEQ ID NO: 181 - DNA, 30 bp, synthetic construct
  source 1..30: mol_type = other DNA
cccaggaattagtagcgtttcatgcagaat

SEQ ID NO: 182 - DNA, 30 bp, synthetic construct
  source 1..30: mol_type = other DNA
accaggcggaaccataaagtgatacctatc

SEQ ID NO: 183 - DNA, 30 bp, synthetic construct
  source 1..30: mol_type = other DNA
tattggaaagccgctcaagatatagtatag

SEQ ID NO: 184 - DNA, 30 bp, synthetic construct
  source 1..30: mol_type = other DNA
agtgaacttctagtgtatcccaaagcgtgg

SEQ ID NO: 185 - DNA, 30 bp, synthetic construct
  source 1..30: mol_type = other DNA
gattctacagccaacattacacttctccaa

SEQ ID NO: 186 - DNA, 30 bp, synthetic construct
  source 1..30: mol_type = other DNA
cggtaaatcccactatgctacatgtaagcg

SEQ ID NO: 187 - DNA, 30 bp, synthetic construct
  source 1..30: mol_type = other DNA
gtacagcccgatccgaaatggtttaagagt

SEQ ID NO: 188 - DNA, 30 bp, synthetic construct
  source 1..30: mol_type = other DNA
tctgcacgcgactttaagaattggccatag

SEQ ID NO: 189 - DNA, 30 bp, synthetic construct
  source 1..30: mol_type = other DNA
agacacagatacgtatttacttaatccgcg

SEQ ID NO: 190 - DNA, 30 bp, synthetic construct
  source 1..30: mol_type = other DNA
attatagcgtattacccaacacaatgaatt

SEQ ID NO: 191 - DNA, 30 bp, synthetic construct
  source 1..30: mol_type = other DNA
ttacccttgcacctaacacatataactgta

SEQ ID NO: 192 - DNA, 30 bp, synthetic construct
  source 1..30: mol_type = other DNA
cagctgaacactcgcagatcgttttacata

SEQ ID NO: 193 - DNA, 30 bp, synthetic construct
  source 1..30: mol_type = other DNA
gtttaggttctgctactagcgtacatgagg

SEQ ID NO: 194 - DNA, 30 bp, synthetic construct
  source 1..30: mol_type = other DNA
aaaatgacactgccgataaatattgcgtgg

SEQ ID NO: 195 - DNA, 30 bp, synthetic construct
  source 1..30: mol_type = other DNA
cgtctcgaggatggtccattttataactgt

SEQ ID NO: 196 - DNA, 30 bp, synthetic construct
  source 1..30: mol_type = other DNA
aagaatctgcatcggaaatatgagtgttaa

SEQ ID NO: 197 - DNA, 30 bp, synthetic construct
  source 1..30: mol_type = other DNA
cggataaaggcagtggatgggttagaatga

SEQ ID NO: 198 - DNA, 30 bp, synthetic construct
  source 1..30: mol_type = other DNA
cagtaagtccaagatccctaccataatttc

SEQ ID NO: 199 - DNA, 30 bp, synthetic construct
  source 1..30: mol_type = other DNA
ggaagttctgaagggacatgttctaagtga

SEQ ID NO: 200 - DNA, 30 bp, synthetic construct
  source 1..30: mol_type = other DNA
tgtgttttagtgacggcataattaccggcg

SEQ ID NO: 201 - DNA, 30 bp, synthetic construct
  source 1..30: mol_type = other DNA
aaaatatcggacctttagaagtacggtacc

SEQ ID NO: 202 - DNA, 30 bp, synthetic construct
  source 1..30: mol_type = other DNA
ttgtgacataataactagaagccctggctg

SEQ ID NO: 203 - DNA, 30 bp, synthetic construct
  source 1..30: mol_type = other DNA
tgttatcaaaacttgtcgacacatctgaga

SEQ ID NO: 204 - DNA, 30 bp, synthetic construct
  source 1..30: mol_type = other DNA
gacttacttaagaactgctacgcctacaat

SEQ ID NO: 205 - DNA, 20 bp, synthetic construct
  source 1..20: mol_type = other DNA
tgcacttcatcagagatgat

SEQ ID NO: 206 - DNA, 20 bp, synthetic construct
  source 1..20: mol_type = other DNA
tgcacagacacagcattgat

SEQ ID NO: 207 - DNA, 18 bp, synthetic construct
  source 1..18: mol_type = other DNA
tgcacacgcgattctgat

SEQ ID NO: 208 - DNA, 20 bp, synthetic construct
  source 1..20: mol_type = other DNA
tgcacagccagtctgctgat

SEQ ID NO: 209 - DNA, 18 bp, synthetic construct
  source 1..18: mol_type = other DNA
tgcacaagtatcagcgat

SEQ ID NO: 210 - DNA, 18 bp, synthetic construct
  source 1..18: mol_type = other DNA
tgcactagcagtgttgat

SEQ ID NO: 211 - DNA, 20 bp, synthetic construct
  source 1..20: mol_type = other DNA
tgcacgagagcagccatgat

SEQ ID NO: 212 - DNA, 20 bp, synthetic construct
  source 1..20: mol_type = other DNA
tgcacaatcgcatgtgtgat

SEQ ID NO: 213 - DNA, 18 bp, synthetic construct
  source 1..18: mol_type = other DNA
tgcacacatctcgaagat

SEQ ID NO: 214 - DNA, 18 bp, synthetic construct
  source 1..18: mol_type = other DNA
tgcacaacagatagagat

SEQ ID NO: 215 - DNA, 18 bp, synthetic construct
  source 1..18: mol_type = other DNA
tgcacgcataatactgat

SEQ ID NO: 216 - DNA, 18 bp, synthetic construct
  source 1..18: mol_type = other DNA
tgcacatgtgtacttgat

SEQ ID NO: 217 - DNA, 20 bp, synthetic construct
  source 1..20: mol_type = other DNA
tgcacttcatgtgagctgat

SEQ ID NO: 218 - DNA, 20 bp, synthetic construct
  source 1..20: mol_type = other DNA
tgcacatgctcaacagcgat

SEQ ID NO: 219 - DNA, 18 bp, synthetic construct
  source 1..18: mol_type = other DNA
tgcacgtggacatcagat

SEQ ID NO: 220 - DNA, 18 bp, synthetic construct
  source 1..18: mol_type = other DNA
tgcactcacaatgacgat

SEQ ID NO: 221 - DNA, 18 bp, synthetic construct
  source 1..18: mol_type = other DNA
tgcacaacgatatgtgat

SEQ ID NO: 222 - DNA, 18 bp, synthetic construct
  source 1..18: mol_type = other DNA
tgcacatcaccacgcgat

SEQ ID NO: 223 - DNA, 20 bp, synthetic construct
  source 1..20: mol_type = other DNA
tgcactgcgaatctgctgat

SEQ ID NO: 224 - DNA, 20 bp, synthetic construct
  source 1..20: mol_type = other DNA
tgcacatataatgctgagat

SEQ ID NO: 225 - DNA, 20 bp, synthetic construct
  source 1..20: mol_type = other DNA
tgcacatgatgccgtctgat

SEQ ID NO: 226 - DNA, 18 bp, synthetic construct
  source 1..18: mol_type = other DNA
tgcactatcgattgagat

SEQ ID NO: 227 - DNA, 20 bp, synthetic construct
  source 1..20: mol_type = other DNA
tgcacagcattgcgcgcgat

SEQ ID NO: 228 - DNA, 18 bp, synthetic construct
  source 1..18: mol_type = other DNA
tgcactctatatgaagat

SEQ ID NO: 229 - DNA, 20 bp, synthetic construct
  source 1..20: mol_type = other DNA
tgcacgcatgtcattatgat

SEQ ID NO: 230 - DNA, 20 bp, synthetic construct
  source 1..20: mol_type = other DNA
tgcacatgcggatcatcgat

SEQ ID NO: 231 - DNA, 18 bp, synthetic construct
  source 1..18: mol_type = other DNA
tgcacacaatcactagat

SEQ ID NO: 232 - DNA, 22 bp, synthetic construct
  source 1..22: mol_type = other DNA
tgcacacagccatatgcatgat

SEQ ID NO: 233 - DNA, 20 bp, synthetic construct
  source 1..20: mol_type = other DNA
tgcacacaagacatgctgat

SEQ ID NO: 234 - DNA, 20 bp, synthetic construct
  source 1..20: mol_type = other DNA
tgcacatgcttcactctgat

SEQ ID NO: 235 - DNA, 20 bp, synthetic construct
  source 1..20: mol_type = other DNA
tgcacatcagcagttatgat

SEQ ID NO: 236 - DNA, 20 bp, synthetic construct
  source 1..20: mol_type = other DNA
tgcactatatcatgattgat

SEQ ID NO: 237 - DNA, 20 bp, synthetic construct
  source 1..20: mol_type = other DNA
tgcactgcgatgattctgat

SEQ ID NO: 238 - DNA, 18 bp, synthetic construct
  source 1..18: mol_type = other DNA
tgcacacatatatccgat

SEQ ID NO: 239 - DNA, 20 bp, synthetic construct
  source 1..20: mol_type = other DNA
tgcacatagatatgattgat

SEQ ID NO: 240 - DNA, 22 bp, synthetic construct
  source 1..22: mol_type = other DNA
tgcactgtgctggcatcatgat

SEQ ID NO: 241 - DNA, 20 bp, synthetic construct
  source 1..20: mol_type = other DNA
tgcacactcttcatgctgat

SEQ ID NO: 242 - DNA, 20 bp, synthetic construct
  source 1..20: mol_type = other DNA
tgcacagcataggctctgat

SEQ ID NO: 243 - DNA, 20 bp, synthetic construct
  source 1..20: mol_type = other DNA
tgcacatggcatagcgagat

SEQ ID NO: 244 - DNA, 18 bp, synthetic construct
  source 1..18: mol_type = other DNA
tgcactacaccacatgat

SEQ ID NO: 245 - DNA, 18 bp, synthetic construct
  source 1..18: mol_type = other DNA
tgcacagtgatccgtgat

SEQ ID NO: 246 - DNA, 20 bp, synthetic construct
  source 1..20: mol_type = other DNA
tgcacagagcgaatcatgat

SEQ ID NO: 247 - DNA, 22 bp, synthetic construct
  source 1..22: mol_type = other DNA
tgcacatggcagctagcatgat

SEQ ID NO: 248 - DNA, 20 bp, synthetic construct
  source 1..20: mol_type = other DNA
tgcacagcagattatgcgat

SEQ ID NO: 249 - DNA, 20 bp, synthetic construct
  source 1..20: mol_type = other DNA
tgcacaatgcatacagtgat

SEQ ID NO: 250 - DNA, 22 bp, synthetic construct
  source 1..22: mol_type = other DNA
tgcacagcatgcaccatcagat

SEQ ID NO: 251 - DNA, 18 bp, synthetic construct
  source 1..18: mol_type = other DNA
tgcacaagtcagtgtgat

SEQ ID NO: 252 - DNA, 22 bp, synthetic construct
  source 1..22: mol_type = other DNA
tgcacatgcagcatcaagagat

SEQ ID NO: 253 - DNA, 18 bp, synthetic construct
  source 1..18: mol_type = other DNA
tgcacgctgcagtaagat

SEQ ID NO: 254 - DNA, 18 bp, synthetic construct
  source 1..18: mol_type = other DNA
tgcactgacagtcaagat

SEQ ID NO: 255 - DNA, 20 bp, synthetic construct
  source 1..20: mol_type = other DNA
tgcacaatcacatatgcgat

SEQ ID NO: 256 - DNA, 20 bp, synthetic construct
  source 1..20: mol_type = other DNA
tgcacatgcgtggctctgat

SEQ ID NO: 257 - DNA, 22 bp, synthetic construct
  source 1..22: mol_type = other DNA
tgcacagccagcagatgctgat

SEQ ID NO: 258 - DNA, 20 bp, synthetic construct
  source 1..20: mol_type = other DNA
tgcacactagcatcattgat

SEQ ID NO: 259 - DNA, 20 bp, synthetic construct
  source 1..20: mol_type = other DNA
tgcactgcatgtgagaagat

SEQ ID NO: 260 - DNA, 18 bp, synthetic construct
  source 1..18: mol_type = other DNA
tgcactcgtcatgccgat

SEQ ID NO: 261 - DNA, 20 bp, synthetic construct
  source 1..20: mol_type = other DNA
tgcactgcaccagctgcgat

SEQ ID NO: 262 - DNA, 20 bp, synthetic construct
  source 1..20: mol_type = other DNA
tgcacatggcgagctatgat

SEQ ID NO: 263 - DNA, 20 bp, synthetic construct
  source 1..20: mol_type = other DNA
tgcacttctgatctcatgat

SEQ ID NO: 264 - DNA, 18 bp, synthetic construct
  source 1..18: mol_type = other DNA
tgcactatcgctgccgat

SEQ ID NO: 265 - DNA, 22 bp, synthetic construct
  source 1..22: mol_type = other DNA
tgcacatgcaatatgagcagat

SEQ ID NO: 266 - DNA, 18 bp, synthetic construct
  source 1..18: mol_type = other DNA
tgcacagataatagagat

SEQ ID NO: 267 - DNA, 18 bp, synthetic construct
  source 1..18: mol_type = other DNA
tgcacttatcagcgtgat

SEQ ID NO: 268 - DNA, 18 bp, synthetic construct
  source 1..18: mol_type = other DNA
tgcactaggcatcgcgat

SEQ ID NO: 269 - DNA, 20 bp, synthetic construct
  source 1..20: mol_type = other DNA
tgcactctaggatgcatgat

SEQ ID NO: 270 - DNA, 22 bp, synthetic construct
  source 1..22: mol_type = other DNA
tgcactgcctcatcatgcagat

SEQ ID NO: 271 - DNA, 22 bp, synthetic construct
  source 1..22: mol_type = other DNA
tgcacagcatgcctgcactgat

SEQ ID NO: 272 - DNA, 22 bp, synthetic construct
  source 1..22: mol_type = other DNA
tgcacatgcactgcgccatgat

SEQ ID NO: 273 - DNA, 20 bp, synthetic construct
  source 1..20: mol_type = other DNA
tgcactctgagcaggctgat

SEQ ID NO: 274 - DNA, 18 bp, synthetic construct
  source 1..18: mol_type = other DNA
tgcacataagctagcgat

SEQ ID NO: 275 - DNA, 20 bp, synthetic construct
  source 1..20: mol_type = other DNA
tgcacaagcacatgagtgat

SEQ ID NO: 276 - DNA, 18 bp, synthetic construct
  source 1..18: mol_type = other DNA
tgcacgactggatgcgat

SEQ ID NO: 277 - DNA, 20 bp, synthetic construct
  source 1..20: mol_type = other DNA
tgcacatgcgtgcaagcgat

SEQ ID NO: 278 - DNA, 20 bp, synthetic construct
  source 1..20: mol_type = other DNA
tgcacatgagctgcctagat

SEQ ID NO: 279 - DNA, 20 bp, synthetic construct
  source 1..20: mol_type = other DNA
tgcactctgcctgctatgat

SEQ ID NO: 280 - DNA, 18 bp, synthetic construct
  source 1..18: mol_type = other DNA
tgcactatgtacattgat

SEQ ID NO: 281 - DNA, 18 bp, synthetic construct
  source 1..18: mol_type = other DNA
tgcacactgttatgcgat

SEQ ID NO: 282 - DNA, 20 bp, synthetic construct
  source 1..20: mol_type = other DNA
tgcactgccatagtgctgat

SEQ ID NO: 283 - DNA, 20 bp, synthetic construct
  source 1..20: mol_type = other DNA
tgcactcaacatctcatgat

SEQ ID NO: 284 - DNA, 20 bp, synthetic construct
  source 1..20: mol_type = other DNA
tgcacatgcgctcgcttgat

SEQ ID NO: 285 - DNA, 20 bp, synthetic construct
  source 1..20: mol_type = other DNA
tgcacagacgcttgcatgat

SEQ ID NO: 286 - DNA, 20 bp, synthetic construct
  source 1..20: mol_type = other DNA
tgcacaagagctctgcagat

SEQ ID NO: 287 - DNA, 20 bp, synthetic construct
  source 1..20: mol_type = other DNA
tgcactcttcagctgctgat

SEQ ID NO: 288 - DNA, 20 bp, synthetic construct
  source 1..20: mol_type = other DNA
tgcacatatgcaatacagat

SEQ ID NO: 289 - DNA, 18 bp, synthetic construct
  source 1..18: mol_type = other DNA
tgcactatcttgtatgat

SEQ ID NO: 290 - DNA, 20 bp, synthetic construct
  source 1..20: mol_type = other DNA
tgcactctgttgctgctgat

SEQ ID NO: 291 - DNA, 18 bp, synthetic construct
  source 1..18: mol_type = other DNA
tgcacactcatccgtgat

SEQ ID NO: 292 - DNA, 22 bp, synthetic construct
  source 1..22: mol_type = other DNA
tgcactgcatgcattatgcgat

SEQ ID NO: 293 - DNA, 18 bp, synthetic construct
  source 1..18: mol_type = other DNA
tgcacagcgctagaagat

SEQ ID NO: 294 - DNA, 20 bp, synthetic construct
  source 1..20: mol_type = other DNA
tgcacgatagctgcattgat

SEQ ID NO: 295 - DNA, 20 bp, synthetic construct
  source 1..20: mol_type = other DNA
tgcacgagcttgcatatgat

SEQ ID NO: 296 - DNA, 18 bp, synthetic construct
  source 1..18: mol_type = other DNA
tgcacactgtgtcaagat

SEQ ID NO: 297 - DNA, 20 bp, synthetic construct
  source 1..20: mol_type = other DNA
tgcacgcatgcgcagttgat

SEQ ID NO: 298 - DNA, 18 bp, synthetic construct
  source 1..18: mol_type = other DNA
tgcacggacatctgtgat

SEQ ID NO: 299 - DNA, 18 bp, synthetic construct
  source 1..18: mol_type = other DNA
tgcacagacttgtgagat

SEQ ID NO: 300 - DNA, 20 bp, synthetic construct
  source 1..20: mol_type = other DNA
tgcacgatatgcattctgat

SEQ ID NO: 301 - DNA, 20 bp, synthetic construct
  source 1..20: mol_type = other DNA
tgcacaatagatctgctgat

SEQ ID NO: 302 - DNA, 20 bp, synthetic construct
  source 1..20: mol_type = other DNA
tgcacgatgctcctgatgat

SEQ ID NO: 303 - DNA, 20 bp, synthetic construct
  source 1..20: mol_type = other DNA
tgcactgccgagcatgcgat

SEQ ID NO: 304 - DNA, 20 bp, synthetic construct
  source 1..20: mol_type = other DNA
tgcactgcactctggctgat

SEQ ID NO: 305 - DNA, 20 bp, synthetic construct
  source 1..20: mol_type = other DNA
tgcacggcatgctgagcgat

SEQ ID NO: 306 - DNA, 20 bp, synthetic construct
  source 1..20: mol_type = other DNA
tgcacaatatctgctctgat

SEQ ID NO: 307 - DNA, 22 bp, synthetic construct
  source 1..22: mol_type = other DNA
tgcactgaatcatgctgatgat

SEQ ID NO: 308 - DNA, 20 bp, synthetic construct
  source 1..20: mol_type = other DNA
tgcacttgcatgacatagat

SEQ ID NO: 309 - DNA, 22 bp, synthetic construct
  source 1..22: mol_type = other DNA
tgcacatgatgcgcctgatgat

SEQ ID NO: 310 - DNA, 20 bp, synthetic construct
  source 1..20: mol_type = other DNA
tgcacatgacatgccgtgat

SEQ ID NO: 311 - DNA, 20 bp, synthetic construct
  source 1..20: mol_type = other DNA
tgcacatgctcgatcaagat

SEQ ID NO: 312 - DNA, 20 bp, synthetic construct
  source 1..20: mol_type = other DNA
tgcactcacacaatgcagat

SEQ ID NO: 313 - DNA, 20 bp, synthetic construct
  source 1..20: mol_type = other DNA
tgcactgccacgagcatgat

SEQ ID NO: 314 - DNA, 18 bp, synthetic construct
  source 1..18: mol_type = other DNA
tgcacttactcatgcgat

SEQ ID NO: 315 - DNA, 20 bp, synthetic construct
  source 1..20: mol_type = other DNA
tgcacgcgctcagcattgat

SEQ ID NO: 316 - DNA, 20 bp, synthetic construct
  source 1..20: mol_type = other DNA
tgcacagcgcagatattgat

SEQ ID NO: 317 - DNA, 20 bp, synthetic construct
  source 1..20: mol_type = other DNA
tgcactgcagaggcagagat

SEQ ID NO: 318 - DNA, 20 bp, synthetic construct
  source 1..20: mol_type = other DNA
tgcacggcagtgcatgtgat

SEQ ID NO: 319 - DNA, 22 bp, synthetic construct
  source 1..22: mol_type = other DNA
tgcactcatggcatgagcagat

SEQ ID NO: 320 - DNA, 20 bp, synthetic construct
  source 1..20: mol_type = other DNA
tgcacatgagtcagattgat

SEQ ID NO: 321 - DNA, 20 bp, synthetic construct
  source 1..20: mol_type = other DNA
tgcacaagcatctcagtgat

SEQ ID NO: 322 - DNA, 22 bp, synthetic construct
  source 1..22: mol_type = other DNA
tgcacatcatcaagcacatgat

SEQ ID NO: 323 - DNA, 18 bp, synthetic construct
  source 1..18: mol_type = other DNA
tgcacttagcgcatcgat

SEQ ID NO: 324 - DNA, 20 bp, synthetic construct
  source 1..20: mol_type = other DNA
tgcacacaatgcgctgtgat

SEQ ID NO: 325 - DNA, 20 bp, synthetic construct
  source 1..20: mol_type = other DNA
tgcacaatatgcgatcagat

SEQ ID NO: 326 - DNA, 18 bp, synthetic construct
  source 1..18: mol_type = other DNA
tgcacttacatgtgcgat

SEQ ID NO: 327 - DNA, 20 bp, synthetic construct
  source 1..20: mol_type = other DNA
tgcactgtgtgcctgcagat

SEQ ID NO: 328 - DNA, 22 bp, synthetic construct
  source 1..22: mol_type = other DNA
tgcacaatgctgctcatgcgat

SEQ ID NO: 329 - DNA, 20 bp, synthetic construct
  source 1..20: mol_type = other DNA
tgcacgatctcatcattgat

SEQ ID NO: 330 - DNA, 20 bp, synthetic construct
  source 1..20: mol_type = other DNA
tgcacaatcagatgctagat

SEQ ID NO: 331 - DNA, 18 bp, synthetic construct
  source 1..18: mol_type = other DNA
tgcacaagagtgatagat

SEQ ID NO: 332 - DNA, 20 bp, synthetic construct
  source 1..20: mol_type = other DNA
tgcacttgcgtcatcatgat

SEQ ID NO: 333 - DNA, 20 bp, synthetic construct
  source 1..20: mol_type = other DNA
tgcacatgtccgcacatgat

SEQ ID NO: 334 - DNA, 20 bp, synthetic construct
  source 1..20: mol_type = other DNA
tgcacacaagcagctgcgat

SEQ ID NO: 335 - DNA, 20 bp, synthetic construct
  source 1..20: mol_type = other DNA
tgcactattgtgcgcatgat

SEQ ID NO: 336 - DNA, 22 bp, synthetic construct
  source 1..22: mol_type = other DNA
tgcacaacatgcatcatgtgat

SEQ ID NO: 337 - DNA, 20 bp, synthetic construct
  source 1..20: mol_type = other DNA
tgcacaactgcagatgagat

SEQ ID NO: 338 - DNA, 18 bp, synthetic construct
  source 1..18: mol_type = other DNA
tgcacatcctgtcgcgat

SEQ ID NO: 339 - DNA, 22 bp, synthetic construct
  source 1..22: mol_type = other DNA
tgcactgcagctgccatgcgat

SEQ ID NO: 340 - DNA, 18 bp, synthetic construct
  source 1..18: mol_type = other DNA
tgcactatattgctcgat

SEQ ID NO: 341 - DNA, 20 bp, synthetic construct
  source 1..20: mol_type = other DNA
tgcacaatactgatgctgat

SEQ ID NO: 342 - DNA, 20 bp, synthetic construct
  source 1..20: mol_type = other DNA
tgcacatgtatgatgaagat

SEQ ID NO: 343 - DNA, 20 bp, synthetic construct
  source 1..20: mol_type = other DNA
tgcactgaagcagacatgat

SEQ ID NO: 344 - DNA, 20 bp, synthetic construct
  source 1..20: mol_type = other DNA
tgcacaatgcgagcgcagat

SEQ ID NO: 345 - DNA, 20 bp, synthetic construct
  source 1..20: mol_type = other DNA
tgcacatgactcatcaagat

SEQ ID NO: 346 - DNA, 20 bp, synthetic construct
  source 1..20: mol_type = other DNA
tgcacacatcgccatctgat

SEQ ID NO: 347 - DNA, 20 bp, synthetic construct
  source 1..20: mol_type = other DNA
tgcactgctgtggatatgat

SEQ ID NO: 348 - DNA, 18 bp, synthetic construct
  source 1..18: mol_type = other DNA
tgcactcgcgcttgagat

SEQ ID NO: 349 - DNA, 20 bp, synthetic construct
  source 1..20: mol_type = other DNA
tgcacacacttgctgctgat

SEQ ID NO: 350 - DNA, 18 bp, synthetic construct
  source 1..18: mol_type = other DNA
tgcacttgtctgctcgat

SEQ ID NO: 351 - DNA, 20 bp, synthetic construct
  source 1..20: mol_type = other DNA
tgcactcagacagcattgat

SEQ ID NO: 352 - DNA, 18 bp, synthetic construct
  source 1..18: mol_type = other DNA
tgcacatagttcacagat

SEQ ID NO: 353 - DNA, 18 bp, synthetic construct
  source 1..18: mol_type = other DNA
tgcacataactgcgcgat

SEQ ID NO: 354 - DNA, 20 bp, synthetic construct
  source 1..20: mol_type = other DNA
tgcacacttgcgcatgcgat

SEQ ID NO: 355 - DNA, 19 bp, synthetic construct
  source 1..19: mol_type = other DNA
tgcactgcctgactgagat

SEQ ID NO: 356 - DNA, 18 bp, synthetic construct
  source 1..18: mol_type = other DNA
tgcactcagaatagcgat

SEQ ID NO: 357 - DNA, 18 bp, synthetic construct
  source 1..18: mol_type = other DNA
tgcacggcgtgatgcgat

SEQ ID NO: 358 - DNA, 22 bp, synthetic construct
  source 1..22: mol_type = other DNA
tgcacatgctgcatgccgtgat

SEQ ID NO: 359 - DNA, 20 bp, synthetic construct
  source 1..20: mol_type = other DNA
tgcactatgcattcagagat

SEQ ID NO: 360 - DNA, 18 bp, synthetic construct
  source 1..18: mol_type = other DNA
tgcacatggtcgcgtgat

SEQ ID NO: 361 - DNA, 20 bp, synthetic construct
  source 1..20: mol_type = other DNA
tgcacacgaggatgcatgat

SEQ ID NO: 362 - DNA, 18 bp, synthetic construct
  source 1..18: mol_type = other DNA
tgcactcgcacaagcgat

SEQ ID NO: 363 - DNA, 18 bp, synthetic construct
  source 1..18: mol_type = other DNA
tgcacacagttgtgcgat

SEQ ID NO: 364 - DNA, 20 bp, synthetic construct
  source 1..20: mol_type = other DNA
tgcacacggctgcatatgat

SEQ ID NO: 365 - DNA, 20 bp, synthetic construct
  source 1..20: mol_type = other DNA
tgcactgctgatgccgagat

SEQ ID NO: 366 - DNA, 18 bp, synthetic construct
  source 1..18: mol_type = other DNA
tgcacaactatagcagat

SEQ ID NO: 367 - DNA, 18 bp, synthetic construct
  source 1..18: mol_type = other DNA
tgcacgatattctatgat

SEQ ID NO: 368 - DNA, 18 bp, synthetic construct
  source 1..18: mol_type = other DNA
tgcacgagcagtgccgat

SEQ ID NO: 369 - DNA, 20 bp, synthetic construct
  source 1..20: mol_type = other DNA
tgcactgttgcgctgctgat

SEQ ID NO: 370 - DNA, 20 bp, synthetic construct
  source 1..20: mol_type = other DNA
tgcacatatggcacactgat

SEQ ID NO: 371 - DNA, 18 bp, synthetic construct
  source 1..18: mol_type = other DNA
tgcacagtaccagatgat

SEQ ID NO: 372 - DNA, 20 bp, synthetic construct
  source 1..20: mol_type = other DNA
tgcacgctgccatctgtgat

SEQ ID NO: 373 - DNA, 22 bp, synthetic construct
  source 1..22: mol_type = other DNA
tgcacagctgcatcatgaagat

SEQ ID NO: 374 - DNA, 22 bp, synthetic construct
  source 1..22: mol_type = other DNA
tgcacatgatattcatcatgat

SEQ ID NO: 375 - DNA, 18 bp, synthetic construct
  source 1..18: mol_type = other DNA
tgcacaatacatgtagat

SEQ ID NO: 376 - DNA, 20 bp, synthetic construct
  source 1..20: mol_type = other DNA
tgcactcaatgagatgcgat

SEQ ID NO: 377 - DNA, 20 bp, synthetic construct
  source 1..20: mol_type = other DNA
tgcacagtgaatatgatgat

SEQ ID NO: 378 - DNA, 22 bp, synthetic construct
  source 1..22: mol_type = other DNA
tgcacagatcatgaatcatgat

SEQ ID NO: 379 - DNA, 22 bp, synthetic construct
  source 1..22: mol_type = other DNA
tgcacatcatcatcctgctgat

SEQ ID NO: 380 - DNA, 18 bp, synthetic construct
  source 1..18: mol_type = other DNA
tgcacgcttcgacatgat

SEQ ID NO: 381 - DNA, 18 bp, synthetic construct
  source 1..18: mol_type = other DNA
tgcacagaactgtctgat

SEQ ID NO: 382 - DNA, 22 bp, synthetic construct
  source 1..22: mol_type = other DNA
tgcacttgctgcatgcacagat

SEQ ID NO: 383 - DNA, 22 bp, synthetic construct
  source 1..22: mol_type = other DNA
tgcacatgcagacatgattgat

SEQ ID NO: 384 - DNA, 20 bp, synthetic construct
  source 1..20: mol_type = other DNA
tgcacgctgcattgactgat

SEQ ID NO: 385 - DNA, 20 bp, synthetic construct
  source 1..20: mol_type = other DNA
tgcacgattatatgcatgat

SEQ ID NO: 386 - DNA, 20 bp, synthetic construct
  source 1..20: mol_type = other DNA
tgcactctattgctgatgat

SEQ ID NO: 387 - DNA, 20 bp, synthetic construct
  source 1..20: mol_type = other DNA
tgcacacagccgtgcatgat

SEQ ID NO: 388 - DNA, 20 bp, synthetic construct
  source 1..20: mol_type = other DNA
tgcactgctcctgcgctgat

SEQ ID NO: 389 - DNA, 18 bp, synthetic construct
  source 1..18: mol_type = other DNA
tgcacgctagcacaagat

SEQ ID NO: 390 - DNA, 18 bp, synthetic construct
  source 1..18: mol_type = other DNA
tgcacggcatcgatagat

SEQ ID NO: 391 - DNA, 20 bp, synthetic construct
  source 1..20: mol_type = other DNA
tgcacatgatgctcgccgat

SEQ ID NO: 392 - DNA, 20 bp, synthetic construct
  source 1..20: mol_type = other DNA
tgcactgatatgattcagat

SEQ ID NO: 393 - DNA, 18 bp, synthetic construct
  source 1..18: mol_type = other DNA
tgcacttcgctcacagat

SEQ ID NO: 394 - DNA, 18 bp, synthetic construct
  source 1..18: mol_type = other DNA
tgcacgctaccatatgat

SEQ ID NO: 395 - DNA, 20 bp, synthetic construct
  source 1..20: mol_type = other DNA
tgcacatatagcattgtgat

SEQ ID NO: 396 - DNA, 18 bp, synthetic construct
  source 1..18: mol_type = other DNA
tgcactgccgcgcgtgat

SEQ ID NO: 397 - DNA, 20 bp, synthetic construct
  source 1..20: mol_type = other DNA
tgcactgaatcgatgctgat

SEQ ID NO: 398 - DNA, 22 bp, synthetic construct
  source 1..22: mol_type = other DNA
tgcacatgcagccagcatagat

SEQ ID NO: 399 - DNA, 22 bp, synthetic construct
  source 1..22: mol_type = other DNA
tgcacatggacagcatgatgat

SEQ ID NO: 400 - DNA, 18 bp, synthetic construct
  source 1..18: mol_type = other DNA
tgcacgctatgcgccgat

SEQ ID NO: 401 - DNA, 18 bp, synthetic construct
  source 1..18: mol_type = other DNA
tgcacgtgcacttgcgat

SEQ ID NO: 402 - DNA, 20 bp, synthetic construct
  source 1..20: mol_type = other DNA
tgcacatatggtgatctgat

SEQ ID NO: 403 - DNA, 22 bp, synthetic construct
  source 1..22: mol_type = other DNA
tgcacatgcatgcagaagcgat

SEQ ID NO: 404 - DNA, 18 bp, synthetic construct
  source 1..18: mol_type = other DNA
tgcacaatagacgcagat

SEQ ID NO: 405 - DNA, 20 bp, synthetic construct
  source 1..20: mol_type = other DNA
tgcacatctggagctcagat

SEQ ID NO: 406 - DNA, 18 bp, synthetic construct
  source 1..18: mol_type = other DNA
tgcacttatatgagagat

SEQ ID NO: 407 - DNA, 20 bp, synthetic construct
  source 1..20: mol_type = other DNA
tgcacacacaatcagatgat

SEQ ID NO: 408 - DNA, 22 bp, synthetic construct
  source 1..22: mol_type = other DNA
tgcacatgtctgcatgattgat

SEQ ID NO: 409 - DNA, 20 bp, synthetic construct
  source 1..20: mol_type = other DNA
tgcacatgtgacatgccgat

SEQ ID NO: 410 - DNA, 20 bp, synthetic construct
  source 1..20: mol_type = other DNA
tgcactgcattgatgacgat

SEQ ID NO: 411 - DNA, 20 bp, synthetic construct
  source 1..20: mol_type = other DNA
tgcacacaatgcatgtagat

SEQ ID NO: 412 - DNA, 18 bp, synthetic construct
  source 1..18: mol_type = other DNA
tgcacaagtgagtctgat

SEQ ID NO: 413 - DNA, 20 bp, synthetic construct
  source 1..20: mol_type = other DNA
tgcacatgtctcattctgat

SEQ ID NO: 414 - DNA, 20 bp, synthetic construct
  source 1..20: mol_type = other DNA
tgcacagagccacatgagat

SEQ ID NO: 415 - DNA, 22 bp, synthetic construct
  source 1..22: mol_type = other DNA
tgcactattgatgcatgcagat

SEQ ID NO: 416 - DNA, 22 bp, synthetic construct
  source 1..22: mol_type = other DNA
tgcacagtgccatgcatgtgat

SEQ ID NO: 417 - DNA, 20 bp, synthetic construct
  source 1..20: mol_type = other DNA
tgcacagttgctctgctgat

SEQ ID NO: 418 - DNA, 18 bp, synthetic construct
  source 1..18: mol_type = other DNA
tgcacggcacagtgtgat

SEQ ID NO: 419 - DNA, 20 bp, synthetic construct
  source 1..20: mol_type = other DNA
tgcacttgcacacagctgat

SEQ ID NO: 420 - DNA, 20 bp, synthetic construct
  source 1..20: mol_type = other DNA
tgcacatgcgacacattgat

SEQ ID NO: 421 - DNA, 22 bp, synthetic construct
  source 1..22: mol_type = other DNA
tgcacagtcatgcatggcagat

SEQ ID NO: 422 - DNA, 20 bp, synthetic construct
  source 1..20: mol_type = other DNA
tgcactgaacatgagctgat

SEQ ID NO: 423 - DNA, 22 bp, synthetic construct
  source 1..22: mol_type = other DNA
tgcacagcatatgccatcagat

SEQ ID NO: 424 - DNA, 18 bp, synthetic construct
  source 1..18: mol_type = other DNA
tgcacaatgtacatagat

SEQ ID NO: 425 - DNA, 22 bp, synthetic construct
  source 1..22: mol_type = other DNA
tgcacatcatgtcatggctgat

SEQ ID NO: 426 - DNA, 20 bp, synthetic construct
  source 1..20: mol_type = other DNA
tgcacatcatctgtgccgat

SEQ ID NO: 427 - DNA, 18 bp, synthetic construct
  source 1..18: mol_type = other DNA
tgcacttctctatcagat

SEQ ID NO: 428 - DNA, 22 bp, synthetic construct
  source 1..22: mol_type = other DNA
tgcacatgcttagcatgatgat

SEQ ID NO: 429 - DNA, 20 bp, synthetic construct
  source 1..20: mol_type = other DNA
tgcacgatcatcagattgat

SEQ ID NO: 430 - DNA, 20 bp, synthetic construct
  source 1..20: mol_type = other DNA
tgcactgtgcctatgcagat

SEQ ID NO: 431 - DNA, 22 bp, synthetic construct
  source 1..22: mol_type = other DNA
tgcacagatgcataatgcagat

SEQ ID NO: 432 - DNA, 20 bp, synthetic construct
  source 1..20: mol_type = other DNA
tgcacagccatctcatagat

SEQ ID NO: 433 - DNA, 18 bp, synthetic construct
  source 1..18: mol_type = other DNA
tgcactgcatcgtccgat

SEQ ID NO: 434 - DNA, 20 bp, synthetic construct
  source 1..20: mol_type = other DNA
tgcacaatgatgacagagat

SEQ ID NO: 435 - DNA, 20 bp, synthetic construct
  source 1..20: mol_type = other DNA
tgcactgctagctcattgat

SEQ ID NO: 436 - DNA, 18 bp, synthetic construct
  source 1..18: mol_type = other DNA
tgcacgatacatgaagat

SEQ ID NO: 437 - DNA, 22 bp, synthetic construct
  source 1..22: mol_type = other DNA
tgcactgttgcatcagcatgat

SEQ ID NO: 438 - DNA, 20 bp, synthetic construct
  source 1..20: mol_type = other DNA
tgcacatatcaggcgcagat

SEQ ID NO: 439 - DNA, 20 bp, synthetic construct
  source 1..20: mol_type = other DNA
tgcacatgcgcagaagagat

SEQ ID NO: 440 - DNA, 20 bp, synthetic construct
  source 1..20: mol_type = other DNA
tgcacatggtcatacatgat

SEQ ID NO: 441 - DNA, 22 bp, synthetic construct
  source 1..22: mol_type = other DNA
tgcacacatgcatctcattgat

SEQ ID NO: 442 - DNA, 22 bp, synthetic construct
  source 1..22: mol_type = other DNA
tgcacatcagatgagccatgat

SEQ ID NO: 443 - DNA, 18 bp, synthetic construct
  source 1..18: mol_type = other DNA
tgcacggtgctgtcagat

SEQ ID NO: 444 - DNA, 18 bp, synthetic construct
  source 1..18: mol_type = other DNA
tgcacgcaacagacagat

SEQ ID NO: 445 - DNA, 20 bp, synthetic construct
  source 1..20: mol_type = other DNA
tgcacagctgctcggatgat

SEQ ID NO: 446 - DNA, 20 bp, synthetic construct
  source 1..20: mol_type = other DNA
tgcacatctgatcaagagat

SEQ ID NO: 447 - DNA, 20 bp, synthetic construct
  source 1..20: mol_type = other DNA
tgcacagtgcagctcaagat

SEQ ID NO: 448 - DNA, 18 bp, synthetic construct
  source 1..18: mol_type = other DNA
tgcacgcggtgcactgat

SEQ ID NO: 449 - DNA, 20 bp, synthetic construct
  source 1..20: mol_type = other DNA
tgcacatgatgtgagttgat

SEQ ID NO: 450 - DNA, 20 bp, synthetic construct
  source 1..20: mol_type = other DNA
tgcactgagctgagattgat

SEQ ID NO: 451 - DNA, 18 bp, synthetic construct
  source 1..18: mol_type = other DNA
tgcacgcatgtccacgat

SEQ ID NO: 452 - DNA, 20 bp, synthetic construct
  source 1..20: mol_type = other DNA
tgcacagctgcatagccgat

SEQ ID NO: 453 - DNA, 20 bp, synthetic construct
  source 1..20: mol_type = other DNA
tgcactcagcctgcatagat

SEQ ID NO: 454 - DNA, 20 bp, synthetic construct
  source 1..20: mol_type = other DNA
tgcacacatgatatattgat

SEQ ID NO: 455 - DNA, 18 bp, synthetic construct
  source 1..18: mol_type = other DNA
tgcactctctgaagagat

SEQ ID NO: 456 - DNA, 22 bp, synthetic construct
  source 1..22: mol_type = other DNA
tgcacatggatcatcagcagat

SEQ ID NO: 457 - DNA, 20 bp, synthetic construct
  source 1..20: mol_type = other DNA
tgcacagtgtgccagctgat

SEQ ID NO: 458 - DNA, 18 bp, synthetic construct
  source 1..18: mol_type = other DNA
tgcacatacgcttcagat

SEQ ID NO: 459 - DNA, 20 bp, synthetic construct
  source 1..20: mol_type = other DNA
tgcacactgctgcaagagat

SEQ ID NO: 460 - DNA, 20 bp, synthetic construct
  source 1..20: mol_type = other DNA
tgcacacatgcttatctgat

SEQ ID NO: 461 - DNA, 18 bp, synthetic construct
  source 1..18: mol_type = other DNA
tgcacatccgctgacgat

SEQ ID NO: 462 - DNA, 18 bp, synthetic construct
  source 1..18: mol_type = other DNA
tgcacgcttatctcagat

SEQ ID NO: 463 - DNA, 22 bp, synthetic construct
  source 1..22: mol_type = other DNA
tgcacatgcgcatgaccatgat

SEQ ID NO: 464 - DNA, 20 bp, synthetic construct
  source 1..20: mol_type = other DNA
tgcacatgacattctgcgat

SEQ ID NO: 465 - DNA, 20 bp, synthetic construct
  source 1..20: mol_type = other DNA
tgcacatgtccatgctagat

SEQ ID NO: 466 - DNA, 20 bp, synthetic construct
  source 1..20: mol_type = other DNA
tgcacatcagcttcgcagat

SEQ ID NO: 467 - DNA, 20 bp, synthetic construct
  source 1..20: mol_type = other DNA
tgcactatgctccatctgat

SEQ ID NO: 468 - DNA, 20 bp, synthetic construct
  source 1..20: mol_type = other DNA
tgcacataatcactcatgat

SEQ ID NO: 469 - DNA, 18 bp, synthetic construct
  source 1..18: mol_type = other DNA
tgcactgagctgaccgat

SEQ ID NO: 470 - DNA, 20 bp, synthetic construct
  source 1..20: mol_type = other DNA
tgcacaagagatagcatgat

SEQ ID NO: 471 - DNA, 22 bp, synthetic construct
  source 1..22: mol_type = other DNA
tgcacagaatgatgcgcatgat

SEQ ID NO: 472 - DNA, 18 bp, synthetic construct
  source 1..18: mol_type = other DNA
tgcacagttatgatcgat

SEQ ID NO: 473 - DNA, 20 bp, synthetic construct
  source 1..20: mol_type = other DNA
tgcacagccactctgatgat

SEQ ID NO: 474 - DNA, 20 bp, synthetic construct
  source 1..20: mol_type = other DNA
tgcactgccagactgcagat

SEQ ID NO: 475 - DNA, 18 bp, synthetic construct
  source 1..18: mol_type = other DNA
tgcacatcgcgcgccgat

SEQ ID NO: 476 - DNA, 18 bp, synthetic construct
  source 1..18: mol_type = other DNA
tgcacggagatatatgat

SEQ ID NO: 477 - DNA, 18 bp, synthetic construct
  source 1..18: mol_type = other DNA
tgcacttcgcagtgcgat

SEQ ID NO: 478 - DNA, 20 bp, synthetic construct
  source 1..20: mol_type = other DNA
tgcacgtcatgatggctgat

SEQ ID NO: 479 - DNA, 20 bp, synthetic construct
  source 1..20: mol_type = other DNA
tgcacgtgcatgatattgat

SEQ ID NO: 480 - DNA, 18 bp, synthetic construct
  source 1..18: mol_type = other DNA
tgcacgagcacaactgat

SEQ ID NO: 481 - DNA, 18 bp, synthetic construct
  source 1..18: mol_type = other DNA
tgcacatagtgagaagat

SEQ ID NO: 482 - DNA, 20 bp, synthetic construct
  source 1..20: mol_type = other DNA
tgcactgaatgcacacagat

SEQ ID NO: 483 - DNA, 20 bp, synthetic construct
  source 1..20: mol_type = other DNA
tgcacgctgcaccatctgat

SEQ ID NO: 484 - DNA, 22 bp, synthetic construct
  source 1..22: mol_type = other DNA
tgcacaatgatgcacatgtgat

SEQ ID NO: 485 - DNA, 20 bp, synthetic construct
  source 1..20: mol_type = other DNA
tgcacgatgcattcgctgat

SEQ ID NO: 486 - DNA, 20 bp, synthetic construct
  source 1..20: mol_type = other DNA
tgcacacacactgccatgat

SEQ ID NO: 487 - DNA, 20 bp, synthetic construct
  source 1..20: mol_type = other DNA
tgcactgcgctgcggcagat

SEQ ID NO: 488 - DNA, 18 bp, synthetic construct
  source 1..18: mol_type = other DNA
tgcacaacacagtcagat

SEQ ID NO: 489 - DNA, 18 bp, synthetic construct
  source 1..18: mol_type = other DNA
tgcactctcataagcgat

SEQ ID NO: 490 - DNA, 22 bp, synthetic construct
  source 1..22: mol_type = other DNA
tgcactgatgcatgcaactgat

SEQ ID NO: 491 - DNA, 20 bp, synthetic construct
  source 1..20: mol_type = other DNA
tgcacaatcgctatgcagat

SEQ ID NO: 492 - DNA, 20 bp, synthetic construct
  source 1..20: mol_type = other DNA
tgcacatgtggatgcgcgat

SEQ ID NO: 493 - DNA, 22 bp, synthetic construct
  source 1..22: mol_type = other DNA
tgcacatcgcatcatggatgat

SEQ ID NO: 494 - DNA, 22 bp, synthetic construct
  source 1..22: mol_type = other DNA
tgcacatatgatgcaccatgat

SEQ ID NO: 495 - DNA, 18 bp, synthetic construct
  source 1..18: mol_type = other DNA
tgcacgcaactcagcgat

SEQ ID NO: 496 - DNA, 18 bp, synthetic construct
  source 1..18: mol_type = other DNA
tgcacgcgcggctatgat

SEQ ID NO: 497 - DNA, 18 bp, synthetic construct
  source 1..18: mol_type = other DNA
tgcactagtgcaagcgat

SEQ ID NO: 498 - DNA, 20 bp, synthetic construct
  source 1..20: mol_type = other DNA
tgcacagagttgcacatgat

SEQ ID NO: 499 - DNA, 20 bp, synthetic construct
  source 1..20: mol_type = other DNA
tgcactcatcagtgcaagat

SEQ ID NO: 500 - DNA, 22 bp, synthetic construct
  source 1..22: mol_type = other DNA
tgcacagcatgagaatgatgat

SEQ ID NO: 501 - DNA, 18 bp, synthetic construct
  source 1..18: mol_type = other DNA
tgcacacagacgattgat

SEQ ID NO: 502 - DNA, 18 bp, synthetic construct
  source 1..18: mol_type = other DNA
tgcacgtgcacagttgat

SEQ ID NO: 503 - DNA, 20 bp, synthetic construct
  source 1..20: mol_type = other DNA
tgcacatggtgatgacagat

SEQ ID NO: 504 - DNA, 20 bp, synthetic construct
  source 1..20: mol_type = other DNA
tgcacagagagctgcttgat

SEQ ID NO: 505 - DNA, 18 bp, synthetic construct
  source 1..18: mol_type = other DNA
tgcactgtcagcaccgat

SEQ ID NO: 506 - DNA, 20 bp, synthetic construct
  source 1..20: mol_type = other DNA
tgcactgtgactgccatgat

SEQ ID NO: 507 - DNA, 20 bp, synthetic construct
  source 1..20: mol_type = other DNA
tgcacagatccagatgagat

SEQ ID NO: 508 - DNA, 18 bp, synthetic construct
  source 1..18: mol_type = other DNA
tgcacagagtgtgttgat

SEQ ID NO: 509 - DNA, 20 bp, synthetic construct
  source 1..20: mol_type = other DNA
tgcacatgcagacagccgat

SEQ ID NO: 510 - DNA, 18 bp, synthetic construct
  source 1..18: mol_type = other DNA
tgcacacaagtcactgat

SEQ ID NO: 511 - DNA, 18 bp, synthetic construct
  source 1..18: mol_type = other DNA
tgcactatgacttgcgat

SEQ ID NO: 512 - DNA, 20 bp, synthetic construct
  source 1..20: mol_type = other DNA
tgcactgcactgtcattgat

SEQ ID NO: 513 - DNA, 20 bp, synthetic construct
  source 1..20: mol_type = other DNA
tgcacatccatcgtcatgat

SEQ ID NO: 514 - DNA, 20 bp, synthetic construct
  source 1..20: mol_type = other DNA
tgcacatcacatacattgat

SEQ ID NO: 515 - DNA, 20 bp, synthetic construct
  source 1..20: mol_type = other DNA
tgcactacctgcatgatgat

SEQ ID NO: 516 - DNA, 20 bp, synthetic construct
  source 1..20: mol_type = other DNA
tgcacaatgagcagatcgat

SEQ ID NO: 517 - DNA, 20 bp, synthetic construct
  source 1..20: mol_type = other DNA
tgcacagtattgcacatgat

SEQ ID NO: 518 - DNA, 20 bp, synthetic construct
  source 1..20: mol_type = other DNA
tgcacacataatgacatgat

SEQ ID NO: 519 - DNA, 20 bp, synthetic construct
  source 1..20: mol_type = other DNA
tgcacgatgcctatgctgat

SEQ ID NO: 520 - DNA, 20 bp, synthetic construct
  source 1..20: mol_type = other DNA
tgcacatgcaagtgactgat

SEQ ID NO: 521 - DNA, 20 bp, synthetic construct
  source 1..20: mol_type = other DNA
tgcacacatggtgcacagat

SEQ ID NO: 522 - DNA, 20 bp, synthetic construct
  source 1..20: mol_type = other DNA
tgcacttgctcatcgatgat

SEQ ID NO: 523 - DNA, 19 bp, synthetic construct
  source 1..19: mol_type = other DNA
tgcacaagctgacagagat

SEQ ID NO: 524 - DNA, 20 bp, synthetic construct
  source 1..20: mol_type = other DNA
tgcactgccacagctatgat

SEQ ID NO: 525 - DNA, 20 bp, synthetic construct
  source 1..20: mol_type = other DNA
tgcactcaactgcagcagat

SEQ ID NO: 526 - DNA, 20 bp, synthetic construct
  source 1..20: mol_type = other DNA
tgcacaagcatgtatcagat

SEQ ID NO: 527 - DNA, 20 bp, synthetic construct
  source 1..20: mol_type = other DNA
tgcactattcatatgcagat

SEQ ID NO: 528 - DNA, 20 bp, synthetic construct
  source 1..20: mol_type = other DNA
tgcacacagctcatgaagat

SEQ ID NO: 529 - DNA, 20 bp, synthetic construct
  source 1..20: mol_type = other DNA
tgcacaatgtgagtgatgat

SEQ ID NO: 530 - DNA, 20 bp, synthetic construct
  source 1..20: mol_type = other DNA
tgcacatgaccatctatgat

SEQ ID NO: 531 - DNA, 18 bp, synthetic construct
  source 1..18: mol_type = other DNA
tgcacttagcatatcgat

SEQ ID NO: 532 - DNA, 20 bp, synthetic construct
  source 1..20: mol_type = other DNA
tgcactattgcagcatagat

SEQ ID NO: 533 - DNA, 20 bp, synthetic construct
  source 1..20: mol_type = other DNA
tgcactgcacattcacagat

SEQ ID NO: 534 - DNA, 20 bp, synthetic construct
  source 1..20: mol_type = other DNA
tgcacatggctagcgctgat

SEQ ID NO: 535 - DNA, 22 bp, synthetic construct
  source 1..22: mol_type = other DNA
tgcacagatgcatgctgccgat

SEQ ID NO: 536 - DNA, 20 bp, synthetic construct
  source 1..20: mol_type = other DNA
tgcacatcattcagatagat

SEQ ID NO: 537 - DNA, 20 bp, synthetic construct
  source 1..20: mol_type = other DNA
tgcacttgcatacgcatgat

SEQ ID NO: 538 - DNA, 20 bp, synthetic construct
  source 1..20: mol_type = other DNA
tgcacatccatgagactgat

SEQ ID NO: 539 - DNA, 20 bp, synthetic construct
  source 1..20: mol_type = other DNA
tgcacttatgcagagctgat

SEQ ID NO: 540 - DNA, 20 bp, synthetic construct
  source 1..20: mol_type = other DNA
tgcacatccatgctctcgat

SEQ ID NO: 541 - DNA, 18 bp, synthetic construct
  source 1..18: mol_type = other DNA
tgcacaacgctcagcgat

SEQ ID NO: 542 - DNA, 20 bp, synthetic construct
  source 1..20: mol_type = other DNA
tgcacagacatggcatagat

SEQ ID NO: 543 - DNA, 20 bp, synthetic construct
  source 1..20: mol_type = other DNA
tgcacgctatgttgcatgat

SEQ ID NO: 544 - DNA, 20 bp, synthetic construct
  source 1..20: mol_type = other DNA
tgcacagctgattgctcgat

SEQ ID NO: 545 - DNA, 18 bp, synthetic construct
  source 1..18: mol_type = other DNA
tgcactgtgttgtgtgat

SEQ ID NO: 546 - DNA, 20 bp, synthetic construct
  source 1..20: mol_type = other DNA
tgcactaccatgcatctgat

SEQ ID NO: 547 - DNA, 18 bp, synthetic construct
  source 1..18: mol_type = other DNA
tgcactgatccgctagat

SEQ ID NO: 548 - DNA, 20 bp, synthetic construct
  source 1..20: mol_type = other DNA
tgcactaggatgcagatgat

SEQ ID NO: 549 - DNA, 20 bp, synthetic construct
  source 1..20: mol_type = other DNA
tgcacgcgcaagcgcatgat

SEQ ID NO: 550 - DNA, 18 bp, synthetic construct
  source 1..18: mol_type = other DNA
tgcactctcttgtgtgat

SEQ ID NO: 551 - DNA, 20 bp, synthetic construct
  source 1..20: mol_type = other DNA
tgcactagcacaatgatgat

SEQ ID NO: 552 - DNA, 20 bp, synthetic construct
  source 1..20: mol_type = other DNA
tgcacagagcaggcacagat

SEQ ID NO: 553 - DNA, 19 bp, synthetic construct
  source 1..19: mol_type = other DNA
tgcacaagatgatcgagat

SEQ ID NO: 554 - DNA, 20 bp, synthetic construct
  source 1..20: mol_type = other DNA
tgcacacactgccacatgat

SEQ ID NO: 555 - DNA, 20 bp, synthetic construct
  source 1..20: mol_type = other DNA
tgcacagcctatgcagtgat

SEQ ID NO: 556 - DNA, 20 bp, synthetic construct
  source 1..20: mol_type = other DNA
tgcacatatgtggctgcgat

SEQ ID NO: 557 - DNA, 20 bp, synthetic construct
  source 1..20: mol_type = other DNA
tgcacagatcaggctatgat

SEQ ID NO: 558 - DNA, 22 bp, synthetic construct
  source 1..22: mol_type = other DNA
tgcacatgtcattgcagctgat

SEQ ID NO: 559 - DNA, 20 bp, synthetic construct
  source 1..20: mol_type = other DNA
tgcactgagtgcctgatgat

SEQ ID NO: 560 - DNA, 22 bp, synthetic construct
  source 1..22: mol_type = other DNA
tgcactgcatcaatgctctgat

SEQ ID NO: 561 - DNA, 20 bp, synthetic construct
  source 1..20: mol_type = other DNA
tgcactgctggagagcagat

SEQ ID NO: 562 - DNA, 20 bp, synthetic construct
  source 1..20: mol_type = other DNA
tgcactgcatctcagaagat

SEQ ID NO: 563 - DNA, 20 bp, synthetic construct
  source 1..20: mol_type = other DNA
tgcacatgattatcgatgat

SEQ ID NO: 564 - DNA, 20 bp, synthetic construct
  source 1..20: mol_type = other DNA
tgcacagctctgattgagat

SEQ ID NO: 565 - DNA, 22 bp, synthetic construct
  source 1..22: mol_type = other DNA
tgcactgcatgccagatatgat

SEQ ID NO: 566 - DNA, 22 bp, synthetic construct
  source 1..22: mol_type = other DNA
tgcacagcatgcagctgaagat

SEQ ID NO: 567 - DNA, 18 bp, synthetic construct
  source 1..18: mol_type = other DNA
tgcacgcttcatcacgat

SEQ ID NO: 568 - DNA, 18 bp, synthetic construct
  source 1..18: mol_type = other DNA
tgcacataacgtgatgat

SEQ ID NO: 569 - DNA, 22 bp, synthetic construct
  source 1..22: mol_type = other DNA
tgcacatgcacatgtgattgat

SEQ ID NO: 570 - DNA, 20 bp, synthetic construct
  source 1..20: mol_type = other DNA
tgcacagcatcaacagagat

SEQ ID NO: 571 - DNA, 20 bp, synthetic construct
  source 1..20: mol_type = other DNA
tgcactcagatgcaactgat

SEQ ID NO: 572 - DNA, 20 bp, synthetic construct
  source 1..20: mol_type = other DNA
tgcactgtgcagctgaagat

SEQ ID NO: 573 - DNA, 20 bp, synthetic construct
  source 1..20: mol_type = other DNA
tgcactgcagtatgcttgat

SEQ ID NO: 574 - DNA, 20 bp, synthetic construct
  source 1..20: mol_type = other DNA
tgcacatcgcgctcattgat

SEQ ID NO: 575 - DNA, 22 bp, synthetic construct
  source 1..22: mol_type = other DNA
tgcacagcatcgcatggcagat

SEQ ID NO: 576 - DNA, 18 bp, synthetic construct
  source 1..18: mol_type = other DNA
tgcacaagtcgcatagat

SEQ ID NO: 577 - DNA, 22 bp, synthetic construct
  source 1..22: mol_type = other DNA
tgcacagcatgttgcagatgat

SEQ ID NO: 578 - DNA, 20 bp, synthetic construct
  source 1..20: mol_type = other DNA
tgcacagctccgcatgtgat

SEQ ID NO: 579 - DNA, 18 bp, synthetic construct
  source 1..18: mol_type = other DNA
tgcacgagcgattgtgat

SEQ ID NO: 580 - DNA, 20 bp, synthetic construct
  source 1..20: mol_type = other DNA
tgcacacatgagcttatgat

SEQ ID NO: 581 - DNA, 20 bp, synthetic construct
  source 1..20: mol_type = other DNA
tgcacagtcatggagatgat

SEQ ID NO: 582 - DNA, 22 bp, synthetic construct
  source 1..22: mol_type = other DNA
tgcacatgcagcatgcgccgat

SEQ ID NO: 583 - DNA, 20 bp, synthetic construct
  source 1..20: mol_type = other DNA
tgcacagccgcagcagcgat

SEQ ID NO: 584 - DNA, 18 bp, synthetic construct
  source 1..18: mol_type = other DNA
tgcacggcagcgtcagat

SEQ ID NO: 585 - DNA, 20 bp, synthetic construct
  source 1..20: mol_type = other DNA
tgcacacagagcaccatgat

SEQ ID NO: 586 - DNA, 20 bp, synthetic construct
  source 1..20: mol_type = other DNA
tgcacaactcatgcatagat

SEQ ID NO: 587 - DNA, 20 bp, synthetic construct
  source 1..20: mol_type = other DNA
tgcacatcagtggcgctgat

SEQ ID NO: 588 - DNA, 22 bp, synthetic construct
  source 1..22: mol_type = other DNA
tgcactgcacatgaatgcagat

SEQ ID NO: 589 - DNA, 20 bp, synthetic construct
  source 1..20: mol_type = other DNA
tgcacatgagcttgtgcgat

SEQ ID NO: 590 - DNA, 20 bp, synthetic construct
  source 1..20: mol_type = other DNA
tgcacaatgacagtgctgat

SEQ ID NO: 591 - DNA, 20 bp, synthetic construct
  source 1..20: mol_type = other DNA
tgcacaagtgtcatcatgat

SEQ ID NO: 592 - DNA, 20 bp, synthetic construct
  source 1..20: mol_type = other DNA
tgcacgatgctgcaatcgat

SEQ ID NO: 593 - DNA, 22 bp, synthetic construct
  source 1..22: mol_type = other DNA
tgcacaatgcacatgctcagat

SEQ ID NO: 594 - DNA, 20 bp, synthetic construct
  source 1..20: mol_type = other DNA
tgcacgatgatgtcattgat

SEQ ID NO: 595 - DNA, 20 bp, synthetic construct
  source 1..20: mol_type = other DNA
tgcacgatgcacatattgat

SEQ ID NO: 596 - DNA, 22 bp, synthetic construct
  source 1..22: mol_type = other DNA
tgcacatcatgcgccagctgat

SEQ ID NO: 597 - DNA, 22 bp, synthetic construct
  source 1..22: mol_type = other DNA
tgcacatgcaatgatatctgat

SEQ ID NO: 598 - DNA, 18 bp, synthetic construct
  source 1..18: mol_type = other DNA
tgcacagtcgctgccgat

SEQ ID NO: 599 - DNA, 20 bp, synthetic construct
  source 1..20: mol_type = other DNA
tgcacgcaatcgctcatgat

SEQ ID NO: 600 - DNA, 20 bp, synthetic construct
  source 1..20: mol_type = other DNA
tgcactatgacatccatgat

SEQ ID NO: 601 - DNA, 18 bp, synthetic construct
  source 1..18: mol_type = other DNA
tgcactacagaggcagat

SEQ ID NO: 602 - DNA, 20 bp, synthetic construct
  source 1..20: mol_type = other DNA
tgcacatgaggcgagctgat

SEQ ID NO: 603 - DNA, 18 bp, synthetic construct
  source 1..18: mol_type = other DNA
tgcacaacgcatatcgat

SEQ ID NO: 604 - DNA, 20 bp, synthetic construct
  source 1..20: mol_type = other DNA
tgcactctctgcaccatgat

SEQ ID NO: 605 - DNA, 18 bp, synthetic construct
  source 1..18: mol_type = other DNA
tgcacactcgcggctgat

SEQ ID NO: 606 - DNA, 22 bp, synthetic construct
  source 1..22: mol_type = other DNA
tgcacatctgcacagcattgat

SEQ ID NO: 607 - DNA, 20 bp, synthetic construct
  source 1..20: mol_type = other DNA
tgcacgatgcatgaagtgat

SEQ ID NO: 608 - DNA, 20 bp, synthetic construct
  source 1..20: mol_type = other DNA
tgcacttgcatctgagtgat

SEQ ID NO: 609 - DNA, 20 bp, synthetic construct
  source 1..20: mol_type = other DNA
tgcacatggcacgcacagat

SEQ ID NO: 610 - DNA, 18 bp, synthetic construct
  source 1..18: mol_type = other DNA
tgcacacagctagttgat

SEQ ID NO: 611 - DNA, 18 bp, synthetic construct
  source 1..18: mol_type = other DNA
tgcacacttctatatgat

SEQ ID NO: 612 - DNA, 20 bp, synthetic construct
  source 1..20: mol_type = other DNA
tgcactcataatgtcatgat

SEQ ID NO: 613 - DNA, 20 bp, synthetic construct
  source 1..20: mol_type = other DNA
tgcactgagatgcaatagat

SEQ ID NO: 614 - DNA, 18 bp, synthetic construct
  source 1..18: mol_type = other DNA
tgcacaagatgtagcgat

SEQ ID NO: 615 - DNA, 22 bp, synthetic construct
  source 1..22: mol_type = other DNA
tgcacaatgcatgtcacatgat

SEQ ID NO: 616 - DNA, 20 bp, synthetic construct
  source 1..20: mol_type = other DNA
tgcacaatgtcactgcagat

SEQ ID NO: 617 - DNA, 22 bp, synthetic construct
  source 1..22: mol_type = other DNA
tgcacatgatgctgcacaagat

SEQ ID NO: 618 - DNA, 18 bp, synthetic construct
  source 1..18: mol_type = other DNA
tgcacacaatagagagat

SEQ ID NO: 619 - DNA, 20 bp, synthetic construct
  source 1..20: mol_type = other DNA
tgcactgcatgcgacttgat

SEQ ID NO: 620 - DNA, 20 bp, synthetic construct
  source 1..20: mol_type = other DNA
tgcacatatccacagatgat

SEQ ID NO: 621 - DNA, 22 bp, synthetic construct
  source 1..22: mol_type = other DNA
tgcacatcattgagcagatgat

SEQ ID NO: 622 - DNA, 20 bp, synthetic construct
  source 1..20: mol_type = other DNA
tgcacagtgcgcgccatgat

SEQ ID NO: 623 - DNA, 19 bp, synthetic construct
  source 1..19: mol_type = other DNA
tgcacatcatcttagcgat

SEQ ID NO: 624 - DNA, 18 bp, synthetic construct
  source 1..18: mol_type = other DNA
tgcacttatgtctgtgat

SEQ ID NO: 625 - DNA, 20 bp, synthetic construct
  source 1..20: mol_type = other DNA
tgcactcaatcagcagcgat

SEQ ID NO: 626 - DNA, 20 bp, synthetic construct
  source 1..20: mol_type = other DNA
tgcacacagcatgccgtgat

SEQ ID NO: 627 - DNA, 20 bp, synthetic construct
  source 1..20: mol_type = other DNA
tgcactagcatggctcagat

SEQ ID NO: 628 - DNA, 20 bp, synthetic construct
  source 1..20: mol_type = other DNA
tgcacaatgatcagagtgat

SEQ ID NO: 629 - DNA, 20 bp, synthetic construct
  source 1..20: mol_type = other DNA
tgcactcaagctcagatgat

SEQ ID NO: 630 - DNA, 22 bp, synthetic construct
  source 1..22: mol_type = other DNA
tgcacatggctgctgtgatgat

SEQ ID NO: 631 - DNA, 20 bp, synthetic construct
  source 1..20: mol_type = other DNA
tgcactgcgcactcattgat

SEQ ID NO: 632 - DNA, 18 bp, synthetic construct
  source 1..18: mol_type = other DNA
tgcactacagatattgat

SEQ ID NO: 633 - DNA, 20 bp, synthetic construct
  source 1..20: mol_type = other DNA
tgcacatccgcatgcgagat

SEQ ID NO: 634 - DNA, 20 bp, synthetic construct
  source 1..20: mol_type = other DNA
tgcactgccgcatatcagat

SEQ ID NO: 635 - DNA, 18 bp, synthetic construct
  source 1..18: mol_type = other DNA
tgcacaacagcacacgat

SEQ ID NO: 636 - DNA, 18 bp, synthetic construct
  source 1..18: mol_type = other DNA
tgcacatatctatccgat

SEQ ID NO: 637 - DNA, 18 bp, synthetic construct
  source 1..18: mol_type = other DNA
tgcactgtattctgagat

SEQ ID NO: 638 - DNA, 22 bp, synthetic construct
  source 1..22: mol_type = other DNA
tgcacagagcatgccagctgat

SEQ ID NO: 639 - DNA, 22 bp, synthetic construct
  source 1..22: mol_type = other DNA
tgcacatatcatgaatgatgat

SEQ ID NO: 640 - DNA, 22 bp, synthetic construct
  source 1..22: mol_type = other DNA
tgcacatccatctgctgatgat

SEQ ID NO: 641 - DNA, 20 bp, synthetic construct
  source 1..20: mol_type = other DNA
tgcacatagcaccatgcgat

SEQ ID NO: 642 - DNA, 20 bp, synthetic construct
  source 1..20: mol_type = other DNA
tgcactctgcgccagcagat

SEQ ID NO: 643 - DNA, 20 bp, synthetic construct
  source 1..20: mol_type = other DNA
tgcacagctctgcgcaagat

SEQ ID NO: 644 - DNA, 18 bp, synthetic construct
  source 1..18: mol_type = other DNA
tgcacaacagtctatgat

SEQ ID NO: 645 - DNA, 20 bp, synthetic construct
  source 1..20: mol_type = other DNA
tgcacagcattgtgtgagat

SEQ ID NO: 646 - DNA, 20 bp, synthetic construct
  source 1..20: mol_type = other DNA
tgcacaatcatgagtgagat

SEQ ID NO: 647 - DNA, 20 bp, synthetic construct
  source 1..20: mol_type = other DNA
tgcactctgcatgtcttgat

SEQ ID NO: 648 - DNA, 20 bp, synthetic construct
  source 1..20: mol_type = other DNA
tgcactgctgctcacttgat

SEQ ID NO: 649 - DNA, 20 bp, synthetic construct
  source 1..20: mol_type = other DNA
tgcactgatgtggatgcgat

SEQ ID NO: 650 - DNA, 20 bp, synthetic construct
  source 1..20: mol_type = other DNA
tgcacttgcagatcagtgat

SEQ ID NO: 651 - DNA, 22 bp, synthetic construct
  source 1..22: mol_type = other DNA
tgcacagcgccatcatgctgat

SEQ ID NO: 652 - DNA, 18 bp, synthetic construct
  source 1..18: mol_type = other DNA
tgcactgatacttcagat

SEQ ID NO: 653 - DNA, 20 bp, synthetic construct
  source 1..20: mol_type = other DNA
tgcacgaggctgtgcatgat

SEQ ID NO: 654 - DNA, 20 bp, synthetic construct
  source 1..20: mol_type = other DNA
tgcacagccatgtcgcagat

SEQ ID NO: 655 - DNA, 20 bp, synthetic construct
  source 1..20: mol_type = other DNA
tgcacatgtgcggagatgat

SEQ ID NO: 656 - DNA, 20 bp, synthetic construct
  source 1..20: mol_type = other DNA
tgcacatagcctgcgatgat

SEQ ID NO: 657 - DNA, 20 bp, synthetic construct
  source 1..20: mol_type = other DNA
tgcactgagctccatgagat

SEQ ID NO: 658 - DNA, 20 bp, synthetic construct
  source 1..20: mol_type = other DNA
tgcactcgctctgccatgat

SEQ ID NO: 659 - DNA, 18 bp, synthetic construct
  source 1..18: mol_type = other DNA
tgcacagcattacacgat

SEQ ID NO: 660 - DNA, 20 bp, synthetic construct
  source 1..20: mol_type = other DNA
tgcactattatgatgctgat

SEQ ID NO: 661 - DNA, 20 bp, synthetic construct
  source 1..20: mol_type = other DNA
tgcacgatgttgctgctgat

SEQ ID NO: 662 - DNA, 20 bp, synthetic construct
  source 1..20: mol_type = other DNA
tgcactgagccatgtatgat

SEQ ID NO: 663 - DNA, 22 bp, synthetic construct
  source 1..22: mol_type = other DNA
tgcacatgtgcaatcatatgat

SEQ ID NO: 664 - DNA, 18 bp, synthetic construct
  source 1..18: mol_type = other DNA
tgcacaagatactgtgat

SEQ ID NO: 665 - DNA, 20 bp, synthetic construct
  source 1..20: mol_type = other DNA
tgcacagcgaagcagatgat

SEQ ID NO: 666 - DNA, 20 bp, synthetic construct
  source 1..20: mol_type = other DNA
tgcacatgtaagcagcagat

SEQ ID NO: 667 - DNA, 20 bp, synthetic construct
  source 1..20: mol_type = other DNA
tgcactgttctgcagcagat

SEQ ID NO: 668 - DNA, 18 bp, synthetic construct
  source 1..18: mol_type = other DNA
tgcacatgtacacttgat

SEQ ID NO: 669 - DNA, 20 bp, synthetic construct
  source 1..20: mol_type = other DNA
tgcacagtgcagaggctgat

SEQ ID NO: 670 - DNA, 20 bp, synthetic construct
  source 1..20: mol_type = other DNA
tgcacactgaacatgatgat

SEQ ID NO: 671 - DNA, 20 bp, synthetic construct
  source 1..20: mol_type = other DNA
tgcactgcagcagtcttgat

SEQ ID NO: 672 - DNA, 18 bp, synthetic construct
  source 1..18: mol_type = other DNA
tgcacggagcatagcgat

SEQ ID NO: 673 - DNA, 20 bp, synthetic construct
  source 1..20: mol_type = other DNA
tgcacagtatctgccatgat

SEQ ID NO: 674 - DNA, 20 bp, synthetic construct
  source 1..20: mol_type = other DNA
tgcactgtgatgcaagtgat

SEQ ID NO: 675 - DNA, 22 bp, synthetic construct
  source 1..22: mol_type = other DNA
tgcacatgctgagcatcttgat

SEQ ID NO: 676 - DNA, 22 bp, synthetic construct
  source 1..22: mol_type = other DNA
tgcactgcgcattgcatgagat

SEQ ID NO: 677 - DNA, 18 bp, synthetic construct
  source 1..18: mol_type = other DNA
tgcacgcgagacattgat

SEQ ID NO: 678 - DNA, 20 bp, synthetic construct
  source 1..20: mol_type = other DNA
tgcactatgccatctatgat

SEQ ID NO: 679 - DNA, 18 bp, synthetic construct
  source 1..18: mol_type = other DNA
tgcactgctagagttgat

SEQ ID NO: 680 - DNA, 18 bp, synthetic construct
  source 1..18: mol_type = other DNA
tgcacaagacagcgagat

SEQ ID NO: 681 - DNA, 22 bp, synthetic construct
  source 1..22: mol_type = other DNA
tgcactgccagatgcgcatgat

SEQ ID NO: 682 - DNA, 18 bp, synthetic construct
  source 1..18: mol_type = other DNA
tgcacttgtatatatgat

SEQ ID NO: 683 - DNA, 18 bp, synthetic construct
  source 1..18: mol_type = other DNA
tgcacttcagcgagcgat

SEQ ID NO: 684 - DNA, 18 bp, synthetic construct
  source 1..18: mol_type = other DNA
tgcacaacagactcagat

SEQ ID NO: 685 - DNA, 18 bp, synthetic construct
  source 1..18: mol_type = other DNA
tgcacaagcagctacgat

SEQ ID NO: 686 - DNA, 20 bp, synthetic construct
  source 1..20: mol_type = other DNA
tgcacatggagcatcacgat

SEQ ID NO: 687 - DNA, 20 bp, synthetic construct
  source 1..20: mol_type = other DNA
tgcacgactgcaagcatgat

SEQ ID NO: 688 - DNA, 20 bp, synthetic construct
  source 1..20: mol_type = other DNA
tgcactatgctgcaagtgat

SEQ ID NO: 689 - DNA, 20 bp, synthetic construct
  source 1..20: mol_type = other DNA
tgcactcttatcagcatgat

SEQ ID NO: 690 - DNA, 22 bp, synthetic construct
  source 1..22: mol_type = other DNA
tgcactcaagcatgcatgtgat

SEQ ID NO: 691 - DNA, 20 bp, synthetic construct
  source 1..20: mol_type = other DNA
tgcactcagcatatattgat

SEQ ID NO: 692 - DNA, 18 bp, synthetic construct
  source 1..18: mol_type = other DNA
tgcacagttcatatagat

SEQ ID NO: 693 - DNA, 20 bp, synthetic construct
  source 1..20: mol_type = other DNA
tgcactcatgaggcacagat

SEQ ID NO: 694 - DNA, 19 bp, synthetic construct
  source 1..19: mol_type = other DNA
tgcacgctgcgatccagat

SEQ ID NO: 695 - DNA, 18 bp, synthetic construct
  source 1..18: mol_type = other DNA
tgcacggcgctcatagat

SEQ ID NO: 696 - DNA, 18 bp, synthetic construct
  source 1..18: mol_type = other DNA
tgcacagagagagaagat

SEQ ID NO: 697 - DNA, 20 bp, synthetic construct
  source 1..20: mol_type = other DNA
tgcacgctgtattgcatgat

SEQ ID NO: 698 - DNA, 20 bp, synthetic construct
  source 1..20: mol_type = other DNA
tgcactgcattgcacgagat

SEQ ID NO: 699 - DNA, 20 bp, synthetic construct
  source 1..20: mol_type = other DNA
tgcactcggctgagcatgat

SEQ ID NO: 700 - DNA, 18 bp, synthetic construct
  source 1..18: mol_type = other DNA
tgcactctgccgctcgat

SEQ ID NO: 701 - DNA, 20 bp, synthetic construct
  source 1..20: mol_type = other DNA
tgcacgtcaggcagcatgat

SEQ ID NO: 702 - DNA, 19 bp, synthetic construct
  source 1..19: mol_type = other DNA
tgcacggctgcagtgcgat

SEQ ID NO: 703 - DNA, 18 bp, synthetic construct
  source 1..18: mol_type = other DNA
tgcacaagcgcgtgtgat

SEQ ID NO: 704 - DNA, 18 bp, synthetic construct
  source 1..18: mol_type = other DNA
tgcacggcactacatgat

SEQ ID NO: 705 - DNA, 20 bp, synthetic construct
  source 1..20: mol_type = other DNA
tgcactagcgcagccatgat

SEQ ID NO: 706 - DNA, 18 bp, synthetic construct
  source 1..18: mol_type = other DNA
tgcacttctgtgtgagat

SEQ ID NO: 707 - DNA, 22 bp, synthetic construct
  source 1..22: mol_type = other DNA
tgcacacaagatgcatgatgat

SEQ ID NO: 708 - DNA, 22 bp, synthetic construct
  source 1..22: mol_type = other DNA
tgcacatgctgcattagatgat

SEQ ID NO: 709 - DNA, 20 bp, synthetic construct
  source 1..20: mol_type = other DNA
tgcactgcatattatgtgat

SEQ ID NO: 710 - DNA, 18 bp, synthetic construct
  source 1..18: mol_type = other DNA
tgcacacgagcatccgat

SEQ ID NO: 711 - DNA, 20 bp, synthetic construct
  source 1..20: mol_type = other DNA
tgcacatgatgtcttgagat

SEQ ID NO: 712 - DNA, 22 bp, synthetic construct
  source 1..22: mol_type = other DNA
tgcacagcatgatgtgcttgat

SEQ ID NO: 713 - DNA, 22 bp, synthetic construct
  source 1..22: mol_type = other DNA
tgcacatgcttgcagatgtgat

SEQ ID NO: 714 - DNA, 20 bp, synthetic construct
  source 1..20: mol_type = other DNA
tgcactatcacatggctgat

SEQ ID NO: 715 - DNA, 20 bp, synthetic construct
  source 1..20: mol_type = other DNA
tgcacttgcgcagagctgat

SEQ ID NO: 716 - DNA, 20 bp, synthetic construct
  source 1..20: mol_type = other DNA
tgcacagaatcgcagctgat

SEQ ID NO: 717 - DNA, 18 bp, synthetic construct
  source 1..18: mol_type = other DNA
tgcacataagagagcgat

SEQ ID NO: 718 - DNA, 19 bp, synthetic construct
  source 1..19: mol_type = other DNA
tgcacatcaggtcagagat

SEQ ID NO: 719 - DNA, 20 bp, synthetic construct
  source 1..20: mol_type = other DNA
tgcacaatctctcgcatgat

SEQ ID NO: 720 - DNA, 22 bp, synthetic construct
  source 1..22: mol_type = other DNA
tgcacatgcatcatatcaagat

SEQ ID NO: 721 - DNA, 22 bp, synthetic construct
  source 1..22: mol_type = other DNA
tgcacactgccatgcatctgat

SEQ ID NO: 722 - DNA, 20 bp, synthetic construct
  source 1..20: mol_type = other DNA
tgcactcggcagcagatgat

SEQ ID NO: 723 - DNA, 18 bp, synthetic construct
  source 1..18: mol_type = other DNA
tgcacttgagcgatagat

SEQ ID NO: 724 - DNA, 20 bp, synthetic construct
  source 1..20: mol_type = other DNA
tgcactatagcagccatgat

SEQ ID NO: 725 - DNA, 20 bp, synthetic construct
  source 1..20: mol_type = other DNA
tgcacgcgcatgctgccgat

SEQ ID NO: 726 - DNA, 22 bp, synthetic construct
  source 1..22: mol_type = other DNA
tgcacatcatatcatgcaagat

SEQ ID NO: 727 - DNA, 18 bp, synthetic construct
  source 1..18: mol_type = other DNA
tgcactgtagcgattgat

SEQ ID NO: 728 - DNA, 20 bp, synthetic construct
  source 1..20: mol_type = other DNA
tgcactcgattgcatatgat

SEQ ID NO: 729 - DNA, 18 bp, synthetic construct
  source 1..18: mol_type = other DNA
tgcacagagcgctccgat

SEQ ID NO: 730 - DNA, 20 bp, synthetic construct
  source 1..20: mol_type = other DNA
tgcacgcatcctatgcagat

SEQ ID NO: 731 - DNA, 18 bp, synthetic construct
  source 1..18: mol_type = other DNA
tgcacgcatataatagat

SEQ ID NO: 732 - DNA, 20 bp, synthetic construct
  source 1..20: mol_type = other DNA
tgcacagaatgtgctatgat

SEQ ID NO: 733 - DNA, 20 bp, synthetic construct
  source 1..20: mol_type = other DNA
tgcacagcgatcattatgat

SEQ ID NO: 734 - DNA, 18 bp, synthetic construct
  source 1..18: mol_type = other DNA
tgcactcgcacataagat

SEQ ID NO: 735 - DNA, 20 bp, synthetic construct
  source 1..20: mol_type = other DNA
tgcacactgctggtgctgat

SEQ ID NO: 736 - DNA, 18 bp, synthetic construct
  source 1..18: mol_type = other DNA
tgcacggtgatcactgat

SEQ ID NO: 737 - DNA, 20 bp, synthetic construct
  source 1..20: mol_type = other DNA
tgcacatagagcctgatgat

SEQ ID NO: 738 - DNA, 20 bp, synthetic construct
  source 1..20: mol_type = other DNA
tgcactatcatggacatgat

SEQ ID NO: 739 - DNA, 20 bp, synthetic construct
  source 1..20: mol_type = other DNA
tgcacaatcatctgactgat

SEQ ID NO: 740 - DNA, 18 bp, synthetic construct
  source 1..18: mol_type = other DNA
tgcacatccagcgtcgat

SEQ ID NO: 741 - DNA, 22 bp, synthetic construct
  source 1..22: mol_type = other DNA
tgcacatgcagcattctgtgat

SEQ ID NO: 742 - DNA, 18 bp, synthetic construct
  source 1..18: mol_type = other DNA
tgcacagttgagcacgat

SEQ ID NO: 743 - DNA, 20 bp, synthetic construct
  source 1..20: mol_type = other DNA
tgcacacggcatcgcatgat

SEQ ID NO: 744 - DNA, 20 bp, synthetic construct
  source 1..20: mol_type = other DNA
tgcacatcacaggctctgat

SEQ ID NO: 745 - DNA, 20 bp, synthetic construct
  source 1..20: mol_type = other DNA
tgcacttcatgctgcacgat

SEQ ID NO: 746 - DNA, 20 bp, synthetic construct
  source 1..20: mol_type = other DNA
tgcacaatgcgctgtcagat

SEQ ID NO: 747 - DNA, 22 bp, synthetic construct
  source 1..22: mol_type = other DNA
tgcactgctgtgcaagcatgat

SEQ ID NO: 748 - DNA, 20 bp, synthetic construct
  source 1..20: mol_type = other DNA
tgcacagatggctacatgat

SEQ ID NO: 749 - DNA, 20 bp, synthetic construct
  source 1..20: mol_type = other DNA
tgcactgctcacaccatgat

SEQ ID NO: 750 - DNA, 18 bp, synthetic construct
  source 1..18: mol_type = other DNA
tgcactgtcaacagagat

SEQ ID NO: 751 - DNA, 20 bp, synthetic construct
  source 1..20: mol_type = other DNA
tgcacagttgcatcacagat

SEQ ID NO: 752 - DNA, 20 bp, synthetic construct
  source 1..20: mol_type = other DNA
tgcacgacattgctcatgat

SEQ ID NO: 753 - DNA, 19 bp, synthetic construct
  source 1..19: mol_type = other DNA
tgcacatctcaggacagat

SEQ ID NO: 754 - DNA, 20 bp, synthetic construct
  source 1..20: mol_type = other DNA
tgcacacaatatctgatgat

SEQ ID NO: 755 - DNA, 18 bp, synthetic construct
  source 1..18: mol_type = other DNA
tgcactatctgaagcgat

SEQ ID NO: 756 - DNA, 18 bp, synthetic construct
  source 1..18: mol_type = other DNA
tgcacggatctgtgtgat

SEQ ID NO: 757 - DNA, 18 bp, synthetic construct
  source 1..18: mol_type = other DNA
tgcactatgttgctagat

SEQ ID NO: 758 - DNA, 20 bp, synthetic construct
  source 1..20: mol_type = other DNA
tgcacggcatgagagctgat

SEQ ID NO: 759 - DNA, 18 bp, synthetic construct
  source 1..18: mol_type = other DNA
tgcacagaagatcgcgat

SEQ ID NO: 760 - DNA, 20 bp, synthetic construct
  source 1..20: mol_type = other DNA
tgcactgagtgcattctgat

SEQ ID NO: 761 - DNA, 18 bp, synthetic construct
  source 1..18: mol_type = other DNA
tgcacacttgacactgat

SEQ ID NO: 762 - DNA, 22 bp, synthetic construct
  source 1..22: mol_type = other DNA
tgcacataatgcatgctgtgat

SEQ ID NO: 763 - DNA, 18 bp, synthetic construct
  source 1..18: mol_type = other DNA
tgcacagtcttgtatgat

SEQ ID NO: 764 - DNA, 18 bp, synthetic construct
  source 1..18: mol_type = other DNA
tgcacgcacgcgcaagat

SEQ ID NO: 765 - DNA, 18 bp, synthetic construct
  source 1..18: mol_type = other DNA
tgcacatccgatacagat

SEQ ID NO: 766 - DNA, 20 bp, synthetic construct
  source 1..20: mol_type = other DNA
tgcactgcctctgatctgat

SEQ ID NO: 767 - DNA, 18 bp, synthetic construct
  source 1..18: mol_type = other DNA
tgcacggctgctatcgat

SEQ ID NO: 768 - DNA, 18 bp, synthetic construct
  source 1..18: mol_type = other DNA
tgcactgttgtcagcgat

SEQ ID NO: 769 - DNA, 18 bp, synthetic construct
  source 1..18: mol_type = other DNA
tgcacacgagcttgagat

SEQ ID NO: 770 - DNA, 20 bp, synthetic construct
  source 1..20: mol_type = other DNA
tgcactcactattgcatgat

SEQ ID NO: 771 - DNA, 18 bp, synthetic construct
  source 1..18: mol_type = other DNA
tgcacactagctgttgat

SEQ ID NO: 772 - DNA, 22 bp, synthetic construct
  source 1..22: mol_type = other DNA
tgcacagcatcataagcatgat

SEQ ID NO: 773 - DNA, 20 bp, synthetic construct
  source 1..20: mol_type = other DNA
tgcacatcgtcaagcatgat

SEQ ID NO: 774 - DNA, 20 bp, synthetic construct
  source 1..20: mol_type = other DNA
tgcacagctggagcatagat

SEQ ID NO: 775 - DNA, 20 bp, synthetic construct
  source 1..20: mol_type = other DNA
tgcacagatgcaagcgtgat

SEQ ID NO: 776 - DNA, 18 bp, synthetic construct
  source 1..18: mol_type = other DNA
tgcacagtgttgagagat

SEQ ID NO: 777 - DNA, 20 bp, synthetic construct
  source 1..20: mol_type = other DNA
tgcactgctcagatgccgat

SEQ ID NO: 778 - DNA, 20 bp, synthetic construct
  source 1..20: mol_type = other DNA
tgcacgaggcatgatcagat

SEQ ID NO: 779 - DNA, 20 bp, synthetic construct
  source 1..20: mol_type = other DNA
tgcacgcgcgcaatgctgat

SEQ ID NO: 780 - DNA, 22 bp, synthetic construct
  source 1..22: mol_type = other DNA
tgcacatgagcactgccatgat

SEQ ID NO: 781 - DNA, 19 bp, synthetic construct
  source 1..19: mol_type = other DNA
tgcactgcgccatcgagat

SEQ ID NO: 782 - DNA, 18 bp, synthetic construct
  source 1..18: mol_type = other DNA
tgcacttcacatagtgat

SEQ ID NO: 783 - DNA, 18 bp, synthetic construct
  source 1..18: mol_type = other DNA
tgcacacagagaagtgat

SEQ ID NO: 784 - DNA, 18 bp, synthetic construct
  source 1..18: mol_type = other DNA
tgcactcaacgatcagat

SEQ ID NO: 785 - DNA, 20 bp, synthetic construct
  source 1..20: mol_type = other DNA
tgcactgcgatggagatgat

SEQ ID NO: 786 - DNA, 20 bp, synthetic construct
  source 1..20: mol_type = other DNA
tgcacatctcatctattgat

SEQ ID NO: 787 - DNA, 22 bp, synthetic construct
  source 1..22: mol_type = other DNA
tgcacacatgatgatgcaagat

SEQ ID NO: 788 - DNA, 20 bp, synthetic construct
  source 1..20: mol_type = other DNA
tgcacactgatgtggatgat

SEQ ID NO: 789 - DNA, 18 bp, synthetic construct
  source 1..18: mol_type = other DNA
tgcactgacggcagcgat

SEQ ID NO: 790 - DNA, 18 bp, synthetic construct
  source 1..18: mol_type = other DNA
tgcacatacggacatgat

SEQ ID NO: 791 - DNA, 20 bp, synthetic construct
  source 1..20: mol_type = other DNA
tgcactctatgccatcagat

SEQ ID NO: 792 - DNA, 20 bp, synthetic construct
  source 1..20: mol_type = other DNA
tgcacttgctctagcatgat

SEQ ID NO: 793 - DNA, 18 bp, synthetic construct
  source 1..18: mol_type = other DNA
tgcacgtcatgttgagat

SEQ ID NO: 794 - DNA, 20 bp, synthetic construct
  source 1..20: mol_type = other DNA
tgcacatatgatcttcagat

SEQ ID NO: 795 - DNA, 18 bp, synthetic construct
  source 1..18: mol_type = other DNA
tgcacgcttacagctgat

SEQ ID NO: 796 - DNA, 18 bp, synthetic construct
  source 1..18: mol_type = other DNA
tgcacaatgcacgtagat

SEQ ID NO: 797 - DNA, 20 bp, synthetic construct
  source 1..20: mol_type = other DNA
tgcacatgatcaatgacgat

SEQ ID NO: 798 - DNA, 20 bp, synthetic construct
  source 1..20: mol_type = other DNA
tgcacactcggatgcatgat

SEQ ID NO: 799 - DNA, 20 bp, synthetic construct
  source 1..20: mol_type = other DNA
tgcacatggcgctgagtgat

SEQ ID NO: 800 - DNA, 22 bp, synthetic construct
  source 1..22: mol_type = other DNA
tgcacatgcatggatctgcgat

SEQ ID NO: 801 - DNA, 18 bp, synthetic construct
  source 1..18: mol_type = other DNA
tgcacaatgtcgtgagat

SEQ ID NO: 802 - DNA, 20 bp, synthetic construct
  source 1..20: mol_type = other DNA
tgcactgcaccgcatctgat

SEQ ID NO: 803 - DNA, 20 bp, synthetic construct
  source 1..20: mol_type = other DNA
tgcactgcagataccatgat

SEQ ID NO: 804 - DNA, 18 bp, synthetic construct
  source 1..18: mol_type = other DNA
tgcacagtcgaggcagat

SEQ ID NO: 805 - DNA, 20 bp, synthetic construct
  source 1..20: mol_type = other DNA
tgcactatgcctatgatgat

SEQ ID NO: 806 - DNA, 20 bp, synthetic construct
  source 1..20: mol_type = other DNA
tgcacttcagcatctgagat

SEQ ID NO: 807 - DNA, 22 bp, synthetic construct
  source 1..22: mol_type = other DNA
tgcacatatgcatcgccatgat

SEQ ID NO: 808 - DNA, 20 bp, synthetic construct
  source 1..20: mol_type = other DNA
tgcacagcacctgcagagat

SEQ ID NO: 809 - DNA, 20 bp, synthetic construct
  source 1..20: mol_type = other DNA
tgcacttgatcatatctgat

SEQ ID NO: 810 - DNA, 18 bp, synthetic construct
  source 1..18: mol_type = other DNA
tgcactatcaatcacgat

SEQ ID NO: 811 - DNA, 20 bp, synthetic construct
  source 1..20: mol_type = other DNA
tgcacacatctgagattgat

SEQ ID NO: 812 - DNA, 20 bp, synthetic construct
  source 1..20: mol_type = other DNA
tgcactctcaagcatatgat

SEQ ID NO: 813 - DNA, 20 bp, synthetic construct
  source 1..20: mol_type = other DNA
tgcacatcacagcttgagat

SEQ ID NO: 814 - DNA, 18 bp, synthetic construct
  source 1..18: mol_type = other DNA
tgcacgatgacaagcgat

SEQ ID NO: 815 - DNA, 18 bp, synthetic construct
  source 1..18: mol_type = other DNA
tgcacggctcagctcgat

SEQ ID NO: 816 - DNA, 20 bp, synthetic construct
  source 1..20: mol_type = other DNA
tgcacacatccacatgcgat

SEQ ID NO: 817 - DNA, 22 bp, synthetic construct
  source 1..22: mol_type = other DNA
tgcacaatgagatgctgcagat

SEQ ID NO: 818 - DNA, 20 bp, synthetic construct
  source 1..20: mol_type = other DNA
tgcacggctgatcgcatgat

SEQ ID NO: 819 - DNA, 20 bp, synthetic construct
  source 1..20: mol_type = other DNA
tgcacgctgtgctcattgat

SEQ ID NO: 820 - DNA, 20 bp, synthetic construct
  source 1..20: mol_type = other DNA
tgcacactcatgtggcagat

SEQ ID NO: 821 - DNA, 18 bp, synthetic construct
  source 1..18: mol_type = other DNA
tgcacttgtgagatagat

SEQ ID NO: 822 - DNA, 18 bp, synthetic construct
  source 1..18: mol_type = other DNA
tgcacacttgtagatgat

SEQ ID NO: 823 - DNA, 20 bp, synthetic construct
  source 1..20: mol_type = other DNA
tgcacaacatgatgcgagat

SEQ ID NO: 824 - DNA, 18 bp, synthetic construct
  source 1..18: mol_type = other DNA
tgcacacaactgtatgat

SEQ ID NO: 825 - DNA, 20 bp, synthetic construct
  source 1..20: mol_type = other DNA
tgcacaatatgtcatatgat

SEQ ID NO: 826 - DNA, 20 bp, synthetic construct
  source 1..20: mol_type = other DNA
tgcacaatcatatgcacgat

SEQ ID NO: 827 - DNA, 20 bp, synthetic construct
  source 1..20: mol_type = other DNA
tgcacatcatctcaacagat

SEQ ID NO: 828 - DNA, 20 bp, synthetic construct
  source 1..20: mol_type = other DNA
tgcacaacatacagcatgat

SEQ ID NO: 829 - DNA, 20 bp, synthetic construct
  source 1..20: mol_type = other DNA
tgcactcatggagctctgat

SEQ ID NO: 830 - DNA, 20 bp, synthetic construct
  source 1..20: mol_type = other DNA
tgcactctgcatcggctgat

SEQ ID NO: 831 - DNA, 18 bp, synthetic construct
  source 1..18: mol_type = other DNA
tgcacaatatacgctgat

SEQ ID NO: 832 - DNA, 18 bp, synthetic construct
  source 1..18: mol_type = other DNA
tgcacgctgcgaagagat

SEQ ID NO: 833 - DNA, 18 bp, synthetic construct
  source 1..18: mol_type = other DNA
tgcacagagacaagtgat

SEQ ID NO: 834 - DNA, 20 bp, synthetic construct
  source 1..20: mol_type = other DNA
tgcactctcatcattgtgat

SEQ ID NO: 835 - DNA, 18 bp, synthetic construct
  source 1..18: mol_type = other DNA
tgcactcttgctcgagat

SEQ ID NO: 836 - DNA, 19 bp, synthetic construct
  source 1..19: mol_type = other DNA
tgcacttgatcgagatgat

SEQ ID NO: 837 - DNA, 18 bp, synthetic construct
  source 1..18: mol_type = other DNA
tgcacatggtagctcgat

SEQ ID NO: 838 - DNA, 22 bp, synthetic construct
  source 1..22: mol_type = other DNA
tgcacatgtgcatggatgcgat

SEQ ID NO: 839 - DNA, 20 bp, synthetic construct
  source 1..20: mol_type = other DNA
tgcactgcagcttgtgtgat

SEQ ID NO: 840 - DNA, 22 bp, synthetic construct
  source 1..22: mol_type = other DNA
tgcacatatcgccatgcatgat

SEQ ID NO: 841 - DNA, 20 bp, synthetic construct
  source 1..20: mol_type = other DNA
tgcacgaccatcatgcagat

SEQ ID NO: 842 - DNA, 22 bp, synthetic construct
  source 1..22: mol_type = other DNA
tgcacatcatgccagcatcgat

SEQ ID NO: 843 - DNA, 22 bp, synthetic construct
  source 1..22: mol_type = other DNA
tgcactgatcagcatccatgat

SEQ ID NO: 844 - DNA, 18 bp, synthetic construct
  source 1..18: mol_type = other DNA
tgcacgagcgctgaagat

SEQ ID NO: 845 - DNA, 20 bp, synthetic construct
  source 1..20: mol_type = other DNA
tgcacatgctgtagcaagat

SEQ ID NO: 846 - DNA, 20 bp, synthetic construct
  source 1..20: mol_type = other DNA
tgcacttcagagatcatgat

SEQ ID NO: 847 - DNA, 20 bp, synthetic construct
  source 1..20: mol_type = other DNA
tgcacacgattgctcatgat

SEQ ID NO: 848 - DNA, 20 bp, synthetic construct
  source 1..20: mol_type = other DNA
tgcacatctatgaggatgat

SEQ ID NO: 849 - DNA, 20 bp, synthetic construct
  source 1..20: mol_type = other DNA
tgcacgcaatcactgcagat

SEQ ID NO: 850 - DNA, 18 bp, synthetic construct
  source 1..18: mol_type = other DNA
tgcacaatcgagcgtgat

SEQ ID NO: 851 - DNA, 20 bp, synthetic construct
  source 1..20: mol_type = other DNA
tgcactcacagatggctgat

SEQ ID NO: 852 - DNA, 20 bp, synthetic construct
  source 1..20: mol_type = other DNA
tgcactgtatgagccatgat

SEQ ID NO: 853 - DNA, 22 bp, synthetic construct
  source 1..22: mol_type = other DNA
tgcacacatgcagagccatgat

SEQ ID NO: 854 - DNA, 22 bp, synthetic construct
  source 1..22: mol_type = other DNA
tgcacataacatgcatgcagat

SEQ ID NO: 855 - DNA, 18 bp, synthetic construct
  source 1..18: mol_type = other DNA
tgcacttgctgacgcgat

SEQ ID NO: 856 - DNA, 20 bp, synthetic construct
  source 1..20: mol_type = other DNA
tgcactgacaatgcagagat

SEQ ID NO: 857 - DNA, 18 bp, synthetic construct
  source 1..18: mol_type = other DNA
tgcacgtgcttgcgcgat

SEQ ID NO: 858 - DNA, 20 bp, synthetic construct
  source 1..20: mol_type = other DNA
tgcacagctacatggcagat

SEQ ID NO: 859 - DNA, 22 bp, synthetic construct
  source 1..22: mol_type = other DNA
tgcacatgctatgcctgcagat

SEQ ID NO: 860 - DNA, 20 bp, synthetic construct
  source 1..20: mol_type = other DNA
tgcacacaatgtgctctgat

SEQ ID NO: 861 - DNA, 18 bp, synthetic construct
  source 1..18: mol_type = other DNA
tgcacttgtgtctatgat

SEQ ID NO: 862 - DNA, 18 bp, synthetic construct
  source 1..18: mol_type = other DNA
tgcactgcactaatcgat

SEQ ID NO: 863 - DNA, 18 bp, synthetic construct
  source 1..18: mol_type = other DNA
tgcacgatgatatccgat

SEQ ID NO: 864 - DNA, 20 bp, synthetic construct
  source 1..20: mol_type = other DNA
tgcacacataagatgctgat

SEQ ID NO: 865 - DNA, 20 bp, synthetic construct
  source 1..20: mol_type = other DNA
tgcactgcagatatgaagat

SEQ ID NO: 866 - DNA, 20 bp, synthetic construct
  source 1..20: mol_type = other DNA
tgcacagcgcgccacatgat

SEQ ID NO: 867 - DNA, 22 bp, synthetic construct
  source 1..22: mol_type = other DNA
tgcacataatgctgagcatgat

SEQ ID NO: 868 - DNA, 20 bp, synthetic construct
  source 1..20: mol_type = other DNA
tgcactcatcattgcgagat

SEQ ID NO: 869 - DNA, 20 bp, synthetic construct
  source 1..20: mol_type = other DNA
tgcacagagctatgattgat

SEQ ID NO: 870 - DNA, 18 bp, synthetic construct
  source 1..18: mol_type = other DNA
tgcacttctgctagtgat

SEQ ID NO: 871 - DNA, 18 bp, synthetic construct
  source 1..18: mol_type = other DNA
tgcacggtgtcatgtgat

SEQ ID NO: 872 - DNA, 18 bp, synthetic construct
  source 1..18: mol_type = other DNA
tgcactcactgtgaagat

SEQ ID NO: 873 - DNA, 20 bp, synthetic construct
  source 1..20: mol_type = other DNA
tgcacttcgatatgcatgat

SEQ ID NO: 874 - DNA, 20 bp, synthetic construct
  source 1..20: mol_type = other DNA
tgcactcatccatcatagat

SEQ ID NO: 875 - DNA, 18 bp, synthetic construct
  source 1..18: mol_type = other DNA
tgcacatagtgttcagat

SEQ ID NO: 876 - DNA, 20 bp, synthetic construct
  source 1..20: mol_type = other DNA
tgcacatgattgagctagat

SEQ ID NO: 877 - DNA, 22 bp, synthetic construct
  source 1..22: mol_type = other DNA
tgcacatcatgcgcattatgat

SEQ ID NO: 878 - DNA, 20 bp, synthetic construct
  source 1..20: mol_type = other DNA
tgcacatgtatgtccatgat

SEQ ID NO: 879 - DNA, 22 bp, synthetic construct
  source 1..22: mol_type = other DNA
tgcactgccatctgcatatgat

SEQ ID NO: 880 - DNA, 19 bp, synthetic construct
  source 1..19: mol_type = other DNA
tgcactgactcaagatgat

SEQ ID NO: 881 - DNA, 20 bp, synthetic construct
  source 1..20: mol_type = other DNA
tgcacaagctatgctatgat

SEQ ID NO: 882 - DNA, 18 bp, synthetic construct
  source 1..18: mol_type = other DNA
tgcacaagactcgctgat

SEQ ID NO: 883 - DNA, 20 bp, synthetic construct
  source 1..20: mol_type = other DNA
tgcactctgcgcattgtgat

SEQ ID NO: 884 - DNA, 18 bp, synthetic construct
  source 1..18: mol_type = other DNA
tgcacggtcactgatgat

SEQ ID NO: 885 - DNA, 22 bp, synthetic construct
  source 1..22: mol_type = other DNA
tgcacatctcatattgcatgat

SEQ ID NO: 886 - DNA, 20 bp, synthetic construct
  source 1..20: mol_type = other DNA
tgcacgcatgactcattgat

SEQ ID NO: 887 - DNA, 20 bp, synthetic construct
  source 1..20: mol_type = other DNA
tgcactgatggtcatcagat

SEQ ID NO: 888 - DNA, 20 bp, synthetic construct
  source 1..20: mol_type = other DNA
tgcactctgcattacatgat

SEQ ID NO: 889 - DNA, 20 bp, synthetic construct
  source 1..20: mol_type = other DNA
tgcacacacatgcggcagat

SEQ ID NO: 890 - DNA, 20 bp, synthetic construct
  source 1..20: mol_type = other DNA
tgcacgattgtgatgatgat

SEQ ID NO: 891 - DNA, 20 bp, synthetic construct
  source 1..20: mol_type = other DNA
tgcacagatgcatctaagat

SEQ ID NO: 892 - DNA, 20 bp, synthetic construct
  source 1..20: mol_type = other DNA
tgcacagcagatcttgagat

SEQ ID NO: 893 - DNA, 22 bp, synthetic construct
  source 1..22: mol_type = other DNA
tgcactctgcatgatgattgat

SEQ ID NO: 894 - DNA, 18 bp, synthetic construct
  source 1..18: mol_type = other DNA
tgcacgtgcggatgcgat

SEQ ID NO: 895 - DNA, 20 bp, synthetic construct
  source 1..20: mol_type = other DNA
tgcacgctctcaatgatgat

SEQ ID NO: 896 - DNA, 20 bp, synthetic construct
  source 1..20: mol_type = other DNA
tgcactgatctcattgtgat

SEQ ID NO: 897 - DNA, 22 bp, synthetic construct
  source 1..22: mol_type = other DNA
tgcacatgtggcagatcatgat

SEQ ID NO: 898 - DNA, 20 bp, synthetic construct
  source 1..20: mol_type = other DNA
tgcacaagctcagatctgat

SEQ ID NO: 899 - DNA, 22 bp, synthetic construct
  source 1..22: mol_type = other DNA
tgcactgctgcacatgattgat

SEQ ID NO: 900 - DNA, 20 bp, synthetic construct
  source 1..20: mol_type = other DNA
tgcacatggcatgtgtcgat

SEQ ID NO: 901 - DNA, 20 bp, synthetic construct
  source 1..20: mol_type = other DNA
tgcacaatgtgctgatagat

SEQ ID NO: 902 - DNA, 20 bp, synthetic construct
  source 1..20: mol_type = other DNA
tgcacatagcagcgcttgat

SEQ ID NO: 903 - DNA, 20 bp, synthetic construct
  source 1..20: mol_type = other DNA
tgcacatcgtgcatgaagat

SEQ ID NO: 904 - DNA, 18 bp, synthetic construct
  source 1..18: mol_type = other DNA
tgcacggatagcagtgat

SEQ ID NO: 905 - DNA, 20 bp, synthetic construct
  source 1..20: mol_type = other DNA
tgcactcaatgcacagtgat

SEQ ID NO: 906 - DNA, 20 bp, synthetic construct
  source 1..20: mol_type = other DNA
tgcactcatgtctgcaagat

SEQ ID NO: 907 - DNA, 20 bp, synthetic construct
  source 1..20: mol_type = other DNA
tgcacatcgcatcttctgat

SEQ ID NO: 908 - DNA, 20 bp, synthetic construct
  source 1..20: mol_type = other DNA
tgcacatccactcatgtgat

SEQ ID NO: 909 - DNA, 20 bp, synthetic construct
  source 1..20: mol_type = other DNA
tgcactagcatagcattgat

SEQ ID NO: 910 - DNA, 18 bp, synthetic construct
  source 1..18: mol_type = other DNA
tgcacttatcgcacagat

SEQ ID NO: 911 - DNA, 18 bp, synthetic construct
  source 1..18: mol_type = other DNA
tgcacagcgccgcgagat

SEQ ID NO: 912 - DNA, 20 bp, synthetic construct
  source 1..20: mol_type = other DNA
tgcacagtcacatccatgat

SEQ ID NO: 913 - DNA, 20 bp, synthetic construct
  source 1..20: mol_type = other DNA
tgcacatacatcagcttgat

SEQ ID NO: 914 - DNA, 18 bp, synthetic construct
  source 1..18: mol_type = other DNA
tgcacaacatgtcgtgat

SEQ ID NO: 915 - DNA, 20 bp, synthetic construct
  source 1..20: mol_type = other DNA
tgcacatgtctgtgcttgat

SEQ ID NO: 916 - DNA, 20 bp, synthetic construct
  source 1..20: mol_type = other DNA
tgcacactcatgagcttgat

SEQ ID NO: 917 - DNA, 18 bp, synthetic construct
  source 1..18: mol_type = other DNA
tgcactcggatgcgcgat

SEQ ID NO: 918 - DNA, 20 bp, synthetic construct
  source 1..20: mol_type = other DNA
tgcacatgtataatgctgat

SEQ ID NO: 919 - DNA, 20 bp, synthetic construct
  source 1..20: mol_type = other DNA
tgcacagctccatatgtgat

SEQ ID NO: 920 - DNA, 20 bp, synthetic construct
  source 1..20: mol_type = other DNA
tgcacttatgatcagctgat

SEQ ID NO: 921 - DNA, 22 bp, synthetic construct
  source 1..22: mol_type = other DNA
tgcacatatggcatgcagcgat

SEQ ID NO: 922 - DNA, 20 bp, synthetic construct
  source 1..20: mol_type = other DNA
tgcacagagaatcatcagat

SEQ ID NO: 923 - DNA, 20 bp, synthetic construct
  source 1..20: mol_type = other DNA
tgcacaatgctagacatgat

SEQ ID NO: 924 - DNA, 20 bp, synthetic construct
  source 1..20: mol_type = other DNA
tgcacaatctgtatcatgat

SEQ ID NO: 925 - DNA, 20 bp, synthetic construct
  source 1..20: mol_type = other DNA
tgcacagatatgattgtgat

SEQ ID NO: 926 - DNA, 22 bp, synthetic construct
  source 1..22: mol_type = other DNA
tgcacagatgaccatgcatgat

SEQ ID NO: 927 - DNA, 20 bp, synthetic construct
  source 1..20: mol_type = other DNA
tgcacatcgcgaatgatgat

SEQ ID NO: 928 - DNA, 18 bp, synthetic construct
  source 1..18: mol_type = other DNA
tgcacagactgccacgat

SEQ ID NO: 929 - DNA, 18 bp, synthetic construct
  source 1..18: mol_type = other DNA
tgcacgtctcattgcgat

SEQ ID NO: 930 - DNA, 18 bp, synthetic construct
  source 1..18: mol_type = other DNA
tgcacagacaatactgat

SEQ ID NO: 931 - DNA, 18 bp, synthetic construct
  source 1..18: mol_type = other DNA
tgcacagctgtctaagat

SEQ ID NO: 932 - DNA, 18 bp, synthetic construct
  source 1..18: mol_type = other DNA
tgcacgattctgacagat

SEQ ID NO: 933 - DNA, 22 bp, synthetic construct
  source 1..22: mol_type = other DNA
tgcacatgctgccagctgcgat

SEQ ID NO: 934 - DNA, 22 bp, synthetic construct
  source 1..22: mol_type = other DNA
tgcacatgcatcactggatgat

SEQ ID NO: 935 - DNA, 18 bp, synthetic construct
  source 1..18: mol_type = other DNA
tgcacagtgactgaagat

SEQ ID NO: 936 - DNA, 22 bp, synthetic construct
  source 1..22: mol_type = other DNA
tgcacatgatatgccatgcgat

SEQ ID NO: 937 - DNA, 20 bp, synthetic construct
  source 1..20: mol_type = other DNA
tgcacggcagcatctcagat

SEQ ID NO: 938 - DNA, 18 bp, synthetic construct
  source 1..18: mol_type = other DNA
tgcacgcactgcctagat

SEQ ID NO: 939 - DNA, 20 bp, synthetic construct
  source 1..20: mol_type = other DNA
tgcactcgagctgcattgat

SEQ ID NO: 940 - DNA, 20 bp, synthetic construct
  source 1..20: mol_type = other DNA
tgcacagctatatcattgat

SEQ ID NO: 941 - DNA, 20 bp, synthetic construct
  source 1..20: mol_type = other DNA
tgcacttgcgcgtgcatgat

SEQ ID NO: 942 - DNA, 20 bp, synthetic construct
  source 1..20: mol_type = other DNA
tgcactatgagatgattgat

SEQ ID NO: 943 - DNA, 20 bp, synthetic construct
  source 1..20: mol_type = other DNA
tgcacatatgtatgcaagat

SEQ ID NO: 944 - DNA, 20 bp, synthetic construct
  source 1..20: mol_type = other DNA
tgcacatgcagacttctgat

SEQ ID NO: 945 - DNA, 22 bp, synthetic construct
  source 1..22: mol_type = other DNA
tgcacatgtgcgcaatgcagat

SEQ ID NO: 946 - DNA, 22 bp, synthetic construct
  source 1..22: mol_type = other DNA
tgcacatctcatgcagcaagat

SEQ ID NO: 947 - DNA, 20 bp, synthetic construct
  source 1..20: mol_type = other DNA
tgcacgtctgctgccatgat

SEQ ID NO: 948 - DNA, 18 bp, synthetic construct
  source 1..18: mol_type = other DNA
tgcacatataagtgcgat

SEQ ID NO: 949 - DNA, 20 bp, synthetic construct
  source 1..20: mol_type = other DNA
tgcactgtcagttgcatgat

SEQ ID NO: 950 - DNA, 18 bp, synthetic construct
  source 1..18: mol_type = other DNA
tgcacttctatctgcgat

SEQ ID NO: 951 - DNA, 18 bp, synthetic construct
  source 1..18: mol_type = other DNA
tgcacatactatattgat

SEQ ID NO: 952 - DNA, 20 bp, synthetic construct
  source 1..20: mol_type = other DNA
tgcactaggctgatcatgat

SEQ ID NO: 953 - DNA, 18 bp, synthetic construct
  source 1..18: mol_type = other DNA
tgcacagctctcctcgat

SEQ ID NO: 954 - DNA, 20 bp, synthetic construct
  source 1..20: mol_type = other DNA
tgcactgcgccgcatcagat

SEQ ID NO: 955 - DNA, 20 bp, synthetic construct
  source 1..20: mol_type = other DNA
tgcactgcgatgctcaagat

SEQ ID NO: 956 - DNA, 18 bp, synthetic construct
  source 1..18: mol_type = other DNA
tgcacgaccgcatcagat

SEQ ID NO: 957 - DNA, 19 bp, synthetic construct
  source 1..19: mol_type = other DNA
tgcactcaagagctgagat

SEQ ID NO: 958 - DNA, 18 bp, synthetic construct
  source 1..18: mol_type = other DNA
tgcacgatatgttgcgat

SEQ ID NO: 959 - DNA, 18 bp, synthetic construct
  source 1..18: mol_type = other DNA
tgcactcacaacgatgat

SEQ ID NO: 960 - DNA, 22 bp, synthetic construct
  source 1..22: mol_type = other DNA
tgcacgatgccagcatgctgat

SEQ ID NO: 961 - DNA, 20 bp, synthetic construct
  source 1..20: mol_type = other DNA
tgcactgctccatgtatgat

SEQ ID NO: 962 - DNA, 22 bp, synthetic construct
  source 1..22: mol_type = other DNA
tgcacatctatgcatggctgat

SEQ ID NO: 963 - DNA, 18 bp, synthetic construct
  source 1..18: mol_type = other DNA
tgcactgagaagcgcgat

SEQ ID NO: 964 - DNA, 20 bp, synthetic construct
  source 1..20: mol_type = other DNA
tgcacactgagccagcagat

SEQ ID NO: 965 - DNA, 20 bp, synthetic construct
  source 1..20: mol_type = other DNA
tgcactgagagcctcatgat

SEQ ID NO: 966 - DNA, 18 bp, synthetic construct
  source 1..18: mol_type = other DNA
tgcacaactgcgagtgat

SEQ ID NO: 967 - DNA, 20 bp, synthetic construct
  source 1..20: mol_type = other DNA
tgcactctgagcatgaagat

SEQ ID NO: 968 - DNA, 20 bp, synthetic construct
  source 1..20: mol_type = other DNA
tgcactcagtgctccatgat

SEQ ID NO: 969 - DNA, 20 bp, synthetic construct
  source 1..20: mol_type = other DNA
tgcacagaacagcatgcgat

SEQ ID NO: 970 - DNA, 20 bp, synthetic construct
  source 1..20: mol_type = other DNA
tgcacgacagatgccatgat

SEQ ID NO: 971 - DNA, 20 bp, synthetic construct
  source 1..20: mol_type = other DNA
tgcacatatgcataagtgat

SEQ ID NO: 972 - DNA, 20 bp, synthetic construct
  source 1..20: mol_type = other DNA
tgcactgcgctccatgtgat

SEQ ID NO: 973 - DNA, 22 bp, synthetic construct
  source 1..22: mol_type = other DNA
tgcacatgcatctcctgcagat

SEQ ID NO: 974 - DNA, 20 bp, synthetic construct
  source 1..20: mol_type = other DNA
tgcactcatgatcggatgat

SEQ ID NO: 975 - DNA, 18 bp, synthetic construct
  source 1..18: mol_type = other DNA
tgcacgatcacaatcgat

SEQ ID NO: 976 - DNA, 20 bp, synthetic construct
  source 1..20: mol_type = other DNA
tgcacgcatgcgtggctgat

SEQ ID NO: 977 - DNA, 20 bp, synthetic construct
  source 1..20: mol_type = other DNA
tgcacggatgtcagcatgat

SEQ ID NO: 978 - DNA, 18 bp, synthetic construct
  source 1..18: mol_type = other DNA
tgcactcacggcgctgat

SEQ ID NO: 979 - DNA, 19 bp, synthetic construct
  source 1..19: mol_type = other DNA
tgcactgatctcctcagat

SEQ ID NO: 980 - DNA, 18 bp, synthetic construct
  source 1..18: mol_type = other DNA
tgcacagtcacggctgat

SEQ ID NO: 981 - DNA, 20 bp, synthetic construct
  source 1..20: mol_type = other DNA
tgcacagccgtgcatcagat

SEQ ID NO: 982 - DNA, 18 bp, synthetic construct
  source 1..18: mol_type = other DNA
tgcacatacacaatagat

SEQ ID NO: 983 - DNA, 18 bp, synthetic construct
  source 1..18: mol_type = other DNA
tgcactctcgcgattgat

SEQ ID NO: 984 - DNA, 20 bp, synthetic construct
  source 1..20: mol_type = other DNA
tgcacttgcagtgcacagat

SEQ ID NO: 985 - DNA, 18 bp, synthetic construct
  source 1..18: mol_type = other DNA
tgcactcttgtgacagat

SEQ ID NO: 986 - DNA, 22 bp, synthetic construct
  source 1..22: mol_type = other DNA
tgcacatgcatgcggctcagat

SEQ ID NO: 987 - DNA, 18 bp, synthetic construct
  source 1..18: mol_type = other DNA
tgcacgtgagcgcttgat

SEQ ID NO: 988 - DNA, 22 bp, synthetic construct
  source 1..22: mol_type = other DNA
tgcacatcattgcagtgctgat

SEQ ID NO: 989 - DNA, 18 bp, synthetic construct
  source 1..18: mol_type = other DNA
tgcacgatacaccatgat

SEQ ID NO: 990 - DNA, 18 bp, synthetic construct
  source 1..18: mol_type = other DNA
tgcacgctattcagagat

SEQ ID NO: 991 - DNA, 20 bp, synthetic construct
  source 1..20: mol_type = other DNA
tgcactgcatgtcggctgat

SEQ ID NO: 992 - DNA, 18 bp, synthetic construct
  source 1..18: mol_type = other DNA
tgcacagaagtgcgagat

SEQ ID NO: 993 - DNA, 20 bp, synthetic construct
  source 1..20: mol_type = other DNA
tgcactgttgagcacatgat

SEQ ID NO: 994 - DNA, 20 bp, synthetic construct
  source 1..20: mol_type = other DNA
tgcactgatatgcgcaagat

SEQ ID NO: 995 - DNA, 18 bp, synthetic construct
  source 1..18: mol_type = other DNA
tgcacagactagcaagat

SEQ ID NO: 996 - DNA, 20 bp, synthetic construct
  source 1..20: mol_type = other DNA
tgcacgcaagagcatatgat

SEQ ID NO: 997 - DNA, 20 bp, synthetic construct
  source 1..20: mol_type = other DNA
tgcacatcgtgatggctgat

SEQ ID NO: 998 - DNA, 20 bp, synthetic construct
  source 1..20: mol_type = other DNA
tgcactgagcctcagctgat

SEQ ID NO: 999 - DNA, 18 bp, synthetic construct
  source 1..18: mol_type = other DNA
tgcacacagcgaagagat

SEQ ID NO: 1000 - DNA, 20 bp, synthetic construct
  source 1..20: mol_type = other DNA
tgcactgaagctctcatgat

SEQ ID NO: 1001 - DNA, 20 bp, synthetic construct
  source 1..20: mol_type = other DNA
tgcacacatcatcaactgat

SEQ ID NO: 1002 - DNA, 18 bp, synthetic construct
  source 1..18: mol_type = other DNA
tgcacagaatagtcagat

SEQ ID NO: 1003 - DNA, 20 bp, synthetic construct
  source 1..20: mol_type = other DNA
tgcactgtcatctcattgat

SEQ ID NO: 1004 - DNA, 22 bp, synthetic construct
  source 1..22: mol_type = other DNA
tgcacatcgccatgcatgcgat

SEQ ID NO: 1005 - DNA, 22 bp, synthetic construct
  source 1..22: mol_type = other DNA
tgcacacagcatgcattcagat

SEQ ID NO: 1006 - DNA, 20 bp, synthetic construct
  source 1..20: mol_type = other DNA
tgcacatgcgattgatagat

SEQ ID NO: 1007 - DNA, 18 bp, synthetic construct
  source 1..18: mol_type = other DNA
tgcacgagtcattgcgat

SEQ ID NO: 1008 - DNA, 20 bp, synthetic construct
  source 1..20: mol_type = other DNA
tgcactcagatccatcagat

SEQ ID NO: 1009 - DNA, 18 bp, synthetic construct
  source 1..18: mol_type = other DNA
tgcacataatacatcgat

SEQ ID NO: 1010 - DNA, 20 bp, synthetic construct
  source 1..20: mol_type = other DNA
tgcacaagcactatgctgat

SEQ ID NO: 1011 - DNA, 18 bp, synthetic construct
  source 1..18: mol_type = other DNA
tgcacaactcgcacagat

SEQ ID NO: 1012 - DNA, 18 bp, synthetic construct
  source 1..18: mol_type = other DNA
tgcacaacactcatcgat

SEQ ID NO: 1013 - DNA, 18 bp, synthetic construct
  source 1..18: mol_type = other DNA
tgcacatgctattacgat

SEQ ID NO: 1014 - DNA, 22 bp, synthetic construct
  source 1..22: mol_type = other DNA
tgcactgcattcagcatgtgat

SEQ ID NO: 1015 - DNA, 18 bp, synthetic construct
  source 1..18: mol_type = other DNA
tgcacatacagcaccgat

SEQ ID NO: 1016 - DNA, 18 bp, synthetic construct
  source 1..18: mol_type = other DNA
tgcactctgagttgtgat

SEQ ID NO: 1017 - DNA, 18 bp, synthetic construct
  source 1..18: mol_type = other DNA
tgcacgcgctctcttgat

SEQ ID NO: 1018 - DNA, 18 bp, synthetic construct
  source 1..18: mol_type = other DNA
tgcacgatcagagccgat

SEQ ID NO: 1019 - DNA, 18 bp, synthetic construct
  source 1..18: mol_type = other DNA
tgcacatagaagatagat

SEQ ID NO: 1020 - DNA, 18 bp, synthetic construct
  source 1..18: mol_type = other DNA
tgcactcatctcgccgat

SEQ ID NO: 1021 - DNA, 20 bp, synthetic construct
  source 1..20: mol_type = other DNA
tgcacttgacatcgcatgat

SEQ ID NO: 1022 - DNA, 20 bp, synthetic construct
  source 1..20: mol_type = other DNA
tgcacagcctgagatgcgat

SEQ ID NO: 1023 - DNA, 18 bp, synthetic construct
  source 1..18: mol_type = other DNA
tgcactgtatcaatcgat

SEQ ID NO: 1024 - DNA, 20 bp, synthetic construct
  source 1..20: mol_type = other DNA
tgcacatatctcatgaagat

SEQ ID NO: 1025 - DNA, 20 bp, synthetic construct
  source 1..20: mol_type = other DNA
tgcacacatagcctgcagat

SEQ ID NO: 1026 - DNA, 20 bp, synthetic construct
  source 1..20: mol_type = other DNA
tgcactcgcttgcatctgat

SEQ ID NO: 1027 - DNA, 20 bp, synthetic construct
  source 1..20: mol_type = other DNA
tgcacatgcggcacagtgat

SEQ ID NO: 1028 - DNA, 20 bp, synthetic construct
  source 1..20: mol_type = other DNA
tgcactgctgattagctgat

SEQ ID NO: 1029 - DNA, 20 bp, synthetic construct
  source 1..20: mol_type = other DNA
tgcacatgcactgaagcgat

SEQ ID NO: 1030 - DNA, 20 bp, synthetic construct
  source 1..20: mol_type = other DNA
tgcacactgtcttgcatgat

SEQ ID NO: 1031 - DNA, 20 bp, synthetic construct
  source 1..20: mol_type = other DNA
tgcacacttatgcgcatgat

SEQ ID NO: 1032 - DNA, 20 bp, synthetic construct
  source 1..20: mol_type = other DNA
tgcacacaacagcagctgat

SEQ ID NO: 1033 - DNA, 20 bp, synthetic construct
  source 1..20: mol_type = other DNA
tgcacatgctcatggtcgat

SEQ ID NO: 1034 - DNA, 22 bp, synthetic construct
  source 1..22: mol_type = other DNA
tgcacatccacatgcatatgat

SEQ ID NO: 1035 - DNA, 20 bp, synthetic construct
  source 1..20: mol_type = other DNA
tgcacatggctctgcacgat

SEQ ID NO: 1036 - DNA, 20 bp, synthetic construct
  source 1..20: mol_type = other DNA
tgcacttgatgcactctgat

SEQ ID NO: 1037 - DNA, 20 bp, synthetic construct
  source 1..20: mol_type = other DNA
tgcacatcctctgcagtgat

SEQ ID NO: 1038 - DNA, 18 bp, synthetic construct
  source 1..18: mol_type = other DNA
tgcacaagatcgatcgat

SEQ ID NO: 1039 - DNA, 20 bp, synthetic construct
  source 1..20: mol_type = other DNA
tgcactgccatgatgtagat

SEQ ID NO: 1040 - DNA, 20 bp, synthetic construct
  source 1..20: mol_type = other DNA
tgcacactgcctcatcagat

SEQ ID NO: 1041 - DNA, 20 bp, synthetic construct
  source 1..20: mol_type = other DNA
tgcacatcatacatattgat

SEQ ID NO: 1042 - DNA, 20 bp, synthetic construct
  source 1..20: mol_type = other DNA
tgcacatatagaatcatgat

SEQ ID NO: 1043 - DNA, 20 bp, synthetic construct
  source 1..20: mol_type = other DNA
tgcacgcttgctctgcagat

SEQ ID NO: 1044 - DNA, 20 bp, synthetic construct
  source 1..20: mol_type = other DNA
tgcacaatgctctctgtgat

SEQ ID NO: 1045 - DNA, 20 bp, synthetic construct
  source 1..20: mol_type = other DNA
tgcactagtccatgcatgat

SEQ ID NO: 1046 - DNA, 20 bp, synthetic construct
  source 1..20: mol_type = other DNA
tgcacaatcatgctatagat

SEQ ID NO: 1047 - DNA, 22 bp, synthetic construct
  source 1..22: mol_type = other DNA
tgcacatgcgcaacatgcagat

SEQ ID NO: 1048 - DNA, 20 bp, synthetic construct
  source 1..20: mol_type = other DNA
tgcactcatatggcagtgat

SEQ ID NO: 1049 - DNA, 20 bp, synthetic construct
  source 1..20: mol_type = other DNA
tgcacagcacattatatgat

SEQ ID NO: 1050 - DNA, 20 bp, synthetic construct
  source 1..20: mol_type = other DNA
tgcacatctgcactgaagat

SEQ ID NO: 1051 - DNA, 20 bp, synthetic construct
  source 1..20: mol_type = other DNA
tgcacatcgccagcactgat

SEQ ID NO: 1052 - DNA, 22 bp, synthetic construct
  source 1..22: mol_type = other DNA
tgcacagcctcagctgcatgat

SEQ ID NO: 1053 - DNA, 18 bp, synthetic construct
  source 1..18: mol_type = other DNA
tgcacgcggcacagagat

SEQ ID NO: 1054 - DNA, 20 bp, synthetic construct
  source 1..20: mol_type = other DNA
tgcacagactgcattgtgat

SEQ ID NO: 1055 - DNA, 22 bp, synthetic construct
  source 1..22: mol_type = other DNA
tgcacaatctgcatgatcagat

SEQ ID NO: 1056 - DNA, 22 bp, synthetic construct
  source 1..22: mol_type = other DNA
tgcacaatgcagcgctgctgat

SEQ ID NO: 1057 - DNA, 20 bp, synthetic construct
  source 1..20: mol_type = other DNA
tgcacagtcatgcttctgat

SEQ ID NO: 1058 - DNA, 20 bp, synthetic construct
  source 1..20: mol_type = other DNA
tgcactcagtgaatgatgat

SEQ ID NO: 1059 - DNA, 22 bp, synthetic construct
  source 1..22: mol_type = other DNA
tgcacagcatgatcaggctgat

SEQ ID NO: 1060 - DNA, 20 bp, synthetic construct
  source 1..20: mol_type = other DNA
tgcacatactctgcattgat

SEQ ID NO: 1061 - DNA, 20 bp, synthetic construct
  source 1..20: mol_type = other DNA
tgcacagccgcgatgcagat

SEQ ID NO: 1062 - DNA, 20 bp, synthetic construct
  source 1..20: mol_type = other DNA
tgcacatggtgcacgatgat

SEQ ID NO: 1063 - DNA, 18 bp, synthetic construct
  source 1..18: mol_type = other DNA
tgcacttcactgctcgat

SEQ ID NO: 1064 - DNA, 20 bp, synthetic construct
  source 1..20: mol_type = other DNA
tgcacagcacatcttctgat

SEQ ID NO: 1065 - DNA, 20 bp, synthetic construct
  source 1..20: mol_type = other DNA
tgcacacagtgaatcatgat

SEQ ID NO: 1066 - DNA, 18 bp, synthetic construct
  source 1..18: mol_type = other DNA
tgcactgttatcgctgat

SEQ ID NO: 1067 - DNA, 20 bp, synthetic construct
  source 1..20: mol_type = other DNA
tgcactagatgtgccatgat

SEQ ID NO: 1068 - DNA, 18 bp, synthetic construct
  source 1..18: mol_type = other DNA
tgcacggctatatgcgat

SEQ ID NO: 1069 - DNA, 18 bp, synthetic construct
  source 1..18: mol_type = other DNA
tgcacgtgcgcaactgat

SEQ ID NO: 1070 - DNA, 20 bp, synthetic construct
  source 1..20: mol_type = other DNA
tgcacatgtgctctcaagat

SEQ ID NO: 1071 - DNA, 20 bp, synthetic construct
  source 1..20: mol_type = other DNA
tgcacaagcgcagctctgat

SEQ ID NO: 1072 - DNA, 18 bp, synthetic construct
  source 1..18: mol_type = other DNA
tgcactctatattctgat

SEQ ID NO: 1073 - DNA, 19 bp, synthetic construct
  source 1..19: mol_type = other DNA
tgcacttgagctgcgagat

SEQ ID NO: 1074 - DNA, 20 bp, synthetic construct
  source 1..20: mol_type = other DNA
tgcacacatggctagcagat

SEQ ID NO: 1075 - DNA, 18 bp, synthetic construct
  source 1..18: mol_type = other DNA
tgcacgtgagattgtgat

SEQ ID NO: 1076 - DNA, 20 bp, synthetic construct
  source 1..20: mol_type = other DNA
tgcactagcttgctgctgat

SEQ ID NO: 1077 - DNA, 22 bp, synthetic construct
  source 1..22: mol_type = other DNA
tgcactgatgcaatctgcagat

SEQ ID NO: 1078 - DNA, 22 bp, synthetic construct
  source 1..22: mol_type = other DNA
tgcacatgcataatgatatgat

SEQ ID NO: 1079 - DNA, 18 bp, synthetic construct
  source 1..18: mol_type = other DNA
tgcacagtgcctgacgat

SEQ ID NO: 1080 - DNA, 20 bp, synthetic construct
  source 1..20: mol_type = other DNA
tgcacatagagcattctgat

SEQ ID NO: 1081 - DNA, 20 bp, synthetic construct
  source 1..20: mol_type = other DNA
tgcacatgatctgcgaagat

SEQ ID NO: 1082 - DNA, 20 bp, synthetic construct
  source 1..20: mol_type = other DNA
tgcacatctgattctgtgat

SEQ ID NO: 1083 - DNA, 22 bp, synthetic construct
  source 1..22: mol_type = other DNA
tgcacatgcaagctgatatgat

SEQ ID NO: 1084 - DNA, 20 bp, synthetic construct
  source 1..20: mol_type = other DNA
tgcacacagcattgactgat

SEQ ID NO: 1085 - DNA, 18 bp, synthetic construct
  source 1..18: mol_type = other DNA
tgcacagtcaatcgagat

SEQ ID NO: 1086 - DNA, 20 bp, synthetic construct
  source 1..20: mol_type = other DNA
tgcactgatggcatatagat

SEQ ID NO: 1087 - DNA, 18 bp, synthetic construct
  source 1..18: mol_type = other DNA
tgcacactcgctgaagat

SEQ ID NO: 1088 - DNA, 20 bp, synthetic construct
  source 1..20: mol_type = other DNA
tgcactgaagagatgcagat

SEQ ID NO: 1089 - DNA, 20 bp, synthetic construct
  source 1..20: mol_type = other DNA
tgcacgaggatctgcatgat

SEQ ID NO: 1090 - DNA, 20 bp, synthetic construct
  source 1..20: mol_type = other DNA
tgcacaacatctgctcagat

SEQ ID NO: 1091 - DNA, 22 bp, synthetic construct
  source 1..22: mol_type = other DNA
tgcacgctgaatgcatgctgat

SEQ ID NO: 1092 - DNA, 20 bp, synthetic construct
  source 1..20: mol_type = other DNA
tgcacatctccacatcagat

SEQ ID NO: 1093 - DNA, 22 bp, synthetic construct
  source 1..22: mol_type = other DNA
tgcacatgagatgatcattgat

SEQ ID NO: 1094 - DNA, 20 bp, synthetic construct
  source 1..20: mol_type = other DNA
tgcactgctctggctgagat

SEQ ID NO: 1095 - DNA, 20 bp, synthetic construct
  source 1..20: mol_type = other DNA
tgcacataatgtgtgatgat

SEQ ID NO: 1096 - DNA, 18 bp, synthetic construct
  source 1..18: mol_type = other DNA
tgcactcatgtatccgat

SEQ ID NO: 1097 - DNA, 18 bp, synthetic construct
  source 1..18: mol_type = other DNA
tgcacgctgtgcgttgat

SEQ ID NO: 1098 - DNA, 18 bp, synthetic construct
  source 1..18: mol_type = other DNA
tgcacggcatcacgtgat

SEQ ID NO: 1099 - DNA, 20 bp, synthetic construct
  source 1..20: mol_type = other DNA
tgcactgacatatgattgat

SEQ ID NO: 1100 - DNA, 20 bp, synthetic construct
  source 1..20: mol_type = other DNA
tgcacaagtgcatatctgat

SEQ ID NO: 1101 - DNA, 20 bp, synthetic construct
  source 1..20: mol_type = other DNA
tgcacagcgcattgtgagat

SEQ ID NO: 1102 - DNA, 18 bp, synthetic construct
  source 1..18: mol_type = other DNA
tgcacgtccagatcagat

SEQ ID NO: 1103 - DNA, 20 bp, synthetic construct
  source 1..20: mol_type = other DNA
tgcacatgcggtgcgatgat

SEQ ID NO: 1104 - DNA, 18 bp, synthetic construct
  source 1..18: mol_type = other DNA
tgcacaatatatagcgat

SEQ ID NO: 1105 - DNA, 18 bp, synthetic construct
  source 1..18: mol_type = other DNA
tgcacaagtgctagtgat

SEQ ID NO: 1106 - DNA, 18 bp, synthetic construct
  source 1..18: mol_type = other DNA
tgcactgtgaatagagat

SEQ ID NO: 1107 - DNA, 20 bp, synthetic construct
  source 1..20: mol_type = other DNA
tgcacgattgatgcacagat

SEQ ID NO: 1108 - DNA, 18 bp, synthetic construct
  source 1..18: mol_type = other DNA
tgcacttgtgcgacagat

SEQ ID NO: 1109 - DNA, 18 bp, synthetic construct
  source 1..18: mol_type = other DNA
tgcacaactgtgactgat

SEQ ID NO: 1110 - DNA, 18 bp, synthetic construct
  source 1..18: mol_type = other DNA
tgcactagcgcttatgat

SEQ ID NO: 1111 - DNA, 18 bp, synthetic construct
  source 1..18: mol_type = other DNA
tgcacagatatcctcgat

SEQ ID NO: 1112 - DNA, 20 bp, synthetic construct
  source 1..20: mol_type = other DNA
tgcacgtggctgagcatgat

SEQ ID NO: 1113 - DNA, 22 bp, synthetic construct
  source 1..22: mol_type = other DNA
tgcactgatgcatggtgctgat

SEQ ID NO: 1114 - DNA, 20 bp, synthetic construct
  source 1..20: mol_type = other DNA
tgcacacttgcatgcgcgat

SEQ ID NO: 1115 - DNA, 20 bp, synthetic construct
  source 1..20: mol_type = other DNA
tgcacagccatgcgacagat

SEQ ID NO: 1116 - DNA, 18 bp, synthetic construct
  source 1..18: mol_type = other DNA
tgcactcttctctctgat

SEQ ID NO: 1117 - DNA, 20 bp, synthetic construct
  source 1..20: mol_type = other DNA
tgcacatggcgtatgatgat

SEQ ID NO: 1118 - DNA, 20 bp, synthetic construct
  source 1..20: mol_type = other DNA
tgcactattctcagcatgat

SEQ ID NO: 1119 - DNA, 20 bp, synthetic construct
  source 1..20: mol_type = other DNA
tgcacagttctatgcatgat

SEQ ID NO: 1120 - DNA, 22 bp, synthetic construct
  source 1..22: mol_type = other DNA
tgcacagcatcatgcttgtgat

SEQ ID NO: 1121 - DNA, 18 bp, synthetic construct
  source 1..18: mol_type = other DNA
tgcacgtaatgctgtgat

SEQ ID NO: 1122 - DNA, 20 bp, synthetic construct
  source 1..20: mol_type = other DNA
tgcacgcaatagatcatgat

SEQ ID NO: 1123 - DNA, 22 bp, synthetic construct
  source 1..22: mol_type = other DNA
tgcacttctgcatgctgcagat

SEQ ID NO: 1124 - DNA, 20 bp, synthetic construct
  source 1..20: mol_type = other DNA
tgcacagcacatgtgccgat

SEQ ID NO: 1125 - DNA, 18 bp, synthetic construct
  source 1..18: mol_type = other DNA
tgcacagcgataactgat

SEQ ID NO: 1126 - DNA, 18 bp, synthetic construct
  source 1..18: mol_type = other DNA
tgcacagttgtgtgcgat

SEQ ID NO: 1127 - DNA, 20 bp, synthetic construct
  source 1..20: mol_type = other DNA
tgcacacatgaggcgctgat

SEQ ID NO: 1128 - DNA, 20 bp, synthetic construct
  source 1..20: mol_type = other DNA
tgcacatgcgctattctgat

SEQ ID NO: 1129 - DNA, 20 bp, synthetic construct
  source 1..20: mol_type = other DNA
tgcacgagcaatgcgatgat

SEQ ID NO: 1130 - DNA, 20 bp, synthetic construct
  source 1..20: mol_type = other DNA
tgcactgccatgctctagat

SEQ ID NO: 1131 - DNA, 20 bp, synthetic construct
  source 1..20: mol_type = other DNA
tgcacaagcatgcgtatgat

SEQ ID NO: 1132 - DNA, 20 bp, synthetic construct
  source 1..20: mol_type = other DNA
tgcacagaagctcatctgat

SEQ ID NO: 1133 - DNA, 20 bp, synthetic construct
  source 1..20: mol_type = other DNA
tgcactacagctgcattgat

SEQ ID NO: 1134 - DNA, 20 bp, synthetic construct
  source 1..20: mol_type = other DNA
tgcacatcgcattagctgat

SEQ ID NO: 1135 - DNA, 18 bp, synthetic construct
  source 1..18: mol_type = other DNA
tgcacaacatatcgcgat

SEQ ID NO: 1136 - DNA, 18 bp, synthetic construct
  source 1..18: mol_type = other DNA
tgcacgtatggcatagat

SEQ ID NO: 1137 - DNA, 20 bp, synthetic construct
  source 1..20: mol_type = other DNA
tgcacgatcttgcatgagat

SEQ ID NO: 1138 - DNA, 20 bp, synthetic construct
  source 1..20: mol_type = other DNA
tgcactcttgcgatcatgat

SEQ ID NO: 1139 - DNA, 20 bp, synthetic construct
  source 1..20: mol_type = other DNA
tgcactctgtcatgattgat

SEQ ID NO: 1140 - DNA, 19 bp, synthetic construct
  source 1..19: mol_type = other DNA
tgcacagctgtaatatgat

SEQ ID NO: 1141 - DNA, 20 bp, synthetic construct
  source 1..20: mol_type = other DNA
tgcacagtgctggctgagat

SEQ ID NO: 1142 - DNA, 19 bp, synthetic construct
  source 1..19: mol_type = other DNA
tgcacgctgcctcagagat

SEQ ID NO: 1143 - DNA, 20 bp, synthetic construct
  source 1..20: mol_type = other DNA
tgcacgagattgatgctgat

SEQ ID NO: 1144 - DNA, 18 bp, synthetic construct
  source 1..18: mol_type = other DNA
tgcacataatatatagat

SEQ ID NO: 1145 - DNA, 20 bp, synthetic construct
  source 1..20: mol_type = other DNA
tgcacagtagattgcatgat

SEQ ID NO: 1146 - DNA, 20 bp, synthetic construct
  source 1..20: mol_type = other DNA
tgcactcagcacctgctgat

SEQ ID NO: 1147 - DNA, 18 bp, synthetic construct
  source 1..18: mol_type = other DNA
tgcacatgtctaagcgat

SEQ ID NO: 1148 - DNA, 20 bp, synthetic construct
  source 1..20: mol_type = other DNA
tgcactgtgatggctctgat

SEQ ID NO: 1149 - DNA, 18 bp, synthetic construct
  source 1..18: mol_type = other DNA
tgcacggtagcatgcgat

SEQ ID NO: 1150 - DNA, 20 bp, synthetic construct
  source 1..20: mol_type = other DNA
tgcactgcatgcgttgagat

SEQ ID NO: 1151 - DNA, 20 bp, synthetic construct
  source 1..20: mol_type = other DNA
tgcactcatgctgtcaagat

SEQ ID NO: 1152 - DNA, 18 bp, synthetic construct
  source 1..18: mol_type = other DNA
tgcacacggtgctgtgat

SEQ ID NO: 1153 - DNA, 22 bp, synthetic construct
  source 1..22: mol_type = other DNA
tgcacaagcatgcatgagagat

SEQ ID NO: 1154 - DNA, 18 bp, synthetic construct
  source 1..18: mol_type = other DNA
tgcacacagaagtctgat

SEQ ID NO: 1155 - DNA, 18 bp, synthetic construct
  source 1..18: mol_type = other DNA
tgcacttagatgacagat

SEQ ID NO: 1156 - DNA, 18 bp, synthetic construct
  source 1..18: mol_type = other DNA
tgcacatacaactgtgat

SEQ ID NO: 1157 - DNA, 18 bp, synthetic construct
  source 1..18: mol_type = other DNA
tgcacttgagagagtgat

SEQ ID NO: 1158 - DNA, 20 bp, synthetic construct
  source 1..20: mol_type = other DNA
tgcacatctacttgcatgat

SEQ ID NO: 1159 - DNA, 20 bp, synthetic construct
  source 1..20: mol_type = other DNA
tgcacttgcatgctacagat

SEQ ID NO: 1160 - DNA, 20 bp, synthetic construct
  source 1..20: mol_type = other DNA
tgcactcagcagcggcagat

SEQ ID NO: 1161 - DNA, 18 bp, synthetic construct
  source 1..18: mol_type = other DNA
tgcactgtacattgtgat

SEQ ID NO: 1162 - DNA, 18 bp, synthetic construct
  source 1..18: mol_type = other DNA
tgcacttgtgcatacgat

SEQ ID NO: 1163 - DNA, 18 bp, synthetic construct
  source 1..18: mol_type = other DNA
tgcacaacgtgagcagat

SEQ ID NO: 1164 - DNA, 20 bp, synthetic construct
  source 1..20: mol_type = other DNA
tgcacacatgcacggcagat

SEQ ID NO: 1165 - DNA, 20 bp, synthetic construct
  source 1..20: mol_type = other DNA
tgcacaagcgtgtgcatgat

SEQ ID NO: 1166 - DNA, 20 bp, synthetic construct
  source 1..20: mol_type = other DNA
tgcacatgcacttatgagat

SEQ ID NO: 1167 - DNA, 20 bp, synthetic construct
  source 1..20: mol_type = other DNA
tgcacgcgacattgcatgat

SEQ ID NO: 1168 - DNA, 20 bp, synthetic construct
  source 1..20: mol_type = other DNA
tgcacggatgatgctgagat

SEQ ID NO: 1169 - DNA, 20 bp, synthetic construct
  source 1..20: mol_type = other DNA
tgcactgatgagcaatcgat

SEQ ID NO: 1170 - DNA, 18 bp, synthetic construct
  source 1..18: mol_type = other DNA
tgcacacgctacattgat

SEQ ID NO: 1171 - DNA, 22 bp, synthetic construct
  source 1..22: mol_type = other DNA
tgcacgcaatgctgatcatgat

SEQ ID NO: 1172 - DNA, 20 bp, synthetic construct
  source 1..20: mol_type = other DNA
tgcacgcgcataatgatgat

SEQ ID NO: 1173 - DNA, 18 bp, synthetic construct
  source 1..18: mol_type = other DNA
tgcacacacgcataagat

SEQ ID NO: 1174 - DNA, 22 bp, synthetic construct
  source 1..22: mol_type = other DNA
tgcacagatggtgcatgcagat

SEQ ID NO: 1175 - DNA, 20 bp, synthetic construct
  source 1..20: mol_type = other DNA
tgcacgcacatggatctgat

SEQ ID NO: 1176 - DNA, 20 bp, synthetic construct
  source 1..20: mol_type = other DNA
tgcacatgcaacgcagtgat

SEQ ID NO: 1177 - DNA, 20 bp, synthetic construct
  source 1..20: mol_type = other DNA
tgcacagcaggagatcagat

SEQ ID NO: 1178 - DNA, 20 bp, synthetic construct
  source 1..20: mol_type = other DNA
tgcacacatcaggtgatgat

SEQ ID NO: 1179 - DNA, 20 bp, synthetic construct
  source 1..20: mol_type = other DNA
tgcacgctcaatgagcagat

SEQ ID NO: 1180 - DNA, 18 bp, synthetic construct
  source 1..18: mol_type = other DNA
tgcactgtgcagtccgat

SEQ ID NO: 1181 - DNA, 20 bp, synthetic construct
  source 1..20: mol_type = other DNA
tgcactgatgcgcagaagat

SEQ ID NO: 1182 - DNA, 20 bp, synthetic construct
  source 1..20: mol_type = other DNA
tgcacaatatgcatgtcgat

SEQ ID NO: 1183 - DNA, 19 bp, synthetic construct
  source 1..19: mol_type = other DNA
tgcacagatgagtggagat

SEQ ID NO: 1184 - DNA, 22 bp, synthetic construct
  source 1..22: mol_type = other DNA
tgcacatggagatgcatgtgat

SEQ ID NO: 1185 - DNA, 20 bp, synthetic construct
  source 1..20: mol_type = other DNA
tgcacatcattcgatgtgat

SEQ ID NO: 1186 - DNA, 18 bp, synthetic construct
  source 1..18: mol_type = other DNA
tgcacgctcggcagcgat

SEQ ID NO: 1187 - DNA, 18 bp, synthetic construct
  source 1..18: mol_type = other DNA
tgcacgctagcttgagat

SEQ ID NO: 1188 - DNA, 18 bp, synthetic construct
  source 1..18: mol_type = other DNA
tgcacacgcaatctagat

SEQ ID NO: 1189 - DNA, 18 bp, synthetic construct
  source 1..18: mol_type = other DNA
tgcacacgcaactgtgat

SEQ ID NO: 1190 - DNA, 20 bp, synthetic construct
  source 1..20: mol_type = other DNA
tgcacacgcacaatcatgat

SEQ ID NO: 1191 - DNA, 22 bp, synthetic construct
  source 1..22: mol_type = other DNA
tgcacgcatctgcaatgctgat

SEQ ID NO: 1192 - DNA, 20 bp, synthetic construct
  source 1..20: mol_type = other DNA
tgcacgctcagatgattgat

SEQ ID NO: 1193 - DNA, 22 bp, synthetic construct
  source 1..22: mol_type = other DNA
tgcacgcatatgcatggatgat

SEQ ID NO: 1194 - DNA, 22 bp, synthetic construct
  source 1..22: mol_type = other DNA
tgcacgcatgcatggcatagat

SEQ ID NO: 1195 - DNA, 20 bp, synthetic construct
  source 1..20: mol_type = other DNA
tgcacgcatccagcactgat

SEQ ID NO: 1196 - DNA, 18 bp, synthetic construct
  source 1..18: mol_type = other DNA
tgcacggagctcacagat

SEQ ID NO: 1197 - DNA, 20 bp, synthetic construct
  source 1..20: mol_type = other DNA
tgcacgctctgcattcagat

SEQ ID NO: 1198 - DNA, 20 bp, synthetic construct
  source 1..20: mol_type = other DNA
tgcacatagcagaggatgat

SEQ ID NO: 1199 - DNA, 22 bp, synthetic construct
  source 1..22: mol_type = other DNA
tgcactgcaggcgcatgatgat

SEQ ID NO: 1200 - DNA, 20 bp, synthetic construct
  source 1..20: mol_type = other DNA
tgcacgcatatctggctgat

SEQ ID NO: 1201 - DNA, 20 bp, synthetic construct
  source 1..20: mol_type = other DNA
tgcactgctgtgcatccgat

SEQ ID NO: 1202 - DNA, 20 bp, synthetic construct
  source 1..20: mol_type = other DNA
tgcacatggcagtcatagat

SEQ ID NO: 1203 - DNA, 22 bp, synthetic construct
  source 1..22: mol_type = other DNA
tgcactgcatgaatgctgtgat

SEQ ID NO: 1204 - DNA, 20 bp, synthetic construct
  source 1..20: mol_type = other DNA
tgcacgcgcatgtccatgat

SEQ ID NO: 1205 - DNA, 20 bp, synthetic construct
  source 1..20: mol_type = other DNA
tgcacgcacacatccatgat

SEQ ID NO: 1206 - DNA, 20 bp, synthetic construct
  source 1..20: mol_type = other DNA
tgcactgcaggagtgatgat

SEQ ID NO: 1207 - DNA, 18 bp, synthetic construct
  source 1..18: mol_type = other DNA
tgcacgcgtggcagagat

SEQ ID NO: 1208 - DNA, 22 bp, synthetic construct
  source 1..22: mol_type = other DNA
tgcacgcatggcatcacatgat

SEQ ID NO: 1209 - DNA, 20 bp, synthetic construct
  source 1..20: mol_type = other DNA
tgcactagcaatgatgagat

SEQ ID NO: 1210 - DNA, 22 bp, synthetic construct
  source 1..22: mol_type = other DNA
tgcactgcattagcatgcagat

SEQ ID NO: 1211 - DNA, 20 bp, synthetic construct
  source 1..20: mol_type = other DNA
tgcactatgcagtcattgat

SEQ ID NO: 1212 - DNA, 20 bp, synthetic construct
  source 1..20: mol_type = other DNA
tgcacactgcagtcattgat

SEQ ID NO: 1213 - DNA, 20 bp, synthetic construct
  source 1..20: mol_type = other DNA
tgcacatcagtgcaatcgat

SEQ ID NO: 1214 - DNA, 19 bp, synthetic construct
  source 1..19: mol_type = other DNA
tgcacgcaagctgagagat

SEQ ID NO: 1215 - DNA, 18 bp, synthetic construct
  source 1..18: mol_type = other DNA
tgcacggcgagatgagat

SEQ ID NO: 1216 - DNA, 22 bp, synthetic construct
  source 1..22: mol_type = other DNA
tgcacatgcagttgatgcagat

SEQ ID NO: 1217 - DNA, 20 bp, synthetic construct
  source 1..20: mol_type = other DNA
tgcacagatgagacattgat

SEQ ID NO: 1218 - DNA, 20 bp, synthetic construct
  source 1..20: mol_type = other DNA
tgcacgcttgatgatcagat

SEQ ID NO: 1219 - DNA, 20 bp, synthetic construct
  source 1..20: mol_type = other DNA
tgcacggcgcatgcagtgat

SEQ ID NO: 1220 - DNA, 20 bp, synthetic construct
  source 1..20: mol_type = other DNA
tgcactgagacatgcttgat

SEQ ID NO: 1221 - DNA, 20 bp, synthetic construct
  source 1..20: mol_type = other DNA
tgcacgcatctggcgatgat

SEQ ID NO: 1222 - DNA, 20 bp, synthetic construct
  source 1..20: mol_type = other DNA
tgcacagcatgcagtccgat

SEQ ID NO: 1223 - DNA, 20 bp, synthetic construct
  source 1..20: mol_type = other DNA
tgcacggatgcagtgatgat

SEQ ID NO: 1224 - DNA, 20 bp, synthetic construct
  source 1..20: mol_type = other DNA
tgcacgattgcagcgcagat

SEQ ID NO: 1225 - DNA, 18 bp, synthetic construct
  source 1..18: mol_type = other DNA
tgcacgtgaggtgcagat

SEQ ID NO: 1226 - DNA, 20 bp, synthetic construct
  source 1..20: mol_type = other DNA
tgcacgcacaagcatgagat

SEQ ID NO: 1227 - DNA, 22 bp, synthetic construct
  source 1..22: mol_type = other DNA
tgcacatcaggcgcatgcagat

SEQ ID NO: 1228 - DNA, 22 bp, synthetic construct
  source 1..22: mol_type = other DNA
tgcacagcattctgtgcatgat

SEQ ID NO: 1229 - DNA, 18 bp, synthetic construct
  source 1..18: mol_type = other DNA
tgcactagcggtgcagat

SEQ ID NO: 1230 - DNA, 20 bp, synthetic construct
  source 1..20: mol_type = other DNA
tgcacgcatgattagatgat

SEQ ID NO: 1231 - DNA, 22 bp, synthetic construct
  source 1..22: mol_type = other DNA
tgcacgcatgcaatgtgcagat

SEQ ID NO: 1232 - DNA, 20 bp, synthetic construct
  source 1..20: mol_type = other DNA
tgcacgcagcacattgcgat

SEQ ID NO: 1233 - DNA, 20 bp, synthetic construct
  source 1..20: mol_type = other DNA
tgcacgcagcacctgatgat

SEQ ID NO: 1234 - DNA, 20 bp, synthetic construct
  source 1..20: mol_type = other DNA
tgcacgcagcctgcgctgat

SEQ ID NO: 1235 - DNA, 20 bp, synthetic construct
  source 1..20: mol_type = other DNA
tgcacgcatggagctgcgat

SEQ ID NO: 1236 - DNA, 20 bp, synthetic construct
  source 1..20: mol_type = other DNA
tgcacgcggatgctgatgat

SEQ ID NO: 1237 - DNA, 20 bp, synthetic construct
  source 1..20: mol_type = other DNA
tgcacgtgcgcatggatgat

SEQ ID NO: 1238 - DNA, 20 bp, synthetic construct
  source 1..20: mol_type = other DNA
tgcacggtgcatgcagagat

SEQ ID NO: 1239 - DNA, 20 bp, synthetic construct
  source 1..20: mol_type = other DNA
tgcacgcacttgatgatgat

SEQ ID NO: 1240 - DNA, 18 bp, synthetic construct
  source 1..18: mol_type = other DNA
tgcacttctcacgcagat

SEQ ID NO: 1241 - DNA, 20 bp, synthetic construct
  source 1..20: mol_type = other DNA
tgcacgcaatgagcgatgat

SEQ ID NO: 1242 - DNA, 22 bp, synthetic construct
  source 1..22: mol_type = other DNA
tgcacatcatgaatgatgtgat

SEQ ID NO: 1243 - DNA, 20 bp, synthetic construct
  source 1..20: mol_type = other DNA
tgcacgcagcagagattgat

SEQ ID NO: 1244 - DNA, 18 bp, synthetic construct
  source 1..18: mol_type = other DNA
tgcacgtggatgagcgat

SEQ ID NO: 1245 - DNA, 22 bp, synthetic construct
  source 1..22: mol_type = other DNA
tgcacgcttgcagcatgcagat

SEQ ID NO: 1246 - DNA, 20 bp, synthetic construct
  source 1..20: mol_type = other DNA
tgcacgcagatcattctgat

SEQ ID NO: 1247 - DNA, 19 bp, synthetic construct
  source 1..19: mol_type = other DNA
tgcacgcagaatctgcgat

SEQ ID NO: 1248 - DNA, 20 bp, synthetic construct
  source 1..20: mol_type = other DNA
tgcacgcagatccagcagat

SEQ ID NO: 1249 - DNA, 22 bp, synthetic construct
  source 1..22: mol_type = other DNA
tgcacatggatgagatgctgat

SEQ ID NO: 1250 - DNA, 20 bp, synthetic construct
  source 1..20: mol_type = other DNA
tgcacgcagtgctgcaagat

SEQ ID NO: 1251 - DNA, 20 bp, synthetic construct
  source 1..20: mol_type = other DNA
tgcacgcagtgagccatgat

SEQ ID NO: 1252 - DNA, 20 bp, synthetic construct
  source 1..20: mol_type = other DNA
tgcacacggatcatgcagat

SEQ ID NO: 1253 - DNA, 18 bp, synthetic construct
  source 1..18: mol_type = other DNA
tgcacggctcgtgcagat

SEQ ID NO: 1254 - DNA, 20 bp, synthetic construct
  source 1..20: mol_type = other DNA
tgcacacgcatggagatgat

SEQ ID NO: 1255 - DNA, 18 bp, synthetic construct
  source 1..18: mol_type = other DNA
tgcacacacgcggatgat

SEQ ID NO: 1256 - DNA, 20 bp, synthetic construct
  source 1..20: mol_type = other DNA
tgcacatcgaatgtgcagat

SEQ ID NO: 1257 - DNA, 18 bp, synthetic construct
  source 1..18: mol_type = other DNA
tgcacacgcagacaagat

SEQ ID NO: 1258 - DNA, 20 bp, synthetic construct
  source 1..20: mol_type = other DNA
tgcacacacatggatgagat

SEQ ID NO: 1259 - DNA, 22 bp, synthetic construct
  source 1..22: mol_type = other DNA
tgcacatgattcatgtgcagat

SEQ ID NO: 1260 - DNA, 24 bp, synthetic construct
  source 1..24: mol_type = other DNA
atctcatccctgcgtgtctccgac

SEQ ID NO: 1261 - DNA, 44 bp, synthetic construct
  source 1..44: mol_type = other DNA
  modified_base 43: mod_base = OTHER; note = Nucleotide with phosphorothioate linkage
atctcatccctgcgtgtctccgactgcacgtgcgcatggatgat

SEQ ID NO: 1262 - DNA, 12 bp, synthetic construct
  source 1..12: mol_type = other DNA
tatggtcgtcga

SEQ ID NO: 1263 - DNA, 27 bp, synthetic construct
  source 1..27: mol_type = other DNA
  modified_base 1: mod_base = OTHER; note = Biotin attached to uracil
  modified_base 5: mod_base = OTHER; note = Uracil
  modified_base 10: mod_base = OTHER; note = Uracil
  modified_base 22: mod_base = OTHER; note = Uracil
tccatctcatccctgcgtgtctccgac

SEQ ID NO: 1264 - DNA, 10 bp, synthetic construct
  source 1..10: mol_type = other DNA
aagagacctg

(19) United States
(12) Patent Application Publication
(10) Pub. No.: US 2026/0009023 A1
(43) Pub. Date: Jan. 8, 2026

(54) SYSTEMS AND METHODS FOR LIBRARY PREPARATION ADAPTERS

(71) Applicant: ULTIMA GENOMICS, INC., Fremont, CA (US)

(72) Inventors: Tyson CLARK, Menlo Park, CA (US); Zohar SHIPONY, Rehovot (IL); Tommie Lloyd LINCECUM, JR., Carlsbad, CA (US); Na Kyung LEE, East Palo Alto, CA (US); Daniel MAZUR, San Diego, CA (US); Yoav ETZIONI, Tel Aviv (IL); Florian OBERSTRASS, Redwood City, CA (US); Omer BARAD, Mazkeret Batya (IL); Elan A. SHATOFF, Oak Park, IL (US); Edward PERELMAN, Lehavim (IL); Jennifer M. KILZER, San Marcos, CA (US); Mark GESHEL, Kfar Saba (IL); Ron SAAR DOVER, Palo Alto, CA (US); Tsu-Ju FU, Poway, CA (US)

(21) Appl. No.: 19/261,379

(22) Filed: Jul. 7, 2025

Related U.S. Application Data
(63) Continuation of application No. PCT/US2024/011506, filed on Jan. 12, 2024.
(60) Provisional application No. 63/438,780, filed on Jan. 12, 2023.

(51) Int. Cl. C12N 15/10

(52) U.S. Cl. CPC C12N 15/1093

(57) ABSTRACT

Provided herein are systems, methods, compositions, and kits for library preparation. In some cases, multiple distinct types of adapter molecules may be provided to a template nucleic acid molecule. In some cases, a single type of adapter molecule may be provided to a template molecule. In some cases, multiple distinct types of adapter molecules may be sequentially provided to a template molecule to form multi-adapter template complexes.

### CROSS-REFERENCE

This application is a continuation of International Patent Application No. PCT/US2024/011506, filed Jan. 12, 2024, which claims the benefit of U.S. Patent Application No. 63/438,780, filed Jan. 12, 2023, each of which is incorporated by reference herein in its entirety.

### REFERENCE TO AN ELECTRONIC SEQUENCE LISTING

The instant application contains a Sequence Listing which has been submitted electronically in XML format and is hereby incorporated by reference in its entirety. Said XML copy, created on Jun. 11, 2025, is named Ultima 51024-779.301 SL.XML and is 1.06 MB in size.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

Despite the advance of sequencing technology, analyzing samples with high throughput and efficiency still requires laborious efforts.

### SUMMARY

Preparation of libraries for sequencing can require comparatively large amounts of genetic material (e.g., deoxyribonucleic acid (DNA), ribonucleic acid (RNA), etc.) of interest (e.g., from a sample of a subject). This genetic material is, in some cases, difficult to collect or inherently limited in availability (e.g., complementary DNA (cDNA)). Thus, recognized herein is a need for preparing libraries for sequencing in an efficient manner, maximizing use of sample genetic material. Provided herein are systems, methods, compositions, and kits for library preparation that address at least the abovementioned needs.

Provided herein, are nucleic acid compositions, kits, and methods.

In one aspect, a composition is provided, comprising a non-naturally occurring nucleic acid molecule comprising a first strand comprising SEQ ID No:1 and a second strand comprising SEQ ID No. 2.

In some embodiments, the non-naturally occurring nucleic acid molecule is coupled to a template nucleic acid molecule. In some embodiments, the coupling is via ligation. In some embodiments, the non-naturally occurring nucleic acid molecule further comprises a barcode sequence selected from any one of SEQ ID Nos: 205-1259. In some embodiments, the barcode sequence selected from any one of SEQ ID Nos: 205-1259 is disposed 3′ of SEQ ID No: 1, and a reverse complementary sequence of the selected barcode is disposed 5′ of SEQ ID No: 2. In some embodiments, the first strand further comprises GAT at 3′ end, and the second strand further comprises CT at the 5′ end.

In another aspect, a kit is provided, comprising a plurality of non-naturally occurring nucleic acid molecules, each comprising a first strand comprising SEQ ID No: 1 and a second strand comprising SEQ ID No. 2.

In some embodiments, each of the plurality of non-naturally occurring nucleic acid molecules further comprises a barcode sequence selected from any one of SEQ ID Nos: 205-1259. In some embodiments, the plurality of non-naturally occurring nucleic acid molecules comprises at least 96 subsets, wherein each subset of non-naturally occurring nucleic acid molecules comprises a different barcode sequence selected from any one of SEQ ID Nos: 205-1259.

In another aspect, a composition is provided, comprising a non-naturally occurring nucleic acid molecule comprising a sequence selected from any one of SEQ ID Nos: 5-104.

In some embodiments, the non-naturally occurring nucleic acid molecule is coupled to a support. In some embodiments, the support is a bead. In some embodiments, the non-naturally occurring nucleic acid molecule is further coupled to a template nucleic acid molecule. In some embodiments, the coupling comprises hybridization.

In another aspect, a kit is provided, comprising at least two non-naturally occurring nucleic acid molecules each comprising a different sequence selected from any one of SEQ ID Nos: 5-104.

In some embodiments, each non-naturally occurring nucleic acid molecule is coupled to a support. In some embodiments, the support is a bead.

In another aspect, a composition is provided, comprising a non-naturally occurring nucleic acid molecule comprising a sequence selected from any one of SEQ ID Nos: 105-204.

In some embodiments, the non-naturally occurring nucleic acid molecule is coupled to a support. In some embodiments, the support is a bead. In some embodiments, the non-naturally occurring nucleic acid molecule is further coupled to a template nucleic acid molecule. In some embodiments, the coupling comprises hybridization.

In another aspect, a kit is provided, comprising at least two non-naturally occurring nucleic acid molecules each comprising a different sequence selected from any one of SEQ ID Nos: 105-204.

In some embodiments, each non-naturally occurring nucleic acid molecule is coupled to a support. In some embodiments, the support is a bead.

In another aspect, a composition is provided, comprising a non-naturally occurring nucleic acid molecule comprising a sequence selected from any one of SEQ ID Nos 205-1259.

In some embodiments, 3′ T of the non-naturally occurring nucleic acid molecule is phosphorylated. In some embodiments, the non-naturally occurring nucleic acid molecule further comprises SEQ ID No. 1260 positioned 5′ to the selected sequence.

In another aspect, a kit is provided, comprising at least one non-naturally occurring nucleic acid molecule comprising a sequence selected from any one of SEQ ID Nos: 205-1259.

In some embodiments, the non-naturally occurring nucleic acid molecule further comprises SEQ ID No. 1260 positioned 5′ to the selected sequence. In some embodiments, the 3′ T of the non-naturally occurring nucleic acid molecule is phosphorylated.

In another aspect, a kit is provided, comprising at least 96 non-naturally occurring nucleic acid molecules each comprising a different sequence selected from any one of SEQ ID Nos: 205-1259.

In some embodiments, each non-naturally occurring nucleic acid molecule further comprises SEQ ID No. 1260 positioned 5′ to the selected sequence. In some embodiments, the 3′ T of each non-naturally occurring nucleic acid molecule is phosphorylated.

In another aspect, a kit is provided, comprising at least 256 non-naturally occurring nucleic acid molecules each comprising a different sequence selected from any one of SEQ ID Nos: 205-1259.

In some embodiments, each non-naturally occurring nucleic acid molecule further comprises SEQ ID No. 1260 positioned 5′ to the selected sequence. In some embodiments, the 3′ T of each non-naturally occurring nucleic acid molecule is phosphorylated.

In another aspect, a method is provided, comprising: providing a plurality of template molecules and a first plurality of adapters, wherein adapters in the first plurality of adapters comprise a double-stranded region and a single-stranded region; for each template molecule in the plurality of template molecules, coupling an adapter from the first plurality of adapters to each end of the respective template molecule; providing a second plurality of adapters, wherein the second plurality of adapters each comprise a single strand; and for each template molecule in the plurality of template molecules, coupling an adapter from the second plurality of adapters to the single-stranded regions of previously coupled adapters, wherein the resulting template-adapter molecules do not comprise identical adapters sequences.

In some embodiments, the single-stranded region of adapters in the first plurality of adapters comprises an overhang.

In some embodiments, the double-stranded region of adapters in the first plurality of adapters comprises a first strand and a second strand hybridized to each other.

In some embodiments, the first strand and the second strand are reverse complements of each other.

In some embodiments, the first strand and the second strand are not reverse complements of each other. In some embodiments, there is at least a single base mismatch between the first strand and the second strand.

In some embodiments, a first adapter and a second adapter in the first plurality of adapters comprise different sequences. In some embodiments, there is at least a single base mismatch between the first adapter and the second adapter. In some embodiments, there is no more than a single base mismatch between the first adapter and the second adapter.

In some embodiments, the second plurality of adapters comprise at least a first subset of adapters and a second subset wherein the first and second subsets do not have identical sequences. In some embodiments, there is at least a single base mismatch between adapters in the first subset and second subset. In some embodiments, there is no more than a single base mismatch between adapters in the first subset and the second subset.

In some embodiments, adapters in the second plurality of adapters have identical sequences.

In some embodiments, coupling in step (b) comprises ligating adapters in the first plurality of adapters to library molecules.

In some embodiments, coupling in step (d) comprises (i) hybridizing a first region of adapters in the second plurality of adapters to at least a portion of the single-stranded region of an adapter in the first plurality of adapters, and (ii) ligating 3′ end of the first region to the double-stranded region of the adapter in the first plurality of adapters.

In some embodiments, the coupling in step (b) and step (d) are preformed concurrently.

In some embodiments, the coupling in step (b) and step (d) are preformed sequentially.

In some embodiments, the method further comprises amplifying the template-adapter molecules with a plurality of primers.

In some embodiments, primers in the plurality of primers have identical sequences.

In some embodiments, a first primer and a second primer in the plurality of primers have different sequences. In some embodiments, there is at least a single base mismatch between the first primer and the second primer. In some embodiments, there is no more than a single base mismatch between the first primer and the second primer.

In another aspect, a method for generating barcode sequences is provided, comprising: constructing a barcode sequence of N bases by selecting a nucleotide base type alternatively from (1) a first set of nucleotide base types (K) and (2) a second set of nucleotide base types (M) for N consecutive base positions, wherein base types in the first set of nucleotide base types (K) and the second set of nucleotide base types (M) are mutually exclusive; repeating (a) to construct a plurality of barcode sequences, wherein each of the plurality of barcode sequences is N bases in length and is unique within the plurality of barcode sequences; and electronically outputting the plurality of barcode sequences.

In another aspect, a method for generating barcode sequences is provided, comprising: constructing a barcode sequence of N bases by selecting a nucleotide base type from (1) a first set of nucleotide base types (K) and (2) a second set of nucleotide base types (M) for N consecutive base positions, wherein base types in the first set of nucleotide base types (K) and the second set of nucleotide base types (M) are mutually exclusive, wherein, within the barcode sequence, any base type of the first set of nucleotide base types is only adjacent to any base type of the second set of nucleotide base types; repeating (a) to construct a plurality of barcode sequences, wherein each of the plurality of barcode sequences is N bases in length and is unique within the plurality of barcode sequences; and electronically outputting the plurality of barcode sequences.

In another aspect, a method for generating barcode sequences, is provided comprising: constructing a barcode sequence of length X by selecting one or more nucleotides of a nucleotide base type alternatively from (1) a first set of nucleotide base types (K) and (2) a second set of nucleotide base types (M) for N consecutive times, wherein: base types in the first set of nucleotide base types (K) and the second set of nucleotide base types (M) are mutually exclusive, and X>=N; repeating (a) to construct a plurality of barcode sequences, wherein each of the plurality of barcode sequences is unique within the plurality of barcode sequences; and electronically outputting the plurality of barcode sequences.

In another aspect, a method for generating a set of barcode sequences, is provided comprising: for each respective barcode sequence selecting alternately, for each base position in a plurality of base positions, a nucleotide base type from a first set of nucleotide base types or from a second set of nucleotide base types, wherein: the first set of nucleotide base types comprises a first nucleotide base type and a second nucleotide base type from a first portion of a flow order, and the second set of nucleotide base types comprises a third nucleotide base type and a fourth nucleotide base type from a second portion of the flow order, wherein the flow order comprises an ordered set of the four canonical base types (A, T, C, and G), the plurality of base positions comprises a same number (N) of base positions for each barcode sequence, each base position in a respective barcode sequence comprises a single nucleotide of the selected nucleotide base type, and each respective barcode sequence is distinct from all other barcode sequences in the set of barcode sequences; and electronically outputting the set of barcode sequences.

In some embodiments, the first set of nucleotide base types comprises a first nucleotide base type and a second nucleotide base type. In some embodiments, the second set of nucleotide base types comprises a third nucleotide base type and a fourth nucleotide base type. In some embodiments, the first set of nucleotide base types comprises thymidine and guanine. In some embodiments, the second set of nucleotide base types comprises cytidine and adenine.

In some embodiments, N is an even number. In some embodiments, N is at least 10.

In some embodiments, the set of barcode sequences comprises 2^{N} barcode sequences.

In some embodiments, a first barcode sequence in the set of barcode sequences comprises a nucleotide base type selected from the first set of nucleotides (K) in a first base position of the N consecutive base positions.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example workflow for processing a sample for sequencing.

FIG. 2 illustrates an example flow sequencing method that can be used to generate the sequencing data described herein.

FIG. 3 illustrates an example flowgram.

FIG. 4 illustrates examples of individually addressable locations distributed on substrates, as described herein.

FIGS. 5A-5B illustrate multiplexed stations in a sequencing system.

FIG. 6 illustrates a computer system that is programmed or otherwise configured to implement methods provided herein.

FIG. 7 shows an example image of a substrate with a hexagonal lattice of beads, as described herein.

FIG. 8A illustrates a non-limiting schematic of high-efficiency adapters.

FIG. 8B illustrates a non-limiting example of a high-efficiency adapter, where FIG. 8C and FIG. 8D illustrate non-limiting examples of amplification primers compatible with said high-efficiency adapter. Figure discloses SEQ ID NOS 1-2, 2, 1, 1, 1263, 4, and 2, respectively, in order of appearance.

FIG. 9 illustrates a non-limiting schematic of multi-molecular adapters.

FIG. 10A illustrates non-limiting examples of partially-double-stranded adapters, where each adapter differs by one or more nucleotide bases in the single-stranded region(s).

FIG. 10B illustrates non-limiting examples of partially double-stranded adapters, where each adapter differs by one or more nucleotide bases in the double-stranded region.

FIG. 11 illustrates a non-limiting example of partially double-stranded adapters and multiple species of amplification primers, where each primer differs by one or more nucleotide bases.

FIG. 12 illustrates non-limiting examples of sequencing beads where subpopulations of beads have distinct oligo capture sequences.

FIG. 13 illustrates an example flowgram illustrating that sequences of different lengths may be determined by a same number of nucleotide flows.

FIG. 14 illustrates example flowgrams for SEQ ID Nos: 205 and 311.

FIG. 15 illustrates example flowgrams.

FIG. 16 illustrates example flowgrams. Figure discloses SEQ ID NO: 1264

### DETAILED DESCRIPTION

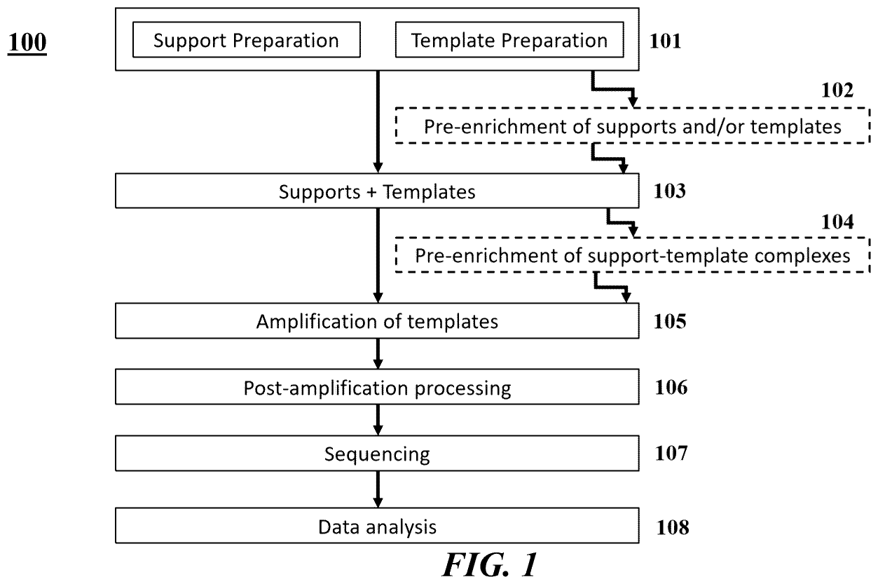

Provided herein are devices, systems, methods, compositions, and kits for library preparation. Such devices, systems, methods, compositions, and kits can be applied alternatively or in addition to sequencing operations described with respect to sequencing workflow 100 of FIG. 1. In addition, such devices, systems, methods, compositions, and kits can be applied alternatively or in addition to template preparation operations described with respect to sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a native sample derived from a subject or specimen.

The term “subject,” as used herein, generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as having at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), 10 Mb, 100 Mb, 1 gigabase or more. A nucleic acid may comprise A nucleic acid can comprise a sequence of four natural nucleotide bases: adenine (A); cytosine (C); guanine (G); and thymine (T) (uracil (U) for thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “terminator” as used herein with respect to a nucleotide may generally refer to a moiety that is capable of terminating primer extension. A terminator may be a reversible terminator. A reversible terminator may comprise a blocking or capping group that is attached to the 3′-oxygen atom of a sugar moiety (e.g., a pentose) of a nucleotide or nucleotide analog. Such moieties are referred to as 3′-O-blocked reversible terminators. Examples of 3′-O-blocked reversible terminators include, for example, 3′-ONH₂ reversible terminators, 3′-O-allyl reversible terminators, and 3′-O-aziomethyl reversible terminators. Alternatively, a reversible terminator may comprise a blocking group in a linker (e.g., a cleavable linker) and/or dye moiety of a nucleotide analog. 3′-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp. and the “lightning terminator” developed by Michael L. Metzker et al. Cleavage of a reversible terminator may be achieved by, for example, irradiating a nucleic acid molecule including the reversible terminator.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA, or a cDNA derived from the mRNA, or another derivative thereof. In another example, the analyte can be a protein, and the template nucleic acid is an oligonucleotide that is conjugated to an antibody that binds to the protein, or derivative thereof. Sequencing may be single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., A base-containing solution) to a sequencing reaction space at a first time point and (ii) providing a nucleotide-containing reagent (e.g., G-base containing solution) to a sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of canonical base types (A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:

(e.g., [A T G C A T G C A T G A T G A T G A T G C
A T G C]).

Such one-dimensional matrix or linear array of bases in the flow order may also be referred to herein as a “flow space.” A flow order may have any number of nucleotide flows. A “flow position,” as used herein, generally refers to the sequential position of a given nucleotide flow in the flow space. A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A T G C], [A A T T G G C C], [A T], [A/T A/G], [A A], [A], [A T G], etc.). A flow cycle may have any number of nucleotide flows. A given flow cycle may be repeated one or more times in the flow cycle, consecutively or non-consecutively. Accordingly, the term “flow cycle order,” as used herein, generally refers to an order of flow cycles within the flow order and can be expressed in units of flow cycles. For example, where [A TG C] is identified as a 1^{st} flow cycle, and [A T G] is identified as a 2^{nd} flow cycle, the flow order of [ATGCATGCATGATGATGATG CA TG C] may be described as having a flow-cycle order of [1^{st} flow cycle; 1^{st} flow cycle; 2^{nd} flow cycle; 2^{nd} flow cycle; 2^{nd} flow cycle; 1^{st} flow cycle; 1^{st} flow cycle].

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence-based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C.C. PNAS, 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28: E87 (2000); Pemov et al., Nucl. Acids Res. 33: e11 (2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65 (2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). Amplification products from a nucleic acid may be identical or substantially identical. A nucleic acid colony resulting from amplification may have identical or substantially identical sequences.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3): 443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; see, e.g., Altschul S F, Gish W, Miller W, Myers E W, Lipman D J, “Basic local alignment search tool” (1990) J Mol Biol 215 (3): 403-410). As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity as such sequences would generally be considered “identical.”

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle. A reversible coupling may comprise, for example, a releasable coupling (e.g., in which a first object may be released from a second object to which it is coupled). A first object releasably coupled to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. Coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a particle may be coupled to a planar support via an electrostatic interaction, a magnetic interaction, or a covalent interaction. Similarly, a nucleic acid molecule may be coupled to a particle via a covalent interaction or a via a non-covalent interaction. A coupling between a first object and a second object may comprise a labile moiety, such as a moiety comprising an ester, vicinal diol, phosphodiester, peptide, glycosidic, sulfone, Diels-Alder, or similar linkage. The strength of a coupling between a first object and a second object may be indicated by a dissociation constant, K_{d}, which indicates the inclination of a coupled object comprising a first object and a second object to dissociate into the uncoupled first and second objects and may be expressed as a ratio of dissociated (e.g., uncoupled) objects to coupled objects.

### Sample Processing Methods

Described herein are devices, systems, methods, compositions, and kits for processing samples, such as to prepare a sample for sequencing, to sequence a sample, and/or to analyze sequencing data. FIG. 1 illustrates an example sequencing workflow 100, according to the devices, systems, methods, compositions, and kits of the present disclosure.

Supports and/or template nucleic acids may be prepared and/or provided (101) to be compatible with downstream sequencing operations (e.g., 107). A support (e.g., bead) may be used to help facilitate sequencing of a template nucleic acid on a substrate. The support may help immobilize a template nucleic acid to a substrate, such as when the template nucleic acid is coupled to the support, and the support is in turn immobilized to the substrate. The support may further function as a binding entity to retain molecules of a colony of the template nucleic acid (e.g., copies comprising identical or substantially identical sequences as the template nucleic acid) together for any downstream processing, such as for sequencing operations. This may be particularly useful in distinguishing a colony from other colonies (e.g., on other supports) and generating amplified sequencing signals for a template nucleic acid sequence.

A support that is prepared and/or provided may comprise an oligonucleotide comprising one or more functional nucleic acid sequences. For example, the support may comprise a capture sequence configured to capture or be coupled to a template nucleic acid (or processed template nucleic acid). For example, the support may comprise the capture sequence, a primer sequence, a barcode sequence, a sample index sequence, a unique molecular identifier (UMI), a flow cell adapter sequence, an adapter sequence, a binding sequence for any molecule (e.g., splint, primer, template nucleic acid, capture sequence, etc.), or any other functional sequence useful for a downstream operation, or any combination thereof. The oligonucleotide may be single-stranded, double-stranded, or partially double-stranded.

A support may comprise one or more capture entities, where an affinity tagis configured for capture by a capturing entity. An affinity tag may be coupled to an oligonucleotide coupled to the support. An affinity tag may be coupled to the support. For example, the capturing entity may comprise streptavidin (SA) when the affinity tag comprises biotin. In another example, the capturing entity may comprise a complementary capture sequence when the affinity tag comprises a capture sequence (e.g., a capture oligonucleotide that is complementary to the complementary capture sequence). In another example, the capturing entity may comprise an apparatus, system, or device configured to apply a magnetic field when the affinity tag comprises a magnetic particle. In another example, the capturing entity may comprise an apparatus, system, or device configured to apply an electrical field when the affinity tag comprises a charged particle. In some instances, the capturing entity may comprise one or more other mechanisms configured to capture the affinity tag. An affinity tag and capturing entity may bind, couple, hybridize, or otherwise associate with each other. The association may comprise formation of a covalent bond, non-covalent bond, and/or releasable bond (e.g., cleavable bond that is cleavable upon application of a stimulus). In some cases, the association may not form any bond. For example, the association may increase a physical proximity (or decrease a physical distance) between the capturing entity and affinity tag. In some instances, a single affinity tag may be capable of associating with a single capturing entity. Alternatively, a single affinity tag may be capable of associating with multiple capturing entities. Alternatively or in addition, a single capturing entity may be capable of associating with multiple capture entities. The affinity tag may be capable of linking to a nucleotide. Chemically modified bases comprising biotin, an azide, cyclooctyne, tetrazole, and a thiol, and many others are suitable as capture entities. The affinity tag/capturing entity pair may be any combination. The pair may include, but is not limited to, biotin/streptavidin, azide/cyclooctyne, and thiol/maleimide. It will be appreciated that either of the pair may be used as either the affinity tag or the capturing entity. In some instances, the capturing entity may comprise a secondary affinity tag, for example, for subsequent capture by a secondary capturing entity. The secondary affinity tag and secondary capturing entity may comprise any one or more of the capturing mechanisms described elsewhere herein (e.g., biotin and streptavidin, complementary capture sequences, etc.). In some instances, the secondary affinity tag can comprise a magnetic particle (e.g., magnetic bead) and the secondary capturing entity can comprise a magnetic system (e.g., magnet, apparatus, system, or device configured to apply a magnetic field, etc.). In some instances, the secondary affinity tag can comprise a charged particle (e.g., charged bead carrying an electrical charge) and the secondary capturing entity can comprise an electrical system (e.g., magnet, apparatus, system, or device configured to apply an electric field, etc.).

A support may comprise one or more cleaving moieties. The cleavable moiety may be part of or attached to an oligonucleotide coupled to the support. The cleavable moiety may be coupled to the support. A cleavable moiety may comprise any useful cleavable or excisable moiety that can be used to cleave an oligonucleotide (or portion thereof) from the support. For example, the cleavable moiety may comprise a uracil, a ribonucleotide, or other modified nucleotide that is excisable or cleavable using an enzyme (e.g., uracil D glycosylase (UDG), RNAse, endonuclease, exonuclease, etc.). The cleavable moiety may comprise an abasic site or an analog of an abasic site (e.g., dSpacer), a dideoxyribose. The cleavable moiety may comprise a spacer, e.g., C3 spacer, hexanediol, triethylene glycol spacer (e.g., Spacer 9), hexa-ethylene glycol spacer (e.g., Spacer 18), or combinations or analogs thereof. The cleavable moiety may comprise a photocleavable moiety. The cleavable moiety may comprise a modified nucleotide, e.g., a methylated nucleotide. The modified nucleotide may be recognized specifically by an enzyme (e.g., a methylated nucleotide may be recognized by MspJI). The cleavable moiety may be cleaved enzymatically (e.g., using an enzyme such as UDG, RNAse, APEI, MspJI, etc.). Alternatively, or in addition to, the cleavable moiety may be cleavable using one or more stimuli, e.g., photo-stimulus, chemical stimulus, thermal stimulus, etc.

In some examples, a single support comprises copies of a single species of oligonucleotide, which are identical or substantially identical to each other. In some examples, a single support comprises copies of at least two species of oligonucleotides (e.g., comprising different sequences). For example, a single support may comprise a first subset of oligonucleotides configured to capture a first adapter sequence of a template nucleic acid and a second subset of oligonucleotides configured to capture a second adapter sequence of a template nucleic acid.

In some examples, a population of a single species of supports may be prepared and/or provided, where all supports within a species of supports is identical (e.g., has identical oligonucleotide composition (e.g., sequence), etc.). In some examples, a population of multiple species of supports may be prepared and/or provided. For example, a population of supports may be prepared to comprise a plurality of unique support species, where each unique support species comprises a primer sequence unique to said support species. When attaching template nucleic acids to supports, only a template nucleic acid comprising a given adapter sequence compatible with (e.g., at least partially complementary to) a given primer sequence may be capable of attaching to a given support of a support species comprising the given primer sequence. In another example, a population of supports may be prepared, such that each unique support species comprises a plurality of primer sequences (e.g., a pair of primer sequences) unique to said support species. In some embodiments, the systems and methods disclosed herein can include a population of supports that comprise two, three, four, five, six, seven, eight, nine, ten or more unique support species. Each unique support species can comprise a unique primer sequence that allows selective interactions between the respective support species with an intended binding partner (e.g., a complementary nucleic acid sequence within an adapter region of a template nucleic acid or an intermediary primer sequence which can subsequently bind to a complementary nucleic acid sequence within an adapter region of a sample nucleic acid). A population of multiple species of supports may be prepared by first preparing distinct populations of a single species of supports, all different, and mixing such distinct populations of single species of supports to result in the final population of multiple species of supports. A concentration of the different support species within the final mixture may be adjusted accordingly. Devices, systems, methods, compositions, and kits for preparing and using support species are described in further detail in International Pub. No. WO2020/167656 and International App. No. PCT/US2021/046951, each of which is entirely incorporated herein by reference for all purposes.

A template nucleic acid may include an insert sequence sourced from a biological sample. In some cases, the insert sequence may be derived from a larger nucleic acid in the biological sample (e.g., an endogenous nucleic acid), or reverse complement thereof, for example by fragmenting, transposing, and/or replicating from the larger nucleic acid. The template nucleic acid may be derived from any nucleic acid of the biological sample and result from any number of nucleic acid processing operations, such as but not limited to fragmentation, degradation or digestion, transposition, ligation, reverse transcription, extension, etc. A template nucleic acid that is prepared and/or provided may comprise one or more functional nucleic acid sequences. In some cases, the one or more functional nucleic acid sequences may be disposed at one end of the insert sequence. In some cases, the one or more functional nucleic acid sequences may be separated and disposed at both ends of an insert sequence, such as to sandwich the insert sequence. In some cases, a nucleic acid molecule comprising the insert sequence, or complement thereof, may be ligated to one or more adapter oligonucleotides that comprise such functional nucleic acid sequence(s). In some cases, a nucleic acid molecule comprising the insert sequence, or complement thereof, may be hybridized to a primer comprising such functional nucleic acid sequence(s) and extended to generate a template nucleic acid comprising such functional nucleic acid sequence(s). In some cases, a nucleic acid molecule comprising the insert sequence, or complement thereof, may be hybridized to a primer comprising one or more functional nucleic acid sequence(s) and extended to generate an intermediary molecule, and the intermediary molecule hybridized to a primer comprising additional functional nucleic acid sequence(s) and extended, and so on for any number of extension reactions, to generate a template nucleic acid comprising one or more functional nucleic acid sequence(s). For example, the template nucleic acid may comprise an adapter sequence configured to be captured by a capture sequence on an oligonucleotide coupled to a support. For example, the template nucleic acid may comprise a capture sequence, a primer sequence, a barcode sequence, a sample index sequence, a unique molecular identifier (UMI), a flow cell adapter sequence, the adapter sequence, a binding sequence for any molecule (e.g., splint, primer, template nucleic acid, capture sequence, etc.), or any other functional sequence useful for a downstream operation, or any combination thereof. The template nucleic acid may be single-stranded, double-stranded, or partially double-stranded.

A template nucleic acid may comprise one or more capture entities that are described elsewhere herein. In some cases, in the workflow, only the supports comprise capture entities and the template nucleic acids do not comprise capture entities. In other cases, in the workflow, only the template nucleic acids comprise capture entities and the supports do not comprise capture entities. In other cases, both the template nucleic acids and the supports comprise capture entities. In other cases, neither the supports nor the template nucleic acids comprise capture entities.

A template nucleic acid may comprise one or more cleaving moieties that are described elsewhere herein. In some cases, in the workflow, only the supports comprise cleavable moieties and the template nucleic acids do not comprise cleavable moieties. In other cases, in the workflow, only the template nucleic acids comprise cleavable moieties and the supports do not comprise cleavable moieties. In other cases, both the template nucleic acids and the supports comprise cleavable moieties. In other cases, neither the supports nor the template nucleic acids comprise cleavable moieties. A cleavable moiety may be strategically placed based on a desired downstream amplification workflow, for example.

In some examples, a library of insert sequences is processed to provide a population of template sequences with identical configurations, such as with identical sequences and/or locations of one or more functional sequences. For example, a population of template sequences may comprise a plurality of nucleic acid molecules each comprising an identical first adapter sequence ligated to a same end. In some examples, a library of insert sequences is processed to provide a population of template sequences with varying configurations, such as with varying sequences and/or locations of one or more functional sequences. For example, a population of template sequences may comprise a first subset of nucleic acid molecules each comprising an identical first adapter sequence at a first end, and a second subset of nucleic acid molecules each comprising an identical second adapter sequence at the second end, where the second adapter sequence is different form the first adapter sequence. In some instances, a population of template sequences with varying configurations (e.g., varying adapter sequences) may be used in conjunction with a population of multiple species of supports, such as to reduce polyclonality problems during downstream amplification. A population of multiple configurations of template nucleic acids may be prepared by first preparing distinct populations of a single configuration of template nucleic acids, all different, and mixing such distinct populations of single configurations of template nucleic acids to result in the final population of multiple configurations of template nucleic acids. A concentration of the different configurations of template nucleic acids within the final mixture may be adjusted accordingly.

Optionally, the supports and/or template nucleic acids may be pre-enriched (102). For example, a support comprising a distinct oligonucleotide sequence is isolated from a mixture comprising support(s) that do not have the distinct oligonucleotide sequence. Alternatively, a support population may be provided to comprise substantially uniform supports, where each support comprises an identical surface primer molecule immobilized thereto. For example, template nucleic acids comprising a distinct configuration (e.g., comprising a particular adapter sequence) are isolated from a mixture comprising template nucleic acids that do not have the distinct configuration. Alternatively, a template nucleic acid population may be provided to comprise substantially uniform configurations. In some cases, the capture entit (ies) on the supports and/or template nucleic acids are used for pre-enrichment.

Subsequent to preparation of the supports and template nucleic acids, the two may be attached (103). A template nucleic acid may be coupled to a support via any method(s) that results in a stable association between the template nucleic acid and the support. For example, the template nucleic acid may hybridize to an oligonucleotide on the support. In another example, the template nucleic acid may hybridize to one or more intermediary molecules, such as a splint, bridge, and/or primer molecule, which hybridizes to an oligonucleotide on the support. Alternatively or in addition, a template nucleic acid may be ligated to one or more nucleic acids on or coupled to the support. Alternatively or in addition, a template nucleic acid may be hybridized to an oligonucleotide on a support, which oligonucleotide comprises a primer sequence, and subsequent extension form the primer sequence is performed. Once attached, a plurality of support-template complexes may be generated.

Optionally, support-template complexes may be pre-enriched (104), wherein a support-template complex is isolated from a mixture comprising support(s) and/or template nucleic acid(s) that are not attached to each other. In some cases, the capture entit (ies) on the supports and/or template nucleic acids are used for pre-enrichment.

Subsequent to attachment of the template nucleic acid molecule to the support, the template nucleic acids may be subjected to amplification reactions (105) to generate a plurality of amplification products immobilized to the support. For example, such amplification reactions may comprise performing polymerase chain reaction (PCR) or any other amplification methods described herein, including but not limited to emulsion PCR (ePCR or emPCR), isothermal amplification (e.g., recombinase polymerase amplification (RPA)), bridge amplification, template walking, etc. In some cases, amplification reactions can occur while the support is immobilized to a substrate. In other cases, amplification reactions can occur off the substrate, such as in solution, or on a different surface or platform. In some cases, amplification reactions can occur in isolated reaction volumes, such as within multiple droplets (e.g., partitions) in an emulsion during emulsion PCR (ePCR or emPCR), or in wells. Emulsion PCR methods are described in further detail in International Pub. No. WO2020/167656 and International App. No. PCT/US2021/046951, each of which is entirely incorporated by reference herein.

Subsequent to amplification, the supports (e.g., comprising the template nucleic acids) may be subjected to post-amplification processing (106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described in U.S. Pub. No. 2021/0277464 and International App. No. PCT/US2021/046951, each of which is entirely incorporated by reference herein. For example, an on-substrate enrichment procedure may immobilize only the positive supports onto the substrate surface to isolate the positive supports. In some instances, the positive supports may be immobilized to desired locations on the substrate surface (e.g., individually addressable locations), as distinguished from undesired locations (e.g., spacers between the individually addressable locations). In some instances, positive supports and/or negative supports may be processed to selectively remove unamplified surface primers (on the support(s)), such that a resulting positive support retains the template nucleic acid molecule, and a resulting negative support is stripped of the unamplified surface primers. Subsequently, the template nucleic acid(s) on the positive supports may be used to enrich for the positive supports, e.g., by capturing the template nucleic acids.

Subsequent to post-amplification processing, the template nucleic acids may be subject to sequencing (107). The template nucleic acid(s) may be sequenced while attached to the support. Alternatively, the template nucleic acid molecules may be free of the support when sequenced and/or analyzed. In some instances, the template nucleic acids may be sequenced while attached to the support which is immobilized to a substrate. Examples of substrate-based sample processing systems are described elsewhere herein. Any sequencing method described elsewhere herein may be used. In some cases, sequencing by synthesis (SBS) is performed.

In one example (Example A), an SBS method comprises flowing nucleotide reagents according to a flow order comprising a repeat of one 4-base flow (e.g., [A/T/G/C]), where each nucleotide is reversibly terminated (e.g., dideoxynucleotide), and where each base is labeled with a different dye (yielding different optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of each base can be detected by interrogating the different dyes in 4 channels. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example B), an SBS method comprises flowing nucleotide reagents according to a flow order comprising a repeat of a flow cycle of 4 single base flows (e.g., [A T G C]), where each nucleotide is reversibly terminated, and where each base is labeled with a same dye (yielding same frequency optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of the particular base in that flow can be detected by interrogating the wavelength of the dye. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example C), an SBS method comprises flowing nucleotide reagents according to a flow order comprising a repeat of a flow cycle of 4 single base flows (e.g., [A T G C]), where each nucleotide is not terminated, and where each base is labeled with a same dye (yielding same frequency optical signals). With each flow, other sequencing reagents, e.g., sequencing primer, polymerase, buffer, etc. are present to provide sufficient conditions for incorporation of the labeled nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example D), an SBS method comprises flowing nucleotide reagents according to a flow order comprising a repeat of a flow cycle of 4 single base flows (e.g., [A T G C]), where each nucleotide is not terminated, and where only a fraction of the bases in each flow (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals). With each flow, other sequencing reagents, e.g., sequencing primer, polymerase, buffer, etc. are present to provide sufficient conditions for incorporation of the nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example E), an SBS method comprises flowing nucleotide reagents according to a flow order comprising a repeat of a flow cycle of 8 single base flows, with each of the 4 canonical base types flowed twice consecutively within the flow cycle, (e.g., [A A T T G G C C]), where each nucleotide is not terminated, and where only a fraction of the bases in every other flow in the flow cycle (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals) and the nucleotides in the alternating other flow is unlabeled. With each flow, other sequencing reagents, e.g., sequencing primer, polymerase, buffer, etc. are present to provide sufficient conditions for incorporation of the nucleotide into a growing strand hybridized to a template nucleic acid. After one or both of the flows for each canonical base type, an incorporation event or lack thereof of the particular base in that flow can be detected by interrogating the wavelength of the dye. Because the nucleotides are not terminated, if the growing strand is extending through a homopolymer region (e.g., polyT region) of the template nucleic acid, multiple nucleotides may be incorporated during one flow. A first flow of a canonical base type (e.g., A) followed by a second flow of the same canonical base type (e.g., A) may help facilitate completion of incorporation reactions across each growing strand such as to reduce phasing problems. After each or one or more detection events, the labels may be removed (e.g., dyes are cleaved) to reduce signal noise for the next detection.

Labeled nucleotides may comprise a dye, fluorophore, or quantum dot. Non-limiting examples of dyes include SYBR green, SYBR blue, DAPI, propidium iodine, Hoechst, SYBR gold, ethidium bromide, acridine, proflavine, acridine orange, acriflavine, fluorocoumarin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40,-41,-42,-43,-44,-45 (blue), SYTO-13,-16,-24,-21,-23,-12,-11,-20,-22,-15,-14,-25 (green), SYTO-81,-80,-82, -83,-84,-85 (orange), SYTO-64,-17,-59,-61,-62,-60,-63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5-(or 6-) iodoacetamidofluorescein, 5-{[2 (and 3)-5-(Acetylmercapto)-succinyl] amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, Atto 390, 425, 465, 488, 495, 532, 565, 594, 633, 647, 647N, 665, 680 and 700 dyes, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; Cy5Q and Cy7Q and Dark Cyanine dyes (GE Healthcare); Dy-Quenchers (Dyomics), such as DYQ-660 and DYQ-661; and ATTO fluorescent quenchers (ATTO-TEC GmbH), such as ATTO 540Q, 580Q, 612Q. In some cases, the label may be one with linkers. For instance, a label may have a disulfide linker attached to the label. Non-limiting examples of such labels include Cy5-azide, Cy-2-azide, Cy-3-azide, Cy-3.5-azide, Cy5.5-azide and Cy-7-azide. In some cases, a linker may be a cleavable linker. In some cases, the label may be a type that does not self-quench or exhibit proximity quenching. Non-limiting examples of a label type that does not self-quench or exhibit proximity quenching include Bimane derivatives such as Monobromobimane. Alternatively, the label may be a type that self-quenches or exhibits proximity quenching. Non-limiting examples of such labels include Cy5-azide, Cy-2-azide, Cy-3-azide, Cy-3.5-azide, Cy5.5-azide and Cy-7-azide. In some instances, a blocking group of a reversible terminator may comprise the dye.

It will be appreciated that the combinations of termination states on the nucleotides, label types (e.g., types of dye or other detectable moiety), fraction of labeled nucleotides within a flow, type of nucleotide bases in each flow, type of nucleotide bases in each flow cycle, and/or the order of flows in a flow cycle and/or flow order, other than enumerated in Examples A-E, can be varied for different SBS methods.

Subsequent to sequencing, the sequencing signals collected and/or generated may be subjected to data analysis (108). The sequencing signals may be processed to generate base calls and/or sequencing reads. In some cases, the sequencing reads may be processed to generate diagnostics data to the biological sample, or the subject from which the biological sample was derived from.

While the sequencing workflow 100 with respect to FIG. 1 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

It will be appreciated that in some instances, the different operations described in the sequencing workflow 100 may be performed in a different order. It will be appreciated that in some instances, one or more operations described in the sequencing workflow 100 may be omitted or replaced with other comparable operation(s). It will be appreciated that in some instances, one or more additional operations described in the sequencing workflow 100 may be performed.

The different operations described with respect to sequencing workflow 100 may be performed with the help of open substrate systems described herein.

### Sequencing Methods

During sequencing by synthesis, a sequencing primer may be hybridized to a template (e.g., to a primer binding site on the template) and extended in a stepwise manner by, in each extension step, contacting the complex with nucleotide reagents of known canonical base type(s). The extended or extending sequencing primer may also be referred to herein as a growing strand. An extension step may be a bright step (also referred to herein, in some cases, as labeled step, hot step, or detected step) or a dark step (also referred to herein, in some cases, as an unlabeled step, cold step, or undetected step). A sequencing method may comprise only bright steps. Alternatively, a sequencing method may comprise a mix of bright step(s) and dark step(s). For a bright step, the growing strand may be contacted with nucleotide reagents that include labeled nucleotides (of known canonical base type(s)) and signals indicative of incorporation of the labeled nucleotides, or lack thereof, may be detected to determine a base or sequence of the template. Alternatively or in addition, for a bright step, the growing strand may be contacted with a mixture of labeled and unlabeled nucleotide reagents. For a dark step, the growing strand may be contacted with solely unlabeled nucleotide reagents. Alternatively or in addition, for a dark step, the growing strand may be contacted with labeled nucleotide reagents and detection omitted. Sequencing data can be generated from the signals collected after one or more extension steps. A sequencing by synthesis method may comprise any number of bright steps and any number of dark steps. A sequencing by synthesis method may comprise any number of bright regions (consecutive bright steps) and any number of dark regions (consecutive dark steps). In some cases, the dark steps or dark regions may be used to accelerate or fast forward through certain regions of the template during sequencing. In some cases, the dark steps or dark regions may be advantageous to correct phasing problems.

Sequencing methods of the present disclosure may comprise flow-based sequencing, non-terminated sequencing, and/or terminated sequencing. Sequencing methods of the present disclosure may be applied to colony-based sequencing where template strands are provided in clusters, each cluster comprising copies of a single template strand, concatemer-based sequencing where template strands are provided as concatemers, each concatemer comprising multiple copies of a single template insert, or single molecule-based sequencing where template strands are provided as single molecules as opposed to colonies, clusters, or concatemers. For non-single molecule-based sequencing methods, multiple sequencing primers may be simultaneously bound to multiple primer binding sites across multiple copies of a template insert (in clusters or in a concatemer), extended in parallel, and provide synchronized and cumulative signals from the multiple copies at bright steps.

### Terminated Sequencing

In terminated sequencing methods, a bright step may comprise terminated nucleotides (e.g., reversibly terminated nucleotides). In some cases, a bright step may comprise a single nucleotide base type (e.g., A, C, G, T, U) or a mixture of nucleotide base types (e.g., 2, 3, 4, or more base types). A dark step may comprise terminated nucleotides, unterminated nucleotides, or a mixture thereof. A dark step may comprise a single nucleotide base type. Alternatively, a dark step may comprise a mixture of nucleotide base types. In an extension step comprising solely reversibly terminated nucleotides, at most a single nucleotide base may be incorporated into a growing strand. In an extension step comprising a mixture of reversibly terminated and unterminated nucleotides, more than one nucleotide base may be incorporated into a growing strand, the last incorporation being of a terminated nucleotide.

### Non-Terminated Sequencing, Flow-Based Sequencing

Sequencing data can be generated using flow-based sequencing methods that include extending a primer bound to a template nucleic acid according to a pre-determined flow cycle and/or flow order where, in one or more flow positions, known canonical base type(s) of nucleotides (e.g., A, C, G, T, U) is accessible to the extending primer. At least some of the nucleotides may include a label, which labeled nucleotides upon incorporation into the extending primer render a detectable signal. The resulting sequence by which nucleotides are incorporated into the extended primer is expected to be the reverse complement of the sequence of the template nucleic acid. A method for sequencing can comprise using a flow sequencing method that includes (1) extending a primer using labeled nucleotides in a flow, and (2) detecting the presence or absence of a labeled nucleotide incorporated into the extending primer to generate sequencing data. Flow sequencing methods may also be referred to as “natural sequencing-by-synthesis,”“mostly natural sequencing-by-synthesis,” or “non-terminated sequencing-by-synthesis” methods. Example methods are described in U.S. Pat. No. 8,772,473 and U.S. patent Ser. No. 11/459,609, each of which is incorporated herein by reference in its entirety.

In flow sequencing, iterative nucleotide flows are used to extend the primer hybridized to the template nucleic acid, with detection of incorporated nucleotides between one or more flows. The nucleotides may be, for example, non-terminating nucleotides such that more than one consecutive base can be incorporated into the extending primer strand if more than one consecutive complementary base (or homopolymer region) is present in the template strand. At least a portion of the nucleotides can be labeled so that incorporation can be detected. Generally, only a single nucleotide type is introduced in a flow, although two or three different types of nucleotides may be simultaneously introduced in certain embodiments. This methodology can be contrasted with sequencing methods that use a reversible terminator, where primer extension is stopped after extension of every single base before the terminator is reversed (e.g., by removing a 3′ blocking group) to allow incorporation of the next succeeding base.

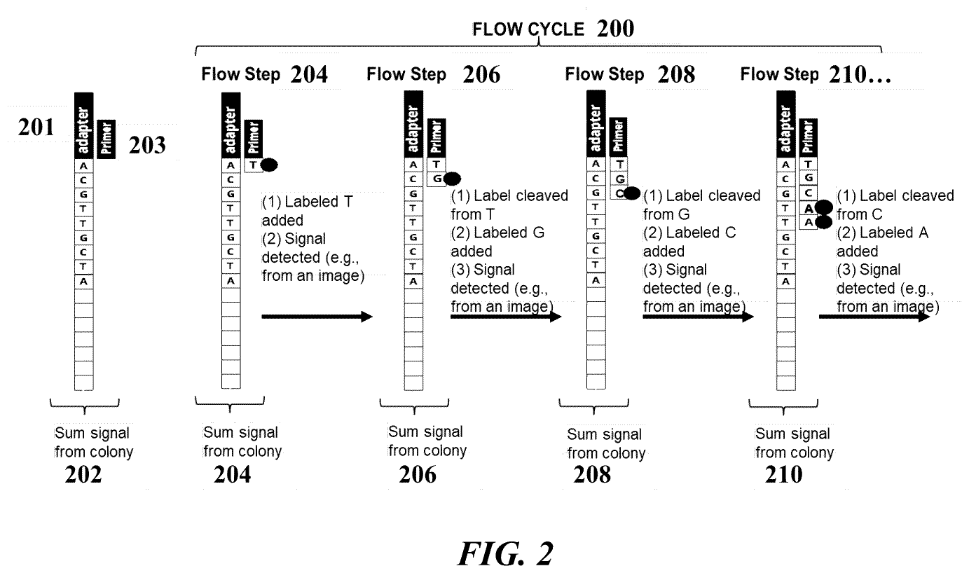

FIG. 2 illustrates an example flow sequencing method that can be used to generate the sequencing data described herein. Template nucleic acids may be immobilized to a surface (e.g., the surface of a bead attached to a substrate or directly to a substrate), as described in detail herein. In this example, the template nucleic acid includes an adaptor sequence 201 followed by an insert sequence (e.g., “ACGTTGCTA . . . ”). The adaptor sequence 201 can include a sequencing primer hybridization site. At operation 202, a sequencing primer 203 is hybridized to the adapter sequence 201 at the sequencing primer hybridization site. The sequencing primer 203 is then extended in a series of flows according to flow cycle 200 with flow order: [T G C A]. In this example, the flow cycle 200 includes four flow steps 204, 206, 208, 210, and in a given flow step, a single base type is provided to the template-primer hybrid. In flow step 204, nucleotides comprising labeled T nucleotides are provided; in flow step 206, nucleotides comprising labeled G nucleotides are provided; in flow step 208, nucleotides comprising labeled C nucleotides are provided; in flow step 210, nucleotides comprising labeled A nucleotides are provided. Nucleotides in a single-base flow may comprise a mixture of labeled and unlabeled nucleotides of the single base. At flow step 204, a labeled T nucleotide is incorporated by the extending sequencing primer 203 opposite the A base in the template strand. Then, a signal indicative of the incorporation of the labeled T nucleotide can be detected. For example, the signal may be detected by imaging the surface the template nucleic acids are immobilized on and analyzing the resulting image(s). The sequencing platform may be washed with a wash buffer to remove unincorporated nucleotides prior to signal detection. In some cases, prior to the next flow step (e.g., 206), the label may be removed from the incorporated labeled T nucleotide (e.g., by cleaving the label from the nucleotide), before proceeding. Nucleotide flow, detection, and optionally cleavage, may be repeated according to a flow order that may or may not include repeating the flow cycle 200 for any number of times. Flow step 210 illustrates incorporation of two labeled A bases by the extending sequencing primer 203 opposite the two T bases in the template strand, per the non-terminated nature of the flown nucleotides. The detected signal intensity indicating the incorporation of two A nucleotides may be greater than the signal intensity indicating the incorporation of one nucleotide. For simplicity, this Figure illustrates incorporation of two labeled A nucleotides in the same hybrid. However, flow-based sequencing may be performed on colonies of amplified molecules, e.g., each bead representing one colony, where an optically resolvable location contains multiple copies of the same template nucleic acid molecule (e.g., a location contains one amplified bead), such that the signal detected at an optically resolvable location represents an aggregate signal from the multiple copies of molecules. Thus, when using a nucleotide flow mixture containing labeled and unlabeled nucleotides of a same base type, the incorporation of the labeled nucleotides can be distributed across the multiple copies of the molecules, and the aggregate signal from the multiple copies detected. In some cases, for a majority of hybrids, at most a single labeled nucleotide may be incorporated into a single homopolymer stretch in a hybrid—the longer the homopolymer stretch, the more likely that more hybrids of the plurality of copies of hybrids in an optically resolvable location will incorporate one labeled nucleotide.

While each flow step in the example flow sequencing method in FIG. 2 results in incorporation of one or more nucleotides (and thus a detected signal indicating such incorporation), it should be appreciated that not all flow steps result in incorporation of nucleotides. In some flow steps, no nucleotide base may be incorporated (for example, in the absence of a complementary base in the template).

A nucleotide mixture that is provided during any one flow may comprise only labeled nucleotides, only unlabeled nucleotides, or a mixture of labeled and unlabeled nucleotides. The mixture of labeled and unlabeled nucleotides may be of any fraction of labeled nucleotides, such as at least or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. Labeled nucleotides may comprise a dye, fluorophore, or quantum dot, multiples thereof, and/or combination thereof. In some cases, nucleotides of different canonical base types may be labeled and detectable at a single frequency (e.g., using the same or different dyes). In other cases, nucleotides of different canonical base types may be labeled and detectable at different frequencies (e.g., using the same or different dyes). Labeled nucleotides may comprise an optical moiety (e.g., dye, fluorophore, quantum dot, label, etc.) coupled to a nucleobase via a linker, and the label from the labeled nucleotides may be removed by cleaving the linker to remove the optical moiety. Cleaving may comprise one or more stimuli, such as exposure to a chemical (e.g., reducing agent), an enzyme, light (e.g., UV light), or temperature change (e.g., heat).

Flow-based sequencing may comprise providing non-detected nucleotide flow(s), for example to skip sequencing of a region(s) of the template nucleic acid; to ensure completion of incorporation reactions across all template-primer hybrids in the reaction space; and/or phasing or re-phasing. A non-detected nucleotide flow may be referred to herein as a “dark flow”, “dark tap”, or “dark tap flow.” A detected nucleotide flow may be referred to herein as a “bright flow”, “bright tap”, or “bright tap flow.” Incorporation reactions may be incomplete in the reaction space when not all available incorporation sites in the template-primer hybrids have incorporated a complementary base, such as due to reaction kinetics and/or insufficient incubation time or reagents. In some cases, single-base flows of the same canonical base type may be provided consecutively (without intervening flow of a different nucleotide base type) for any number of consecutive flows, to ensure completion of incorporation reactions. A consecutive same-base flow may be referred to herein as a “double tap” or “double tap flow” if there are two consecutive flows, a “triple tap” or “triple tap flow” if there are three consecutive flows, or a “nth tap” or “nth tap flow” if there are n consecutive flows of the same base type. A double tap, triple tap, or nth tap flow may or may not be detected. Labels in a flow may or may not be removed (e.g., cleaved) prior to the double tap, triple tap, or nth tap flow. Detection of labeled nucleotides from a particular flow may be performed prior to, during, or subsequent to the double tap, triple tap, or nth tap flow. Accordingly, below are non-limiting examples of flow cycles that can be used in a larger flow order of flow-based sequencing methods, which may or may not be repeated and/or mixed and matched with other flow cycles, where * after a base represents a detected flow step and/between bases represents a mixed base flow:
Single-base flow: e.g., [T* A* C * G*]
Single-base flow with double tap: e.g., [T* T A* A C* C G* G]
Mixed base flow, all labeled: e.g., [T* A*/C*/G*]
Mixed base flow, some unlabeled: e.g., [T* A/C*/G]
Mixed base flow, some unlabeled: e.g., [T A*/C*/G*]
Skip region base flow: e.g., [T/A/C G/A/T]
Three base flow cycle: e.g., [T A C]

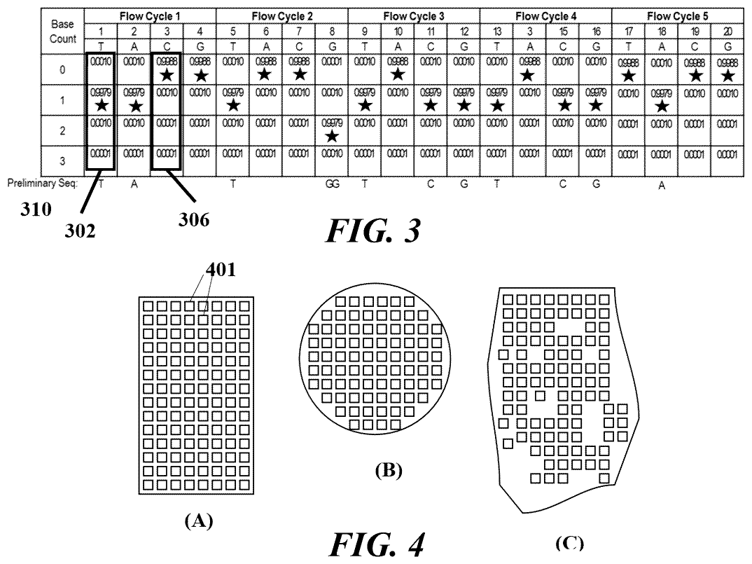

FIG. 3 illustrates an example flowgram of signals detected after five exemplary flow cycles of [T A C G] are performed to extend a sequencing primer, in accordance with some cases. Each column in the flowgram corresponds to a detected flow step (e.g., 302, 306), and the values in each column collectively represent the detected signal intensity in the flow step. In each detected flow step, the flow signal can be determined from an analog signal that is detected during the sequencing process, such as a fluorescent signal of the one or more bases incorporated. In some cases, for a flow step, the detected signal intensity can be expressed in probabilistic terms. Specifically, the detected signal intensity can be expressed in a series of likelihood values corresponding to different integer homopolymer base lengths (e.g., 0 base, 1 base, 2 bases, 3 bases, etc.) for the flow position. For flow step 302, the detected signal intensity is expressed by a first likelihood value of 0.001 for 0 base, a second likelihood value of 0.9979 for 1 base, a third likelihood value of 0.001 for 3 bases, and a fourth likelihood value of 0.0001 for 4 bases. This can be interpreted to indicate that there is a high statistical likelihood that one nucleotide base has been incorporated. In this flow step, a single T was determined to be incorporated, which means there is an A in the template. Similarly, for flow step 306, the column values can collectively indicate that there is a high statistical likelihood that no base has been incorporated (with 0.9988 likelihood value for 0 bases). With similar analyses performed at each flow position, a preliminary sequence 310 (TATGGTCGTCGA (SEQ ID NO: 1262)) of the extending primer can be determined, and reverse complement (i.e., the template strand sequence) readily determined from the preliminary sequence. For example, the most likely sequence can be determined by selecting the base count with the highest likelihood at each flow position, as shown by the stars in the flowgram. Further, the likelihood of this sequencing data set can be determined as the product of the selected likelihood at each flow position. Accordingly, the flowgram may be formatted as a sparse matrix, with a flow signal represented by a plurality of likelihood values indicative of a plurality of base homopolymer length counts at each flow position. The homopolymer length likelihood may vary, for example, based on the noise or other artifacts present during detection of the analog signal during sequencing. In some cases, if the homopolymer length likelihood statistical parameter or likelihood is below a predetermined threshold, the parameter may be set to a predetermined non-zero value that is substantially zero (i.e., some very small value or negligible value) to aid the downstream statistical analysis further discussed herein, wherein a true zero value may give rise to a computational error or insufficiently differentiate between levels of unlikelihood, e.g., very unlikely (0.0001) and inconceivable (0). Thus, a method for sequencing may comprise generating a flowgram using analog signals (e.g., fluorescent signals) detected from a template nucleic acid or derivative thereof and generating base calls and/or sequencing reads using the flowgram.

As will be appreciated, in flow-based sequencing, the signal for any flow position in the sequencing data is flow order-dependent in that the same flow position for a same template nucleic acid may express different flow signals for different flow orders. Any useful predetermined flow cycles and/or flow orders may be designed to sequence a template nucleic acid and/or more accurately or precisely detect a particular type of sequence (e.g., single nucleotide polymorphisms (SNPs)) within the template nucleic acid (e.g., of a genome).

A flowgram may be binary or non-binary. A binary flowgram detects the presence (1) or absence (0) of an incorporated nucleotide. A non-binary flowgram, such as shown in FIG. 3, can more quantitatively determine a number of incorporated nucleotides at each flow position.

### Re-Sequencing

In some cases, a method for sequencing may comprise sequencing a same template strand multiple times to generate robust sequencing data (e.g., a high-quality sequencing read) corresponding to the template strand. In some cases, a method for sequencing may comprise sequencing a same template strand multiple times and sequencing a same reverse complement strand of the template strand multiple times (e.g., both forward and reverse strands) to generate robust sequencing data (e.g., a high-quality paired end read) corresponding to the template strand. A method for re-sequencing a template strand (which may be a forward strand or reverse strand) may comprise annealing a first sequencing primer to the template strand, extending the first sequencing primer through at least a first portion of the template strand via any combination of bright steps and/or dark steps to generate first sequencing data, denaturing the extended strand from the template strand, annealing a second sequencing primer to the template strand, and extending the second sequencing primer through at least a second portion of the template strand via any combination of bright steps and/or dark steps to generate second sequencing data, and processing (e.g., combining, comparing, matching, aligning, resolving, etc.) the first sequencing data and the second sequencing data to generate a sequencing read of the template strand. A template strand may be denatured and re-sequenced any number of times, such as about, at least about, and/or at most about 1, 2, 3, 4, 5, 6, 7, 8, 9, 10 or more times, such as by annealing an nth sequencing primer to the template strand and extending the nth sequencing primer through at least an nth portion of the template strand. The different n sequencing primers may comprise the same or different sequences which may bind to same or different primer binding sites on the template strand, respectively. The different nth portions on the template strand may refer to the same portions or different portions on the template strand. Two portions on the template strand (that are extended through) may be partially overlapping, completely overlapping (for one or both portions), or non-overlapping. The respective extensions through the template strand in the different sequencing runs may use the same or different nucleotide reagents (e.g., non-terminated nucleotides during a first sequencing run, terminated during a second sequencing run; green dye-labeled nucleotides during a first sequencing run, red dye-labeled nucleotides during a second sequencing run; labeled A-, T-, G-bases and unlabeled C-base nucleotides during a first sequencing run, labeled A-, T-, C-bases and unlabeled G-base nucleotides during a second sequencing run; 5% labeled A bases during a first sequencing run; 100% labeled A bases during a second sequencing run; etc.). The respective extensions through the template strand in the different sequencing runs may have the same flow order or flow cycle of nucleotide reagents. The respective extensions through the template strand in the different sequencing runs may have different flow orders or flow cycles of nucleotide reagents (e.g., A->T->G->C single base flow cycle order during a first sequencing run, T->A->G->C single base flow cycle order during a second sequencing run; A/T/G/C 4-base flow cycle order during a first sequencing run; A/T/G->A/T/C 3-base flow cycle order during a second sequencing run, etc.). Denaturing may comprise contacting the double-stranded nucleic acid molecule with denaturing agents, such as sodium hydroxide (NaOH) or ethylene carbonate. An entire substrate may be subjected to resequencing by, after a first sequencing run, contacting the entire surface with a solution comprising a denaturing agent, contacting the entire surface with a solution comprising sequencing primers under conditions sufficient to anneal them to template nucleic acid strands immobilized to the substrate, and subjecting them to extension reactions. In some cases, denaturing may comprise applying heat to the double-stranded nucleic acid molecule.

Additional sequencing schemes are described in U.S. Pat. Pub. Nos. 2021/0017593A1, 2022/0064728A1, and 2022/0154272A1, each of which is entirely incorporated herein by reference for all purposes.

### Sequencing Systems

The sequencing methods described herein may be performed using any sequencing platform, such as a substrate-based system. The substrate-based system may comprise a closed substrate such as a flow cell comprising one or more fluidic or microfluidic channels, wells, and/or microwells. For example, template nucleic acids on or off a bead may be immobilized to a surface in a flow cell, and reagents flowed in and out of the flow cell through channels in the flow cell to contact the template nucleic acids. The channels may be flushed with wash buffers between different reagent cycles. The substrate-based system may comprise an open substrate. For example, template nucleic acids on or off a bead may be immobilized to a surface of an open substrate, and reagents directed to the surface, such as via nozzles (e.g., across an air gap), to contact the template nucleic acids. The open substrate may be washed with wash buffers between different reagent cycles.

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of analytes and reagents onto the substrate. The sample processing may permit highly efficient imaging of one or more analytes, or signals corresponding thereto, on the substrate. The sample processing system may comprise an imaging system comprising a detector. Substrates, detectors, and sample processing hardware that can be used in the sample processing system are described in further detail in U.S. Patent Pub. No. 20200326327A1, U.S. Patent Pub. No. 20210079464A1, International Patent Pub. No. WO2022072652A1, U.S. Patent Pub. No. 20210354126A1, and International Patent Pub. No. WO2023 192403 A2, each of which is entirely incorporated herein by reference for all purposes.

A substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). A surface of the substrate may be textured or patterned. For example, the substrate may comprise grooves, troughs, hills, pillars, wells, cavities (e.g., micro-scale cavities or nano-scale cavities), channels, wedges, cuboids, cylinders, spheroids, hemispheres, etc. A substrate surface may comprise chemical groups such as amines, esters, hydroxyls, epoxides, and the like, or a combination thereof. A substrate surface may comprise any of the binders or linkers described herein, such as to help immobilize analytes thereto. The substrate may be textured or patterned such that all features are at or above a reference level of the surface (no features below a reference level of the surface, such as a well), or such that all features are at or below a reference level of the surface (no features below a reference level of the surface, such as a pillar). In some instances, a texture of the substrate may comprise structures having a maximum dimension of at most about 500%, 400%, 300%, 200%, 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. Alternatively, the substrate may be untextured and/or unpatterned.

The substrate may have the general form of a cylinder, a cylindrical shell or disk, a rectangular prism, or any other geometric form. The substrate may have a thickness (e.g., a minimum dimension) of at least and/or at most about 100 micrometers (μm), 200 μm, 500 μm, 1 millimeter (mm), 2 mm, 5 mm, 10 mm, 15 mm, 20 mm, 25 mm, 30 mm, 35 mm, 40 mm, 45 mm, 50 or mm. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least and/or at most about 1 mm, 2 mm, 5 mm, 10 mm, 20 mm, 30 mm, 40 mm, 50 mm, 100 mm, 150 mm, 200 mm, 300 mm, 400 mm, 500 mm, 1,000 mm, 1,500 mm, 2,000 mm, 2,500 mm, 3,000 mm, 4,000 mm, 5,000 mm or more.

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing. In some cases, the individually addressable locations may be defined by physical features of the substrate (e.g., on a modified surface) to distinguish such locations from each other and from non-individually addressable locations. In some cases, the individually addressable locations may not be defined by physical features of the substrate, and instead may be defined digitally (e.g., by indexing) and/or via the analytes and/or reagents that are loaded on the substrate (e.g., the locations in which analytes are immobilized on the substrate). The plurality of individually addressable locations may be arranged as an array, randomly, or according to any pattern, on the substrate. FIG. 4 illustrates different substrates (from a top view) comprising different arrangements of individually addressable locations 401, with panel A showing a substantially rectangular substrate with regular linear arrays, panel B showing a substantially circular substrate with regular linear arrays, and panel C showing an arbitrarily shaped substrate with irregular arrays.

The substrate may have any number of individually addressable locations, for example, on the order of 1, 101, 102, 103, 104, 105, 106, 107, 108, 109, 1010, 1011, 1012, 1013 or more individually addressable locations. Each individually addressable location may have any shape or form, for example the general shape or form of a circle, oval, square, rectangle, polygonal, or non-polygonal shape when viewed from the top. A plurality of individually addressable locations can have uniform shape or form, or different shapes or forms. An individually addressable location may have any size. In some cases, an individually addressable location may have an area of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5, 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 7, 8, 9, 10 square micron (μm²), or more. The individually addressable locations may be distributed on a substrate with a pitch determined by the distance between the center of a first location and the center of the closest or neighboring individually addressable location. Locations may be spaced with a pitch of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5, 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, or 10 micron (μm). In some cases, the pitch between two individually addressable locations may be determined as a function of a size of a loading object (e.g., bead). For example, where the loading object is a bead having a maximum diameter, the pitch may be at least about the maximum diameter of the loading object.

An individually addressable location may be capable of immobilizing thereto an analyte (e.g., a nucleic acid, a protein, a carbohydrate, etc.) or a reagent (e.g., a nucleic acid, a probe molecule, a barcode molecule, an antibody molecule, a primer molecule, a bead, etc.). In some cases, an analyte or reagent may be immobilized to an individually addressable location via a support, such as a bead. In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location. A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.). In some cases, an individually addressable location may comprise a distinct surface chemistry. The distinct surface chemistry may distinguish between different addressable locations and/or distinguish an individually addressable location from surrounding locations. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location.

A substrate may comprise a binder or linker configured to immobilize an analyte or reagent to an individually addressable location. The binders may be integral to or added to the substrate. The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions, such as hybridization between two nucleic acid molecules (an oligonucleotide binder and a template nucleic acid). For example, the binders may comprise one or more of antibodies, oligonucleotides, nucleic acid molecules, aptamers, affinity binding proteins, lipids, carbohydrates, and the like.

The substrate may be rotatable about an axis, referred to herein as a rotational axis. The rotational axis may or may not be an axis through the center of the substrate. The systems, devices, and apparatus described herein may further comprise an automated or manual rotational unit configured to rotate the substrate. The rotational unit may comprise a motor and/or a rotor. For instance, the substrate may be affixed to a chuck (such as a vacuum chuck). The substrate may be rotated at a rotational speed of at least about 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with different rotational velocities during different operations described herein, for example with higher velocities during reagent dispense and with lower velocities during analyte loading and imaging operations. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For example, bead loading may be performed with controlled dispensing. For controlled dispensing, the substrate may be rotating with a rotational frequency of no more than 60, 50, 40, 30, 25, 20, 15, 14, 13, 12, 11, 10, 9, 8, 7, 6, 5, 4, 3, 2, 1 rpm or less. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, low speed for analyte or reagent incubation, etc.).

In some cases, the substrate may be movable in any vector or direction. For example, such motion may be non-linear (e.g., in rotation about an axis), linear (e.g., on a rail track), or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

Reagents and/or analytes may be delivered to the surface of the substrate using one or more fluid nozzles. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles. In some cases, the reagents and/or analytes are delivered across a non-solid gap, such as an air gap. There may be any number of dispensing nozzles, such as 1, 2, 3, 4, 5, 6, 7, 8, 9, 10, 11, 12, 13, 14, 15, 16, 17, 18, 19, 20 or more dispensing nozzles. In some cases, different reagents (e.g., nucleotide solutions of different types, different probes, washing solutions, etc.) may be dispensed via different nozzles, such as to prevent contamination where each nozzle may be connected to a dedicated fluidic line or fluidic valve, which may further prevent contamination. Alternatively, some nozzles may share a fluidic line or fluidic valve, such as for pre-dispense mixing and/or to dispensing to multiple locations.

In some cases, a solution may be dispensed on the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate. The substrate may then be subjected to vibration, which may spread the reagent to different locations across the substrate. Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

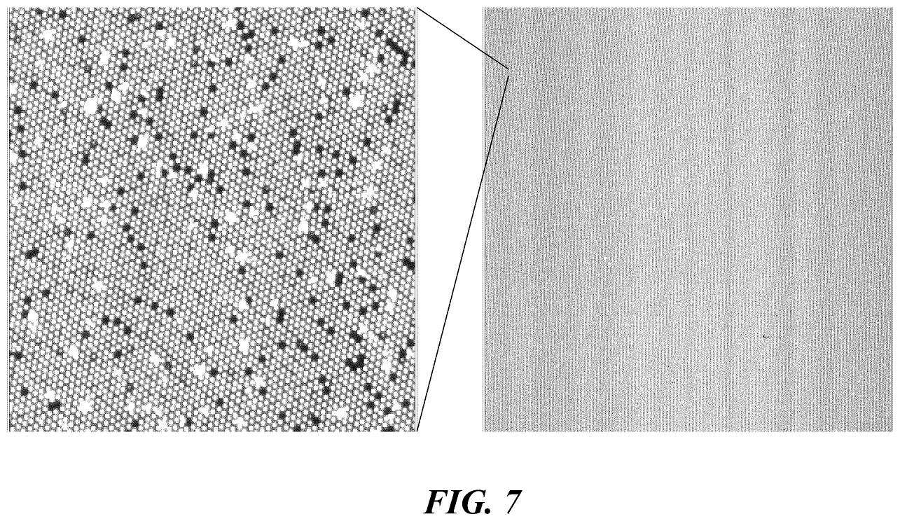

A sample may comprise beads, as described elsewhere herein, for example beads comprising nucleic acid colonies bound thereto. In some cases, an order of magnitude of at least and/or at most about 10¹, 10², 10³, 10⁴, 10⁵, 10⁶, 10⁷, 10⁸, 10⁹, 10¹⁰, 10¹¹, 10¹², 10¹³ or more beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag (e.g., barcode) that identifies a bead amongst a plurality of beads. FIG. 7 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface.

Dispense mechanisms described herein may be operated by a fluid flow unit which may be controlled by one or more controllers, individually or collectively. The fluid flow unit may comprise any of the hardware and software components described with respect to the dispense mechanisms herein.

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A signal may be an optical signal (e.g., fluorescent signal), electronic signal, or any detectable signal. The signal may be detected during rotation of the substrate or following termination of the rotation. The signal may be detected while the analyte is in fluid contact with a solution. The signal may be detected following washing of the solution. In some instances, after the detection, the signal may be muted, such as by cleaving a label from a probe and/or the analyte, and/or modifying the probe and/or the analyte. Such cleaving and/or modification may be performed by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times by the system. Such operations may be repeated in an iterative manner. For example, the same analyte immobilized to a given location in the array may interact with multiple solutions in multiple cycles and for each iteration, the additional signals detected may provide incremental, or final, data about the analyte during the processing. For example, when sequencing a nucleic acid molecule, additional signals detected for each iteration may be indicative of one or more bases in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly, electronically or computationally, advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar function by high speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, tube lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors, CCD cameras, CMOS cameras, TDI cameras (e.g., TDI line-scan cameras), pseudo-TDI rapid frame rate sensors, or CMOS TDI or hybrid cameras. The optical system may further comprise any one or more optical sources (e.g., lasers, LED light sources, etc.). In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g., interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise one or more controllers operatively coupled to the one or more sensors, individually or collectively programmed to process optical signals from the one or more sensors, such as for each region of the rotating substrate.

In some cases, the optical system may comprise an immersion objective lens. The immersion objective lens may be in contact with an immersion fluid that is in contact with the open substrate. The immersion fluid may comprise any suitable immersion medium for imaging (e.g., water, aqueous, organic solution). In some cases, an enclosure may partially or completely surround a sample-facing end of the optical imaging objective. The enclosure may be configured to contain the immersion fluid. The enclosure may not be in contact with the substrate; for example, a gap between the enclosure and the substrate may be filled by the fluid contained by the enclosure (e.g., the enclosure can retain the fluid via surface tension). In some cases, an electric field may be used to regulate a hydrophobicity of one or more surfaces of the container to retain at least a portion of the fluid contacting the immersion objective lens and the open substrate. In some cases, the immersion fluid may be continuously replenished or recycled via an inlet and outlet to the enclosure.

One or more surfaces of the substrate may be exposed to and accessible from a surrounding open environment. In some cases, the surrounding open environment may be controlled and/or confined in a larger controlled environment. An open substrate may be processed within a modular local sample processing environment. A barrier comprising a fluid barrier may be maintained between a sample processing environment and an exterior environment during certain processing operations, such as reagent dispensing and detecting. Systems and methods comprising a fluid barrier are described in further detail in U.S. Patent Pub. No. 20210354126A1, which is entirely incorporated herein by reference. A modular local sample processing environment may be defined by a chamber and a lid plate, where the lid plate is not in contact with the chamber, and the gap between the lid plate and the chamber may comprise the fluid barrier. The fluid barrier may comprise fluid (e.g., air) from the sample processing environment and/or the exterior environment and may have lower pressure than the sample processing environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit (ies), or ranges thereof, within the sample processing environment during various processing operations.

The systems described herein, or any element thereof, may be environmentally controlled. For instance, the systems may be maintained at a specified temperature or humidity.

For an operation, the systems (or any element thereof) may be maintained at a temperature of at least and/or at most 20 degrees Celsius (C), 25° C., 30° C., 35° C., 40° C., 45° C., 50° C., 55° C., 60° C., 65° C., 70° C., 75° C., 80° C., 85° C., 90° C., 95° C., 100° C., or more. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

An open substrate may be retained in the same or approximately the same physical location during processing of an analyte and subsequent detection of a signal associated with the processed analyte. Alternatively, different operations on or with the open substrate may be performed in different stations disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. The open substrate may transition between different stations by transporting the sample processing environment comprising the chamber containing the open substrate between the different stations. One or more mechanical components or mechanisms, such as a robotic arm, elevator mechanism, actuators, rails, and the like, or other mechanisms may be used to transport the sample processing environment.

One or more environmental units (e.g., humidifiers, heaters, heat exchangers, compressors, etc.) may be configured to, individually or collectively, regulate one or more operating conditions in one or more stations. In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

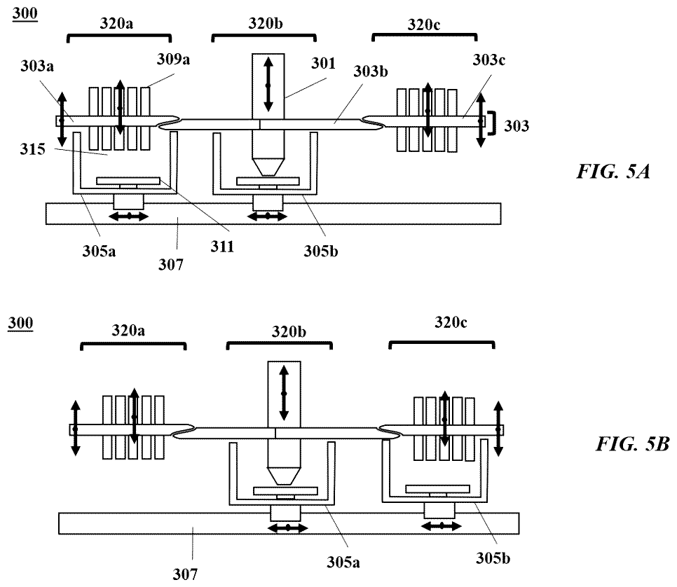

One or more modular sample environment systems (each having its own barrier system, e.g., fluid barrier) can be used between the different stations. In some instances, the systems described herein may be scaled up to include two or more of a same station type. For example, a sequencing system may include multiple processing and/or detection stations. FIGS. 5A-5B illustrate a system 500 that multiplexes two modular sample environment systems in a three-station system. In FIG. 5A, a first chemistry station (e.g., 520a) can operate (e.g., dispense reagents, e.g., to incorporate nucleotides to perform sequencing by synthesis) via at least a first operating unit (e.g., fluid dispenser 509a) on a first substrate (e.g., 511) in a first sample environment system (e.g., 505a) while substantially simultaneously, a detection station (e.g., 520b) can operate (e.g., scan) on a second substrate in a second sample environment system (e.g., 505b) via at least a second operating unit (e.g., detector 501), while substantially simultaneously, a second chemistry station (e.g., 520c) sits idle. An idle station may not operate on a substrate. An idle station (e.g., 520c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 5B, where the second substrate in the second sample environment system (e.g., 505b) is re-stationed from the detection station (e.g., 520b) to the second chemistry station (e.g., 520c) for operation (e.g., dispensing of reagents, e.g., to incorporate nucleotides to perform sequencing by synthesis) by the second chemistry station, and the first substrate in the first sample environment system (e.g., 505a) is re-stationed from the first chemistry station (e.g., 520a) to the detection station (e.g., 520b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 507) to the different stations and/or the different stations may be physically moved to the different sample environment systems. One or more components of a station, such as modular plates 503a, 503b, 503c of plate 503 (e.g., lid plate) defining a particular station(s), may be physically moved to allow a sample environment system to exit the station, enter the station, or cross through the station. During processing of a substrate at station, the environment of a sample environment region (e.g., 515) of a sample environment system (e.g., 505a) may be controlled and/or regulated according to the station's requirements. After the next operating cycle is complete, the sample environment systems can be re-stationed again, such as back to the configuration of FIG. 5A, and this re-stationing can be repeated (e.g., between the configurations of FIGS. 5A and 5B) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations substantially simultaneously while the detection station is kept idle.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g, such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

Provided herein are devices, systems, methods, compositions, and kits for use with library preparation. Such devices, systems, methods, compositions, and kits can be applied alternatively or in addition to at least the preparation 101 and amplification 105 operations described with respect to sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein.

### Single Adapter Species

One issue with the construction of libraries for sequencing is the inevitable loss of some sample material during library preparation, especially due to the attachment of adapters to library molecules. For instance, where template molecules are desired to be coupled to a first type of adapter on one end and a second type of adapter on the other end (e.g., where the first and second adapters serve different downstream purposes, such as bead attachment vs. sequencing primer), only about 50% of the resulting library molecules will be ligated to one of each type of adapter (where about 25% of the resulting library molecules will be ligated to the first type of adapter at each end and about 25% of the resulting library molecules will be ligated to the second type of adapter at each end). Thus, there is a significant advantage in terms of library preparation efficiency if a single species of adapter can be used to serve each distinct downstream purpose. The devices, systems, methods, compositions, and kits provided herein may allow for the efficient preparation of template nucleic acid molecules for sequencing (e.g., library preparation for methylation sequencing) by the use of a single adapter species.

The use of a single adapter species can reduce the loss of sample material during library preparation for other versions of sequencing (e.g., whole-genome sequence, targeted sequence, non-methylation sequencing, methylation sequencing, etc.). By using a single adapter species, the entirety (or at least the majority) of a population of sample molecules may be successfully converted into library molecules. The efficient usage of sample material is essential in cases where very small amounts of sample are available (e.g., cell-free DNA from biological samples). The loss of even a very small fraction of the molecules available in the sample can prevent accurate detection of mutations and hence reduce the efficacy of minimal residual disease detection, disease screening, or other medical tests.

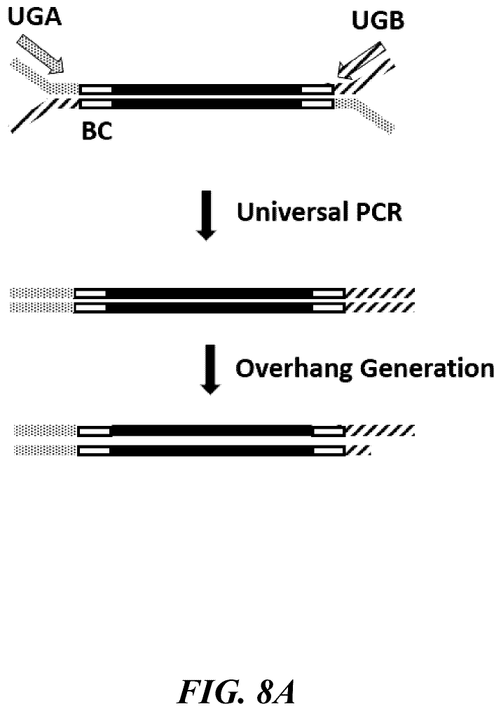

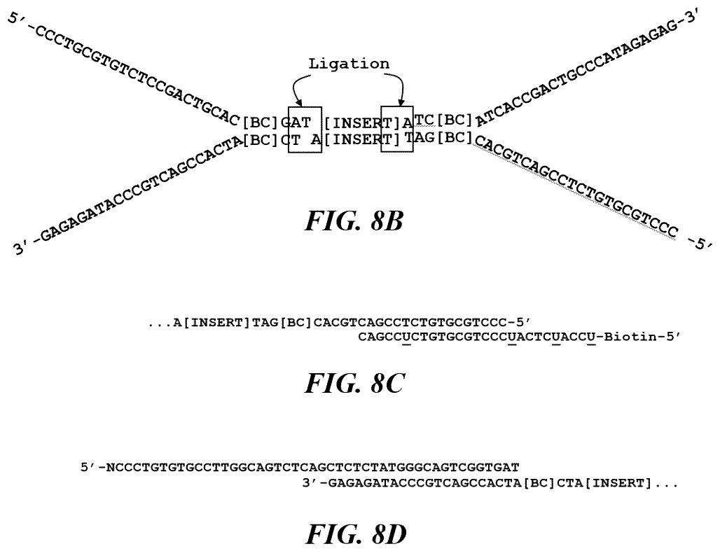

FIG. 8A illustrates one example schematic of using a single adapter species. After ligation of the one adapter species to insert molecules, a single PCR operation may be performed, using two distinct PCR primers. FIGS. 8B, 8C, and 8D illustrate example sequences that may be used in accordance with the FIG. 8A schematic. As shown in FIG. 8B, a single, partially double-stranded adapter species may be ligated to each end of a double-stranded insert molecule. The adapter species may comprise a first, single-stranded region, and a second, double-stranded region. Here, the single-stranded region of the adapter comprises the sequences on a first stand and a second strand, respectively:

(SEQ ID No. 1)
5′-CCCTGCGTGTCTCCGACTGCAC-3′,
and
(SEQ ID No. 2)
5′-ATCACCGACTGCCCATAGAGAG-3;

and the double-stranded region of the adapter comprises a barcode sequence with complementarity between the first strand and the second strand.

For the library amplification, a first primer sequence (5′-UCCAUCTCAUCCCTGCGTGTCUCCGAC-3′, SEQ ID No. 3) may anneal to the single-stranded region of the first strand. A second primer sequence (5′-NCCCTGTGTGCCTTGGCAGTCTCAGCTCTCTATGGGCAGTCGGTGAT-3′, SEQ ID No. 4) may anneal to single-stranded region of the second strand. In some instances, the first primer sequence may comprise a 5′ biotin and one or more cleavable sites. The second primer sequence comprises a first region that is complementary to the single-stranded region of the second strand and a second overhang region. By the use of two distinct primers, non-symmetrical amplified library molecules may be produced.

In some cases, the first or second primer may comprise one or more cleavable moieties. In some cases, only the first primer may comprise one or more cleavable moieties. As shown in FIG. 8C, the one or more cleavable moieties in the second primer may all comprise uracils. In some cases, the first and/or the second primer may comprise multiple types of cleavable moieties. In some cases, the first or second strand of the adapter may further comprise one or more additional cleavable moieties. By the use of additional cleavable moieties, free adapters (e.g., those not coupled to a support such as a sequencing bead) may be degraded. This degradation of free adapter sequences helps reduce the rate of polyclonality on sequencing beads by preventing unattached library molecules that do mistakenly enter a reaction mixture (e.g., oil droplets during ePCR) from hybridizing to beads and being subsequently amplified. These additional cleavable moieties are distinct from the one or more cleavable sites that release adapters from streptavidin/biotin complexes (e.g., Us in the second primer, SEQ ID No. 4).

In some cases, the cleavable moiety(ies) comprises uracil, ribonucleotide, spacer(s), or methylated nucleotide(s). In some cases, the spacer is a dSpacer or a C3 spacer. In some cases, cleaving the cleavable moiety(ies) comprises using APEI enzyme to cleave the spacer(s). In some cases, the cleavable moiety(ies) is a methylated nucleotide(s) and cleaving the cleavable moiety(ies) comprises using MspJI to cleave the methylated nucleotide(s). In some cases, the cleavable moiety(ies) is a uracil and cleaving the cleavable moiety(ies) comprises using a uracil D glycosylase (UDG) to cleave the uracil (e.g., in some cases the cleavage conditions comprise a mixture of UDG and Endonuclease VIII, e.g., USER). In some cases, the cleavable moiety(ies) is a ribonucleotide(s) and cleaving the cleavable moiety(ies) comprises using a RNase to cleave the ribonucleotide(s). In some instances, each cleavable moiety in a respective strand of an adapter molecule is a same type (e.g., all uracils, all ribonucleotides, etc.).

In some cases, the first strand comprising SEQ ID No. 1 may further comprise a barcode sequence located 3′ of SEQ ID No. 1. In some cases, the barcode sequence is selected from any one of SEQ ID Nos: 207-1261 described elsewhere herein. In some cases, the barcode sequence may be any other sequence (e.g., a KM barcode as described herein) that is suitable. In some cases, the first strand may further comprise a GAT (or other constant sequence of any length suitable for library preparation) located at the 3′ end (see e.g., FIGS. 8B and 8C). In some cases, the 3′ T in strand 1 may be phosphorylated. In some cases, the second strand comprising SEQ ID. No. 2 may further comprise a reverse complement of the barcode sequence in the first strand, wherein the reverse complement sequence is located 5′ of SEQ ID. No. 2. In some cases, the second strand further comprises a CT located at the 5′ end (or any other constant sequence corresponding to the constant sequence in the first strand) (see e.g., FIGS. 8B and 8D).

In some cases, the first and second primer sequences may comprise random nucleotides. For example, the second primer, SEQ ID No. 4, comprises one 5′ random nucleotide (e.g., selected from the set of the four canonical nucleotides) (see FIG. 8D). In some cases, the first primer sequence may comprise 1, 2, 3, 4, 5, 6, 7, or more 5′ random nucleotides. In some cases, the random nucleotides may be located at any position within the first primer sequence. In some cases, the random nucleotides may all be located at the 5′ end. In some cases, the first primer sequence, SEQ ID No. 3 may comprise one or more random nucleotides.

Subsequent to library amplification, amplified molecules may be exposed to conditions sufficient for cleavage of one or more cleavable moieties (e.g., exposure to USER enzyme to cleave the U nucleotides in the example primer sequences here) and/or to different conditions for the cleavage of one or more types of cleavable moieties. Such cleavage may i) remove 5′ biotin (or other 5′ modifications), ii) produce single-stranded overhangs, iii) reduce polyclonality in an amplified library, or a combination thereof.

### Multiple Species of Non-Identical Adapters

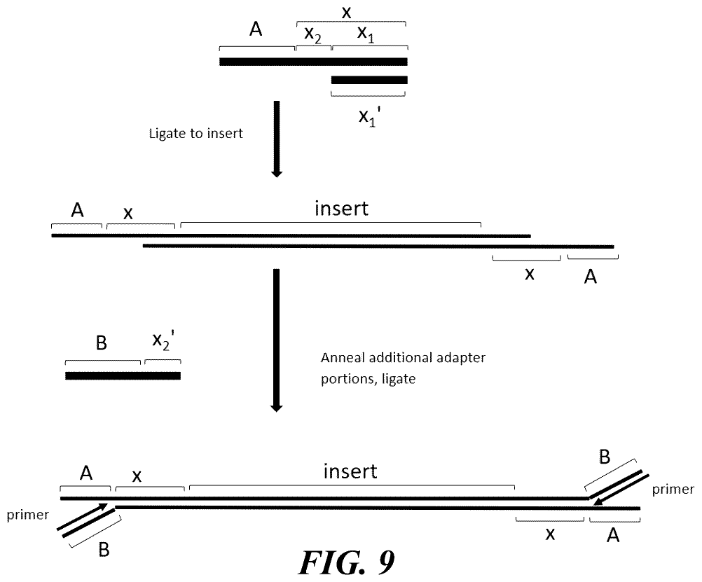

Additional examples of partially double-stranded adapter and primer species combinations are illustrated in FIGS. 9-11. To provide a population of non-identical adapters, the partially double-stranded adapters may differ in the single-stranded region(s), in the double-stranded regions, or both. In some cases, identical or mostly identical adapter molecules may be converted to non-identical adapter regions in a library molecule by amplifying with non-identical primers. Alternatively, non-identical adapter molecules (e.g., mostly identical adapter molecules) may be amplified with identical primers to generate non-identical adapter regions in library molecules.

FIG. 9 illustrates a schematic for assembling identical partially double-stranded adapter regions in library molecules. A population of partially double-stranded first adapters is provided. These first adapters comprise a double-stranded region and an overhang region (e.g., a single strand overhang). After the first adapters are ligated to insert molecules, a population of single-stranded second adapters is provided. These second adapters comprise a first region with complementarity to the overhang region of the first adapters and a second region that lacks complementarity. The second adapters anneal to the first adapter/insert molecules and are ligated, thus providing library molecules comprising identical partially double-stranded adapter regions. As described elsewhere herein, either identical or non-identical primers may be used in amplification and/or other downstream processes. In some instances, the two ligation reactions may be performed simultaneously. In some cases, the two ligation reactions may be performed sequentially.

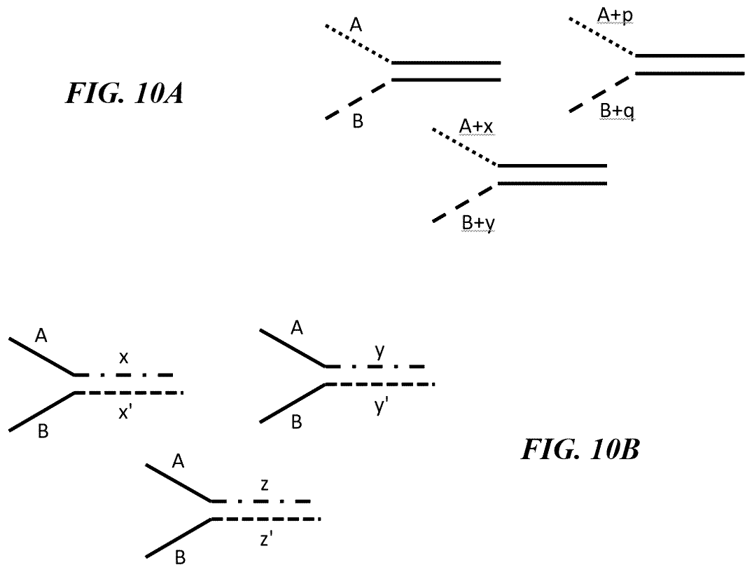

FIG. 10A illustrates an example of multiple species of partially double-stranded adapters. In the single-stranded region(s), each adapter differs by one or more nucleotide bases. Each partially double-stranded adapter is identical to the other adapters along at least a portion of the overall sequence. In some instances, the portion is at least 90%, 95%, 99%, or 100% of the overall sequence length. In some instances, each partially double-stranded adapter comprises at least 1, 2, 3, 4, 5, 6, 7, 8, 9, or 10 unique nucleotide bases, where the unique bases may be consecutive or non-consecutive.

FIG. 10B illustrates another example of non-identical partially double-stranded adapter molecules. In this case, each adapter molecule comprises an identical single-stranded region (e.g., a first strand and a second strand that are non-complementary to each other); however, each adapter differs by one or more nucleotide bases in the double-stranded region (e.g., in length, sequence, and/or a combination thereof). For instance, a first adapter may have a double-stranded region that is 10 bases in length and a second adapter may have a double-stranded region that is 11 bases in length. In another example, a first adapter may comprise a first sequence that is 10 bases in length and a second adapter may comprise a second sequence that is also 10 bases in length but differs from the first sequence by at least one nucleotide base (e.g., a single base mismatch). In some cases, a first adapter and a second adapter may differ by no more than one nucleotide base. In some cases, a first and a second adapter may differ by 1, 2, 3, 4, 5, or more nucleotide bases. A first adapter and a second adapter may each be any suitable length. For example, each may be 4, 5, 6, 7, 8, 9, 10, 11, 12, 13, 14, 15, 16, 17, 18, 19, 20, 25, 30, 35, 40, or more nucleotide bases in length.

In some cases, the methods illustrated in FIGS. 10A and 10B may be combined; that is, a population of non-identical partially double-stranded adapters may differ from each other in the single-stranded region(s) and the double-stranded region. In any of these cases, the adapters must-despite their differences in sequence—at least be i) able to be ligated to inserts of interest and ii) able to anneal to a desired set of primers for amplification and/or sequencing.

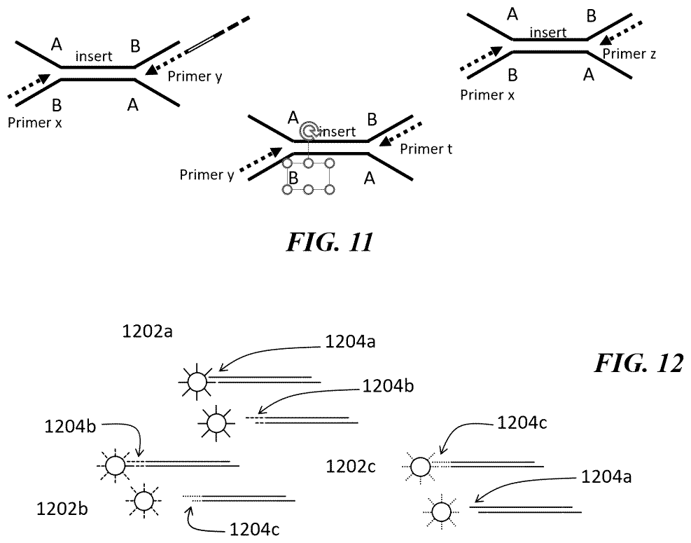

FIG. 11 illustrates an example of a single species of partially double-stranded adapters and multiple species of primers for amplification. Primers in the plurality of non-identical primers differ from each other by one or more nucleotide bases (e.g., in length, sequence, and/or a combination thereof). In one example, a first primer may be 20 bases in length and a second primer may be 25 bases in length. In another example, a first primer may comprise a first sequence of 22 bases in length and a second primer may comprise a second sequence of 22 bases in length, where the first sequence and the second sequence differ by at least one nucleotide base.

In some cases, the bipartite adapter designs described with respect to FIGS. 9-11 may be used in accordance with the high-efficiency adapter method described with respect to FIGS. 11A-8D. That is, in some cases, the high-efficiency adapters may be produced in a bipartite manner as illustrated in FIG. 9, and/or a pool of high-efficiency adapters may comprise one or more base mismatches, as illustrated in FIGS. 10 and 11. In some instances,

### Oligonucleotides for Multiple Bead Species

As discussed elsewhere herein, FIG. 12 illustrates an example of sequencing beads comprising capture oligos (e.g., oligos for the attachment of template sequences), where there are two or more species of beads, and each species of bead comprises a distinct oligo sequence. Three bead species 1202 and three adapter species 1204 are illustrated. These multiple bead species may be used to capture different template nucleic acid molecules of a sample. Multiple species of bead primers may be especially useful in amplification methods comprising emulsion PCR (ePCR) or other droplet-based methods.

In ePCR, a plurality of partitions (e.g., wells or droplets in an emulsion) may be generated, wherein each partition may comprise (i) a plurality of beads and (ii) at least one nucleic acid molecule (e.g., a target nucleic acid molecule of a biological sample). In some cases, a partition may comprise at least two beads. Alternatively or in addition, a partition may comprise at least two target nucleic acid molecules. In ideal conditions, each partition containing a target molecule comprises a single target nucleic acid molecule and a single bead. This reduces polyclonality (e.g., the amplification of multiple target nucleic acid molecules on a single bead which reduces sequencing quality) and also maximizes the throughput of a sequencing reaction (e.g., by ensuring that each target molecule is sequenced only once, instead of multiple times which may happen if a target molecule is amplified onto multiple beads). Methods of performing ePCR are described in U.S. patent application Ser. No. 17/394,692 and International Patent Pub. No. WO2022040557A2, each of which is entirely incorporated herein by reference for all purposes. One issue with typical ePCR amplification is that methods of optimizing for single template/single bead partitions may result in the loss of some template material and in the use of excessive amounts of beads (e.g., to decrease the probability of polyclonality due to the presence of multiple template molecules in a single partition). It is hence advantageous to develop additional solutions for decreasing polyclonality in ePCR. One such method is to use a variety of adapter sequences for template molecules and provide a population of multiple species of beads where each bead species has a different capture sequence. Provided herein in Table 1 and Table 2 are bead capture sequences (e.g., bead-tethered oligonucleotide sequences) that can be used for multiple bead species.

Each oligo in Table 1 and Table 2 was selected to meet the following criteria: i) 30 nucleotides in length, ii) no hairpins (defined as a minimum stem length of 4 and minimum loop length of 3), iii) a melting temperature between 50° C. and 70° C., iv) no guanine or cytosine homopolymers of 4 or more bases, v) last three bases are not identical, and vi) the longest common subsequence with any existing primer or any other primer in the list is less than 10 bases in length. Melting temperatures for the oligonucleotides in Table 1 were calculated using a Na concentration of 50 mM and a DNA concentration of 50 nM. Melting temperatures for the oligonucleotides in Table 2 were calculated using a Na⁺ concentration of 50 mM, a Mg²⁺concentration of 11 mM, and a DNA concentration of 50 nM.

In some cases, two or more barcode sequences are selected from Table 1, Table 2, or a combination thereof. A first barcode sequence may be coupled (e.g., via click chemistry or ligation) to a bead. In some cases, each barcode sequence comprises a 5′ moiety for click chemistry (e.g., a DBCO or an azide). In some cases, each bead in a plurality of beads is coupled to a single type of barcode sequence. In some cases, a bead may be coupled to two different types of barcode sequences. In some cases, a plurality of beads may comprise at least subsets of beads, where beads in each subset are coupled to a respective type of barcode sequence. Each sequence in Table 1 is listed in a 5′ to 3′ direction.

TABLE 1
Bead primer sequences

| SEQ ID No. | Oligo sequence |
|---|---|
| SEQ ID No: 5 | GAATTTACATGCTTTAACAACCGTGGCCTC |
| SEQ ID No: 6 | CTAACGATGAACAACTTCCACCACTGGGCA |
| SEQ ID No: 7 | ATGTCGTCTTTGTCCTTGTTGCTAACTAGA |
| SEQ ID No: 8 | ACGCTACTGTCTTGGAGGGCCACTTCATAT |
| SEQ ID No: 9 | CTAGATGTCGACGAACGGCAGCGCTGATGT |
| SEQ ID No: 10 | GTCTGCATTCTGGCTATAGGTTAGAGTCGA |
| SEQ ID No: 11 | CCTCTTGGGAGCCGTCTCGTAATCCGATCT |
| SEQ ID No: 12 | AGGTACGCGCAACTGATCGAAACCAAGATG |
| SEQ ID No: 13 | ATCGAGAGGAACTACGTCGCCCGGTAAAGA |
| SEQ ID No: 14 | TATCGTAAATACAACTGACCAACATAAGTC |
| SEQ ID No: 15 | GGGTCCGTTCCTGATGCCTACCGCCAATGT |
| SEQ ID No: 16 | GTTGGATGTGAGTTCACTGGTACCACTAAG |
| SEQ ID No: 17 | TCCGACGCGTCTGTATTGATTTGTCTCTGG |
| SEQ ID No: 18 | GAATGAGGTCGAGTAACCCTGGGCCCGTGC |
| SEQ ID No: 19 | CGTGGTAACGTAGGCGCGCACTTCTCAGCA |
| SEQ ID No: 20 | GAAAACGGCTTGAGCTGAGACTCTCCAATT |
| SEQ ID No: 21 | CGCGAATGTTATTTGGGCGAATGACTAAAG |
| SEQ ID No: 22 | TTACCGTATGGCCTATAGACCGAGGTTCTT |
| SEQ ID No: 23 | ACGGCACTCATCCTTACGTGGTCGGGTAGA |
| SEQ ID No: 24 | GGCTCACAACGCCACGCTACTCTCAACCGG |
| SEQ ID No: 25 | TGTAAACAGCGCGGTCATCTGATAGAGGAC |
| SEQ ID No: 26 | CCTTAGTGTAAATCGACCACGTGAGCATCG |
| SEQ ID No: 27 | GGTCCCAGCCACACTCGCGAGCCTTTTAGC |
| SEQ ID No: 28 | TATGATACCTAGGCTGCATCGTTACCCGGC |
| SEQ ID No: 29 | AGCTTCTAAAATACGAGCCACCCAATACGA |
| SEQ ID No: 30 | GGAGTGTCTACCGTAGGAACGATCCACAGC |
| SEQ ID No: 31 | CCGTGGGATCGAGATGGCTAGTAGGAGACA |
| SEQ ID No: 32 | CTAAGCCGATTCGTGTCTCCACAGAAATTA |
| SEQ ID No: 33 | TGGACGCTGGGCTAAGAAGTTGGCCGATAG |
| SEQ ID No: 34 | TTCATGAAGCCGTTATTTCGGACCTACTCG |
| SEQ ID No: 35 | CTACTGACGTATTGAGTTGCCTAAATCTGG |
| SEQ ID No: 36 | GCGAGGTATGACTCCCGCCGCCTTCTCAAG |
| SEQ ID No: 37 | AACCGTTCAAATTTGTGCCGATAGGTGTTA |
| SEQ ID No: 38 | ATCAGCGAGCCGAGACACGAGTTCACTTAT |
| SEQ ID No: 39 | AAAACTTCCCTAATAGCTTAACGTATGAGT |
| SEQ ID No: 40 | ATTATGAGCAGAGCAAACGTCACTAAGTGG |
| SEQ ID No: 41 | CGCACCAGAGGTCTCAATGCTCATGTAAGA |
| SEQ ID No: 42 | TTTAAAGTGGTCTATGCCGAATCGATCTCC |
| SEQ ID No: 43 | GTGTTTAGGATTAGGGACATCACGGCTTCT |
| SEQ ID No: 44 | ACCCGTGGAAAGGTGAGTATACAGCAGCTA |
| SEQ ID No: 45 | AACGAAAATATACTGTCGCCACAAGGAAAC |
| SEQ ID No: 46 | CCGACGGTCCGGACTTTTTCTCACCATCGT |
| SEQ ID No: 47 | GCATTAGCTCAAAGGCCAGACTTATCCGTG |
| SEQ ID No: 48 | AGGCAACTATGTTCACGGTACGTCGGCTCG |
| SEQ ID No: 49 | CACCTATCTGGATCCAGTGAATACCTCCGA |
| SEQ ID No: 50 | TTCGGCGTTCGAGCAGTAGGACCTAACCAA |
| SEQ ID No: 51 | CCATCATTGGTTTGCAGGGTATATAGCGCC |
| SEQ ID No: 52 | GGCGAGTAGCCATAGATCGTCAACGTCGTG |
| SEQ ID No: 53 | GAGCCCGCCAGTCATCGAAACACGCATCAT |
| SEQ ID No: 54 | ACGGAGGTTGGCCCGCACGATATTCAGACA |
| SEQ ID No: 55 | ATAACCTTAATGCACGAGCTGAAACCACAG |
| SEQ ID No: 56 | TTAGCCAGGTGGTCCGCTGCCAGAATGGTC |
| SEQ ID No: 57 | TTTGTGTATTTTCACTCTGTCTTTAGCGGA |
| SEQ ID No: 58 | CGGATGGGACGTGAGCTTAATGGATCTGAT |
| SEQ ID No: 59 | CTGACCGGAAAAGATTCGAGGGCGGAGAAC |
| SEQ ID No: 60 | CTCCATATCTCAATCAACTCTCGTCGAATT |
| SEQ ID No: 61 | GGCGGCAGTGCCTTCCGGGTTCCTTTTTAG |
| SEQ ID No: 62 | GTATTTAGCAAAAGACTCTGAGCCGTCATT |
| SEQ ID No: 63 | TTAGCTCAATACACACGTCAACGTACGGCA |
| SEQ ID No: 64 | AAGAACCATAGCGCATGTATCCCACGACCG |
| SEQ ID No: 65 | CGAGAGACCCACGTCAGTCGTAACTAATTA |
| SEQ ID No: 66 | GGATTTACATGAGTCTGCCAGGTACCGATC |
| SEQ ID No: 67 | TATGTGGGAGGGTTCGCTACAGTGATCCAT |
| SEQ ID No: 68 | GGACCCGGAAGGTGTAAACTACCACTGACA |
| SEQ ID No: 69 | AGGTTGACTACACGGCCGTCCCTAAAAGGT |
| SEQ ID No: 70 | ACGAGAGCGCGGTCGACTAGCAAATTGCCG |
| SEQ ID No: 71 | GAAGACGTCTTGCGACCTATGAGCCGTGCC |
| SEQ ID No: 72 | AGCGGTTTCAATGGGAAGAGTTGGTTGTCC |
| SEQ ID No: 73 | ATTATAGTAAGCGTTCATCTGGTTTATGTC |
| SEQ ID No: 74 | TTGTGTCGCTTATACCCATACATGCCTACC |
| SEQ ID No: 75 | GCATTCGTTACGGGCCTCTCTGAACAAGAA |
| SEQ ID No: 76 | AGCAAGTTAGATGTATGCAACCAATATGGA |
| SEQ ID No: 77 | GGCAAGTCACACGTCTATACCGGTCGGTCC |
| SEQ ID No: 78 | TGCAAACAAGCCGTAGTAGCCAACCTGCCT |
| SEQ ID No: 79 | TACATACGACATACGGTAACTTCTACAGGC |
| SEQ ID No: 80 | CACCCGATAAGATACGATGCCTCCGTCAAT |
| SEQ ID No: 81 | CAGCGCGACTAGGCAGTAATGACTATCCAC |
| SEQ ID No: 82 | ACAGTCGACCGGATTGTAACTCATGTAAAC |
| SEQ ID No: 83 | TCGCTTGGCAATTTCACCGGAATATGTGTA |
| SEQ ID No: 84 | CAATGTTAAAGATGGAAATACGAGGAGCGT |
| SEQ ID No: 85 | GTGGACAAACAGCTGCAGAATCTCTAGTCG |
| SEQ ID No: 86 | CAAAAACGTGCCTGAGTCAATGGGCTTTCA |
| SEQ ID No: 87 | AAAGCAGTGGTACGAGCCGGTGCAACTAAG |
| SEQ ID No: 88 | CACTGAAACTCAACGAGCGATCTAAGGCGG |
| SEQ ID No: 89 | TGCTCAACAACCATGCCACATCGACGTACG |
| SEQ ID No: 90 | GAGGCTATATTCAAGTTTTTACGGGAGAAG |
| SEQ ID No: 91 | GTTTCCGCGAGCTGAAGCAATAGAACGATG |
| SEQ ID No: 92 | TATATACTCCGTTCTTGTTTCCGGTGTGGC |
| SEQ ID No: 93 | AGGTAGGCATCTCGATTGATCTATAGGCTT |
| SEQ ID No: 94 | GTCAGGAAAAGCAGGCATTATACGTAACAG |
| SEQ ID No: 95 | GGGCAATGAACGCGATGGGTCTCTTTCGAT |
| SEQ ID No: 96 | GTGCCGATAGCCCATTCCGTCTACATTGAT |
| SEQ ID No: 97 | GAATACATAAAAGGGATCGTTAACACCCAC |
| SEQ ID No: 98 | TATGACCGGTAGACCTGTCTGAATCCGCTC |
| SEQ ID No: 99 | GTTTCTTTTACTACGACAGGCGATACCGAC |
| SEQ ID No: 100 | TGCGCAGATGGCTTTTCGGATAGAGAATTG |
| SEQ ID No: 101 | AAAATCCCTGGGCTTTAGCTCGATCCAAAT |
| SEQ ID No: 102 | CTGTCGCCAAGGACTGGTCCGGGAAGTACG |
| SEQ ID No: 103 | CCGATCCTAGAGGTGACAATAAATAAGAGC |
| SEQ ID No: 104 | TGGTTAGTTGGTGTCTGTCTTTCTGGATTG |

TABLE 2
Oligo capture sequences

| SEQ ID No. | Oligo sequence |
|---|---|
| SEQ ID No: 105 | AGTATTATGGACATGTGCTGGTTTTCTGGC |
| SEQ ID No: 106 | CCTGATTTACCTAGATTATACATACTTCAC |
| SEQ ID No: 107 | GCCGGATATTAGATGTCTCCAACGCACAAC |
| SEQ ID No: 108 | TATAGGATTAGCTGCATCACCATGAGTAAC |
| SEQ ID No: 109 | CATACCATCTGCCTTTCAAGTGGAGTCACC |
| SEQ ID No: 110 | GTTAAAGCCTGTCCAGTGTTTCTCCCTACG |
| SEQ ID No: 111 | CTTAAAGTCAGGGTTAGCTCAGGGTACGTG |
| SEQ ID No: 112 | GCATAGTTGTTTGTCCTGTCGAGAAGCGAG |
| SEQ ID No: 113 | GCTATGACAACTTAACTCGCTCTCTCTGAC |
| SEQ ID No: 114 | GCGGGAATCACCGAGTACTCAGTTAGGTAA |
| SEQ ID No: 115 | CATAAACGAATTACACTCTAGGTAGTATCG |
| SEQ ID No: 116 | TATACAAGCAACGTTAACGATTGGCAGCAT |
| SEQ ID No: 117 | CTGGGACGCTAGATATCAAGGGCTACAATC |
| SEQ ID No: 118 | TGATAAGAATTGATAGCATGTGCAGTGAGA |
| SEQ ID No: 119 | GGCAACGTGCTTTACTACTGACGCTTCTTA |
| SEQ ID No: 120 | CGTTGCAGCTATTCGTGTCGCTTAATTTGG |
| SEQ ID No: 121 | CACTTAGTAATCCAGTTCATCCATTGGCGA |
| SEQ ID No: 122 | ACCCAAAACATCGAAAAACACCTCAGAACG |
| SEQ ID No: 123 | ACTCGTCATTATGCAAGGATAGAGCTAGCT |
| SEQ ID No: 124 | AAATCATGGTCAACCAATGTGTTCGAGACT |
| SEQ ID No: 125 | GGTATTTTGGTTGTATGCCACTGGGTCTGA |
| SEQ ID No: 126 | AGTACGTACCTATTAATCAACTTCGCCTGA |
| SEQ ID No: 127 | AGAGGACCACCAGCTTGTAAATTCACTAAT |
| SEQ ID No: 128 | CCGACACATAAGGTTGGTGAAGATAGTCCA |
| SEQ ID No: 129 | GAAGGGATGGGCGGCACTTTGGATATTTAG |
| SEQ ID No: 130 | AAGGCATACATAGAAGGTAGCGTGATACGG |
| SEQ ID No: 131 | GACTCGGCATAACCAAAGGTATCCAACGAA |
| SEQ ID No: 132 | TATATTTGTGATCAGGTTCTCATTTATCGC |
| SEQ ID No: 133 | TCGAGTACGTCATATTTTAGTAGTCTGCGT |
| SEQ ID No: 134 | CCATGATAGAGAGTAGAATGAAGAGCACAC |
| SEQ ID No: 135 | GAATTGACTACAGGTTAAGAATACTATACA |
| SEQ ID No: 136 | CGAATTATCTTACGCCGCCGATGTTTTCTA |
| SEQ ID No: 137 | ATCTAGTTACAGTTCCCTACATTCACAAGA |
| SEQ ID No: 138 | GGTTATAAATGGTCTTGGTTTTGGAGTGCT |
| SEQ ID No: 139 | GAGATTATCGTACCTCCACTTGGATGAACA |
| SEQ ID No: 140 | CTCTCGCCATAAGCTTCCCGGATACACTAT |
| SEQ ID No: 141 | GGAGATTATCAGCTTCTATACCCACTGCGC |
| SEQ ID No: 142 | GGCTTTATTTGGCGTACTGTCATATTCGAT |
| SEQ ID No: 143 | GTACCATCATCCTAAAGAGCTCTTCAGTGT |
| SEQ ID No: 144 | GAAATGTATGAGATTGGCACCTTCTTAAAT |
| SEQ ID No: 145 | TCAGGACAACGCGAATCTTATAGCGCATAG |
| SEQ ID No: 146 | AACTCGACGTTCCACATGATATTGCCTCAA |
| SEQ ID No: 147 | CTTGACTGCATTGCTTTTGTAACCTACTGC |
| SEQ ID No: 148 | TTTCACGAATTCGAAGGGCACTGGCTTATT |
| SEQ ID No: 149 | GTGATCAGATAAGTCGTTAAAGCTCCTTCT |
| SEQ ID No: 150 | TCACTTAGTACTCATGAGAGGTAGTTTCGG |
| SEQ ID No: 151 | TCGTACCTGCTATATGTTAAACTTTAGAGA |
| SEQ ID No: 152 | CCCTCACCTTAAAAAGACCTACCTGTATTC |
| SEQ ID No: 153 | CAGGTTAATGATCCATCAAGACGAATCCTA |
| SEQ ID No: 154 | TGAAATTTAGGTATTATCTTGCTACTTGGA |
| SEQ ID No: 155 | ACGCAAGGGCATAGTTGAGTAATATAGAGA |
| SEQ ID No: 156 | GTCACCGAATGAGTACAGAAAGCTAGAGTG |
| SEQ ID No: 157 | TGCTACTTTACGAATTAATATGTCTTACCG |
| SEQ ID No: 158 | AAGGAAATGAGGTTCTTGTGAGTTCTAGTC |
| SEQ ID No: 159 | CCTCAAGGAAACGTACGATGTGACTTACTG |
| SEQ ID No: 160 | CAGTACCGCTCTTATAAAACTTAGTCGAAC |
| SEQ ID No: 161 | ATGATGTCTAACTGGCCATTTGTCCCTTTG |
| SEQ ID No: 162 | GGAAATTAGGACGCCATCTTGACTTTATAC |
| SEQ ID No: 163 | TCGCAAATTCATTATCAAACTCGCCGTGAG |
| SEQ ID No: 164 | GAAATCATGCGTGGCGTTGGTTAAATACGA |
| SEQ ID No: 165 | CCCGAAAGAGCCGTAATCCATTGTAAGCTG |
| SEQ ID No: 166 | ATGATTATCGCTAGTACCGTAAGTATTTTC |
| SEQ ID No: 167 | CATTCGAATAAGATACACGAGGACCATAGG |
| SEQ ID No: 168 | GTGTCAGTTGGATATTGTAACGTCGAAACC |
| SEQ ID No: 169 | ATGAGAGACTATACCGGGTACTGCAATATG |
| SEQ ID No: 170 | GTCTAATTTGCTCAACTCCTCTTCACCCAA |
| SEQ ID No: 171 | AGAAATACCAATCCACCTCGGAAATGTGTA |
| SEQ ID No: 172 | TAGATGTCAACAAACAAGCCAGTCTTCGCT |
| SEQ ID No: 173 | CCTAGACTTATTGACCTTGTTTAACCCGGC |
| SEQ ID No: 174 | TAAATAAGGATATCTGAGTCAACGGACGCC |
| SEQ ID No: 175 | TCTGAGGGATTCTACTTATCGAAGCCAACC |
| SEQ ID No: 176 | TCCAGTTATAAGTAAGAAACCGTCCGTCGT |
| SEQ ID No: 177 | ATTGACATTAAGCCATGCCAGCCCATAACT |
| SEQ ID No: 178 | GGTGAAAAGTTCCCACTACCCATAACAGTC |
| SEQ ID No: 179 | TATAGGCATCACGCAACCGTTTAGCGAATA |
| SEQ ID No: 180 | CACCCTCTCTACTCCAACTAATCCTACGCA |
| SEQ ID No: 181 | CCCAGGAATTAGTAGCGTTTCATGCAGAAT |
| SEQ ID No: 182 | ACCAGGCGGAACCATAAAGTGATACCTATC |
| SEQ ID No: 183 | TATTGGAAAGCCGCTCAAGATATAGTATAG |
| SEQ ID No: 184 | AGTGAACTTCTAGTGTATCCCAAAGCGTGG |
| SEQ ID No: 185 | GATTCTACAGCCAACATTACACTTCTCCAA |
| SEQ ID No: 186 | CGGTAAATCCCACTATGCTACATGTAAGCG |
| SEQ ID No: 187 | GTACAGCCCGATCCGAAATGGTTTAAGAGT |
| SEQ ID No: 188 | TCTGCACGCGACTTTAAGAATTGGCCATAG |
| SEQ ID No: 189 | AGACACAGATACGTATTTACTTAATCCGCG |
| SEQ ID No: 190 | ATTATAGCGTATTACCCAACACAATGAATT |
| SEQ ID No: 191 | TTACCCTTGCACCTAACACATATAACTGTA |
| SEQ ID No: 192 | CAGCTGAACACTCGCAGATCGTTTTACATA |
| SEQ ID No: 193 | GTTTAGGTTCTGCTACTAGCGTACATGAGG |
| SEQ ID No: 194 | AAAATGACACTGCCGATAAATATTGCGTGG |
| SEQ ID No: 195 | CGTCTCGAGGATGGTCCATTTTATAACTGT |
| SEQ ID No: 196 | AAGAATCTGCATCGGAAATATGAGTGTTAA |
| SEQ ID No: 197 | CGGATAAAGGCAGTGGATGGGTTAGAATGA |
| SEQ ID No: 198 | CAGTAAGTCCAAGATCCCTACCATAATTTC |
| SEQ ID No: 199 | GGAAGTTCTGAAGGGACATGTTCTAAGTGA |
| SEQ ID No: 200 | TGTGTTTTAGTGACGGCATAATTACCGGCG |
| SEQ ID No: 201 | AAAATATCGGACCTTTAGAAGTACGGTACC |
| SEQ ID No: 202 | TTGTGACATAATAACTAGAAGCCCTGGCTG |
| SEQ ID No: 203 | TGTTATCAAAACTTGTCGACACATCTGAGA |
| SEQ ID No: 204 | GACTTACTTAAGAACTGCTACGCCTACAAT |

### Flow Invariant Barcodes

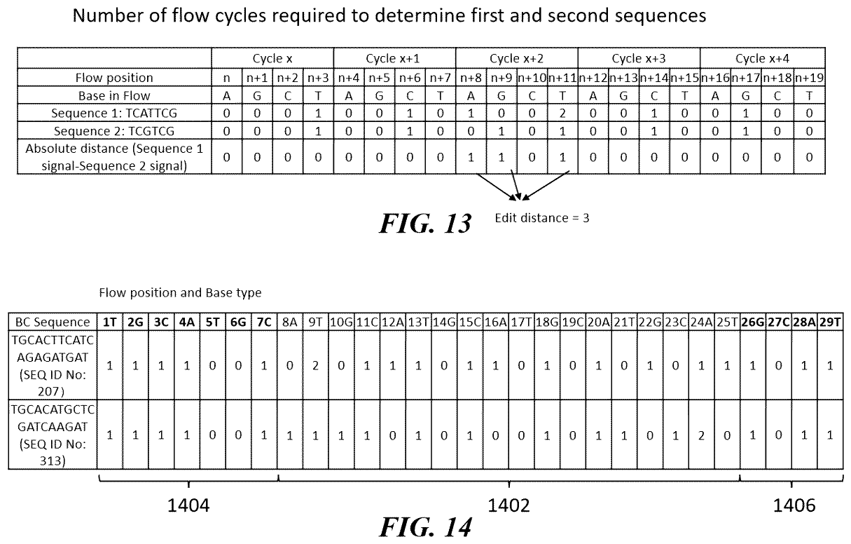

Barcodes are typically sequences of a given length that are used to uniquely identify different template molecules in a sequencing run. This limits the number of distinct barcode sequences available. In flow sequencing methods, unlike in typical next-generation sequencing methods, sequences of different nucleotide base lengths may be suitable as barcodes. This is because more than one nucleotide base may be incorporated in a nucleotide flow (see e.g., FIG. 2 and Example 1). FIG. 13 provides example flowgrams for a first sequence TCATTCG and a second sequence TCGTCG sequenced using a flow cycle order A-G-C-T. Both of these sequences, although they are different lengths, may be sequenced within 18 nucleotide flows. Advantageously, this feature of flow sequencing expands the potential pool of unique sequence barcodes available. Herein a set of barcodes of different sequence lengths but that have an effective length of 29 flows (e.g., are flow invariant) is provided. Methods of filtering sets of potential barcode sequences to meet predefined criteria are provided in International Pub. No. WO2023288018A2, which is entirely incorporated herein by reference for all purposes.

Barcode sequences often begin with a constant sequence (e.g., a preamble), which is determined based on the flow sequence to be used. For example, in sequencing by synthesis (e.g., flow-based sequencing) when the flow cycle sequence is T, G, C, A, the preamble sequence will be T, G, C, A, thereby providing flow cycle analog signal values of 1, 1, 1, 1 for each sequence read. In some instances, such a preamble sequence is of use for identifying sequencing colonies during signal detection and/or in providing a baseline signal level for downstream analog signal analysis. In some cases, different preamble sequences may be used to correspond with different selected flow cycle sequences. In some instances, all barcode sequences after the preamble sequence may start with a single nucleotide of a same type. For example, in all instances, all barcodes after the constant preamble sequence may start with a single A, a single T (or a (/), a single (′, or a single G. In some instances, all barcodes end with a constant sequence to support un-biased library prep. In some instances, the constant sequence is GAT. In some instances, the constant sequence is any series of three nucleotides. In some instances, the constant sequence is a series of more than 3 nucleotides (e.g., 4 or more nucleotides, 5 or more nucleotides, etc.).

An important feature of a barcode set is that each barcode must be distinctly identifiable from each other. That is, two barcodes that differ from each other by only a single base mismatch may be easily confused due to signal error or a single misincorporation event. Therefore, it is advantageous for barcodes to have sequences (or flowgrams) that are as different from each other as possible. One way of measuring this is by determining an edit distance (e.g., between nucleotide base sequences or between flowgrams). As one example, a Hamming distance may be calculated for all pairs of barcodes within a set. In such an example, for any given pair of barcode flowgrams, each flow position (e.g., which may comprise a flow cycle value or H-mer) of the first barcode is compared to the corresponding position of the second barcode. If the values differ for a given position, a value of 1 distance unit is added (e.g., every position in the pair of flowgrams that differs increases the value of the edit distance by 1). By way of example, a first flowgram comprising a 1×5 vector of [0, 0, 1, 1, 2] and a second flowgram comprising a 1×5 vector of [0, 0, 3, 2, 2] have an edit distance of 2, as two positions (the third and fourth elements) within the flowgrams differ in value. Each position in the pair of flowgrams that do not differ in value (e.g., the first, second, and fifth elements in this example) does not increase the edit distance between the corresponding barcode sequences. In the example in FIG. 13, the edit distance between the first and second sequence flowgrams is 3 (i.e., the total number of positions that differ).

Here, barcodes were required to have an effective edit distance of at least 3 from each other (e.g., there was a minimum edit distance of at least 3 between each possible pair of barcodes in the set). In effect, this minimum edit distance is only calculated for the variable sequence portions of each barcode sequence (e.g., because the preamble, constant prefix, and constant post sequences are identical for each barcode in the set). Further, each of the flowgram values for the variable sequence regions was set to 0, 1, or 2 (e.g., there were no homopolymers that are longer than 2 nucleotides long in base space). For each barcode, only one value in flow space was 2 (e.g., no more than one 2-mer was allowed per barcode, and each barcode was required to have one 2-mer).

Table 3 provides a list of distinct barcode sequences that fulfill the above criteria and that may be used simultaneously to label library molecules. These sequences vary in length, e.g., from TGCACACAGCCATATGCATGAT (SEQ ID No. 232) which is 22 nucleotide bases to TGCACACGCGATTCTGAT (SEQ ID No. 207) which is 18 nucleotide bases. FIG. 14 illustrates flowgrams for two other barcode sequences (SEQ ID Nos. 205 and 311) with the regions of the barcodes indicated. In FIG. 14, the distinct positions 1402 of the two barcodes are from flow 8 to flow 25 inclusive and correspond to a variable number of bases. The preamble region 1404 comprises 5 nucleotide bases and 7 flows. The constant 3′ end region 1406 comprises 3 bases and 4 flows.

Each sequence in Table 3 is listed in a 5′ to 3′ direction.

TABLE 3
Flow-invariant barcode sequences

| SEQ ID No. | Barcode sequence |
|---|---|
| SEQ ID No: 205 | TGCACTTCATCAGAGATGAT |
| SEQ ID No: 206 | TGCACAGACACAGCATTGAT |
| SEQ ID No: 207 | TGCACACGCGATTCTGAT |
| SEQ ID No: 208 | TGCACAGCCAGTCTGCTGAT |
| SEQ ID No: 209 | TGCACAAGTATCAGCGAT |
| SEQ ID No: 210 | TGCACTAGCAGTGTTGAT |
| SEQ ID No: 211 | TGCACGAGAGCAGCCATGAT |
| SEQ ID No: 212 | TGCACAATCGCATGTGTGAT |
| SEQ ID No: 213 | TGCACACATCTCGAAGAT |
| SEQ ID No: 214 | TGCACAACAGATAGAGAT |
| SEQ ID No: 215 | TGCACGCATAATACTGAT |
| SEQ ID No: 216 | TGCACATGTGTACTTGAT |
| SEQ ID No: 217 | TGCACTTCATGTGAGCTGAT |
| SEQ ID No: 218 | TGCACATGCTCAACAGCGAT |
| SEQ ID No: 219 | TGCACGTGGACATCAGAT |
| SEQ ID No: 220 | TGCACTCACAATGACGAT |
| SEQ ID No: 221 | TGCACAACGATATGTGAT |
| SEQ ID No: 222 | TGCACATCACCACGCGAT |
| SEQ ID No: 223 | TGCACTGCGAATCTGCTGAT |
| SEQ ID No: 224 | TGCACATATAATGCTGAGAT |
| SEQ ID No: 225 | TGCACATGATGCCGTCTGAT |
| SEQ ID No: 226 | TGCACTATCGATTGAGAT |
| SEQ ID No: 227 | TGCACAGCATTGCGCGCGAT |
| SEQ ID No: 228 | TGCACTCTATATGAAGAT |
| SEQ ID No: 229 | TGCACGCATGTCATTATGAT |
| SEQ ID No: 230 | TGCACATGCGGATCATCGAT |
| SEQ ID No: 231 | TGCACACAATCACTAGAT |
| SEQ ID No: 232 | TGCACACAGCCATATGCATGAT |
| SEQ ID No: 233 | TGCACACAAGACATGCTGAT |
| SEQ ID No: 234 | TGCACATGCTTCACTCTGAT |
| SEQ ID No: 235 | TGCACATCAGCAGTTATGAT |
| SEQ ID No: 236 | TGCACTATATCATGATTGAT |
| SEQ ID No: 237 | TGCACTGCGATGATTCTGAT |
| SEQ ID No: 238 | TGCACACATATATCCGAT |
| SEQ ID No: 239 | TGCACATAGATATGATTGAT |
| SEQ ID No: 240 | TGCACTGTGCTGGCATCATGAT |
| SEQ ID No: 241 | TGCACACTCTTCATGCTGAT |
| SEQ ID No: 242 | TGCACAGCATAGGCTCTGAT |
| SEQ ID No: 243 | TGCACATGGCATAGCGAGAT |
| SEQ ID No: 244 | TGCACTACACCACATGAT |
| SEQ ID No: 245 | TGCACAGTGATCCGTGAT |
| SEQ ID No: 246 | TGCACAGAGCGAATCATGAT |
| SEQ ID No: 247 | TGCACATGGCAGCTAGCATGAT |
| SEQ ID No: 248 | TGCACAGCAGATTATGCGAT |
| SEQ ID No: 249 | TGCACAATGCATACAGTGAT |
| SEQ ID No: 250 | TGCACAGCATGCACCATCAGAT |
| SEQ ID No: 251 | TGCACAAGTCAGTGTGAT |
| SEQ ID No: 252 | TGCACATGCAGCATCAAGAGAT |
| SEQ ID No: 253 | TGCACGCTGCAGTAAGAT |
| SEQ ID No: 254 | TGCACTGACAGTCAAGAT |
| SEQ ID No: 255 | TGCACAATCACATATGCGAT |
| SEQ ID No: 256 | TGCACATGCGTGGCTCTGAT |
| SEQ ID No: 257 | TGCACAGCCAGCAGATGCTGAT |
| SEQ ID No: 258 | TGCACACTAGCATCATTGAT |
| SEQ ID No: 259 | TGCACTGCATGTGAGAAGAT |
| SEQ ID No: 260 | TGCACTCGTCATGCCGAT |
| SEQ ID No: 261 | TGCACTGCACCAGCTGCGAT |
| SEQ ID No: 262 | TGCACATGGCGAGCTATGAT |
| SEQ ID No: 263 | TGCACTTCTGATCTCATGAT |
| SEQ ID No: 264 | TGCACTATCGCTGCCGAT |
| SEQ ID No: 265 | TGCACATGCAATATGAGCAGAT |
| SEQ ID No: 266 | TGCACAGATAATAGAGAT |
| SEQ ID No: 267 | TGCACTTATCAGCGTGAT |
| SEQ ID No: 268 | TGCACTAGGCATCGCGAT |
| SEQ ID No: 269 | TGCACTCTAGGATGCATGAT |
| SEQ ID No: 270 | TGCACTGCCTCATCATGCAGAT |
| SEQ ID No: 271 | TGCACAGCATGCCTGCACTGAT |
| SEQ ID No: 272 | TGCACATGCACTGCGCCATGAT |
| SEQ ID No: 273 | TGCACTCTGAGCAGGCTGAT |
| SEQ ID No: 274 | TGCACATAAGCTAGCGAT |
| SEQ ID No: 275 | TGCACAAGCACATGAGTGAT |
| SEQ ID No: 276 | TGCACGACTGGATGCGAT |
| SEQ ID No: 277 | TGCACATGCGTGCAAGCGAT |
| SEQ ID No: 278 | TGCACATGAGCTGCCTAGAT |
| SEQ ID No: 279 | TGCACTCTGCCTGCTATGAT |
| SEQ ID No: 280 | TGCACTATGTACATTGAT |
| SEQ ID No: 281 | TGCACACTGTTATGCGAT |
| SEQ ID No: 282 | TGCACTGCCATAGTGCTGAT |
| SEQ ID No: 283 | TGCACTCAACATCTCATGAT |
| SEQ ID No: 284 | TGCACATGCGCTCGCTTGAT |
| SEQ ID No: 285 | TGCACAGACGCTTGCATGAT |
| SEQ ID No: 286 | TGCACAAGAGCTCTGCAGAT |
| SEQ ID No: 287 | TGCACTCTTCAGCTGCTGAT |
| SEQ ID No: 288 | TGCACATATGCAATACAGAT |
| SEQ ID No: 289 | TGCACTATCTTGTATGAT |
| SEQ ID No: 290 | TGCACTCTGTTGCTGCTGAT |
| SEQ ID No: 291 | TGCACACTCATCCGTGAT |
| SEQ ID No: 292 | TGCACTGCATGCATTATGCGAT |
| SEQ ID No: 293 | TGCACAGCGCTAGAAGAT |
| SEQ ID No: 294 | TGCACGATAGCTGCATTGAT |
| SEQ ID No: 295 | TGCACGAGCTTGCATATGAT |
| SEQ ID No: 296 | TGCACACTGTGTCAAGAT |
| SEQ ID No: 297 | TGCACGCATGCGCAGTTGAT |
| SEQ ID No: 298 | TGCACGGACATCTGTGAT |
| SEQ ID No: 299 | TGCACAGACTTGTGAGAT |
| SEQ ID No: 300 | TGCACGATATGCATTCTGAT |
| SEQ ID No: 301 | TGCACAATAGATCTGCTGAT |
| SEQ ID No: 302 | TGCACGATGCTCCTGATGAT |
| SEQ ID No: 303 | TGCACTGCCGAGCATGCGAT |
| SEQ ID No: 304 | TGCACTGCACTCTGGCTGAT |
| SEQ ID No: 305 | TGCACGGCATGCTGAGCGAT |
| SEQ ID No: 306 | TGCACAATATCTGCTCTGAT |
| SEQ ID No: 307 | TGCACTGAATCATGCTGATGAT |
| SEQ ID No: 308 | TGCACTTGCATGACATAGAT |
| SEQ ID No: 309 | TGCACATGATGCGCCTGATGAT |
| SEQ ID No: 310 | TGCACATGACATGCCGTGAT |
| SEQ ID No: 311 | TGCACATGCTCGATCAAGAT |
| SEQ ID No: 312 | TGCACTCACACAATGCAGAT |
| SEQ ID No: 313 | TGCACTGCCACGAGCATGAT |
| SEQ ID No: 314 | TGCACTTACTCATGCGAT |
| SEQ ID No: 315 | TGCACGCGCTCAGCATTGAT |
| SEQ ID No: 316 | TGCACAGCGCAGATATTGAT |
| SEQ ID No: 317 | TGCACTGCAGAGGCAGAGAT |
| SEQ ID No: 318 | TGCACGGCAGTGCATGTGAT |
| SEQ ID No: 319 | TGCACTCATGGCATGAGCAGAT |
| SEQ ID No: 320 | TGCACATGAGTCAGATTGAT |
| SEQ ID No: 321 | TGCACAAGCATCTCAGTGAT |
| SEQ ID No: 322 | TGCACATCATCAAGCACATGAT |
| SEQ ID No: 323 | TGCACTTAGCGCATCGAT |
| SEQ ID No: 324 | TGCACACAATGCGCTGTGAT |
| SEQ ID No: 325 | TGCACAATATGCGATCAGAT |
| SEQ ID No: 326 | TGCACTTACATGTGCGAT |
| SEQ ID No: 327 | TGCACTGTGTGCCTGCAGAT |
| SEQ ID No: 328 | TGCACAATGCTGCTCATGCGAT |
| SEQ ID No: 329 | TGCACGATCTCATCATTGAT |
| SEQ ID No: 330 | TGCACAATCAGATGCTAGAT |
| SEQ ID No: 331 | TGCACAAGAGTGATAGAT |
| SEQ ID No: 332 | TGCACTTGCGTCATCATGAT |
| SEQ ID No: 333 | TGCACATGTCCGCACATGAT |
| SEQ ID No: 334 | TGCACACAAGCAGCTGCGAT |
| SEQ ID No: 335 | TGCACTATTGTGCGCATGAT |
| SEQ ID No: 336 | TGCACAACATGCATCATGTGAT |
| SEQ ID No: 337 | TGCACAACTGCAGATGAGAT |
| SEQ ID No: 338 | TGCACATCCTGTCGCGAT |
| SEQ ID No: 339 | TGCACTGCAGCTGCCATGCGAT |
| SEQ ID No: 340 | TGCACTATATTGCTCGAT |
| SEQ ID No: 341 | TGCACAATACTGATGCTGAT |
| SEQ ID No: 342 | TGCACATGTATGATGAAGAT |
| SEQ ID No: 343 | TGCACTGAAGCAGACATGAT |
| SEQ ID No: 344 | TGCACAATGCGAGCGCAGAT |
| SEQ ID No: 345 | TGCACATGACTCATCAAGAT |
| SEQ ID No: 346 | TGCACACATCGCCATCTGAT |
| SEQ ID No: 347 | TGCACTGCTGTGGATATGAT |
| SEQ ID No: 348 | TGCACTCGCGCTTGAGAT |
| SEQ ID No: 349 | TGCACACACTTGCTGCTGAT |
| SEQ ID No: 350 | TGCACTTGTCTGCTCGAT |
| SEQ ID No: 351 | TGCACTCAGACAGCATTGAT |
| SEQ ID No: 352 | TGCACATAGTTCACAGAT |
| SEQ ID No: 353 | TGCACATAACTGCGCGAT |
| SEQ ID No: 354 | TGCACACTTGCGCATGCGAT |
| SEQ ID No: 355 | TGCACTGCCTGACTGAGAT |
| SEQ ID No: 356 | TGCACTCAGAATAGCGAT |
| SEQ ID No: 357 | TGCACGGCGTGATGCGAT |
| SEQ ID No: 358 | TGCACATGCTGCATGCCGTGAT |
| SEQ ID No: 359 | TGCACTATGCATTCAGAGAT |
| SEQ ID No: 360 | TGCACATGGTCGCGTGAT |
| SEQ ID No: 361 | TGCACACGAGGATGCATGAT |
| SEQ ID No: 362 | TGCACTCGCACAAGCGAT |
| SEQ ID No: 363 | TGCACACAGTTGTGCGAT |
| SEQ ID No: 364 | TGCACACGGCTGCATATGAT |
| SEQ ID No: 365 | TGCACTGCTGATGCCGAGAT |
| SEQ ID No: 366 | TGCACAACTATAGCAGAT |
| SEQ ID No: 367 | TGCACGATATTCTATGAT |
| SEQ ID No: 368 | TGCACGAGCAGTGCCGAT |
| SEQ ID No: 369 | TGCACTGTTGCGCTGCTGAT |
| SEQ ID No: 370 | TGCACATATGGCACACTGAT |
| SEQ ID No: 371 | TGCACAGTACCAGATGAT |
| SEQ ID No: 372 | TGCACGCTGCCATCTGTGAT |
| SEQ ID No: 373 | TGCACAGCTGCATCATGAAGAT |
| SEQ ID No: 374 | TGCACATGATATTCATCATGAT |
| SEQ ID No: 375 | TGCACAATACATGTAGAT |
| SEQ ID No: 376 | TGCACTCAATGAGATGCGAT |
| SEQ ID No: 377 | TGCACAGTGAATATGATGAT |
| SEQ ID No: 378 | TGCACAGATCATGAATCATGAT |
| SEQ ID No: 379 | TGCACATCATCATCCTGCTGAT |
| SEQ ID No: 380 | TGCACGCTTCGACATGAT |
| SEQ ID No: 381 | TGCACAGAACTGTCTGAT |
| SEQ ID No: 382 | TGCACTTGCTGCATGCACAGAT |
| SEQ ID No: 383 | TGCACATGCAGACATGATTGAT |
| SEQ ID No: 384 | TGCACGCTGCATTGACTGAT |
| SEQ ID No: 385 | TGCACGATTATATGCATGAT |
| SEQ ID No: 386 | TGCACTCTATTGCTGATGAT |
| SEQ ID No: 387 | TGCACACAGCCGTGCATGAT |
| SEQ ID No: 388 | TGCACTGCTCCTGCGCTGAT |
| SEQ ID No: 389 | TGCACGCTAGCACAAGAT |
| SEQ ID No: 390 | TGCACGGCATCGATAGAT |
| SEQ ID No: 391 | TGCACATGATGCTCGCCGAT |
| SEQ ID No: 392 | TGCACTGATATGATTCAGAT |
| SEQ ID No: 393 | TGCACTTCGCTCACAGAT |
| SEQ ID No: 394 | TGCACGCTACCATATGAT |
| SEQ ID No: 395 | TGCACATATAGCATTGTGAT |
| SEQ ID No: 396 | TGCACTGCCGCGCGTGAT |
| SEQ ID No: 397 | TGCACTGAATCGATGCTGAT |
| SEQ ID No: 398 | TGCACATGCAGCCAGCATAGAT |
| SEQ ID No: 399 | TGCACATGGACAGCATGATGAT |
| SEQ ID No: 400 | TGCACGCTATGCGCCGAT |
| SEQ ID No: 401 | TGCACGTGCACTTGCGAT |
| SEQ ID No: 402 | TGCACATATGGTGATCTGAT |
| SEQ ID No: 403 | TGCACATGCATGCAGAAGCGAT |
| SEQ ID No: 404 | TGCACAATAGACGCAGAT |
| SEQ ID No: 405 | TGCACATCTGGAGCTCAGAT |
| SEQ ID No: 406 | TGCACTTATATGAGAGAT |
| SEQ ID No: 407 | TGCACACACAATCAGATGAT |
| SEQ ID No: 408 | TGCACATGTCTGCATGATTGAT |
| SEQ ID No: 409 | TGCACATGTGACATGCCGAT |
| SEQ ID No: 410 | TGCACTGCATTGATGACGAT |
| SEQ ID No: 411 | TGCACACAATGCATGTAGAT |
| SEQ ID No: 412 | TGCACAAGTGAGTCTGAT |
| SEQ ID No: 413 | TGCACATGTCTCATTCTGAT |
| SEQ ID No: 414 | TGCACAGAGCCACATGAGAT |
| SEQ ID No: 415 | TGCACTATTGATGCATGCAGAT |
| SEQ ID No: 416 | TGCACAGTGCCATGCATGTGAT |
| SEQ ID No: 417 | TGCACAGTTGCTCTGCTGAT |
| SEQ ID No: 418 | TGCACGGCACAGTGTGAT |
| SEQ ID No: 419 | TGCACTTGCACACAGCTGAT |
| SEQ ID No: 420 | TGCACATGCGACACATTGAT |
| SEQ ID No: 421 | TGCACAGTCATGCATGGCAGAT |
| SEQ ID No: 422 | TGCACTGAACATGAGCTGAT |
| SEQ ID No: 423 | TGCACAGCATATGCCATCAGAT |
| SEQ ID No: 424 | TGCACAATGTACATAGAT |
| SEQ ID No: 425 | TGCACATCATGTCATGGCTGAT |
| SEQ ID No: 426 | TGCACATCATCTGTGCCGAT |
| SEQ ID No: 427 | TGCACTTCTCTATCAGAT |
| SEQ ID No: 428 | TGCACATGCTTAGCATGATGAT |
| SEQ ID No: 429 | TGCACGATCATCAGATTGAT |
| SEQ ID No: 430 | TGCACTGTGCCTATGCAGAT |
| SEQ ID No: 431 | TGCACAGATGCATAATGCAGAT |
| SEQ ID No: 432 | TGCACAGCCATCTCATAGAT |
| SEQ ID No: 433 | TGCACTGCATCGTCCGAT |
| SEQ ID No: 434 | TGCACAATGATGACAGAGAT |
| SEQ ID No: 435 | TGCACTGCTAGCTCATTGAT |
| SEQ ID No: 436 | TGCACGATACATGAAGAT |
| SEQ ID No: 437 | TGCACTGTTGCATCAGCATGAT |
| SEQ ID No: 438 | TGCACATATCAGGCGCAGAT |
| SEQ ID No: 439 | TGCACATGCGCAGAAGAGAT |
| SEQ ID No: 440 | TGCACATGGTCATACATGAT |
| SEQ ID No: 441 | TGCACACATGCATCTCATTGAT |
| SEQ ID No: 442 | TGCACATCAGATGAGCCATGAT |
| SEQ ID No: 443 | TGCACGGTGCTGTCAGAT |
| SEQ ID No: 444 | TGCACGCAACAGACAGAT |
| SEQ ID No: 445 | TGCACAGCTGCTCGGATGAT |
| SEQ ID No: 446 | TGCACATCTGATCAAGAGAT |
| SEQ ID No: 447 | TGCACAGTGCAGCTCAAGAT |
| SEQ ID No: 448 | TGCACGCGGTGCACTGAT |
| SEQ ID No: 449 | TGCACATGATGTGAGTTGAT |
| SEQ ID No: 450 | TGCACTGAGCTGAGATTGAT |
| SEQ ID No: 451 | TGCACGCATGTCCACGAT |
| SEQ ID No: 452 | TGCACAGCTGCATAGCCGAT |
| SEQ ID No: 453 | TGCACTCAGCCTGCATAGAT |
| SEQ ID No: 454 | TGCACACATGATATATTGAT |
| SEQ ID No: 455 | TGCACTCTCTGAAGAGAT |
| SEQ ID No: 456 | TGCACATGGATCATCAGCAGAT |
| SEQ ID No: 457 | TGCACAGTGTGCCAGCTGAT |
| SEQ ID No: 458 | TGCACATACGCTTCAGAT |
| SEQ ID No: 459 | TGCACACTGCTGCAAGAGAT |
| SEQ ID No: 460 | TGCACACATGCTTATCTGAT |
| SEQ ID No: 461 | TGCACATCCGCTGACGAT |
| SEQ ID No: 462 | TGCACGCTTATCTCAGAT |
| SEQ ID No: 463 | TGCACATGCGCATGACCATGAT |
| SEQ ID No: 464 | TGCACATGACATTCTGCGAT |
| SEQ ID No: 465 | TGCACATGTCCATGCTAGAT |
| SEQ ID No: 466 | TGCACATCAGCTTCGCAGAT |
| SEQ ID No: 467 | TGCACTATGCTCCATCTGAT |
| SEQ ID No: 468 | TGCACATAATCACTCATGAT |
| SEQ ID No: 469 | TGCACTGAGCTGACCGAT |
| SEQ ID No: 470 | TGCACAAGAGATAGCATGAT |
| SEQ ID No: 471 | TGCACAGAATGATGCGCATGAT |
| SEQ ID No: 472 | TGCACAGTTATGATCGAT |
| SEQ ID No: 473 | TGCACAGCCACTCTGATGAT |
| SEQ ID No: 474 | TGCACTGCCAGACTGCAGAT |
| SEQ ID No: 475 | TGCACATCGCGCGCCGAT |
| SEQ ID No: 476 | TGCACGGAGATATATGAT |
| SEQ ID No: 477 | TGCACTTCGCAGTGCGAT |
| SEQ ID No: 478 | TGCACGTCATGATGGCTGAT |
| SEQ ID No: 479 | TGCACGTGCATGATATTGAT |
| SEQ ID No: 480 | TGCACGAGCACAACTGAT |
| SEQ ID No: 481 | TGCACATAGTGAGAAGAT |
| SEQ ID No: 482 | TGCACTGAATGCACACAGAT |
| SEQ ID No: 483 | TGCACGCTGCACCATCTGAT |
| SEQ ID No: 484 | TGCACAATGATGCACATGTGAT |
| SEQ ID No: 485 | TGCACGATGCATTCGCTGAT |
| SEQ ID No: 486 | TGCACACACACTGCCATGAT |
| SEQ ID No: 487 | TGCACTGCGCTGCGGCAGAT |
| SEQ ID No: 488 | TGCACAACACAGTCAGAT |
| SEQ ID No: 489 | TGCACTCTCATAAGCGAT |
| SEQ ID No: 490 | TGCACTGATGCATGCAACTGAT |
| SEQ ID No: 491 | TGCACAATCGCTATGCAGAT |
| SEQ ID No: 492 | TGCACATGTGGATGCGCGAT |
| SEQ ID No: 493 | TGCACATCGCATCATGGATGAT |
| SEQ ID No: 494 | TGCACATATGATGCACCATGAT |
| SEQ ID No: 495 | TGCACGCAACTCAGCGAT |
| SEQ ID No: 496 | TGCACGCGCGGCTATGAT |
| SEQ ID No: 497 | TGCACTAGTGCAAGCGAT |
| SEQ ID No: 498 | TGCACAGAGTTGCACATGAT |
| SEQ ID No: 499 | TGCACTCATCAGTGCAAGAT |
| SEQ ID No: 500 | TGCACAGCATGAGAATGATGAT |
| SEQ ID No: 501 | TGCACACAGACGATTGAT |
| SEQ ID No: 502 | TGCACGTGCACAGTTGAT |
| SEQ ID No: 503 | TGCACATGGTGATGACAGAT |
| SEQ ID No: 504 | TGCACAGAGAGCTGCTTGAT |
| SEQ ID No: 505 | TGCACTGTCAGCACCGAT |
| SEQ ID No: 506 | TGCACTGTGACTGCCATGAT |
| SEQ ID No: 507 | TGCACAGATCCAGATGAGAT |
| SEQ ID No: 508 | TGCACAGAGTGTGTTGAT |
| SEQ ID No: 509 | TGCACATGCAGACAGCCGAT |
| SEQ ID No: 510 | TGCACACAAGTCACTGAT |
| SEQ ID No: 511 | TGCACTATGACTTGCGAT |
| SEQ ID No: 512 | TGCACTGCACTGTCATTGAT |
| SEQ ID No: 513 | TGCACATCCATCGTCATGAT |
| SEQ ID No: 514 | TGCACATCACATACATTGAT |
| SEQ ID No: 515 | TGCACTACCTGCATGATGAT |
| SEQ ID No: 516 | TGCACAATGAGCAGATCGAT |
| SEQ ID No: 517 | TGCACAGTATTGCACATGAT |
| SEQ ID No: 518 | TGCACACATAATGACATGAT |
| SEQ ID No: 519 | TGCACGATGCCTATGCTGAT |
| SEQ ID No: 520 | TGCACATGCAAGTGACTGAT |
| SEQ ID No: 521 | TGCACACATGGTGCACAGAT |
| SEQ ID No: 522 | TGCACTTGCTCATCGATGAT |
| SEQ ID No: 523 | TGCACAAGCTGACAGAGAT |
| SEQ ID No: 524 | TGCACTGCCACAGCTATGAT |
| SEQ ID No: 525 | TGCACTCAACTGCAGCAGAT |
| SEQ ID No: 526 | TGCACAAGCATGTATCAGAT |
| SEQ ID No: 527 | TGCACTATTCATATGCAGAT |
| SEQ ID No: 528 | TGCACACAGCTCATGAAGAT |
| SEQ ID No: 529 | TGCACAATGTGAGTGATGAT |
| SEQ ID No: 530 | TGCACATGACCATCTATGAT |
| SEQ ID No: 534 | TGCACATGGCTAGCGCTGAT |
| SEQ ID No: 535 | TGCACAGATGCATGCTGCCGAT |
| SEQ ID No: 536 | TGCACATCATTCAGATAGAT |
| SEQ ID No: 537 | TGCACTTGCATACGCATGAT |
| SEQ ID No: 538 | TGCACATCCATGAGACTGAT |
| SEQ ID No: 539 | TGCACTTATGCAGAGCTGAT |
| SEQ ID No: 540 | TGCACATCCATGCTCTCGAT |
| SEQ ID No: 541 | TGCACAACGCTCAGCGAT |
| SEQ ID No: 542 | TGCACAGACATGGCATAGAT |
| SEQ ID No: 543 | TGCACGCTATGTTGCATGAT |
| SEQ ID No: 544 | TGCACAGCTGATTGCTCGAT |
| SEQ ID No: 545 | TGCACTGTGTTGTGTGAT |
| SEQ ID No: 546 | TGCACTACCATGCATCTGAT |
| SEQ ID No: 547 | TGCACTGATCCGCTAGAT |
| SEQ ID No: 548 | TGCACTAGGATGCAGATGAT |
| SEQ ID No: 549 | TGCACGCGCAAGCGCATGAT |
| SEQ ID No: 550 | TGCACTCTCTTGTGTGAT |
| SEQ ID No: 551 | TGCACTAGCACAATGATGAT |
| SEQ ID No: 552 | TGCACAGAGCAGGCACAGAT |
| SEQ ID No: 553 | TGCACAAGATGATCGAGAT |
| SEQ ID No: 554 | TGCACACACTGCCACATGAT |
| SEQ ID No: 555 | TGCACAGCCTATGCAGTGAT |
| SEQ ID No: 556 | TGCACATATGTGGCTGCGAT |
| SEQ ID No: 557 | TGCACAGATCAGGCTATGAT |
| SEQ ID No: 558 | TGCACATGTCATTGCAGCTGAT |
| SEQ ID No: 559 | TGCACTGAGTGCCTGATGAT |
| SEQ ID No: 560 | TGCACTGCATCAATGCTCTGAT |
| SEQ ID No: 561 | TGCACTGCTGGAGAGCAGAT |
| SEQ ID No: 562 | TGCACTGCATCTCAGAAGAT |
| SEQ ID No: 563 | TGCACATGATTATCGATGAT |
| SEQ ID No: 564 | TGCACAGCTCTGATTGAGAT |
| SEQ ID No: 565 | TGCACTGCATGCCAGATATGAT |
| SEQ ID No: 566 | TGCACAGCATGCAGCTGAAGAT |
| SEQ ID No: 567 | TGCACGCTTCATCACGAT |
| SEQ ID No: 568 | TGCACATAACGTGATGAT |
| SEQ ID No: 569 | TGCACATGCACATGTGATTGAT |
| SEQ ID No: 570 | TGCACAGCATCAACAGAGAT |
| SEQ ID No: 571 | TGCACTCAGATGCAACTGAT |
| SEQ ID No: 572 | TGCACTGTGCAGCTGAAGAT |
| SEQ ID No: 573 | TGCACTGCAGTATGCTTGAT |
| SEQ ID No: 574 | TGCACATCGCGCTCATTGAT |
| SEQ ID No: 575 | TGCACAGCATCGCATGGCAGAT |
| SEQ ID No: 576 | TGCACAAGTCGCATAGAT |
| SEQ ID No: 577 | TGCACAGCATGTTGCAGATGAT |
| SEQ ID No: 578 | TGCACAGCTCCGCATGTGAT |
| SEQ ID No: 579 | TGCACGAGCGATTGTGAT |
| SEQ ID No: 580 | TGCACACATGAGCTTATGAT |
| SEQ ID No: 581 | TGCACAGTCATGGAGATGAT |
| SEQ ID No: 582 | TGCACATGCAGCATGCGCCGAT |
| SEQ ID No: 583 | TGCACAGCCGCAGCAGCGAT |
| SEQ ID No: 584 | TGCACGGCAGCGTCAGAT |
| SEQ ID No: 585 | TGCACACAGAGCACCATGAT |
| SEQ ID No: 586 | TGCACAACTCATGCATAGAT |
| SEQ ID No: 587 | TGCACATCAGTGGCGCTGAT |
| SEQ ID No: 588 | TGCACTGCACATGAATGCAGAT |
| SEQ ID No: 589 | TGCACATGAGCTTGTGCGAT |
| SEQ ID No: 590 | TGCACAATGACAGTGCTGAT |
| SEQ ID No: 591 | TGCACAAGTGTCATCATGAT |
| SEQ ID No: 592 | TGCACGATGCTGCAATCGAT |
| SEQ ID No: 593 | TGCACAATGCACATGCTCAGAT |
| SEQ ID No: 594 | TGCACGATGATGTCATTGAT |
| SEQ ID No: 595 | TGCACGATGCACATATTGAT |
| SEQ ID No: 596 | TGCACATCATGCGCCAGCTGAT |
| SEQ ID No: 597 | TGCACATGCAATGATATCTGAT |
| SEQ ID No: 598 | TGCACAGTCGCTGCCGAT |
| SEQ ID No: 599 | TGCACGCAATCGCTCATGAT |
| SEQ ID No: 600 | TGCACTATGACATCCATGAT |
| SEQ ID No: 601 | TGCACTACAGAGGCAGAT |
| SEQ ID No: 602 | TGCACATGAGGCGAGCTGAT |
| SEQ ID No: 603 | TGCACAACGCATATCGAT |
| SEQ ID No: 604 | TGCACTCTCTGCACCATGAT |
| SEQ ID No: 605 | TGCACACTCGCGGCTGAT |
| SEQ ID No: 606 | TGCACATCTGCACAGCATTGAT |
| SEQ ID No: 607 | TGCACGATGCATGAAGTGAT |
| SEQ ID No: 608 | TGCACTTGCATCTGAGTGAT |
| SEQ ID No: 609 | TGCACATGGCACGCACAGAT |
| SEQ ID No: 610 | TGCACACAGCTAGTTGAT |
| SEQ ID No: 611 | TGCACACTTCTATATGAT |
| SEQ ID No: 612 | TGCACTCATAATGTCATGAT |
| SEQ ID No: 613 | TGCACTGAGATGCAATAGAT |
| SEQ ID No: 614 | TGCACAAGATGTAGCGAT |
| SEQ ID No: 615 | TGCACAATGCATGTCACATGAT |
| SEQ ID No: 616 | TGCACAATGTCACTGCAGAT |
| SEQ ID No: 617 | TGCACATGATGCTGCACAAGAT |
| SEQ ID No: 618 | TGCACACAATAGAGAGAT |
| SEQ ID No: 619 | TGCACTGCATGCGACTTGAT |
| SEQ ID No: 620 | TGCACATATCCACAGATGAT |
| SEQ ID No: 621 | TGCACATCATTGAGCAGATGAT |
| SEQ ID No: 622 | TGCACAGTGCGCGCCATGAT |
| SEQ ID No: 623 | TGCACATCATCTTAGCGAT |
| SEQ ID No: 624 | TGCACTTATGTCTGTGAT |
| SEQ ID No: 625 | TGCACTCAATCAGCAGCGAT |
| SEQ ID No: 626 | TGCACACAGCATGCCGTGAT |
| SEQ ID No: 627 | TGCACTAGCATGGCTCAGAT |
| SEQ ID No: 628 | TGCACAATGATCAGAGTGAT |
| SEQ ID No: 629 | TGCACTCAAGCTCAGATGAT |
| SEQ ID No: 630 | TGCACATGGCTGCTGTGATGAT |
| SEQ ID No: 631 | TGCACTGCGCACTCATTGAT |
| SEQ ID No: 632 | TGCACTACAGATATTGAT |
| SEQ ID No: 633 | TGCACATCCGCATGCGAGAT |
| SEQ ID No: 634 | TGCACTGCCGCATATCAGAT |
| SEQ ID No: 635 | TGCACAACAGCACACGAT |
| SEQ ID No: 636 | TGCACATATCTATCCGAT |
| SEQ ID No: 637 | TGCACTGTATTCTGAGAT |
| SEQ ID No: 638 | TGCACAGAGCATGCCAGCTGAT |
| SEQ ID No: 639 | TGCACATATCATGAATGATGAT |
| SEQ ID No: 640 | TGCACATCCATCTGCTGATGAT |
| SEQ ID No: 641 | TGCACATAGCACCATGCGAT |
| SEQ ID No: 642 | TGCACTCTGCGCCAGCAGAT |
| SEQ ID No: 643 | TGCACAGCTCTGCGCAAGAT |
| SEQ ID No: 644 | TGCACAACAGTCTATGAT |
| SEQ ID No: 645 | TGCACAGCATTGTGTGAGAT |
| SEQ ID No: 646 | TGCACAATCATGAGTGAGAT |
| SEQ ID No: 647 | TGCACTCTGCATGTCTTGAT |
| SEQ ID No: 648 | TGCACTGCTGCTCACTTGAT |
| SEQ ID No: 649 | TGCACTGATGTGGATGCGAT |
| SEQ ID No: 650 | TGCACTTGCAGATCAGTGAT |
| SEQ ID No: 651 | TGCACAGCGCCATCATGCTGAT |
| SEQ ID No: 652 | TGCACTGATACTTCAGAT |
| SEQ ID No: 653 | TGCACGAGGCTGTGCATGAT |
| SEQ ID No: 654 | TGCACAGCCATGTCGCAGAT |
| SEQ ID No: 655 | TGCACATGTGCGGAGATGAT |
| SEQ ID No: 656 | TGCACATAGCCTGCGATGAT |
| SEQ ID No: 657 | TGCACTGAGCTCCATGAGAT |
| SEQ ID No: 658 | TGCACTCGCTCTGCCATGAT |
| SEQ ID No: 659 | TGCACAGCATTACACGAT |
| SEQ ID No: 660 | TGCACTATTATGATGCTGAT |
| SEQ ID No: 661 | TGCACGATGTTGCTGCTGAT |
| SEQ ID No: 662 | TGCACTGAGCCATGTATGAT |
| SEQ ID No: 663 | TGCACATGTGCAATCATATGAT |
| SEQ ID No: 664 | TGCACAAGATACTGTGAT |
| SEQ ID No: 665 | TGCACAGCGAAGCAGATGAT |
| SEQ ID No: 666 | TGCACATGTAAGCAGCAGAT |
| SEQ ID No: 667 | TGCACTGTTCTGCAGCAGAT |
| SEQ ID No: 668 | TGCACATGTACACTTGAT |
| SEQ ID No: 669 | TGCACAGTGCAGAGGCTGAT |
| SEQ ID No: 670 | TGCACACTGAACATGATGAT |
| SEQ ID No: 671 | TGCACTGCAGCAGTCTTGAT |
| SEQ ID No: 672 | TGCACGGAGCATAGCGAT |
| SEQ ID No: 673 | TGCACAGTATCTGCCATGAT |
| SEQ ID No: 674 | TGCACTGTGATGCAAGTGAT |
| SEQ ID No: 675 | TGCACATGCTGAGCATCTTGAT |
| SEQ ID No: 676 | TGCACTGCGCATTGCATGAGAT |
| SEQ ID No: 677 | TGCACGCGAGACATTGAT |
| SEQ ID No: 678 | TGCACTATGCCATCTATGAT |
| SEQ ID No: 679 | TGCACTGCTAGAGTTGAT |
| SEQ ID No: 680 | TGCACAAGACAGCGAGAT |
| SEQ ID No: 681 | TGCACTGCCAGATGCGCATGAT |
| SEQ ID No: 682 | TGCACTTGTATATATGAT |
| SEQ ID No: 683 | TGCACTTCAGCGAGCGAT |
| SEQ ID No: 684 | TGCACAACAGACTCAGAT |
| SEQ ID No: 685 | TGCACAAGCAGCTACGAT |
| SEQ ID No: 686 | TGCACATGGAGCATCACGAT |
| SEQ ID No: 687 | TGCACGACTGCAAGCATGAT |
| SEQ ID No: 688 | TGCACTATGCTGCAAGTGAT |
| SEQ ID No: 689 | TGCACTCTTATCAGCATGAT |
| SEQ ID No: 690 | TGCACTCAAGCATGCATGTGAT |
| SEQ ID No: 691 | TGCACTCAGCATATATTGAT |
| SEQ ID No: 692 | TGCACAGTTCATATAGAT |
| SEQ ID No: 693 | TGCACTCATGAGGCACAGAT |
| SEQ ID No: 694 | TGCACGCTGCGATCCAGAT |
| SEQ ID No: 695 | TGCACGGCGCTCATAGAT |
| SEQ ID No: 696 | TGCACAGAGAGAGAAGAT |
| SEQ ID No: 697 | TGCACGCTGTATTGCATGAT |
| SEQ ID No: 698 | TGCACTGCATTGCACGAGAT |
| SEQ ID No: 699 | TGCACTCGGCTGAGCATGAT |
| SEQ ID No: 700 | TGCACTCTGCCGCTCGAT |
| SEQ ID No: 701 | TGCACGTCAGGCAGCATGAT |
| SEQ ID No: 702 | TGCACGGCTGCAGTGCGAT |
| SEQ ID No: 703 | TGCACAAGCGCGTGTGAT |
| SEQ ID No: 704 | TGCACGGCACTACATGAT |
| SEQ ID No: 705 | TGCACTAGCGCAGCCATGAT |
| SEQ ID No: 706 | TGCACTTCTGTGTGAGAT |
| SEQ ID No: 707 | TGCACACAAGATGCATGATGAT |
| SEQ ID No: 708 | TGCACATGCTGCATTAGATGAT |
| SEQ ID No: 709 | TGCACTGCATATTATGTGAT |
| SEQ ID No: 710 | TGCACACGAGCATCCGAT |
| SEQ ID No: 711 | TGCACATGATGTCTTGAGAT |
| SEQ ID No: 712 | TGCACAGCATGATGTGCTTGAT |
| SEQ ID No: 713 | TGCACATGCTTGCAGATGTGAT |
| SEQ ID No: 714 | TGCACTATCACATGGCTGAT |
| SEQ ID No: 715 | TGCACTTGCGCAGAGCTGAT |
| SEQ ID No: 716 | TGCACAGAATCGCAGCTGAT |
| SEQ ID No: 717 | TGCACATAAGAGAGCGAT |
| SEQ ID No: 718 | TGCACATCAGGTCAGAGAT |
| SEQ ID No: 719 | TGCACAATCTCTCGCATGAT |
| SEQ ID No: 720 | TGCACATGCATCATATCAAGAT |
| SEQ ID No: 721 | TGCACACTGCCATGCATCTGAT |
| SEQ ID No: 722 | TGCACTCGGCAGCAGATGAT |
| SEQ ID No: 723 | TGCACTTGAGCGATAGAT |
| SEQ ID No: 724 | TGCACTATAGCAGCCATGAT |
| SEQ ID No: 725 | TGCACGCGCATGCTGCCGAT |
| SEQ ID No: 726 | TGCACATCATATCATGCAAGAT |
| SEQ ID No: 727 | TGCACTGTAGCGATTGAT |
| SEQ ID No: 728 | TGCACTCGATTGCATATGAT |
| SEQ ID No: 729 | TGCACAGAGCGCTCCGAT |
| SEQ ID No: 730 | TGCACGCATCCTATGCAGAT |
| SEQ ID No: 731 | TGCACGCATATAATAGAT |
| SEQ ID No: 732 | TGCACAGAATGTGCTATGAT |
| SEQ ID No: 733 | TGCACAGCGATCATTATGAT |
| SEQ ID No: 734 | TGCACTCGCACATAAGAT |
| SEQ ID No: 735 | TGCACACTGCTGGTGCTGAT |
| SEQ ID No: 736 | TGCACGGTGATCACTGAT |
| SEQ ID No: 737 | TGCACATAGAGCCTGATGAT |
| SEQ ID No: 738 | TGCACTATCATGGACATGAT |
| SEQ ID No: 739 | TGCACAATCATCTGACTGAT |
| SEQ ID No: 740 | TGCACATCCAGCGTCGAT |
| SEQ ID No: 741 | TGCACATGCAGCATTCTGTGAT |
| SEQ ID No: 742 | TGCACAGTTGAGCACGAT |
| SEQ ID No: 743 | TGCACACGGCATCGCATGAT |
| SEQ ID No: 744 | TGCACATCACAGGCTCTGAT |
| SEQ ID No: 745 | TGCACTTCATGCTGCACGAT |
| SEQ ID No: 746 | TGCACAATGCGCTGTCAGAT |
| SEQ ID No: 747 | TGCACTGCTGTGCAAGCATGAT |
| SEQ ID No: 748 | TGCACAGATGGCTACATGAT |
| SEQ ID No: 749 | TGCACTGCTCACACCATGAT |
| SEQ ID No: 750 | TGCACTGTCAACAGAGAT |
| SEQ ID No: 751 | TGCACAGTTGCATCACAGAT |
| SEQ ID No: 752 | TGCACGACATTGCTCATGAT |
| SEQ ID No: 753 | TGCACATCTCAGGACAGAT |
| SEQ ID No: 754 | TGCACACAATATCTGATGAT |
| SEQ ID No: 755 | TGCACTATCTGAAGCGAT |
| SEQ ID No: 756 | TGCACGGATCTGTGTGAT |
| SEQ ID No: 757 | TGCACTATGTTGCTAGAT |
| SEQ ID No: 758 | TGCACGGCATGAGAGCTGAT |
| SEQ ID No: 759 | TGCACAGAAGATCGCGAT |
| SEQ ID No: 760 | TGCACTGAGTGCATTCTGAT |
| SEQ ID No: 761 | TGCACACTTGACACTGAT |
| SEQ ID No: 762 | TGCACATAATGCATGCTGTGAT |
| SEQ ID No: 763 | TGCACAGTCTTGTATGAT |
| SEQ ID No: 764 | TGCACGCACGCGCAAGAT |
| SEQ ID No: 765 | TGCACATCCGATACAGAT |
| SEQ ID No: 766 | TGCACTGCCTCTGATCTGAT |
| SEQ ID No: 767 | TGCACGGCTGCTATCGAT |
| SEQ ID No: 768 | TGCACTGTTGTCAGCGAT |
| SEQ ID No: 769 | TGCACACGAGCTTGAGAT |
| SEQ ID No: 770 | TGCACTCACTATTGCATGAT |
| SEQ ID No: 771 | TGCACACTAGCTGTTGAT |
| SEQ ID No: 772 | TGCACAGCATCATAAGCATGAT |
| SEQ ID No: 773 | TGCACATCGTCAAGCATGAT |
| SEQ ID No: 774 | TGCACAGCTGGAGCATAGAT |
| SEQ ID No: 775 | TGCACAGATGCAAGCGTGAT |
| SEQ ID No: 776 | TGCACAGTGTTGAGAGAT |
| SEQ ID No: 777 | TGCACTGCTCAGATGCCGAT |
| SEQ ID No: 778 | TGCACGAGGCATGATCAGAT |
| SEQ ID No: 779 | TGCACGCGCGCAATGCTGAT |
| SEQ ID No: 780 | TGCACATGAGCACTGCCATGAT |
| SEQ ID No: 781 | TGCACTGCGCCATCGAGAT |
| SEQ ID No: 782 | TGCACTTCACATAGTGAT |
| SEQ ID No: 783 | TGCACACAGAGAAGTGAT |
| SEQ ID No: 784 | TGCACTCAACGATCAGAT |
| SEQ ID No: 785 | TGCACTGCGATGGAGATGAT |
| SEQ ID No: 786 | TGCACATCTCATCTATTGAT |
| SEQ ID No: 787 | TGCACACATGATGATGCAAGAT |
| SEQ ID No: 788 | TGCACACTGATGTGGATGAT |
| SEQ ID No: 789 | TGCACTGACGGCAGCGAT |
| SEQ ID No: 790 | TGCACATACGGACATGAT |
| SEQ ID No: 791 | TGCACTCTATGCCATCAGAT |
| SEQ ID No: 792 | TGCACTTGCTCTAGCATGAT |
| SEQ ID No: 793 | TGCACGTCATGTTGAGAT |
| SEQ ID No: 794 | TGCACATATGATCTTCAGAT |
| SEQ ID No: 795 | TGCACGCTTACAGCTGAT |
| SEQ ID No: 796 | TGCACAATGCACGTAGAT |
| SEQ ID No: 797 | TGCACATGATCAATGACGAT |
| SEQ ID No: 798 | TGCACACTCGGATGCATGAT |
| SEQ ID No: 799 | TGCACATGGCGCTGAGTGAT |
| SEQ ID No: 800 | TGCACATGCATGGATCTGCGAT |
| SEQ ID No: 801 | TGCACAATGTCGTGAGAT |
| SEQ ID No: 802 | TGCACTGCACCGCATCTGAT |
| SEQ ID No: 803 | TGCACTGCAGATACCATGAT |
| SEQ ID No: 804 | TGCACAGTCGAGGCAGAT |
| SEQ ID No: 805 | TGCACTATGCCTATGATGAT |
| SEQ ID No: 806 | TGCACTTCAGCATCTGAGAT |
| SEQ ID No: 807 | TGCACATATGCATCGCCATGAT |
| SEQ ID No: 808 | TGCACAGCACCTGCAGAGAT |
| SEQ ID No: 809 | TGCACTTGATCATATCTGAT |
| SEQ ID No: 810 | TGCACTATCAATCACGAT |
| SEQ ID No: 811 | TGCACACATCTGAGATTGAT |
| SEQ ID No: 812 | TGCACTCTCAAGCATATGAT |
| SEQ ID No: 813 | TGCACATCACAGCTTGAGAT |
| SEQ ID No: 814 | TGCACGATGACAAGCGAT |
| SEQ ID No: 815 | TGCACGGCTCAGCTCGAT |
| SEQ ID No: 816 | TGCACACATCCACATGCGAT |
| SEQ ID No: 817 | TGCACAATGAGATGCTGCAGAT |
| SEQ ID No: 818 | TGCACGGCTGATCGCATGAT |
| SEQ ID No: 819 | TGCACGCTGTGCTCATTGAT |
| SEQ ID No: 820 | TGCACACTCATGTGGCAGAT |
| SEQ ID No: 821 | TGCACTTGTGAGATAGAT |
| SEQ ID No: 822 | TGCACACTTGTAGATGAT |
| SEQ ID No: 823 | TGCACAACATGATGCGAGAT |
| SEQ ID No: 824 | TGCACACAACTGTATGAT |
| SEQ ID No: 825 | TGCACAATATGTCATATGAT |
| SEQ ID No: 826 | TGCACAATCATATGCACGAT |
| SEQ ID No: 827 | TGCACATCATCTCAACAGAT |
| SEQ ID No: 828 | TGCACAACATACAGCATGAT |
| SEQ ID No: 829 | TGCACTCATGGAGCTCTGAT |
| SEQ ID No: 830 | TGCACTCTGCATCGGCTGAT |
| SEQ ID No: 831 | TGCACAATATACGCTGAT |
| SEQ ID No: 832 | TGCACGCTGCGAAGAGAT |
| SEQ ID No: 833 | TGCACAGAGACAAGTGAT |
| SEQ ID No: 834 | TGCACTCTCATCATTGTGAT |
| SEQ ID No: 835 | TGCACTCTTGCTCGAGAT |
| SEQ ID No: 836 | TGCACTTGATCGAGATGAT |
| SEQ ID No: 837 | TGCACATGGTAGCTCGAT |
| SEQ ID No: 838 | TGCACATGTGCATGGATGCGAT |
| SEQ ID No: 839 | TGCACTGCAGCTTGTGTGAT |
| SEQ ID No: 840 | TGCACATATCGCCATGCATGAT |
| SEQ ID No: 841 | TGCACGACCATCATGCAGAT |
| SEQ ID No: 842 | TGCACATCATGCCAGCATCGAT |
| SEQ ID No: 843 | TGCACTGATCAGCATCCATGAT |
| SEQ ID No: 844 | TGCACGAGCGCTGAAGAT |
| SEQ ID No: 845 | TGCACATGCTGTAGCAAGAT |
| SEQ ID No: 846 | TGCACTTCAGAGATCATGAT |
| SEQ ID No: 847 | TGCACACGATTGCTCATGAT |
| SEQ ID No: 848 | TGCACATCTATGAGGATGAT |
| SEQ ID No: 849 | TGCACGCAATCACTGCAGAT |
| SEQ ID No: 850 | TGCACAATCGAGCGTGAT |
| SEQ ID No: 851 | TGCACTCACAGATGGCTGAT |
| SEQ ID No: 852 | TGCACTGTATGAGCCATGAT |
| SEQ ID No: 853 | TGCACACATGCAGAGCCATGAT |
| SEQ ID No: 854 | TGCACATAACATGCATGCAGAT |
| SEQ ID No: 855 | TGCACTTGCTGACGCGAT |
| SEQ ID No: 856 | TGCACTGACAATGCAGAGAT |
| SEQ ID No: 857 | TGCACGTGCTTGCGCGAT |
| SEQ ID No: 858 | TGCACAGCTACATGGCAGAT |
| SEQ ID No: 859 | TGCACATGCTATGCCTGCAGAT |
| SEQ ID No: 860 | TGCACACAATGTGCTCTGAT |
| SEQ ID No: 861 | TGCACTTGTGTCTATGAT |
| SEQ ID No: 862 | TGCACTGCACTAATCGAT |
| SEQ ID No: 863 | TGCACGATGATATCCGAT |
| SEQ ID No: 864 | TGCACACATAAGATGCTGAT |
| SEQ ID No: 865 | TGCACTGCAGATATGAAGAT |
| SEQ ID No: 866 | TGCACAGCGCGCCACATGAT |
| SEQ ID No: 867 | TGCACATAATGCTGAGCATGAT |
| SEQ ID No: 868 | TGCACTCATCATTGCGAGAT |
| SEQ ID No: 869 | TGCACAGAGCTATGATTGAT |
| SEQ ID No: 870 | TGCACTTCTGCTAGTGAT |
| SEQ ID No: 871 | TGCACGGTGTCATGTGAT |
| SEQ ID No: 872 | TGCACTCACTGTGAAGAT |
| SEQ ID No: 873 | TGCACTTCGATATGCATGAT |
| SEQ ID No: 874 | TGCACTCATCCATCATAGAT |
| SEQ ID No: 875 | TGCACATAGTGTTCAGAT |
| SEQ ID No: 876 | TGCACATGATTGAGCTAGAT |
| SEQ ID No: 877 | TGCACATCATGCGCATTATGAT |
| SEQ ID No: 878 | TGCACATGTATGTCCATGAT |
| SEQ ID No: 879 | TGCACTGCCATCTGCATATGAT |
| SEQ ID No: 880 | TGCACTGACTCAAGATGAT |
| SEQ ID No: 881 | TGCACAAGCTATGCTATGAT |
| SEQ ID No: 882 | TGCACAAGACTCGCTGAT |
| SEQ ID No: 883 | TGCACTCTGCGCATTGTGAT |
| SEQ ID No: 884 | TGCACGGTCACTGATGAT |
| SEQ ID No: 885 | TGCACATCTCATATTGCATGAT |
| SEQ ID No: 886 | TGCACGCATGACTCATTGAT |
| SEQ ID No: 887 | TGCACTGATGGTCATCAGAT |
| SEQ ID No: 888 | TGCACTCTGCATTACATGAT |
| SEQ ID No: 889 | TGCACACACATGCGGCAGAT |
| SEQ ID No: 890 | TGCACGATTGTGATGATGAT |
| SEQ ID No: 891 | TGCACAGATGCATCTAAGAT |
| SEQ ID No: 892 | TGCACAGCAGATCTTGAGAT |
| SEQ ID No: 893 | TGCACTCTGCATGATGATTGAT |
| SEQ ID No: 894 | TGCACGTGCGGATGCGAT |
| SEQ ID No: 895 | TGCACGCTCTCAATGATGAT |
| SEQ ID No: 896 | TGCACTGATCTCATTGTGAT |
| SEQ ID No: 897 | TGCACATGTGGCAGATCATGAT |
| SEQ ID No: 898 | TGCACAAGCTCAGATCTGAT |
| SEQ ID No: 899 | TGCACTGCTGCACATGATTGAT |
| SEQ ID No: 900 | TGCACATGGCATGTGTCGAT |
| SEQ ID No: 901 | TGCACAATGTGCTGATAGAT |
| SEQ ID No: 902 | TGCACATAGCAGCGCTTGAT |
| SEQ ID No: 903 | TGCACATCGTGCATGAAGAT |
| SEQ ID No: 904 | TGCACGGATAGCAGTGAT |
| SEQ ID No: 905 | TGCACTCAATGCACAGTGAT |
| SEQ ID No: 906 | TGCACTCATGTCTGCAAGAT |
| SEQ ID No: 907 | TGCACATCGCATCTTCTGAT |
| SEQ ID No: 908 | TGCACATCCACTCATGTGAT |
| SEQ ID No: 909 | TGCACTAGCATAGCATTGAT |
| SEQ ID No: 910 | TGCACTTATCGCACAGAT |
| SEQ ID No: 911 | TGCACAGCGCCGCGAGAT |
| SEQ ID No: 912 | TGCACAGTCACATCCATGAT |
| SEQ ID No: 913 | TGCACATACATCAGCTTGAT |
| SEQ ID No: 914 | TGCACAACATGTCGTGAT |
| SEQ ID No: 915 | TGCACATGTCTGTGCTTGAT |
| SEQ ID No: 916 | TGCACACTCATGAGCTTGAT |
| SEQ ID No: 917 | TGCACTCGGATGCGCGAT |
| SEQ ID No: 918 | TGCACATGTATAATGCTGAT |
| SEQ ID No: 919 | TGCACAGCTCCATATGTGAT |
| SEQ ID No: 920 | TGCACTTATGATCAGCTGAT |
| SEQ ID No: 921 | TGCACATATGGCATGCAGCGAT |
| SEQ ID No: 922 | TGCACAGAGAATCATCAGAT |
| SEQ ID No: 923 | TGCACAATGCTAGACATGAT |
| SEQ ID No: 924 | TGCACAATCTGTATCATGAT |
| SEQ ID No: 925 | TGCACAGATATGATTGTGAT |
| SEQ ID No: 926 | TGCACAGATGACCATGCATGAT |
| SEQ ID No: 927 | TGCACATCGCGAATGATGAT |
| SEQ ID No: 928 | TGCACAGACTGCCACGAT |
| SEQ ID No: 929 | TGCACGTCTCATTGCGAT |
| SEQ ID No: 930 | TGCACAGACAATACTGAT |
| SEQ ID No: 931 | TGCACAGCTGTCTAAGAT |
| SEQ ID No: 932 | TGCACGATTCTGACAGAT |
| SEQ ID No: 933 | TGCACATGCTGCCAGCTGCGAT |
| SEQ ID No: 934 | TGCACATGCATCACTGGATGAT |
| SEQ ID No: 935 | TGCACAGTGACTGAAGAT |
| SEQ ID No: 936 | TGCACATGATATGCCATGCGAT |
| SEQ ID No: 937 | TGCACGGCAGCATCTCAGAT |
| SEQ ID No: 938 | TGCACGCACTGCCTAGAT |
| SEQ ID No: 939 | TGCACTCGAGCTGCATTGAT |
| SEQ ID No: 940 | TGCACAGCTATATCATTGAT |
| SEQ ID No: 941 | TGCACTTGCGCGTGCATGAT |
| SEQ ID No: 942 | TGCACTATGAGATGATTGAT |
| SEQ ID No: 943 | TGCACATATGTATGCAAGAT |
| SEQ ID No: 944 | TGCACATGCAGACTTCTGAT |
| SEQ ID No: 945 | TGCACATGTGCGCAATGCAGAT |
| SEQ ID No: 946 | TGCACATCTCATGCAGCAAGAT |
| SEQ ID No: 947 | TGCACGTCTGCTGCCATGAT |
| SEQ ID No: 948 | TGCACATATAAGTGCGAT |
| SEQ ID No: 949 | TGCACTGTCAGTTGCATGAT |
| SEQ ID No: 950 | TGCACTTCTATCTGCGAT |
| SEQ ID No: 951 | TGCACATACTATATTGAT |
| SEQ ID No: 952 | TGCACTAGGCTGATCATGAT |
| SEQ ID No: 953 | TGCACAGCTCTCCTCGAT |
| SEQ ID No: 954 | TGCACTGCGCCGCATCAGAT |
| SEQ ID No: 955 | TGCACTGCGATGCTCAAGAT |
| SEQ ID No: 956 | TGCACGACCGCATCAGAT |
| SEQ ID No: 957 | TGCACTCAAGAGCTGAGAT |
| SEQ ID No: 958 | TGCACGATATGTTGCGAT |
| SEQ ID No: 959 | TGCACTCACAACGATGAT |
| SEQ ID No: 960 | TGCACGATGCCAGCATGCTGAT |
| SEQ ID No: 961 | TGCACTGCTCCATGTATGAT |
| SEQ ID No: 962 | TGCACATCTATGCATGGCTGAT |
| SEQ ID No: 963 | TGCACTGAGAAGCGCGAT |
| SEQ ID No: 964 | TGCACACTGAGCCAGCAGAT |
| SEQ ID No: 965 | TGCACTGAGAGCCTCATGAT |
| SEQ ID No: 966 | TGCACAACTGCGAGTGAT |
| SEQ ID No: 967 | TGCACTCTGAGCATGAAGAT |
| SEQ ID No: 968 | TGCACTCAGTGCTCCATGAT |
| SEQ ID No: 969 | TGCACAGAACAGCATGCGAT |
| SEQ ID No: 970 | TGCACGACAGATGCCATGAT |
| SEQ ID No: 971 | TGCACATATGCATAAGTGAT |
| SEQ ID No: 972 | TGCACTGCGCTCCATGTGAT |
| SEQ ID No: 973 | TGCACATGCATCTCCTGCAGAT |
| SEQ ID No: 974 | TGCACTCATGATCGGATGAT |
| SEQ ID No: 975 | TGCACGATCACAATCGAT |
| SEQ ID No: 976 | TGCACGCATGCGTGGCTGAT |
| SEQ ID No: 977 | TGCACGGATGTCAGCATGAT |
| SEQ ID No: 978 | TGCACTCACGGCGCTGAT |
| SEQ ID No: 979 | TGCACTGATCTCCTCAGAT |
| SEQ ID No: 980 | TGCACAGTCACGGCTGAT |
| SEQ ID No: 981 | TGCACAGCCGTGCATCAGAT |
| SEQ ID No: 982 | TGCACATACACAATAGAT |
| SEQ ID No: 983 | TGCACTCTCGCGATTGAT |
| SEQ ID No: 984 | TGCACTTGCAGTGCACAGAT |
| SEQ ID No: 985 | TGCACTCTTGTGACAGAT |
| SEQ ID No: 986 | TGCACATGCATGCGGCTCAGAT |
| SEQ ID No: 987 | TGCACGTGAGCGCTTGAT |
| SEQ ID No: 988 | TGCACATCATTGCAGTGCTGAT |
| SEQ ID No: 989 | TGCACGATACACCATGAT |
| SEQ ID No: 990 | TGCACGCTATTCAGAGAT |
| SEQ ID No: 991 | TGCACTGCATGTCGGCTGAT |
| SEQ ID No: 992 | TGCACAGAAGTGCGAGAT |
| SEQ ID No: 993 | TGCACTGTTGAGCACATGAT |
| SEQ ID No: 994 | TGCACTGATATGCGCAAGAT |
| SEQ ID No: 995 | TGCACAGACTAGCAAGAT |
| SEQ ID No: 996 | TGCACGCAAGAGCATATGAT |
| SEQ ID No: 997 | TGCACATCGTGATGGCTGAT |
| SEQ ID No: 998 | TGCACTGAGCCTCAGCTGAT |
| SEQ ID No: 999 | TGCACACAGCGAAGAGAT |
| SEQ ID No: 1000 | TGCACTGAAGCTCTCATGAT |
| SEQ ID No: 1001 | TGCACACATCATCAACTGAT |
| SEQ ID No: 1002 | TGCACAGAATAGTCAGAT |
| SEQ ID No: 1003 | TGCACTGTCATCTCATTGAT |
| SEQ ID No: 1004 | TGCACATCGCCATGCATGCGAT |
| SEQ ID No: 1005 | TGCACACAGCATGCATTCAGAT |
| SEQ ID No: 1006 | TGCACATGCGATTGATAGAT |
| SEQ ID No: 1007 | TGCACGAGTCATTGCGAT |
| SEQ ID No: 1008 | TGCACTCAGATCCATCAGAT |
| SEQ ID No: 1009 | TGCACATAATACATCGAT |
| SEQ ID No: 1010 | TGCACAAGCACTATGCTGAT |
| SEQ ID No: 1011 | TGCACAACTCGCACAGAT |
| SEQ ID No: 1012 | TGCACAACACTCATCGAT |
| SEQ ID No: 1013 | TGCACATGCTATTACGAT |
| SEQ ID No: 1014 | TGCACTGCATTCAGCATGTGAT |
| SEQ ID No: 1015 | TGCACATACAGCACCGAT |
| SEQ ID No: 1016 | TGCACTCTGAGTTGTGAT |
| SEQ ID No: 1017 | TGCACGCGCTCTCTTGAT |
| SEQ ID No: 1018 | TGCACGATCAGAGCCGAT |
| SEQ ID No: 1019 | TGCACATAGAAGATAGAT |
| SEQ ID No: 1020 | TGCACTCATCTCGCCGAT |
| SEQ ID No: 1021 | TGCACTTGACATCGCATGAT |
| SEQ ID No: 1022 | TGCACAGCCTGAGATGCGAT |
| SEQ ID No: 1023 | TGCACTGTATCAATCGAT |
| SEQ ID No: 1024 | TGCACATATCTCATGAAGAT |
| SEQ ID No: 1025 | TGCACACATAGCCTGCAGAT |
| SEQ ID No: 1026 | TGCACTCGCTTGCATCTGAT |
| SEQ ID No: 1027 | TGCACATGCGGCACAGTGAT |
| SEQ ID No: 1028 | TGCACTGCTGATTAGCTGAT |
| SEQ ID No: 1029 | TGCACATGCACTGAAGCGAT |
| SEQ ID No: 1030 | TGCACACTGTCTTGCATGAT |
| SEQ ID No: 1031 | TGCACACTTATGCGCATGAT |
| SEQ ID No: 1032 | TGCACACAACAGCAGCTGAT |
| SEQ ID No: 1033 | TGCACATGCTCATGGTCGAT |
| SEQ ID No: 1034 | TGCACATCCACATGCATATGAT |
| SEQ ID No: 1035 | TGCACATGGCTCTGCACGAT |
| SEQ ID No: 1036 | TGCACTTGATGCACTCTGAT |
| SEQ ID No: 1037 | TGCACATCCTCTGCAGTGAT |
| SEQ ID No: 1038 | TGCACAAGATCGATCGAT |
| SEQ ID No: 1039 | TGCACTGCCATGATGTAGAT |
| SEQ ID No: 1040 | TGCACACTGCCTCATCAGAT |
| SEQ ID No: 1041 | TGCACATCATACATATTGAT |
| SEQ ID No: 1042 | TGCACATATAGAATCATGAT |
| SEQ ID No: 1043 | TGCACGCTTGCTCTGCAGAT |
| SEQ ID No: 1044 | TGCACAATGCTCTCTGTGAT |
| SEQ ID No: 1045 | TGCACTAGTCCATGCATGAT |
| SEQ ID No: 1046 | TGCACAATCATGCTATAGAT |
| SEQ ID No: 1047 | TGCACATGCGCAACATGCAGAT |
| SEQ ID No: 1048 | TGCACTCATATGGCAGTGAT |
| SEQ ID No: 1049 | TGCACAGCACATTATATGAT |
| SEQ ID No: 1050 | TGCACATCTGCACTGAAGAT |
| SEQ ID No: 1051 | TGCACATCGCCAGCACTGAT |
| SEQ ID No: 1052 | TGCACAGCCTCAGCTGCATGAT |
| SEQ ID No: 1053 | TGCACGCGGCACAGAGAT |
| SEQ ID No: 1054 | TGCACAGACTGCATTGTGAT |
| SEQ ID No: 1055 | TGCACAATCTGCATGATCAGAT |
| SEQ ID No: 1056 | TGCACAATGCAGCGCTGCTGAT |
| SEQ ID No: 1057 | TGCACAGTCATGCTTCTGAT |
| SEQ ID No: 1058 | TGCACTCAGTGAATGATGAT |
| SEQ ID No: 1059 | TGCACAGCATGATCAGGCTGAT |
| SEQ ID No: 1060 | TGCACATACTCTGCATTGAT |
| SEQ ID No: 1061 | TGCACAGCCGCGATGCAGAT |
| SEQ ID No: 1062 | TGCACATGGTGCACGATGAT |
| SEQ ID No: 1063 | TGCACTTCACTGCTCGAT |
| SEQ ID No: 1064 | TGCACAGCACATCTTCTGAT |
| SEQ ID No: 1065 | TGCACACAGTGAATCATGAT |
| SEQ ID No: 1066 | TGCACTGTTATCGCTGAT |
| SEQ ID No: 1067 | TGCACTAGATGTGCCATGAT |
| SEQ ID No: 1068 | TGCACGGCTATATGCGAT |
| SEQ ID No: 1069 | TGCACGTGCGCAACTGAT |
| SEQ ID No: 1070 | TGCACATGTGCTCTCAAGAT |
| SEQ ID No: 1071 | TGCACAAGCGCAGCTCTGAT |
| SEQ ID No: 1072 | TGCACTCTATATTCTGAT |
| SEQ ID No: 1073 | TGCACTTGAGCTGCGAGAT |
| SEQ ID No: 1074 | TGCACACATGGCTAGCAGAT |
| SEQ ID No: 1075 | TGCACGTGAGATTGTGAT |
| SEQ ID No: 1076 | TGCACTAGCTTGCTGCTGAT |
| SEQ ID No: 1077 | TGCACTGATGCAATCTGCAGAT |
| SEQ ID No: 1078 | TGCACATGCATAATGATATGAT |
| SEQ ID No: 1079 | TGCACAGTGCCTGACGAT |
| SEQ ID No: 1080 | TGCACATAGAGCATTCTGAT |
| SEQ ID No: 1081 | TGCACATGATCTGCGAAGAT |
| SEQ ID No: 1082 | TGCACATCTGATTCTGTGAT |
| SEQ ID No: 1083 | TGCACATGCAAGCTGATATGAT |
| SEQ ID No: 1084 | TGCACACAGCATTGACTGAT |
| SEQ ID No: 1085 | TGCACAGTCAATCGAGAT |
| SEQ ID No: 1086 | TGCACTGATGGCATATAGAT |
| SEQ ID No: 1087 | TGCACACTCGCTGAAGAT |
| SEQ ID No: 1088 | TGCACTGAAGAGATGCAGAT |
| SEQ ID No: 1089 | TGCACGAGGATCTGCATGAT |
| SEQ ID No: 1090 | TGCACAACATCTGCTCAGAT |
| SEQ ID No: 1091 | TGCACGCTGAATGCATGCTGAT |
| SEQ ID No: 1092 | TGCACATCTCCACATCAGAT |
| SEQ ID No: 1093 | TGCACATGAGATGATCATTGAT |
| SEQ ID No: 1094 | TGCACTGCTCTGGCTGAGAT |
| SEQ ID No: 1095 | TGCACATAATGTGTGATGAT |
| SEQ ID No: 1096 | TGCACTCATGTATCCGAT |
| SEQ ID No: 1097 | TGCACGCTGTGCGTTGAT |
| SEQ ID No: 1098 | TGCACGGCATCACGTGAT |
| SEQ ID No: 1099 | TGCACTGACATATGATTGAT |
| SEQ ID No: 1100 | TGCACAAGTGCATATCTGAT |
| SEQ ID No: 1101 | TGCACAGCGCATTGTGAGAT |
| SEQ ID No: 1102 | TGCACGTCCAGATCAGAT |
| SEQ ID No: 1103 | TGCACATGCGGTGCGATGAT |
| SEQ ID No: 1104 | TGCACAATATATAGCGAT |
| SEQ ID No: 1105 | TGCACAAGTGCTAGTGAT |
| SEQ ID No: 1106 | TGCACTGTGAATAGAGAT |
| SEQ ID No: 1107 | TGCACGATTGATGCACAGAT |
| SEQ ID No: 1108 | TGCACTTGTGCGACAGAT |
| SEQ ID No: 1109 | TGCACAACTGTGACTGAT |
| SEQ ID No: 1110 | TGCACTAGCGCTTATGAT |
| SEQ ID No: 1111 | TGCACAGATATCCTCGAT |
| SEQ ID No: 1112 | TGCACGTGGCTGAGCATGAT |
| SEQ ID No: 1113 | TGCACTGATGCATGGTGCTGAT |
| SEQ ID No: 1114 | TGCACACTTGCATGCGCGAT |
| SEQ ID No: 1115 | TGCACAGCCATGCGACAGAT |
| SEQ ID No: 1116 | TGCACTCTTCTCTCTGAT |
| SEQ ID No: 1117 | TGCACATGGCGTATGATGAT |
| SEQ ID No: 1118 | TGCACTATTCTCAGCATGAT |
| SEQ ID No: 1119 | TGCACAGTTCTATGCATGAT |
| SEQ ID No: 1120 | TGCACAGCATCATGCTTGTGAT |
| SEQ ID No: 1121 | TGCACGTAATGCTGTGAT |
| SEQ ID No: 1122 | TGCACGCAATAGATCATGAT |
| SEQ ID No: 1123 | TGCACTTCTGCATGCTGCAGAT |
| SEQ ID No: 1124 | TGCACAGCACATGTGCCGAT |
| SEQ ID No: 1125 | TGCACAGCGATAACTGAT |
| SEQ ID No: 1126 | TGCACAGTTGTGTGCGAT |
| SEQ ID No: 1127 | TGCACACATGAGGCGCTGAT |
| SEQ ID No: 1128 | TGCACATGCGCTATTCTGAT |
| SEQ ID No: 1129 | TGCACGAGCAATGCGATGAT |
| SEQ ID No: 1130 | TGCACTGCCATGCTCTAGAT |
| SEQ ID No: 1131 | TGCACAAGCATGCGTATGAT |
| SEQ ID No: 1132 | TGCACAGAAGCTCATCTGAT |
| SEQ ID No: 1133 | TGCACTACAGCTGCATTGAT |
| SEQ ID No: 1134 | TGCACATCGCATTAGCTGAT |
| SEQ ID No: 1135 | TGCACAACATATCGCGAT |
| SEQ ID No: 1136 | TGCACGTATGGCATAGAT |
| SEQ ID No: 1137 | TGCACGATCTTGCATGAGAT |
| SEQ ID No: 1138 | TGCACTCTTGCGATCATGAT |
| SEQ ID No: 1139 | TGCACTCTGTCATGATTGAT |
| SEQ ID No: 1140 | TGCACAGCTGTAATATGAT |
| SEQ ID No: 1141 | TGCACAGTGCTGGCTGAGAT |
| SEQ ID No: 1142 | TGCACGCTGCCTCAGAGAT |
| SEQ ID No: 1143 | TGCACGAGATTGATGCTGAT |
| SEQ ID No: 1144 | TGCACATAATATATAGAT |
| SEQ ID No: 1145 | TGCACAGTAGATTGCATGAT |
| SEQ ID No: 1146 | TGCACTCAGCACCTGCTGAT |
| SEQ ID No: 1147 | TGCACATGTCTAAGCGAT |
| SEQ ID No: 1148 | TGCACTGTGATGGCTCTGAT |
| SEQ ID No: 1149 | TGCACGGTAGCATGCGAT |
| SEQ ID No: 1150 | TGCACTGCATGCGTTGAGAT |
| SEQ ID No: 1151 | TGCACTCATGCTGTCAAGAT |
| SEQ ID No: 1152 | TGCACACGGTGCTGTGAT |
| SEQ ID No: 1153 | TGCACAAGCATGCATGAGAGAT |
| SEQ ID No: 1154 | TGCACACAGAAGTCTGAT |
| SEQ ID No: 1155 | TGCACTTAGATGACAGAT |
| SEQ ID No: 1156 | TGCACATACAACTGTGAT |
| SEQ ID No: 1157 | TGCACTTGAGAGAGTGAT |
| SEQ ID No: 1158 | TGCACATCTACTTGCATGAT |
| SEQ ID No: 1159 | TGCACTTGCATGCTACAGAT |
| SEQ ID No: 1160 | TGCACTCAGCAGCGGCAGAT |
| SEQ ID No: 1161 | TGCACTGTACATTGTGAT |
| SEQ ID No: 1162 | TGCACTTGTGCATACGAT |
| SEQ ID No: 1163 | TGCACAACGTGAGCAGAT |
| SEQ ID No: 1164 | TGCACACATGCACGGCAGAT |
| SEQ ID No: 1165 | TGCACAAGCGTGTGCATGAT |
| SEQ ID No: 1166 | TGCACATGCACTTATGAGAT |
| SEQ ID No: 1167 | TGCACGCGACATTGCATGAT |
| SEQ ID No: 1168 | TGCACGGATGATGCTGAGAT |
| SEQ ID No: 1169 | TGCACTGATGAGCAATCGAT |
| SEQ ID No: 1170 | TGCACACGCTACATTGAT |
| SEQ ID No: 1171 | TGCACGCAATGCTGATCATGAT |
| SEQ ID No: 1172 | TGCACGCGCATAATGATGAT |
| SEQ ID No: 1173 | TGCACACACGCATAAGAT |
| SEQ ID No: 1174 | TGCACAGATGGTGCATGCAGAT |
| SEQ ID No: 1175 | TGCACGCACATGGATCTGAT |
| SEQ ID No: 1176 | TGCACATGCAACGCAGTGAT |
| SEQ ID No: 1177 | TGCACAGCAGGAGATCAGAT |
| SEQ ID No: 1178 | TGCACACATCAGGTGATGAT |
| SEQ ID No: 1179 | TGCACGCTCAATGAGCAGAT |
| SEQ ID No: 1180 | TGCACTGTGCAGTCCGAT |
| SEQ ID No: 1181 | TGCACTGATGCGCAGAAGAT |
| SEQ ID No: 1182 | TGCACAATATGCATGTCGAT |
| SEQ ID No: 1183 | TGCACAGATGAGTGGAGAT |
| SEQ ID No: 1184 | TGCACATGGAGATGCATGTGAT |
| SEQ ID No: 1185 | TGCACATCATTCGATGTGAT |
| SEQ ID No: 1186 | TGCACGCTCGGCAGCGAT |
| SEQ ID No: 1187 | TGCACGCTAGCTTGAGAT |
| SEQ ID No: 1188 | TGCACACGCAATCTAGAT |
| SEQ ID No: 1189 | TGCACACGCAACTGTGAT |
| SEQ ID No: 1190 | TGCACACGCACAATCATGAT |
| SEQ ID No: 1191 | TGCACGCATCTGCAATGCTGAT |
| SEQ ID No: 1192 | TGCACGCTCAGATGATTGAT |
| SEQ ID No: 1193 | TGCACGCATATGCATGGATGAT |
| SEQ ID No: 1194 | TGCACGCATGCATGGCATAGAT |
| SEQ ID No: 1195 | TGCACGCATCCAGCACTGAT |
| SEQ ID No: 1196 | TGCACGGAGCTCACAGAT |
| SEQ ID No: 1197 | TGCACGCTCTGCATTCAGAT |
| SEQ ID No: 1198 | TGCACATAGCAGAGGATGAT |
| SEQ ID No: 1199 | TGCACTGCAGGCGCATGATGAT |
| SEQ ID No: 1200 | TGCACGCATATCTGGCTGAT |
| SEQ ID No: 1201 | TGCACTGCTGTGCATCCGAT |
| SEQ ID No: 1202 | TGCACATGGCAGTCATAGAT |
| SEQ ID No: 1203 | TGCACTGCATGAATGCTGTGAT |
| SEQ ID No: 1204 | TGCACGCGCATGTCCATGAT |
| SEQ ID No: 1205 | TGCACGCACACATCCATGAT |
| SEQ ID No: 1206 | TGCACTGCAGGAGTGATGAT |
| SEQ ID No: 1207 | TGCACGCGTGGCAGAGAT |
| SEQ ID No: 1208 | TGCACGCATGGCATCACATGAT |
| SEQ ID No: 1209 | TGCACTAGCAATGATGAGAT |
| SEQ ID No: 1210 | TGCACTGCATTAGCATGCAGAT |
| SEQ ID No: 1211 | TGCACTATGCAGTCATTGAT |
| SEQ ID No: 1212 | TGCACACTGCAGTCATTGAT |
| SEQ ID No: 1213 | TGCACATCAGTGCAATCGAT |
| SEQ ID No: 1214 | TGCACGCAAGCTGAGAGAT |
| SEQ ID No: 1215 | TGCACGGCGAGATGAGAT |
| SEQ ID No: 1216 | TGCACATGCAGTTGATGCAGAT |
| SEQ ID No: 1217 | TGCACAGATGAGACATTGAT |
| SEQ ID No: 1218 | TGCACGCTTGATGATCAGAT |
| SEQ ID No: 1219 | TGCACGGCGCATGCAGTGAT |
| SEQ ID No: 1220 | TGCACTGAGACATGCTTGAT |
| SEQ ID No: 1221 | TGCACGCATCTGGCGATGAT |
| SEQ ID No: 1222 | TGCACAGCATGCAGTCCGAT |
| SEQ ID No: 1223 | TGCACGGATGCAGTGATGAT |
| SEQ ID No: 1224 | TGCACGATTGCAGCGCAGAT |
| SEQ ID No: 1225 | TGCACGTGAGGTGCAGAT |
| SEQ ID No: 1226 | TGCACGCACAAGCATGAGAT |
| SEQ ID No: 1227 | TGCACATCAGGCGCATGCAGAT |
| SEQ ID No: 1228 | TGCACAGCATTCTGTGCATGAT |
| SEQ ID No: 1229 | TGCACTAGCGGTGCAGAT |
| SEQ ID No: 1230 | TGCACGCATGATTAGATGAT |
| SEQ ID No: 1231 | TGCACGCATGCAATGTGCAGAT |
| SEQ ID No: 1232 | TGCACGCAGCACATTGCGAT |
| SEQ ID No: 1233 | TGCACGCAGCACCTGATGAT |
| SEQ ID No: 1234 | TGCACGCAGCCTGCGCTGAT |
| SEQ ID No: 1235 | TGCACGCATGGAGCTGCGAT |
| SEQ ID No: 1236 | TGCACGCGGATGCTGATGAT |
| SEQ ID No: 1237 | TGCACGTGCGCATGGATGAT |
| SEQ ID No: 1238 | TGCACGGTGCATGCAGAGAT |
| SEQ ID No: 1239 | TGCACGCACTTGATGATGAT |
| SEQ ID No: 1240 | TGCACTTCTCACGCAGAT |
| SEQ ID No: 1241 | TGCACGCAATGAGCGATGAT |
| SEQ ID No: 1242 | TGCACATCATGAATGATGTGAT |
| SEQ ID No: 1243 | TGCACGCAGCAGAGATTGAT |
| SEQ ID No: 1244 | TGCACGTGGATGAGCGAT |
| SEQ ID No: 1245 | TGCACGCTTGCAGCATGCAGAT |
| SEQ ID No: 1246 | TGCACGCAGATCATTCTGAT |
| SEQ ID No: 1247 | TGCACGCAGAATCTGCGAT |
| SEQ ID No: 1248 | TGCACGCAGATCCAGCAGAT |
| SEQ ID No: 1249 | TGCACATGGATGAGATGCTGAT |
| SEQ ID No: 1250 | TGCACGCAGTGCTGCAAGAT |
| SEQ ID No: 1251 | TGCACGCAGTGAGCCATGAT |
| SEQ ID No: 1252 | TGCACACGGATCATGCAGAT |
| SEQ ID No: 1253 | TGCACGGCTCGTGCAGAT |
| SEQ ID No: 1254 | TGCACACGCATGGAGATGAT |
| SEQ ID No: 1255 | TGCACACACGCGGATGAT |
| SEQ ID No: 1256 | TGCACATCGAATGTGCAGAT |
| SEQ ID No: 1257 | TGCACACGCAGACAAGAT |
| SEQ ID No: 1258 | TGCACACACATGGATGAGAT |
| SEQ ID No: 1259 | TGCACATGATTCATGTGCAGAT |

In some cases, 3′ T in a barcode from Table 3 may be phosphorylated. In some cases, a barcode in Table 3 may be concatenated into an adapter with ATCTCATCCCTGCGTGTCTCCGAC (SEQ ID No. 1260), where SEQ ID No. 1260 is positioned 5′ to the respective barcode sequence. For example, a complete adapter sequence comprising SEQ ID No: 1237 barcode from Table 3 may comprise:

(SEQ ID NO. 1261)
ATCTCATCCCTGCGTGTCTCCGACTGCACGTGCGCATGGATGA*T.

### KM Barcodes

As explained above, there are significant advantages to using flow invariant barcode sequences. An alternative method of providing a large set of barcodes that are suitable for flow-based sequencing is described herein. Barcodes designed in this way may be used in combination with adapter sequences described herein, or with any other suitable adapter sequences.

To produce a set of barcodes that are all distinguishable within a same number of flows, it is effective to design within the parameters of a particular flow cycle and use homopolymers, as described with respect to Table 3 barcodes. Alternatively, a set of barcodes may be produced, where the sequences comprise alternating nucleotide base types in accordance with a flow order or a general pattern of flow order. Notably, barcode sets described herein that comprise barcodes all of a same length, may be used for any type of sequencing (e.g., for flow sequencing, sequencing by synthesis, sequencing by binding, etc.). In some cases, a barcode set may be produced and output electronically, e.g., using one or more computer systems as described elsewhere herein).

In some cases, barcode sequences can be constructed by selecting a nucleotide base type alternatively from (1) a first set of nucleotide base types (K) and (2) a second set of nucleotide base types (M) for N consecutive base positions. Base types in the first set of nucleotide base types (K) and the second set of nucleotide base types (M) are mutually exclusive. In some cases, within the barcode sequence, any base type of the first set of nucleotide base types is only adjacent to any base type of the second set of nucleotide base types (e.g., multiple base types from the first set of bases will never be adjacent to each other in the barcode sequence).

Hewing directly to a preselected flow order, in some cases, a barcode sequence may be constructed by selecting alternately, for each base position in a plurality of base positions, a nucleotide base type from a first set of nucleotide base types or from a second set of nucleotide base types. In some cases, the first set of nucleotide base types may comprise a first nucleotide base type and a second nucleotide base type from a first portion of a flow order (e.g., a predetermined flow order), and the second set of nucleotide base types comprises a third nucleotide base type and a fourth nucleotide base type from a second portion of the flow order, wherein the flow order comprises an ordered set of the four canonical base types (A, T, C, and G).

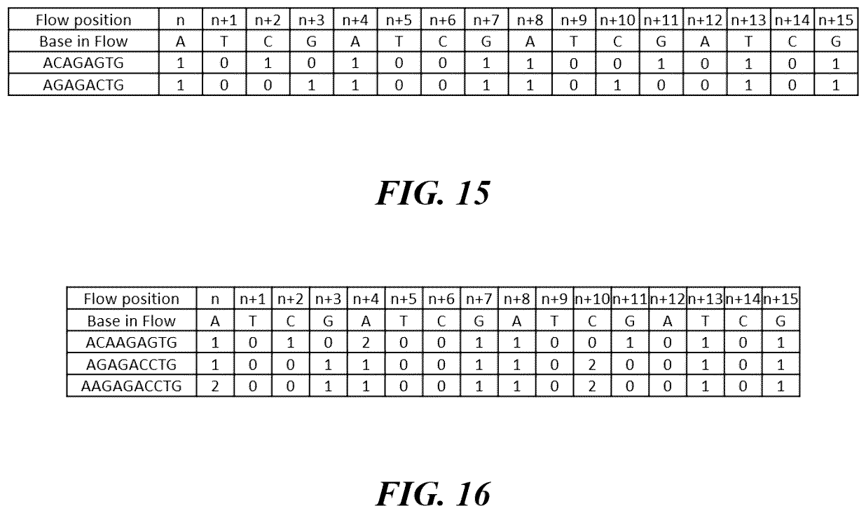

By way of example, if the flow order is A-T-C-G, the first set of base types would be (A, T) and the second set of base types would be (C, G). That is, a barcode sequence would comprise an alternating selection of (A, T) and (C, G). An example barcode sequence suitable for this flow order that is 8 nucleotides longis ACAGAGTG. FIG. 15 illustrates flowgrams for several example barcode sequences selected in accordance with these criteria. During sequencing, each barcode will provide a signal once in every set of two flows (e.g., one signal during each set of A and T flows and one signal during each set of C and G flows).

In some cases a flow order may be any combination of the four canonical base types. In some cases a flow order may be an extended sequencing flow order and may comprise any set of the four canonical base types including duplicates of any one or more base types. One example of an extended flow order is T-C-A-G-A-T-G-C-A-T-G-C-T-A-C-G, comprising 16 flows, where each base type is included four times—that is each base type is included in each four subsets of four flows-and no subset of four base types is duplicated. In such an extended flow order, a barcode sequence may be constructed by selecting alternately, for each base position. Different flow orders are described in U.S. Pat. No. 11,763,915B2, which is incorporated in its entirety by reference for all purposes. It will be understood that any desired flow order may be used and that barcodes may be provided for any flow order.

In some cases, the first set of nucleotide base types comprises a first nucleotide base type and a second nucleotide base type. In some cases, the second set of nucleotide base types comprises a third nucleotide base type and a fourth nucleotide base type. In some cases, the first set of nucleotide base types comprises thymidine and guanine. In some cases, the second set of nucleotide base types comprises cytidine and adenine. Base types in the first set of nucleotide base types (K) and the second set of nucleotide base types (M) are mutually exclusive. By IUPAC conventions, K corresponds to guanine (G) or thymine (T), and M corresponds to adenine (A) and cytosine (C). However, the first set and second of base types may each comprise any two base types, as long as the first and second sets are distinct from each other. For example, the first set of base types may be A and T.

A set of barcodes may be produced in accordance with the described selection criteria by repeating the selection of bases alternatively from the first and second sets of base types to construct a plurality of barcode sequences. In any barcode set, each respective barcode sequence will be distinct from all other barcode sequences in the set.

In some cases, the number of base positions N is a multiple of the length of the flow order (e.g., the length of an extended flow order or a non-extended flow order). In some cases, the number of base positions N may be any suitable number Preferentially, N may be any number from 3 to 30. In some cases, N may be an even number, e.g., 2, 4, 8, 20, etc. In some cases, N is at least 10.

In some cases, each base position in a respective barcode sequence comprises a single nucleotide of the selected nucleotide base type. In such cases, each barcode sequence in a set will be a same length (e.g., all will be N bases in length).

In some cases it may be advantageous to enhance the signal differences between barcodes. One way to do this is to permit multiples of a base type at each base position. This will result in analog signals that are greater than 1 at one or more base positions in a barcode. This may help with uniquely differentiating barcodes during sequence analysis.

For example, a barcode sequence of length X may be constructed by selecting one or more nucleotides of a nucleotide base type alternatively from (1) a first set of nucleotide base types (K) and (2) a second set of nucleotide base types (M) for N consecutive times, where base types in the first set of nucleotide base types (K) and the second set of nucleotide base types (M) are mutually exclusive. In some cases, X is greater than or equal to N; that is the total length of the barcode may be longer than the number of base types selected. FIG. 16 illustrates flowgrams for several example barcode sequences selected in accordance with these criteria.

In some cases, only one base position in each barcode sequence may comprise more than one nucleotide base (e.g., there may be just one homopolymer greater than one in each barcode). In some cases, there may be at least one homopolymer greater than one each barcode. In some cases, any homopolymers may be 2, 3, 4, 5, or any number of bases. In some cases, any homopolymers in a set may all be a same number (e.g., in one set of barcodes each will have one homopolymer of 2).

In some cases, the set of barcode sequences comprises 2\′ barcode sequences. In some cases, the set of barcode sequences comprises at least 96 barcode sequences. In some cases, the set of barcode sequences comprises at least 256 barcode sequences. In some cases, the set of barcode sequences comprises a multiple of 8 barcode sequences.

### Computer Systems

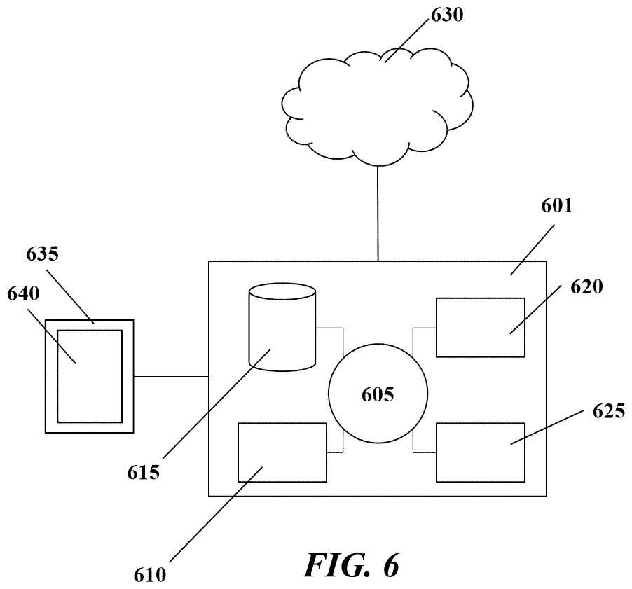

The present disclosure provides computer control systems that are programmed to implement methods of the disclosure. FIG. 6 shows a computer system 601 that is programmed or otherwise configured to implement methods of the disclosure, such as to control the systems described herein (e.g., reagent dispensing, detecting, etc.) and collect, receive, and/or analyze sequencing information. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an isolated or substantially isolated internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server. The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback. The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple®iPad, Samsung® Galaxy Tab), telephones, Smart phones (e.g., Apple® iphone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine-readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610. The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as the main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, results of a nucleic acid sequence (e.g., sequence reads). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, perform error correction on processed sequencing signals.

### NUMBERED EMBODIMENTS

Embodiment 1. A composition, comprising a non-naturally occurring nucleic acid molecule comprising a first strand comprising SEQ ID No: 1 and a second strand comprising SEQ ID No. 2.

Embodiment 2. The composition of embodiment 1, wherein the non-naturally occurring nucleic acid molecule is coupled to a template nucleic acid molecule.

Embodiment 3. The composition of embodiment 2, wherein the coupling is via ligation.

Embodiment 4. The composition of any one of embodiments 1-3, wherein the non-naturally occurring nucleic acid molecule further comprises a barcode sequence selected from any one of SEQ ID Nos: 205-1259.

Embodiment 5. The composition of embodiment 4, wherein the barcode sequence selected from any one of SEQ ID Nos: 205-1259 is disposed 3′ of SEQ ID No: 1, and a reverse complementary sequence of the selected barcode is disposed 5′ of SEQ ID No: 2.

Embodiment 6. The composition of embodiment 4 or embodiment 5, wherein the first strand further comprises GAT at 3′ end, and the second strand further comprises CT at 5′ end.

Embodiment 7. A kit comprising a plurality of non-naturally occurring nucleic acid molecules, each comprising a first strand comprising SEQ ID No: 1 and a second strand comprising SEQ ID No. 2.

Embodiment 8. The kit of embodiment 7, wherein each of the plurality of non-naturally occurring nucleic acid molecules further comprises a barcode sequence selected from any one of SEQ ID Nos: 205-1259.

Embodiment 9. The kit of embodiment 8, wherein the plurality of non-naturally occurring nucleic acid molecules comprises at least 96 subsets, wherein each subset of non-naturally occurring nucleic acid molecules comprises a different barcode sequence selected from any one of SEQ ID Nos: 205-1259.

Embodiment 10. A composition, comprising a non-naturally occurring nucleic acid molecule comprising a sequence selected from any one of SEQ ID Nos: 5-104.

Embodiment 11. The composition of embodiment 10, wherein the non-naturally occurring nucleic acid molecule is coupled to a support.

Embodiment 12. The composition of embodiment 11, wherein the support is a bead.

Embodiment 13. The composition of embodiment 11 or embodiment 12, wherein the non-naturally occurring nucleic acid molecule is further coupled to a template nucleic acid molecule.

Embodiment 14. The composition of embodiment 13, wherein the coupling comprises hybridization.

Embodiment 15. A kit, comprising at least two non-naturally occurring nucleic acid molecules each comprising a different sequence selected from any one of SEQ ID Nos: 5-104.

Embodiment 16. The kit of embodiment 15, wherein each non-naturally occurring nucleic acid molecule is coupled to a support.

Embodiment 17. The kit of embodiment 16, wherein the support is a bead.

Embodiment 18. A composition, comprising a non-naturally occurring nucleic acid molecule comprising a sequence selected from any one of SEQ ID Nos: 105-204.

Embodiment 19. The composition of embodiment 18, wherein the non-naturally occurring nucleic acid molecule is coupled to a support.

Embodiment 20. The composition of embodiment 19, wherein the support is a bead.

Embodiment 21. The composition of embodiment 19 or embodiment 20, wherein the non-naturally occurring nucleic acid molecule is further coupled to a template nucleic acid molecule.

Embodiment 22. The composition of embodiment 21, wherein the coupling comprises hybridization.

Embodiment 23. A kit, comprising at least two non-naturally occurring nucleic acid molecules each comprising a different sequence selected from any one of SEQ ID Nos: 105-204.

Embodiment 24. The kit of embodiment 23, wherein each non-naturally occurring nucleic acid molecule is coupled to a support.

Embodiment 25. The kit of embodiment 24, wherein the support is a bead.

Embodiment 26. A composition, comprising a non-naturally occurring nucleic acid molecule comprising a sequence selected from any one of SEQ ID Nos 205-1259.

Embodiment 27. The composition of embodiment 26, wherein 3′ T of the non-naturally occurring nucleic acid molecule is phosphorylated.

Embodiment 28. The composition of embodiment 26 or embodiment 27, wherein the non-naturally occurring nucleic acid molecule further comprises SEQ ID No. 1260 positioned 5′ to the selected sequence.

Embodiment 29. A kit, comprising at least one non-naturally occurring nucleic acid molecule comprising a sequence selected from any one of SEQ ID Nos: 205-1259.

Embodiment 30. The kit of embodiment 29, wherein the non-naturally occurring nucleic acid molecule further comprises SEQ ID No. 1260 positioned 5′ to the selected sequence.

Embodiment 31. The kit of embodiment 29 or embodiment 30, wherein 3′ T of the non-naturally occurring nucleic acid molecule is phosphorylated.

Embodiment 32. A kit, comprising at least 96 non-naturally occurring nucleic acid molecules each comprising a different sequence selected from any one of SEQ ID Nos: 205-1259.

Embodiment 33. The kit of embodiment 32, wherein each non-naturally occurring nucleic acid molecule further comprises SEQ ID No. 1260 positioned 5′ to the selected sequence.

Embodiment 34. The kit of embodiment 32 or embodiment 33, wherein 3′ T of each non-naturally occurring nucleic acid molecule is phosphorylated.

Embodiment 35. A kit, comprising at least 256 non-naturally occurring nucleic acid molecules each comprising a different sequence selected from any one of SEQ ID Nos: 205-1259.

Embodiment 36. The kit of embodiment 35, wherein each non-naturally occurring nucleic acid molecule further comprises SEQ ID No. 1260 positioned 5′ to the selected sequence.

Embodiment 37. The kit of embodiment 35 or embodiment 36, wherein 3′ T of each non-naturally occurring nucleic acid molecule is phosphorylated.

Embodiment 38. A method, comprising: (a) providing a plurality of template molecules and a first plurality of adapters, wherein adapters in the first plurality of adapters comprise a double-stranded region and a single-stranded region; (b) for each template molecule in the plurality of template molecules, coupling an adapter from the first plurality of adapters to each end of the respective template molecule; (c) providing a second plurality of adapters, wherein the second plurality of adapters each comprise a single strand; and (d) for each template molecule in the plurality of template molecules, coupling an adapter from the second plurality of adapters to the single-stranded regions of previously coupled adapters, wherein the resulting template-adapter molecules do not comprise identical adapters sequences.

Embodiment 39. The method of embodiment 38, wherein the single-stranded region of adapters in the first plurality of adapters comprises an overhang.

Embodiment 40. The method of embodiment 38 or embodiment 39, wherein the double-stranded region of adapters in the first plurality of adapters comprises a first strand and a second strand hybridized to each other.

Embodiment 41. The method of embodiment 40, wherein the first strand and the second strand are reverse complements of each other.

Embodiment 42. The method of embodiment 40, wherein the first strand and the second strand are not reverse complements of each other.

Embodiment 43. The method of embodiment 42, wherein there is at least a single base mismatch between the first strand and the second strand.

Embodiment 44. The method of any one of embodiments 38-43, wherein a first adapter and a second adapter in the first plurality of adapters comprise different sequences.

Embodiment 45. The method of embodiment 44, wherein there is at least a single base mismatch between the first adapter and the second adapter.

Embodiment 46. The method of embodiment 44, wherein there is no more than a single base mismatch between the first adapter and the second adapter.

Embodiment 47. The method of any one of embodiment 38-46, wherein the second plurality of adapters comprise at least a first subset of adapters and a second subset wherein the first and second subsets do not have identical sequences.

Embodiment 48. The method of embodiment 47, wherein there is at least a single base mismatch between adapters in the first subset and second subset.

Embodiment 49. The method of embodiment 47, wherein there is no more than a single base mismatch between adapters in the first subset and the second subset.

Embodiment 50. The method of embodiment 42 or embodiment 43, wherein adapters in the second plurality of adapters have identical sequences.

Embodiment 51. The method of any one of embodiments 38-50, wherein coupling in step (b) comprises ligating adapters in the first plurality of adapters to library molecules.

Embodiment 52. The method of any one of embodiments 38-51, wherein coupling in step (d) comprises (i) hybridizing a first region of adapters in the second plurality of adapters to at least a portion of the single-stranded region of an adapter in the first plurality of adapters, and (ii) ligating 3′ end of the first region to the double-stranded region of the adapter in the first plurality of adapters.

Embodiment 53. The method of any one of embodiments 38-52, wherein the coupling in step (b) and step (d) are preformed concurrently.

Embodiment 54. The method of any one of embodiments 38-52, wherein the coupling in step (b) and step (d) are preformed sequentially.

Embodiment 55. The method of any one of embodiments 38-54, further comprising amplifying the template-adapter molecules with a plurality of primers.

Embodiment 56. The method of embodiment 55, wherein primers in the plurality of primers have identical sequences.

Embodiment 57. The method of embodiment 55, wherein a first primer and a second primer in the plurality of primers have different sequences.

Embodiment 58. The method of embodiment 57, wherein there is at least a single base mismatch between the first primer and the second primer.

Embodiment 59. The method of embodiment 57, wherein there is no more than a single base mismatch between the first primer and the second primer.

Embodiment 60. A method for generating barcode sequences, comprising: (a) constructing a barcode sequence of N bases by selecting a nucleotide base type alternatively from (1) a first set of nucleotide base types (K) and (2) a second set of nucleotide base types (M) for N consecutive base positions, wherein base types in the first set of nucleotide base types (K) and the second set of nucleotide base types (M) are mutually exclusive; (b) repeating (a) to construct a plurality of barcode sequences, wherein each of the plurality of barcode sequences is N bases in length and is unique within the plurality of barcode sequences; and (c) electronically outputting the plurality of barcode sequences.

Embodiment 61. A method for generating barcode sequences, comprising: (a) constructing a barcode sequence of N bases by selecting a nucleotide base type from (1) a first set of nucleotide base types (K) and (2) a second set of nucleotide base types (M) for N consecutive base positions, wherein base types in the first set of nucleotide base types (K) and the second set of nucleotide base types (M) are mutually exclusive, wherein, within the barcode sequence, any base type of the first set of nucleotide base types is only adjacent to any base type of the second set of nucleotide base types; (b) repeating (a) to construct a plurality of barcode sequences, wherein each of the plurality of barcode sequences is N bases in length and is unique within the plurality of barcode sequences; and (c) electronically outputting the plurality of barcode sequences.

Embodiment 62. A method for generating barcode sequences, comprising: (a) constructing a barcode sequence of length X by selecting one or more nucleotides of a nucleotide base type alternatively from (1) a first set of nucleotide base types (K) and (2) a second set of nucleotide base types (M) for N consecutive times, wherein: i) base types in the first set of nucleotide base types (K) and the second set of nucleotide base types (M) are mutually exclusive, and ii) X>=N; (b) repeating (a) to construct a plurality of barcode sequences, wherein each of the plurality of barcode sequences is unique within the plurality of barcode sequences; and (c) electronically outputting the plurality of barcode sequences.

Embodiment 63. A method for generating a set of barcode sequences, comprising: (a) for each respective barcode sequence selecting alternately, for each base position in a plurality of base positions, a nucleotide base type from a first set of nucleotide base types or from a second set of nucleotide base types, wherein: i) the first set of nucleotide base types comprises a first nucleotide base type and a second nucleotide base type from a first portion of a flow order, and the second set of nucleotide base types comprises a third nucleotide base type and a fourth nucleotide base type from a second portion of the flow order, wherein the flow order comprises an ordered set of the four canonical base types (A, T, C, and G), ii) the plurality of base positions comprises a same number (N) of base positions for each barcode sequence, iii) each base position in a respective barcode sequence comprises a single nucleotide of the selected nucleotide base type, and iv) each respective barcode sequence is distinct from all other barcode sequences in the set of barcode sequences; and (b) electronically outputting the set of barcode sequences.

Embodiment 64. The method of any one of embodiments 60-63, wherein the first set of nucleotide base types comprises a first nucleotide base type and a second nucleotide base type.

Embodiment 65. The method of embodiment 64, wherein the second set of nucleotide base types comprises a third nucleotide base type and a fourth nucleotide base type.

Embodiment 66. The method of embodiment 64 or embodiment 65, wherein the first set of nucleotide base types comprises thymidine and guanine.

Embodiment 67. The method of any one of embodiments 64-66, wherein the second set of nucleotide base types comprises cytidine and adenine.

Embodiment 68. The method of any one of embodiments 64-67, wherein N is an even number.

Embodiment 69. The method of embodiment 68, wherein N is at least 10.

Embodiment 70. The method of any one of embodiments 60-69, wherein the set of barcode sequences comprises 2N barcode sequences.

Embodiment 71. The method for any one of embodiments 60-70, wherein a first barcode sequence in the set of barcode sequences comprises a nucleotide base type selected from the first set of nucleotides (K) in a first base position of the N consecutive base positions.

### EXAMPLES

### Example 1: Flowgram for Sequencing by synthesis

Sequencing data, such as a flowgram as described below, can be generated based on the detection of an incorporated nucleotide and the order of nucleotide introduction. For example, a flowgram for the following template sequences is shown in Table 4: CTG and CAG, and a repeating flow cycle of T-A-C-G (that is, sequential addition of T, A, C, and G nucleotides, which would be incorporated into the primer only if a complementary base is present in the template polynucleotide). In Table 4, 1 indicates incorporation of an introduced nucleotide, 0 indicates no incorporation of an introduced nucleotide, and an integer x>1 indicates incorporation of x introduced nucleotides. The flowgram can be used to determine the sequence of the template strand (e.g., the sequence of the template strand may be considered as the complement of the incorporated nucleotides).

TABLE 4

| Flow Cycle | |
|---|---|
| 1 | 2 |

Cycle Step

| | | | | | | | |
|---|---|---|---|---|---|---|---|
| 1 | 2 | 3 | 4 | 1 | 2 | 3 | 4 |

Flow Bases

| | | | | | | | |
|---|---|---|---|---|---|---|---|
| T | A | C | G | T | A | C | G |

Sequence | Number of Bases Incorporated

| | | | | | | | | |
|---|---|---|---|---|---|---|---|---|
| CTG | 0 | 0 | 0 | 1 | 0 | 1 | 1 | 0 |
| CAG | 0 | 0 | 0 | 1 | 1 | 0 | 1 | 0 |
| CCG | 0 | 0 | 0 | 2 | 0 | 0 | 1 | 0 |

A flowgram may be binary or non-binary. A binary flowgram detects the presence (1) or absence (0) of an incorporated nucleotide. A non-binary flowgram, such as shown in Table 1, can more quantitatively determine a number of incorporated nucleotides from each stepwise introduction. A non-binary flowgram also indicates the presence or absence of the base but can provide additional information including the number of bases incorporated at the given step. For example, the sequence of CCG would incorporate two G bases in one flow cycle step (e.g., in flow cycle 1, cycle step 4), and any signal emitted by the two labeled bases would have a greater intensity than the incorporation of a single base.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-71. (canceled)

72. A kit comprising a plurality of non-naturally occurring nucleic acid molecules comprising at least eight non-naturally occurring nucleic acid molecules, wherein the at least eight non-naturally occurring nucleic acid molecules comprise sequences selected from any one of SEQ ID NOs: 205-1259.

73. The kit of claim 72, wherein the at least eight non-naturally occurring nucleic acid molecules further comprise a first strand comprising SEQ ID NO: 1.

74. The kit of claim 73, wherein the SEQ ID NO: 1 is disposed 5′ of a respective sequence selected from any one of SEQ ID NOs: 205-1259.

75. The kit of claim 73, wherein the SEQ ID NO: 1 is disposed 5′ of a reverse complementary sequence of a respective sequence selected from any one of SEQ ID NOs: 205-1259.

76. The kit of claim 73, wherein the at least eight non-naturally occurring nucleic acid molecules further comprises a second strand comprising SEQ ID NO: 2.

77. The kit of claim 76, wherein the SEQ ID NO: 2 is disposed 3′ of a respective sequence selected from any one of SEQ ID NOs: 205-1259.

78. The kit of claim 76, wherein the SEQ ID NO: 2 is disposed 3′ of a reverse complementary sequence of a respective sequence selected from any one of SEQ ID NOs: 205-1259.

79. The kit of claim 72, wherein a 3′ thymine of the at least eight non-naturally occurring nucleic acid molecules is phosphorylated.

80. The kit of claim 72, wherein each of the at least eight non-naturally occurring nucleic acid molecules comprises a different sequence selected from any one of SEQ ID NOs: 205-1259.

81. The kit of claim 72, wherein the plurality of non-naturally occurring nucleic acid molecules comprises at least 96 non-naturally occurring nucleic acid molecules, and wherein each of the at least 96 non-naturally occurring nucleic acid molecules comprises a different sequence selected from any one of SEQ ID NOs: 205-1259.

82. The kit of claim 72, wherein the plurality of non-naturally occurring nucleic acid molecules comprises at least 256 non-naturally occurring nucleic acid molecules, and wherein each of the at least 256 non-naturally occurring nucleic acid molecules comprises a different sequence selected from any one of SEQ ID NOs: 205-1259.

83. A composition comprising a non-naturally occurring nucleic acid molecule comprising a first strand hybridized to a second strand, wherein the first strand comprises a sequence selected from any one of SEQ ID Nos 205-1259.

84. The composition of claim 83, wherein a 3′ thymine of the non-naturally occurring nucleic acid molecule is phosphorylated.

85. The composition of claim 83, wherein the first strand of the non-naturally occurring nucleic acid molecule further comprises SEQ ID No. 1260 positioned 5′ to the sequence selected from any one of SEQ ID NOs: 205-1259.

86. The composition of claim 83, wherein the non-naturally occurring nucleic acid molecule is further coupled to a template nucleic acid molecule.

87. The composition of claim 83, wherein the second stand comprises a reverse complementary sequence of the sequence selected from any one of SEQ ID NOs: 205-1259.

88. The composition of claim 87, wherein the second strand further comprises SEQ ID NO: 2 positioned 3′ to the reverse complementary sequence.
